SEQUENCE LISTING (24 sequences)

SEQ ID NO: 1 - protein, 107 aa, Artificial Sequence
  Synthetic
IEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRS

SEQ ID NO: 2 - protein, 9 aa, Artificial Sequence
  Synthetic
IEVMYPPPY

SEQ ID NO: 3 - protein, 107 aa, Artificial Sequence; identical to SEQ ID NO: 1
  Synthetic

SEQ ID NO: 4 - protein, 770 aa, Homo sapiens
MAQWNQLQQLDTRYLEQLHQLYSDSFPMELRQFLAPWIESQDWAYAASKESHATLVFHNLLGEIDQQYSRFLQESNVLYQHNLRRIKQFLQSRYLEKPMEIARIVARCLWEESRLLQTAATAAQQGGQANHPTAAVVTEKQQMLEQHLQDVRKRVQDLEQKMKVVENLQDDFDFNYKTLKSQGDMQDLNGNNQSVTRQKMQQLEQMLTALDQMRRSIVSELAGLLSAMEYVQKTLTDEELADWKRRQQIACIGGPPNICLDRLENWITSLAESQLQTRQQIKKLEELQQKVSYKGDPIVQHRPMLEERIVELFRNLMKSAFVVERQPCMPMHPDRPLVIKTGVQFTTKVRLLVKFPELNYQLKIKVCIDKDSGDVAALRGSRKFNILGTNTKVMNMEESNNGSLSAEFKHLTLREQRCGNGGRANCDASLIVTEELHLITFETEVYHQGLKIDLETHSLPVVVISNICQMPNAWASILWYNMLTNNPKNVNFFTKPPIGTWDQVAEVLSWQFSSTTKRGLSIEQLTTLAEKLLGPGVNYSGCQITWAKFCKENMAGKGFSFWVWLDNIIDLVKKYILALWNEGYIMGFISKERERAILSTKPPGTFLLRFSESSKEGGVTFTWVEKDISGKTQIQSVEPYTKQQLNNMSFAEIIMGYKIMDATNILVSPLVYLYPDIPKEEAFGKYCRPESQEHPEADPGSAAPYLKTKFICVTPTTCSNTIDLPMSPRTLDSLMQFGNNGEGAEPSAGGQFESLTFDMELTSECATSPM

SEQ ID NO: 5 - DNA, 4978 bp, Homo sapiens
ggtttccggagctgcggcggcgcagactgggagggggagccgggggttccgacgtcgcagccgagggaacaagccccaaccggatcctggacaggcaccccggcttggcgctgtctctccccctcggctcggagaggcccttcggcctgagggagcctcgccgcccgtccccggcacacgcgcagccccggcctctcggcctctgccggagaaacagttgggacccctgattttagcaggatggcccaatggaatcagctacagcagcttgacacacggtacctggagcagctccatcagctctacagtgacagcttcccaatggagctgcggcagtttctggccccttggattgagagtcaagattgggcatatgcggccagcaaagaatcacatgccactttggtgtttcataatctcctgggagagattgaccagcagtatagccgcttcctgcaagagtcgaatgttctctatcagcacaatctacgaagaatcaagcagtttcttcagagcaggtatcttgagaagccaatggagattgcccggattgtggcccggtgcctgtgggaagaatcacgccttctacagactgcagccactgcggcccagcaagggggccaggccaaccaccccacagcagccgtggtgacggagaagcagcagatgctggagcagcaccttcaggatgtccggaagagagtgcaggatctagaacagaaaatgaaagtggtagagaatctccaggatgactttgatttcaactataaaaccctcaagagtcaaggagacatgcaagatctgaatggaaacaaccagtcagtgaccaggcagaagatgcagcagctggaacagatgctcactgcgctggaccagatgcggagaagcatcgtgagtgagctggcggggcttttgtcagcgatggagtacgtgcagaaaactctcacggacgaggagctggctgactggaagaggcggcaacagattgcctgcattggaggcccgcccaacatctgcctagatcggctagaaaactggataacgtcattagcagaatctcaacttcagacccgtcaacaaattaagaaactggaggagttgcagcaaaaagtttcctacaaaggggaccccattgtacagcaccggccgatgctggaggagagaatcgtggagctgtttagaaacttaatgaaaagtgcctttgtggtggagcggcagccctgcatgcccatgcatcctgaccggcccctcgtcatcaagaccggcgtccagttcactactaaagtcaggttgctggtcaaattccctgagttgaattatcagcttaaaattaaagtgtgcattgacaaagactctggggacgttgcagctctcagaggatcccggaaatttaacattctgggcacaaacacaaaagtgatgaacatggaagaatccaacaacggcagcctctctgcagaattcaaacacttgaccctgagggagcagagatgtgggaatgggggccgagccaattgtgatgcttccctgattgtgactgaggagctgcacctgatcacctttgagaccgaggtgtatcaccaaggcctcaagattgacctagagacccactccttgccagttgtggtgatctccaacatctgtcagatgccaaatgcctgggcgtccatcctgtggtacaacatgctgaccaacaatcccaagaatgtaaacttttttaccaagcccccaattggaacctgggatcaagtggccgaggtcctgagctggcagttctcctccaccaccaagcgaggactgagcatcgagcagctgactacactggcagagaaactcttgggacctggtgtgaattattcagggtgtcagatcacatgggctaaattttgcaaagaaaacatggctggcaagggcttctccttctgggtctggctggacaatatcattgaccttgtgaaaaagtacatcctggccctttggaacgaagggtacatcatgggctttatcagtaaggagcgggagcgggccatcttgagcactaagcctccaggcaccttcctgctaagattcagtgaaagcagcaaagaaggaggcgtcactttcacttgggtggagaaggacatcagcggtaagacccagatccagtccgtggaaccatacacaaagcagcagctgaacaacatgtcatttgctgaaatcatcatgggctataagatcatggatgctaccaatatcctggtgtctccactggtctatctctatcctgacattcccaaggaggaggcattcggaaagtattgtcggccagagagccaggagcatcctgaagctgacccaggtagcgctgccccatacctgaagaccaagtttatctgtgtgacaccaacgacctgcagcaataccattgacctgccgatgtccccccgcactttagattcattgatgcagtttggaaataatggtgaaggtgctgaaccctcagcaggagggcagtttgagtccctcacctttgacatggagttgacctcggagtgcgctacctcccccatgtgaggagctgagaacggaagctgcagaaagatacgactgaggcgcctacctgcattctgccacccctcacacagccaaaccccagatcatctgaaactactaactttgtggttccagattttttttaatctcctacttctgctatctttgagcaatctgggcacttttaaaaatagagaaatgagtgaatgtgggtgatctgcttttatctaaatgcaaataaggatgtgttctctgagacccatgatcaggggatgtggcggggggtggctagagggagaaaaaggaaatgtcttgtgttgttttgttcccctgccctcctttctcagcagctttttgttattgttgttgttgttcttagacaagtgcctcctggtgcctgcggcatccttctgcctgtttctgtaagcaaatgccacaggccacctatagctacatactcctggcattgcactttttaaccttgctgacatccaaatagaagataggactatctaagccctaggtttctttttaaattaagaaataataacaattaaagggcaaaaaacactgtatcagcatagcctttctgtatttaagaaacttaagcagccgggcatggtggctcacgcctgtaatcccagcactttgggaggccgaggcggatcataaggtcaggagatcaagaccatcctggctaacacggtgaaaccccgtctctactaaaagtacaaaaaattagctgggtgtggtggtgggcgcctgtagtcccagctactcgggaggctgaggcaggagaatcgcttgaacctgagaggcggaggttgcagtgagccaaaattgcaccactgcacactgcactccatcctgggcgacagtctgagactctgtctcaaaaaaaaaaaaaaaaaaaagaaacttcagttaacagcctccttggtgctttaagcattcagcttccttcaggctggtaatttatataatccctgaaacgggcttcaggtcaaacccttaagacatctgaagctgcaacctggcctttggtgttgaaataggaaggtttaaggagaatctaagcattttagacttttttttataaatagacttattttcctttgtaatgtattggccttttagtgagtaaggctgggcagagggtgcttacaaccttgactccctttctccctggacttgatctgctgtttcagaggctaggttgtttctgtgggtgccttatcagggctgggatacttctgattctggcttccttcctgccccaccctcccgaccccagtccccctgatcctgctagaggcatgtctccttgcgtgtctaaaggtccctcatcctgtttgttttaggaatcctggtctcaggacctcatggaagaagagggggagagagttacaggttggacatgatgcacactatggggccccagcgacgtgtctggttgagctcagggaatatggttcttagccagtttcttggtgatatccagtggcacttgtaatggcgtcttcattcagttcatgcagggcaaaggcttactgataaacttgagtctgccctcgtatgagggtgtatacctggcctccctctgaggctggtgactcctccctgctggggccccacaggtgaggcagaacagctagagggcctccccgcctgcccgccttggctggctagctcgcctctcctgtgcgtatgggaacacctagcacgtgctggatgggctgcctctgactcagaggcatggccggatttggcaactcaaaaccaccttgcctcagctgatcagagtttctgtggaattctgtttgttaaatcaaattagctggtctctgaattaagggggagacgaccttctctaagatgaacagggttcgccccagtcctcctgcctggagacagttgatgtgtcatgcagagctcttacttctccagcaacactcttcagtacataataagcttaactgataaacagaatatttagaaaggtgagacttgggcttaccattgggtttaaatcatagggacctagggcgagggttcagggcttctctggagcagatattgtcaagttcatggccttaggtagcatgtatctggtcttaactctgattgtagcaaaagttctgagaggagctgagccctgttgtggcccattaaagaacagggtcctcaggccctgcccgcttcctgtccactgccccctccccatccccagcccagccgagggaatcccgtgggttgcttacctacctataaggtggtttataagctgctgtcctggccactgcattcaaattccaatgtgtacttcatagtgtaaaaatttatattattgtgaggttttttgtcttttttttttttttttttttttggtatattgctgtatctactttaacttccagaaataaacgttatataggaaccgtaaaaa

SEQ ID NO: 6 - protein, 7 aa, Simian virus 40
PKKKRKV

SEQ ID NO: 7 - protein, 10 aa, Simian virus 40
PKKKRKVEAS

SEQ ID NO: 8 - protein, 16 aa, Artificial Sequence
  Synthetic
KRPAATKKAGQAKKKK

SEQ ID NO: 9 - protein, 9 aa, Artificial Sequence
  Synthetic
PAAKRVKLD

SEQ ID NO: 10 - protein, 11 aa, Artificial Sequence
  Synthetic
RQRRNELKRSP

SEQ ID NO: 11 - protein, 38 aa, Artificial Sequence
  Synthetic
NQSSNFGPMKGGNFGGRSSGPYGGGGQYFAKPRNQGGY

SEQ ID NO: 12 - protein, 42 aa, Artificial Sequence
  Synthetic
RMRIZFKNKGKDTAELRRRRVEVSVELRKAKKDEQILKRRNV

SEQ ID NO: 13 - protein, 8 aa, Artificial Sequence
  Synthetic
VSRKRPRP

SEQ ID NO: 14 - protein, 8 aa, Artificial Sequence
  Synthetic
PPKKARED

SEQ ID NO: 15 - protein, 8 aa, Homo sapiens
PQPKKKPL

SEQ ID NO: 16 - protein, 12 aa, Mus sp.
SALIKKKKKMAP

SEQ ID NO: 17 - protein, 5 aa, Influenza virus
DRLRR

SEQ ID NO: 18 - protein, 7 aa, Influenza virus
PKQKKRK

SEQ ID NO: 19 - protein, 10 aa, Hepatitis virus
RKLKKKIKKL

SEQ ID NO: 20 - protein, 10 aa, Mus sp.
REKKKFLKRR

SEQ ID NO: 21 - protein, 20 aa, Homo sapiens
KRKGDEVDGVDEVAKKKSKK

SEQ ID NO: 22 - protein, 17 aa, Homo sapiens
RKCLQAGMNLEARKTKK

SEQ ID NO: 23 - protein, 288 aa, Artificial Sequence
  Synthetic
MDPIRSRTPSPARELLSGPQPDGVQPTADRGVSPPAGGPLDGLPARRTMSRTRLPSPPAPSPAFSADSFSDLLRQFDPSLFNTSLFDSLPPFGAHHTEAATGEWDEVQSGLRAADAPPPTMRVAVTAARPPRAKPAPRRRAAQPSDASPAAQVDLRTLGYSQQQQEKIKPKVRSTVAQHHEALVGHGFTHAHIVALSQHPAALGTVAVKYQDMIAALPEATHEAIVGVGKQWSGARALEALLTVAGELRGPPLQLDTGQLLKIAKRGGVTAVEAVHAWRNALTGAPLN

SEQ ID NO: 24 - protein, 183 aa, Artificial Sequence
  Synthetic
RPALESIVAQLSRPDPALAALTNDHLVALACLGGRPALDAVKKGLPHAPALIKRTNRRIPERTSHRVADHAQVVRVLGFFQCHSHPAQAFDDAMTQFGMSRHGLLQLFRRVGVTELEARSGTLPPASQRWDRILQASGMKRAKPSPTSTQTPDQASLHAFADSLERDLDAPSPMHEGDQTRAS

(19) United States
(12) Patent Application Publication
(10) Pub. No.: US 2021/0347847 A1
(43) Pub. Date: Nov. 11, 2021

(54) THERAPEUTIC TARGETING OF MALIGNANT CELLS USING TUMOR MARKERS

(71) Applicants: The Broad Institute, Inc., Cambridge, MA (US); Massachusetts Institute of Technology, Cambridge, MA (US); Dana-Farber Cancer Institute, Inc., Boston, MA (US)

(72) Inventors: Aviv Regev, Cambridge, MA (US); Benjamin Izar, Boston, MA (US)

(21) Appl. No.: 17/317,772

(22) Filed: May 11, 2021

Related U.S. Application Data
(60) Provisional application No. 63/023,091, filed on May 11, 2020.

(51) Int. Cl. C07K 14/725; A61K 35/17; C07K 16/28; A61K 45/06; C12N 15/11; C12N 9/22; A61K 47/68; C12N 9/78

(52) U.S. Cl. CPC C07K 14/7051; A61K 35/17; C07K 16/28; A61K 45/06; A61K 38/00; C12N 9/22; A61K 47/6849; C12N 9/78; C12N 15/111

(57) ABSTRACT

The present invention provides for methods and compositions for treating cancer. Disclosed are novel tumor biomarkers and therapeutic targets. Also disclosed are CAR T cells targeting tumor specific surface proteins. Also disclosed are shared expression programs specific to tumor cells.

### CROSS-REFERENCE TO RELATED APPLICATIONS

This application claims the benefit of U.S. Provisional Application No. 63/023,091, filed May 11, 2020. The entire contents of the above-identified applications are hereby fully incorporated herein by reference.

### STATEMENT REGARDING FEDERALLY SPONSORED RESEARCH

This invention was made with government support under Grant Nos. CA222663, CA225088, CA14051, and CA202820 awarded by the National Institutes of Health. The government has certain rights in the invention.

### SEQUENCE LISTING

This application contains a sequence listing filed in electronic form as an ACSII.txt file entitled BROD-4700US_ST25.txt, created on May 10, 2021 and having a size of 23,452 bytes (25 KB on disk). The content of the sequence listing is incorporated herein in its entirety.

### REFERENCE TO AN ELECTRONIC TABLE

The instant application contains a “lengthy” Table which has been submitted electronically in ASCII format and is hereby incorporated by reference in its entirety. Table_2 was created on Apr. 7, 2020 and is 2,400,000 bytes in size.

LENGTHY TABLES
The patent application contains a lengthy table section. A copy of the table is available in electronic form from the USPTO web site (https://seqdata.uspto.gov/?pageRequest=docDetail&DocID=US20210347847A1). An electronic copy of the table will also be available from the USPTO upon request and payment of the fee set forth in 37 CFR 1.19(b)(3).

### TECHNICAL FIELD

The subject matter disclosed herein is generally directed to therapeutically targeting tumor specific genes and biological programs for the treatment of cancer.

### BACKGROUND

Tumors, including ovarian tumors, are complex ecosystems defined by spatiotemporal interactions between heterogeneous cell types, including malignant, immune, and stromal cells (D. Hanahan and R. A. Weinberg, 2011 Cell, 144: 646-674). Each tumor's cellular composition, as well as the interplay between these components, exerts critical roles in cancer development (C. E. Meacham and S. J. Morrison, 2013 Nature, 501: 328-337). However, prior to the invention described herein, the specific components, their salient biological functions, and the means by which they collectively define tumor behavior were incompletely characterized in ovarian cancer.

Resistance to therapy is a major impediment to improving outcomes in ovarian cancer. Due to the lack of effective screening approaches, most patients are diagnosed at an advanced stage (Matulonis et al., 2016 Nat. Rev. Dis. Primer, 2:16061). Advanced-stage ovarian cancer is treated with surgery and chemotherapy with platinum and taxane agents. While 10-15% of patients exhibit intrinsic resistance to initial chemotherapy, most patients typically have a good response and achieve disease remission following chemotherapy; however, residual disease is frequently present and leads to relapse in 80% of patients within months to several years (Matulonis et al., 2016 Nat. Rev. Dis. Primer, 2:16061; Siegel et al., 2016 Cancer J. Clin., 66:7-30).

Despite recent therapeutic advances, recurrent ovarian cancer is incurable and portends a poor prognosis with a median survival of approximately one year⁴. Intra-tumor heterogeneity of ovarian cancer cells and associated non-malignant cells are important factors in driving treatment resistance, but remain poorly understood. Genomic analysis of high-grade serous ovarian cancer (HGSOC), the most common and aggressive histological subtype, revealed TP53 mutations, defects in homologous recombination DNA repair and extensive copy-number aberrations in most tumors⁵, and classified HGSOC into four transcriptional subtypes with distinct prognoses^{5,6}. Ascites, comprised of a diverse collection of cell types, is present in one-third of ovarian cancer patients at the time of diagnosis, and frequently occurs in patients with chemotherapy-resistant disease⁷. Rather than a homogenous suspension of single cells, ascites fluid is comprised of a multicellular collection of cancer cells, immune cells, and fibroblasts which contribute to disease progression (Kipps et al., 2013 Nat. Rev. Cancer, 13:273-282). Ovarian cancer spheroids within ascites are multicellular aggregates that can promote intraperitoneal metastasis and that are associated with chemotherapy resistance (Shield et al., 2009 Gynecology Oncol., 113:143-148).

Ovarian cancer is one of the leading causes of cancer-related deaths in women. Many women with OvCa experience relapse characterized by minimal residual disease following platinum-based chemotherapy, development of malignant abdominal fluid (ascites) with tumor spheroids, and extensive tumor heterogeneity: features not easily captured by current genomic profiling approaches. Resistance to platinum-based therapies and development of ascites constitutes the major life-limiting factor in women with ovarian cancer, and the underlying mechanisms remain unknown. There is a need to further understand genomic programs in ovarian cancer cells and the associated non-malignant cells.

Citation or identification of any document in this application is not an admission that such a document is available as prior art to the present invention.

### SUMMARY

In one aspect, the present invention provides for a population of ex vivo T cells comprising an exogenous nucleic acid sequence encoding a binding protein specific for binding a surface protein selected from Table 2, wherein the surface protein has at least two-fold higher average expression in any one or more of clusters 1 to 5 as compared to the average expression in any one or more of clusters 6 to 18. In certain embodiments, the binding protein is a chimeric antigen receptor (CAR) or a T cell receptor (TCR). In certain embodiments, the T cells are specific for a surface protein selected from the group consisting of CLDN3, CLDN7, CLDN4, EPCAM, TACSTD2, MAL2, LSR, CD9, SPINT2, TM4SF1, TMEM205, TNFRSF12A and CD47. In certain embodiments, the T cells are CAR T cells specific for CLDN3. In certain embodiments, the T cells are CAR T cells specific for CLDN7. In certain embodiments, the T cells are CAR T cells specific for CLDN4. In another aspect, the present invention provides for a method of treating a cancer in a subject in need thereof comprising administering the population of T cells according to any embodiment herein to the subject.

In another aspect, the present invention provides for a method of treating a cancer in a subject in need thereof comprising administering to the subject a therapeutically effective amount of one or more agents capable of binding to or modulating the expression, activity, and/or function of one or more genes or polypeptides selected from Table 2, wherein the one or more genes or polypeptides have at least two-fold higher average expression in any one or more of clusters 1 to 5 as compared to the average expression in any one or more of clusters 6 to 18. In certain embodiments, the one or more agents bind to or modulate the expression, activity, and/or function of CLDN3. In certain embodiments, the one or more agents comprise a CAR T cell that binds CLDN3. In certain embodiments, the one or more agents comprise an antibody that binds CLDN3. In certain embodiments, the antibody is a bi-specific antibody. In certain embodiments, the bi-specific antibody binds CLDN3 and an immune cell marker. In certain embodiments, the immune cell marker is selected from the group consisting of CD3, CD8, CD28 and CD16. In certain embodiments, the bi-specific antibody binds CLDN3 and a surface protein selected from the group consisting of CLDN7, CLDN4, EPCAM, TACSTD2, MAL2, LSR, CD9, SPINT2, TM4SF1, TMEM205, TNFRSF12A and CD47. In certain embodiments, the antibody is an antibody-drug conjugate that binds CLDN3. In certain embodiments, the one or more agents bind to or modulate the expression, activity, and/or function of CLDN7. In certain embodiments, the one or more agents comprise a CAR T cell that binds CLDN7. In certain embodiments, the one or more agents comprise an antibody that binds CLDN7. In certain embodiments, the antibody is a bi-specific antibody. In certain embodiments, the bi-specific antibody binds CLDN7 and an immune cell marker. In certain embodiments, the immune cell marker is selected from the group consisting of CD3, CD8, CD28 and CD16. In certain embodiments, the bi-specific antibody binds CLDN7 and a surface protein selected from the group consisting of CLDN3, CLDN4, EPCAM, TACSTD2, MAL2, LSR, CD9, SPINT2, TM4SF1, TMEM205, TNFRSF12A and CD47. In certain embodiments, the antibody is an antibody-drug conjugate that binds CLDN7. In certain embodiments, the one or more agents bind to or modulate the expression, activity, and/or function of CLDN4. In certain embodiments, the one or more agents comprise a CAR T cell that binds CLDN4. In certain embodiments, the one or more agents comprise an antibody that binds CLDN4. In certain embodiments, the antibody is a bi-specific antibody. In certain embodiments, the bi-specific antibody binds CLDN4 and an immune cell marker. In certain embodiments, the immune cell marker is selected from the group consisting of CD3, CD8, CD28 and CD16. In certain embodiments, the bi-specific antibody binds CLDN4 and a surface protein selected from the group consisting of CLDN7, CLDN3, EPCAM, TACSTD2, MAL2, LSR, CD9, SPINT2, TM4SF1, TMEM205, TNFRSF12A and CD47. In certain embodiments, the antibody is an antibody-drug conjugate that binds CLDN4. In certain embodiments, the one or more agents bind to or modulate the expression, activity, and/or function of CLDN3, CLDN7, and/or CLDN4 in combination with any of the other genes or polypeptides in Table 2. In certain embodiments, the one or more genes or polypeptides are selected from the group consisting of CLDN3, CLDN7, CLDN4, EPCAM, TACSTD2, MAL2, LCN2, CKB, RBP1, CDKN2A, C19orf33, WFDC2, LSR, CRABP2, S100A13, KRT7, CRIP2, MDK, CD9, SPINT2, SLPI, KRT19, KRT18, KRT8, TM4SF1, NGFRAP1, S100A16, PCBD1, OCIAD2, ZNF428, TMEM205, TSTD1, TNFRSF12A, MARCKSL1, IFI27, CD47, POLR2I, CCDC124, PDCD5 and DPY30. In certain embodiments, the one or more genes or polypeptides are selected from the group of surface proteins consisting of CLDN3, CLDN7, CLDN4, EPCAM, TACSTD2, MAL2, LSR, CD9, SPINT2, TM4SF1, TMEM205, TNFRSF12A and CD47.

In another aspect, the present invention provides for a method of treating a cancer in a subject in need thereof comprising administering to the subject a therapeutically effective amount of one or more agents capable of modulating the expression, activity, and/or function of one or more biological programs comprising one or more genes or polypeptides selected from the group consisting of: CLU, TACSTD2, MFAP2, LGALS3BP, FBLN2, LY6E, VTCN1, CLIC5, PTPRS, RAB25, SH3BGRL, BST2, SERPING1, MFGE8, ANKRD65, UCA1, THEM6, HSPG2, MSLN, NDRG2, TAPBP, CDH6, PLD3, RBMS3, COMP, SEPP1, RNF213, CD74, IFIT3 and IFI6; or CCBP2, TACSTD2, OAS1, MX1, GPNMB, OAS2, KRT23, MSLN, TXNIP, C15orf48, UPK3BL, PTGES, LCN2, CD82, SAT1, VTCN1, ITGB2, NCCRP1, CEACAM6, AGR2, PSCA, PARP14, HERC6, GABRP, IFI44L, IFIT3, IFI6, IFI44, ISG15 and IFIT1; or XAF1, MX1, PARP14, DDX60, C19orf66, OAS3, STAT1, OAS1, PARP9, PLSCR1, IFI44, IFIT3, OASL, TRIM22, DTX3L, IFITM1, PSMB8, RTP4, RSAD2, DDX58, IFITM3, NMI, UBE2L6, UBA7, SP100, OAS2, ISG15, IFIT1, IFI44L and IFI6; or TACSTD2, LOC100505633, CLU, HLA-H, CLDN1, HLA-C, CD24, TSPAN15, HLA-B, CCL28, FAM107A, RARRES3, TNC, GPX1, KLK5, IFI27, SEPP1, CCDC3, PLA2G16, MAL, CNN3, CMBL, PSMB10, CRIP1, KRT5, C3orf55, HLA-A, ISG15, IFIT1 and IFI6; or IFIT3, ISG20, IFIT2, MX1, OAS1, HLA-H, RSAD2, C19orf66, PARP14, STAT1, HLA-A, EPSTI1, CMPK2, KRT6A, IFI35, HLA-F, HLA-B, OAS3, B2M, DDX58, CFB, ANXA3, TIMP1, DTX3L, PARP9, GPRC5A, ISG15, IFIT1, IFI44L and OAS2; or LYNX1, LYPD2, CLU, TRIM29, MMP7, MUC4, TSPAN1, ATP6V1B1, SERPINA1, SLPI, KCNN4, SYT8, RARRES3, PTGES, RASAL1, CP, FOLR1, UNC5B, ALOX5, MUC20, FOS, SLC4A11, FXYD3, C3, UNC5B-AS1, DEFB1, COL12A1, CD74, HLA-DRB1 and HLA-DRA; or RARRES1, CRYAB, NNMT, PLA2G16, MGST1, HLA-DRB6, MT1F, CLU, CD14, NFIB, C3, BNIP3L, OAT, SLC34A2, NUPR1, ANXA1, MT1X, CDC42EP2, TUBB6, SAA1, CSTB, VIM, GPX3, MT1E, HLA-DRB1, HLA-DRA, HLA-DRB5, HLA-DMA, CD74 and HLA-DPA1; or GPX3, LCN2, CLDN4, GPR56, DHRS3, SLC44A4, LYPD2, MUC20, TMPRSS4, MUC1, ELF3, SRGAP1, FOLR1, B2M, MDK, WFDC2, CMTM7, FTH1, CP, ESR1, CLDN3, RBP1, TNFAIP2, RNF213, MUC4, TACSTD2, CD74, HLA-DRA, HLA-DRB1 and HLA-DPA1; or SOD2, CXCL3, TNFAIP3, CXCL1, TUBA1A, TPM1, EDN1, TAGLN, CLDN1, CXCL2, TNFAIP2, NEDD9, ADAMTS9, FLNA, ARHGAP29, CTHRC1, RGS10, UBD, THBS1, CALD1, PTX3, RELB, CYR61, NFKBIA, CCL2, TNF, ICAM1, CCL20, IL8 and IL32; or UBD, TAP1, TNFAIP2, CRYAB, MARCO, LGALS14, PSMB9, CD74, TUBB2B, KRT23, IL4I1, TAPBP, C10orf10, CLDN1, SOD2, SELM, JAK3, TNFSF10, B2M, HLA-H, COL4A2, HLA-B, IL8, IL23A, ICAM1, TNF, CCL20, CCL2, IL32 and CD40.

In certain embodiments, the method comprises decreasing expression, activity, and/or function of an interferon response gene program comprising one or more genes or polypeptides selected from the group consisting of: CLU, TACSTD2, MFAP2, LGALS3BP, FBLN2, LY6E, VTCN1, CLIC5, PTPRS, RAB25, SH3BGRL, BST2, SERPING1, MFGE8, ANKRD65, UCA1, THEM6, HSPG2, MSLN, NDRG2, TAPBP, CDH6, PLD3, RBMS3, COMP, SEPP1, RNF213, CD74, IFIT3 and IFI6; or CCBP2, TACSTD2, OAS1, MX1, GPNMB, OAS2, KRT23, MSLN, TXNIP, C15orf48, UPK3BL, PTGES, LCN2, CD82, SAT1, VTCN1, ITGB2, NCCRP1, CEACAM6, AGR2, PSCA, PARP14, HERC6, GABRP, IFI44L, IFIT3, IFI6, IFI44, ISG15 and IFIT1; or XAF1, MX1, PARP14, DDX60, C19orf66, OAS3, STAT1, OAS1, PARP9, PLSCR1, IFI44, IFIT3, OASL, TRIM22, DTX3L, IFITM1, PSMB8, RTP4, RSAD2, DDX58, IFITM3, NMI, UBE2L6, UBA7, SP100, OAS2, ISG15, IFIT1, IFI44L and IFI6; or TACSTD2, LOC100505633, CLU, HLA-H, CLDN1, HLA-C, CD24, TSPAN15, HLA-B, CCL28, FAM107A, RARRES3, TNC, GPX1, KLK5, IFI27, SEPP1, CCDC3, PLA2G16, MAL, CNN3, CMBL, PSMB10, CRIP1, KRT5, C3orf55, HLA-A, ISG15, IFIT1 and IFI6; or IFIT3, ISG20, IFIT2, MX1, OAS1, HLA-H, RSAD2, C19orf66, PARP14, STAT1, HLA-A, EPSTI1, CMPK2, KRT6A, IFI35, HLA-F, HLA-B, OAS3, B2M, DDX58, CFB, ANXA3, TIMP1, DTX3L, PARP9, GPRC5A, ISG15, IFIT1, IFI44L and OAS2.

In certain embodiments, the method comprises increasing expression, activity, and/or function of an MHC class II gene program comprising one or more genes or polypeptides selected from the group consisting of: LYNX1, LYPD2, CLU, TRIM29, MMP7, MUC4, TSPAN1, ATP6V1B1, SERPINA1, SLPI, KCNN4, SYT8, RARRES3, PTGES, RASAL1, CP, FOLR1, UNC5B, ALOX5, MUC20, FOS, SLC4A11, FXYD3, C3, UNC5B-AS1, DEFB1, COL12A1, CD74, HLA-DRB1 and HLA-DRA; or RARRES1, CRYAB, NNMT, PLA2G16, MGST1, HLA-DRB6, MT1F, CLU, CD14, NFIB, C3, BNIP3L, OAT, SLC34A2, NUPR1, ANXA1, MT1X, CDC42EP2, TUBB6, SAA1, CSTB, VIM, GPX3, MT1E, HLA-DRB1, HLA-DRA, HLA-DRB5, HLA-DMA, CD74 and HLA-DPA1; or GPX3, LCN2, CLDN4, GPR56, DHRS3, SLC44A4, LYPD2, MUC20, TMPRSS4, MUC1, ELF3, SRGAP1, FOLR1, B2M, MDK, WFDC2, CMTM7, FTH1, CP, ESR1, CLDN3, RBP1, TNFAIP2, RNF213, MUC4, TACSTD2, CD74, HLA-DRA, HLA-DRB1 and HLA-DPA1. In certain embodiments, the method further comprises administering checkpoint blockade (CPB) therapy.

In certain embodiments, the method comprises decreasing expression, activity, and/or function of an inflammatory cytokine gene program comprising one or more genes or polypeptides selected from the group consisting of: SOD2, CXCL3, TNFAIP3, CXCL1, TUBA1A, TPM1, EDN1, TAGLN, CLDN1, CXCL2, TNFAIP2, NEDD9, ADAMTS9, FLNA, ARHGAP29, CTHRC1, RGS10, UBD, THBS1, CALD1, PTX3, RELB, CYR61, NFKBIA, CCL2, TNF, ICAM1, CCL20, IL8 and IL32; or UBD, TAP1, TNFAIP2, CRYAB, MARCO, LGALS14, PSMB9, CD74, TUBB2B, KRT23, IL4I1, TAPBP, C10orf10, CLDN1, SOD2, SELM, JAK3, TNFSF10, B2M, HLA-H, COL4A2, HLA-B, IL8, IL23A, ICAM1, TNF, CCL20, CCL2, IL32 and CD40.

In certain embodiments, the one or more agents target one or more cell surface exposed genes or polypeptides. In certain embodiments, the one or more agents target one or more receptors or ligands specific for one or more cell surface exposed genes or polypeptides. In certain embodiments, the one or more agents target one or more secreted genes or polypeptides. In certain embodiments, the one or more agents target one or more receptors specific for one or more secreted genes or polypeptides. In certain embodiments, the one or more agents comprise an antibody, antibody-like protein scaffold, aptamer, small molecule, genetic modifying agent, protein, nucleic acid or any combination thereof.

In certain embodiments, the antibody is an antibody-drug conjugate. In certain embodiments, the antibody is a bispecific antibody. In certain embodiments, the bi-specific antibody is capable of targeting an immune cell to the tumor cell. In certain embodiments, the bi-specific antibody is capable of targeting two surface proteins expressed on the tumor cell.

In certain embodiments, the genetic modifying agent comprises a CRISPR system, RNAi system, a zinc finger nuclease system, a TALE system, or a meganuclease. In certain embodiments, the CRISPR system is a Class 1 or Class 2 CRISPR system. In certain embodiments, the Class 2 system comprises a Type II Cas polypeptide. In certain embodiments, the Type II Cas is a Cas9. In certain embodiments, the Class 2 system comprises a Type V Cas polypeptide. In certain embodiments, the Type V Cas is Cas12a, Cas12b, Cas12c, Cas12d (CasY), Cas12e(CasX), or Cas14. In certain embodiments, the Class 2 system comprises a Type VI Cas polypeptide. In certain embodiments, the Type VI Cas is Cas13a, Cas13b, Cas13c or Cas13d. In certain embodiments, the CRISPR system comprises a dCas fused or otherwise linked to a nucleotide deaminase. In certain embodiments, the nucleotide deaminase is a cytidine deaminase or an adenosine deaminase.

In another aspect, the present invention provides for a method for detecting, monitoring or prognosing a cancer in a subject in need thereof comprising detecting in a tumor sample obtained from the subject the expression or activity of one or more genes or polypeptides selected from the group consisting of CLDN3, CLDN7, CLDN4, EPCAM, TACSTD2, MAL2, LCN2, CKB, RBP1, CDKN2A, C19orf33, WFDC2, LSR, CRABP2, S100A13, KRT7, CRIP2, MDK, CD9, SPINT2, SLPI, KRT19, KRT18, KRT8, TM4SF1, NGFRAP1, S100A16, PCBD1, OCIAD2, ZNF428, TMEM205, TSTD1, TNFRSF12A, MARCKSL1, IFI27, CD47, POLR2I, CCDC124, PDCD5 and DPY30. In certain embodiments, the one or more genes or polypeptides are detected in single cells from the biological sample. In certain embodiments, the one or more genes or polypeptides are detected in a tissue sample by immunohistochemistry or RNA FISH.

In another aspect, the present invention provides for a method for detecting, monitoring or prognosing a cancer in a subject in need thereof comprising detecting in a tumor sample obtained from the subject the expression or activity of one or more biological programs comprising one or more genes or polypeptides selected from the group consisting of: CLU, TACSTD2, MFAP2, LGALS3BP, FBLN2, LY6E, VTCN1, CLIC5, PTPRS, RAB25, SH3BGRL, BST2, SERPING1, MFGE8, ANKRD65, UCA1, THEM6, HSPG2, MSLN, NDRG2, TAPBP, CDH6, PLD3, RBMS3, COMP, SEPP1, RNF213, CD74, IFIT3 and IFI6; or CCBP2, TACSTD2, OAS1, MX1, GPNMB, OAS2, KRT23, MSLN, TXNIP, C15orf48, UPK3BL, PTGES, LCN2, CD82, SAT1, VTCN1, ITGB2, NCCRP1, CEACAM6, AGR2, PSCA, PARP14, HERC6, GABRP, IFI44L, IFIT3, IFI6, IFI44, ISG15 and IFIT1; or XAF1, MX1, PARP14, DDX60, C19orf66, OAS3, STAT1, OAS1, PARP9, PLSCR1, IFI44, IFIT3, OASL, TRIM22, DTX3L, IFITM1, PSMB8, RTP4, RSAD2, DDX58, IFITM3, NMI, UBE2L6, UBA7, SP100, OAS2, ISG15, IFIT1, IFI44L and IFI6; or TACSTD2, LOC100505633, CLU, HLA-H, CLDN1, HLA-C, CD24, TSPAN15, HLA-B, CCL28, FAM107A, RARRES3, TNC, GPX1, KLK5, IFI27, SEPP1, CCDC3, PLA2G16, MAL, CNN3, CMBL, PSMB10, CRIP1, KRT5, C3orf55, HLA-A, ISG15, IFIT1 and IFI6; or IFIT3, ISG20, IFIT2, MX1, OAS1, HLA-H, RSAD2, C19orf66, PARP14, STAT1, HLA-A, EPSTI1, CMPK2, KRT6A, IFI35, HLA-F, HLA-B, OAS3, B2M, DDX58, CFB, ANXA3, TIMP1, DTX3L, PARP9, GPRC5A, ISG15, IFIT1, IFI44L and OAS2; or LYNX1, LYPD2, CLU, TRIM29, MMP7, MUC4, TSPAN1, ATP6V1B1, SERPINA1, SLPI, KCNN4, SYT8, RARRES3, PTGES, RASAL1, CP, FOLR1, UNC5B, ALOX5, MUC20, FOS, SLC4A11, FXYD3, C3, UNC5B-AS1, DEFB1, COL12A1, CD74, HLA-DRB1 and HLA-DRA; or RARRES1, CRYAB, NNMT, PLA2G16, MGST1, HLA-DRB6, MT1F, CLU, CD14, NFIB, C3, BNIP3L, OAT, SLC34A2, NUPR1, ANXA1, MT1X, CDC42EP2, TUBB6, SAA1, CSTB, VIM, GPX3, MT1E, HLA-DRB1, HLA-DRA, HLA-DRB5, HLA-DMA, CD74 and HLA-DPA1; or GPX3, LCN2, CLDN4, GPR56, DHRS3, SLC44A4, LYPD2, MUC20, TMPRSS4, MUC1, ELF3, SRGAP1, FOLR1, B2M, MDK, WFDC2, CMTM7, FTH1, CP, ESR1, CLDN3, RBP1, TNFAIP2, RNF213, MUC4, TACSTD2, CD74, HLA-DRA, HLA-DRB1 and HLA-DPA1; or SOD2, CXCL3, TNFAIP3, CXCL1, TUBA1A, TPM1, EDN1, TAGLN, CLDN1, CXCL2, TNFAIP2, NEDD9, ADAMTS9, FLNA, ARHGAP29, CTHRC1, RGS10, UBD, THBS1, CALD1, PTX3, RELB, CYR61, NFKBIA, CCL2, TNF, ICAM1, CCL20, IL8 and IL32; or UBD, TAP1, TNFAIP2, CRYAB, MARCO, LGALS14, PSMB9, CD74, TUBB2B, KRT23, IL4I1, TAPBP, C10orf10, CLDN1, SOD2, SELM, JAK3, TNFSF10, B2M, HLA-H, COL4A2, HLA-B, IL8, IL23A, ICAM1, TNF, CCL20, CCL2, IL32 and CD40.

In certain embodiments, detection of an interferon response gene program comprising one or more genes or polypeptides selected from the group consisting of: CLU, TACSTD2, MFAP2, LGALS3BP, FBLN2, LY6E, VTCN1, CLIC5, PTPRS, RAB25, SH3BGRL, BST2, SERPING1, MFGE8, ANKRD65, UCA1, THEM6, HSPG2, MSLN, NDRG2, TAPBP, CDH6, PLD3, RBMS3, COMP, SEPP1, RNF213, CD74, IFIT3 and IFI6; or CCBP2, TACSTD2, OAS1, MX1, GPNMB, OAS2, KRT23, MSLN, TXNIP, C15orf48, UPK3BL, PTGES, LCN2, CD82, SAT1, VTCN1, ITGB2, NCCRP1, CEACAM6, AGR2, PSCA, PARP14, HERC6, GABRP, IFI44L, IFIT3, IFI6, IFI44, ISG15 and IFIT1; or XAF1, MX1, PARP14, DDX60, C19orf66, OAS3, STAT1, OAS1, PARP9, PLSCR1, IFI44, IFIT3, OASL, TRIM22, DTX3L, IFITM1, PSMB8, RTP4, RSAD2, DDX58, IFITM3, NMI, UBE2L6, UBA7, SP100, OAS2, ISG15, IFIT1, IFI44L and IFI6; or TACSTD2, LOC100505633, CLU, HLA-H, CLDN1, HLA-C, CD24, TSPAN15, HLA-B, CCL28, FAM107A, RARRES3, TNC, GPX1, KLK5, IFI27, SEPP1, CCDC3, PLA2G16, MAL, CNN3, CMBL, PSMB10, CRIP1, KRT5, C3orf55, HLA-A, ISG15, IFIT1 and IFI6; or IFIT3, ISG20, IFIT2, MX1, OAS1, HLA-H, RSAD2, C19orf66, PARP14, STAT1, HLA-A, EPSTI1, CMPK2, KRT6A, IFI35, HLA-F, HLA-B, OAS3, B2M, DDX58, CFB, ANXA3, TIMP1, DTX3L, PARP9, GPRC5A, ISG15, IFIT1, IFI44L and OAS2, indicates that a subject should be treated with a signal transducer and activator of transcription 3 (STAT3) activity inhibitor. In certain embodiments, the method further comprises treating with a STAT3 inhibitor. In certain embodiments, the STAT3 activity inhibitor is administered intraperitoneally. In certain embodiments, the STAT3 activity is selected from the group consisting of STAT3 phosphorylation, STAT3 dimerization, STAT3 binding to a polynucleotide comprising a STAT3 binding site, STAT3 binding to genomic DNA, activation of a STAT3 responsive gene and STAT3 nuclear translocation. In certain embodiments, the STAT3 inhibitor comprises pyrimethamine, atovaquone, pimozide, guanabenz acetate, alprenolol hydrochloride, nifuroxazide, solanine alpha, fluoxetine hydrochloride, ifosfamide, pyrvinium pamoate, moricizine hydrochloride, 3,3′-oxybis[tetrahydrothiophene, 1,1,1′,1′-tetraoxide], 3-(1,3-benzodioxol-5-yl)-1,6-dimethyl-pyrimido[5,4-e]-1,2,4-triazine-5,7(-1H,6H)-dione, 2-(1,8-Naphthyridin-2-yl)phenol, or 3-(2-hydroxyphenyl)-3-phenyl-N,N-dipropylpropanamide, as well as any derivatives of these compounds or analogues thereof. In certain embodiments, the STAT3 activity inhibitor comprises JSI-124 (cucurbitacin I). In certain embodiments, the JSI-124 is administered at a dose of about 0.1 μM.

In certain embodiments, detection of an MHC class II gene program comprising one or more genes or polypeptides selected from the group consisting of: LYNX1, LYPD2, CLU, TRIM29, MMP7, MUC4, TSPAN1, ATP6V1B1, SERPINA1, SLPI, KCNN4, SYT8, RARRES3, PTGES, RASAL1, CP, FOLR1, UNC5B, ALOX5, MUC20, FOS, SLC4A11, FXYD3, C3, UNC5B-AS1, DEFB1, COL12A1, CD74, HLA-DRB1 and HLA-DRA; or RARRES1, CRYAB, NNMT, PLA2G16, MGST1, HLA-DRB6, MT1F, CLU, CD14, NFIB, C3, BNIP3L, OAT, SLC34A2, NUPR1, ANXA1, MT1X, CDC42EP2, TUBB6, SAA1, CSTB, VIM, GPX3, MT1E, HLA-DRB1, HLA-DRA, HLA-DRB5, HLA-DMA, CD74 and HLA-DPA1; or GPX3, LCN2, CLDN4, GPR56, DHRS3, SLC44A4, LYPD2, MUC20, TMPRSS4, MUC1, ELF3, SRGAP1, FOLR1, B2M, MDK, WFDC2, CMTM7, FTH1, CP, ESR1, CLDN3, RBP1, TNFAIP2, RNF213, MUC4, TACSTD2, CD74, HLA-DRA, HLA-DRB1 and HLA-DPA1, indicates the patient is responsive to CPB therapy. In certain embodiments, the method further comprises treating with checkpoint blockade (CPB) therapy.

In certain embodiments, the one or more genes or polypeptides are detected in single cells from the tumor sample. In certain embodiments, the one or more genes or polypeptides are detected in a tissue sample by immunohistochemistry or RNA FISH.

In another aspect, the present invention provides for a method of screening for agents capable of modulating a biological program in ovarian cancer comprising: applying a candidate agent to an ovarian cancer cell or cell population; and detecting modulation of one or more biological programs according to any embodiment herein, thereby identifying the agent. In certain embodiments, the agent is applied to an animal model. In certain embodiments, the animal model is a patient-derived xenograft (PDX).

In another aspect, the present invention provides for a method of treating a subject with ovarian cancer comprising detecting in a tumor sample obtained from the subject the expression or activity of one or more biological programs according to any embodiment herein, wherein if the ovarian tumor expresses one or more of the biological programs, administering a therapeutic regimen that comprises a signal transducer and activator of transcription 3 (STAT3) activity inhibitor, optionally, in combination with chemotherapy; or if the ovarian tumor does not express one or more of the biological programs, administering a therapeutic regimen that comprises chemotherapy and does not comprise a STAT3 inhibitor. In certain embodiments, one or more interferon response gene biological programs are detected. In certain embodiments, the STAT3 activity inhibitor is administered intraperitoneally. In certain embodiments, the STAT3 activity is selected from the group consisting of STAT3 phosphorylation, STAT3 dimerization, STAT3 binding to a polynucleotide comprising a STAT3 binding site, STAT3 binding to genomic DNA, activation of a STAT3 responsive gene and STAT3 nuclear translocation. In certain embodiments, the STAT3 inhibitor comprises pyrimethamine, atovaquone, pimozide, guanabenz acetate, alprenolol hydrochloride, nifuroxazide, solanine alpha, fluoxetine hydrochloride, ifosfamide, pyrvinium pamoate, moricizine hydrochloride, 3,3′-oxybis[tetrahydrothiophene, 1,1,1′,1′-tetraoxide], 3-(1,3-benzodioxol-5-yl)-1,6-dimethyl-pyrimido[5,4-e]-1,2,4-triazine-5,7(-1H,6H)-dione, 2-(1,8-Naphthyridin-2-yl)phenol, or 3-(2-hydroxyphenyl)-3-phenyl-N,N-dipropylpropanamide, as well as any derivatives of these compounds or analogues thereof. In certain embodiments, the STAT3 activity inhibitor comprises JSI-124 (cucurbitacin I). In certain embodiments, the JSI-124 is administered at a dose of about 0.1 μM.

In another aspect, the present invention provides for a method of treating a subject with ovarian cancer comprising detecting in a tumor sample obtained from the subject the expression or activity of one or more MHC class II biological programs according to claim 71, wherein if the ovarian tumor expresses one or more of the biological programs, administering a therapeutic regimen that comprises an immunotherapy; or if the ovarian tumor does not express one or more of the biological programs, administering a therapeutic regimen that does not comprise an immunotherapy. In certain embodiments, the immunotherapy comprises CPB therapy.

In another aspect, the present invention provides for a method of treating a subject with ovarian cancer comprising determining whether the ovarian tumor exhibits a mesenchymal phenotype by detecting CAFs in the tumor, wherein if the ovarian tumor exhibits increased CAFs characteristic of a mesenchymal phenotype, administering a therapeutic regimen that does not comprise an immunotherapy; and if the ovarian tumor does not exhibit increased CAFs characteristic of a mesenchymal phenotype, administering a therapeutic regimen that comprises an immunotherapy. In certain embodiments, the immunotherapy comprises CPB therapy. In certain embodiments, if the ovarian tumor exhibits increased CAFs characteristic of a mesenchymal phenotype, administering a therapeutic regimen that comprises an agent capable of modulating CAF activation. In certain embodiments, the agent modulates the expression, activity and/or function of one or more genes expressed in CAFs associated with the mesenchymal subtype selected from the group consisting of: PDPN, C1QA/B/C, CFB, CXCL12, CXCL1, CXCL2, CXCL10, IL6, IL10, ALDH1A2, ACTA2, COL1A2, LUM, COL3A1, DCN and COL1A1; or clusters 6-9 of Table 2. In certain embodiments, detecting the mesenchymal phenotype comprises detecting in CAFs one or more genes selected from the group consisting of: PDPN, C1QA/B/C, CFB, CXCL12, CXCL1, CXCL2, CXCL10, IL6, IL10, ALDH1A2, ACTA2, COL1A2, LUM, COL3A1, DCN and COL1A1; or clusters 6-9 of Table 2.

In another aspect, the present invention provides for a method of treating a subject with ovarian cancer comprising determining whether the ovarian tumor exhibits an immunoreactive phenotype by detecting macrophages in the tumor, wherein if the ovarian tumor exhibits increased macrophages characteristic of an immunoreactive phenotype, administering a therapeutic regimen that comprises an immunotherapy; and if the ovarian tumor does not exhibit increased macrophages characteristic of an immunoreactive phenotype, administering a therapeutic regimen that does not comprise an immunotherapy. In certain embodiments, the immunotherapy comprises CPB therapy. In certain embodiments, detecting an immunoreactive phenotype comprises detecting in macrophages one or more genes selected from the group consisting of: CD52, CD14, AIF1, CSF1R, C1QB, C1QA, CD163, CD36, FCGR3A, CCL3, CCL4, IFNGR1, CD1D, C2, APOE, APOC1, CTSD, CTSZ, LYZ, FCN1, DDX5, MNDA, C3AR1, VISG4, SERPINA1, HLA-DPA1, HLA-DRA, HLA-DPB1 and HLA-DRB5; or clusters 10-13 of Table 2.

In certain embodiments, the cancer is ovarian cancer. In certain embodiments, the ovarian cancer is high-grade serous ovarian cancer (HGSOC).

In another aspect, the present invention provides for a kit comprising reagents to detect at least one gene or polypeptide according to any embodiment herein.

These and other aspects, objects, features, and advantages of the example embodiments will become apparent to those having ordinary skill in the art upon consideration of the following detailed description of example embodiments.

### BRIEF DESCRIPTION OF THE DRAWINGS

An understanding of the features and advantages of the present invention will be obtained by reference to the following detailed description that sets forth illustrative embodiments, in which the principles of the invention may be utilized, and the accompanying drawings of which:

FIG. 1A-1E—Charting the ovarian cancer ascites landscape by scRNA-seq. (FIG. 1a) Overview of sample collection and profiling strategy. (FIG. 1b, FIG. 1c) Malignant and non-malignant cell clusters in patient ascites by droplet-based scRNA-seq. (b) t-stochastic neighborhood embedding (tSNE) of 9,609 droplet-based scRNA-seq profiles from 8 samples, colored by sample-of-origin and numbered by unsupervised cluster assignment. (FIG. 1c) Fraction of cells (x axis) from each sample (color code, as in b) in each cluster (y axis). Clusters are labeled (right) by their post-hoc annotation based on differentially expressed genes (as in d). (FIG. 1d) Differentially expressed genes. Average expression (log 2(TPM+1), color bar) of the top 30 genes (rows) that are differentially expressed in each cluster (columns). Genes are ordered by hierarchical clustering. (FIG. 1e) An inflammatory subset of CAFs. Comparison of the average expression (log 2(TPM+1)) of each gene in CAF cluster 8 (y axis) vs. CAF clusters 6 and 7 (x axis). Red: immunomodulatory genes.

FIG. 2A-2E—Malignant and non-malignant cell expression profiles help identify cellular basis of TCGA subtypes. (FIG. 2a, FIG. 2b) Malignant cell clusters are enriched in patient ascites by FACS and plate-based scRNA-seq. (a) tSNE of 1,297 single cell profiles from 14 ascites samples profiled by plate-based scRNA-seq, colored and numbered by unsupervised cluster assignment. (b) Fraction of cells (x axis) from each sample (color code, as in a) in each cluster (y axis). Clusters are labeled (right) by their post-hoc annotation based on differentially expressed genes (as in c). (FIG. 2c) Differentially expressed genes. Average expression (log 2(TPM+1), color bar) of the top 30 genes (rows) that are differentially expressed in each cluster (columns). Genes are ordered by hierarchical clustering. (FIG. 2d) The immunoreactive and mesenchymal subtypes reflect macrophages and fibroblast components. Subtype score (color bar), based on average expression of subtype-specific genes (Methods) of each clusters (rows) for each of four TCGA subtypes (column). (FIG. 2e) Immunoreactive and mesenchymal TCGA subtypes have lower overall purity than differentiated and proliferative. Distribution of a purity estimate value (y axis, ABSOLUTE⁴¹; Methods) for TCGA ovarian cancer tumors (n=282) in each subtype (x axis). Horizontal bar: mean; box: interquartile range, whiskers: minimum and maximum. Dots: outliers. ***p<10⁻¹⁰ (two-sided t test).

FIG. 3A-3H—Inflammatory programs in malignant cells from patient ascites predict a role for JAK-STAT signaling. (FIG. 3a-FIG. 3c) Intra-tumoral expression modules. Relative expression (color bar, Methods) of the top 30 module-specific genes (rows) in each module (ordered by module, dashed horizontal lines), as defined by NMF (Methods) across all cancer cells (columns; ordered by hierarchical clustering) from patients 8 (a), 9 (b), or 10 (c). Selected genes are annotated. Top bar (a and b): sample time in 3 sequential samples from the same patient. (FIG. 3d, FIG. 3e) Cell cycle and inflammatory/immune programs recur across patients. (d) Number of top genes (color bar) shared between each pair of modules (rows and columns, ordered by hierarchical clustering). Top: module's patient-of-origin. (e) Module membership in the top 30 (black) or 50 (grey) of selected cell cycle (top) and immune-related (bottom) genes (rows) across all modules (columns), ordered as in (d). (FIG. 3f) MHC-II expressing cancer cells in situ. Representative immunofluorescence staining of HGSOC primary tissue staining for nucleus (blue), pan-keratin (green) and MHC Class II (red). Size bar: 20 μm. Overlay (right): co-expression of pan-keratin and MHC Class II, indicating cancer cell-autonomous expression of MHC Class II in a subset of cancer cells. (FIG. 3g, FIG. 3h) Broad and high expression of JAK-STAT pathway components across malignant cells. (g) Distribution of average expression of genes (x axis, log 2(TPM+1)) for all detected genes (y axis). Red: STAT1/3 expression bin. (h) Mean expression (x axis, log 2(TPM+1)) and percentage of expressing cells (y axis) of signaling genes. Key nodes of the JAK/STAT-pathway are labeled. Line: LOWESS regression curve.

FIG. 4A-4J—JAK/STAT-inhibition reduces viability, spheroid formation, and invasion of HGSOC models ex vivo and in vitro. (FIG. 4a) JSI-124 reduces viability of in OVCAR4 ovarian cancer cell line. Relative (mean) viability compared to control in GILA (y axis) following 2 days of treatment of the OVCAR4 cell line with each of 14 inhibitors of the JAK/STAT-pathway, carboplatin and cisplatin (x axis). (**adjusted p=0.0032 for JSI-124, one-way ANOVA with Holm-Bonferroni correction with Holm-Šidák-extension). Error bars: SD, n=3. (FIG. 4b) JSI-124, but not other compounds routinely used for the treatment of ovarian cancer reduces mean viability of patient-derived ex vivo cultures. Percent viability relative to DMSO treated cells (y axis) in ex vivo cultures derived from patients 3, 5, and DF3291, each treated for 48 hours with increasing doses (x axis, μM) of JSI-124, carboplatin, cisplatin, paclitaxel, or olaparib. Error bars: SD, n=4. Representative of biological duplicates. (FIG. 4c, FIG. 4d) JSI-124 leads to spheroid disintegration. (c) Examples of light microscopy images of spheroids treated with indicated compounds (representative of biological triplicates). (d) Average number of spheroids (relative to DMSO treated control, y axis) formed with five established ovarian cancer cell lines (x axis) treated with two doses of JSI-124 (blue bars) or carboplatin (red bars) (*adjusted p<0.05, one-way ANOVA with Holm-Bonferroni correction with Holm-Šidák-extension). Error bars: SEM, n=3. (FIG. 4e, FIG. 4f) JSI-124 treatment reduces mesothelial clearance by patient-derived spheroids from patient-derived cultures and established cell lines. Mesothelial clearance (y axis) by patient derived cells (NACT8, e) treated with either JSI-124 (for 30 or 120 min) vs. DMSO, or by ovarian cancer cell lines OVCAR8 and TYKNU treated for 30 min. 20 spheroids clusters assessed per iteration. **p<0.01 (one-way ANOVA and post hoc Tukey-Kramer test), two independent experiments with n=20 spheroids/condition. Horizontal bar: mean; box: interquartile range, whiskers: minimum and maximum. (FIG. 4g-FIG. 4j) JSI-124 prevents tumor growth and eliminates established tumors in PDX models. Mean log BLI signal (y axis, log total flux in p/s) from PDX mice injected with DF20 tumor cells and treated with either vehicle (black) or JSI-124 (red) and monitored over time (x axis, days). Error bars: SEM. N=5 mice per group. All statistical tests are two-sided t test comparing mean±SD at Day 15 of treatment. (g) Mice injected intraperitoneally (IP) and started treatment one week later for 14 days. ***p<0.0001. (h) Mice were injected TP, malignant ascites were allowed to form, and treatment started at 21 days, for a total of another 14 days. **p=0.002. (i) Mice injected subcutaneously (SC) and started treatment one week later for 14 days. ***p<0.0001. (j) Mice were injected SC, tumors were allowed to form, and treatment started at 21 days, for a total of another 14 days. **p=0.0028.

FIG. 5A-5C—Patient and sample characteristics. (FIG. 5a) Timing (x axis, days) of therapies (color blocks) and sample collection (arrows) in each patient (y axis). (FIG. 5b) Cell type composition does not group samples by treatment history. Proportion (color bar) of the four major cell types (columns) in each of the ascites samples (rows) profiled by droplet-based scRNA-seq. (FIG. 5c) Cell intrinsic profiles do not group samples by treatment history. Pearson correlation coefficient (color bar) between the mean profiles of cancer cells (left), CAF (middle) or macrophages (right) of each pair of samples (rows, columns) profiled by droplet-based scRNA-seq and having at least 20 cells in each type.

FIG. 6A-6F—Clustering and characterization of malignant and non-malignant cell clusters in patient ascites by droplet scRNA-seq. (FIG. 6a) t-stochastic neighborhood embedding (tSNE) of 9,609 droplet-based scRNA-seq profiles from 8 samples (as in FIG. 1b), colored by unsupervised cluster assignment. (FIG. 6b) Cluster 9 is an inflammatory subset of CAFs. Comparison of the average expression (log 2(TPM+1)) of each gene in CAF cluster 9 (y axis) vs. CAF clusters 6 and 7 (x axis). Red: immunomodulatory genes. (FIG. 6c) CAF diversity observed within a single sample. Differential expression (log₂(TPM+1)) between CAF8 and CAF6/7 cells in patient 5.1 only of the top up- and down-regulated genes from (b). (d-f) Two distinct macrophage programs. (FIG. 6d) Hierarchical clustering of macrophages (rows, columns) from cluster 10 from either Patient 5.0 (left) or Patient 6 (right). Shown are the Pearson correlation coefficients (color bar) between expression profiles of macrophages, ordered by the clustering. Yellow lines highlight the separation into two main clusters. (FIG. 6e) Left: Differential expression (log₂(fold change)) for each gene (dot) between the two clusters identified in (d) for Patient 6 (x axis) or patient 5 (y axis), demonstrating high consistency. Top left corner: Pearson's r. Genes significantly differentially up or down regulated in both patients are marked in red and blue, respectively. Middle and Right: Expression levels (color bar, log₂(fold change)) of the highlighted differentially expressed genes from the left panel (rows) across macrophages from Patient 5 (middle) and Patient 6 (right) sorted by the hierarchical clustering of (d). (FIG. 6f) As in (e) for each other samples tested.

FIG. 7A-7B—Consistent clusters among droplet and plate based scRNA-seq. (FIG. 7a) Pearson correlation coefficient (color bar) between the average expression profiles of 302 cluster marker genes in cells in clusters defined from either droplet-based or plate-based scRNA-seq (rows, columns; ordered by hierarchical clustering). (FIG. 7b) Pearson correlation coefficient of the mean profile of cell type specific clusters comparing droplet based and plate-based scRNA-seq.

FIG. 8—Inferred CNA of single cells from plate based scRNA-seq profiles. Average relative copy number (color bar) in each chromosomal position (y axis) based on the average expression of the 100 genes surrounding that position⁸ in each cell in the malignant cell clusters 1-6 (x axis), compared to non-cancer clusters used as a reference, when using the original data (left) or when randomly ordering the genes across the genome and repeating the analysis (right), as control.

FIG. 9—Mesenchymal and immunoreactive TCGA subtypes reflect CAFs and macrophages by comparison to droplet based scRNA-seq profiles. Subtype score (color bar), based on average expression of subtype-specific genes (Methods) of each cluster from the droplet-based scRNA-seq dataset (rows) for each of four TCGA subtypes (column). Only clusters with >10 cells are represented in this figure.

FIG. 10A-10J—A putative stemness program in Patient 7 modules. (FIG. 10a, FIG. 10b) Intra-tumoral expression modules in patients 7 and 5. Relative expression (color bar, Methods) of the top 30 module-specific genes (rows) in each module (ordered by module, dashed vertical lines), as defined by NMF (Methods) across all cancer cells (columns; ordered by hierarchical clustering) from patients 7 (a), or 5 (b, same as FIG. 4c). Selected genes are annotated. (FIG. 10c) Co-variation of stemness related genes in patient 7. Relative expression of three putative stemness markers (rows) in cells from patient 7, rank ordered by the cell's average expression of the three markers. (FIG. 10d, FIG. 10e) Stemness related co-varying module present in patient 7 but not patient 8. Relative expression of the stemness score of patient 7 (top 20 genes (row) positively (top) or negatively (bottom)) correlated with the average expression of the three stemness genes in (c) in either cells from patient 7 (d) or patient 8 cells (e), with cells ordered by their average expression of the putative stemness score. (FIG. 10f) Stemness program is not detected in other ascites and primary tumor samples from the test cohort. Number of cells (y axis) expressing increasing numbers (x axis) of genes defining the stemness program from Patient 7 (CD24, CD133 (PROM1) and ALDH1A3) in patient cohort 3 (red) or expressing control genes with similar expression pattern in 10,000 simulations (FIG. 10g) Identification of cells expressing MHC Class II as cancer cells. Expression (color bar, log(TP100K+1)) of MHC Class II program, epithelial (cancer cell) markers, and macrophage markers (rows) in cancer cells (defined by marker expression and CNA) and macrophages (columns). Top panel: CNA signal, defined as the square of the inferred copy-number log-ratios, averaged across all genes. (FIG. 10h-FIG. 10j) MHC-II, cytokine and interferon programs are detected in other ascites and primary tumor samples from the test cohort. As in (f) for the three major immune programs defined as (FIG. 10h) MHC Class II (core genes (CD74, HLA-DRA, HL A-DRB1, HLA-DRB5, HLA-DMA, HL A-DPA1), (FIG. 10i) cytokines (core genes TNF, CXCL8, IL32, ICAM1, CCL2, CCL20, NFKBIA); and (FIG. 10j) interferon (IFN) program (core genes IFI6, IFI44, IFIT1, IFIT3, ISG15, MX1). Error bars: SD, *=p<0.05, **=p<0.001; empirical p-value is the fraction of simulations in which an equal number of stemness-program genes are detected as expressed.

FIG. 11A-11G—Some programs in malignant cells recur between patient ascites and PDX. (FIG. 11a) Congruent cancer cell profiles between patient and PDX cells. Left: Pearson correlation coefficient (color bar) between mean profiles (rows, columns) among major cell types discovered by plate-based scRNA-seq (cancer cells, macrophages and CAFs) in patient samples and three patient-derived xenograft models (DF20, DF68 and DF101). Right: Distribution of Pearson correlation coefficient (x axis) between different subsets. n=27 (8 patient samples and 19 PDX samples). (FIG. 11b-FIG. 11d) Intra-tumoral expression modules. Relative expression (color bar, Methods) of the top 30 module-specific genes (rows) in each module (ordered by module, dashed horizontal lines), as defined by NMF (Methods) across all cancer cells (columns; ordered by hierarchical clustering) from PDX models DF20 (b), DF68 (c), and DF101 (d). Selected genes are annotated. Top bar (b and c): cell of origin from individual mice. (FIG. 11e, FIG. 11f) Cell cycle and inflammatory/immune programs recur across PDX models. (e) Number of top genes (color bar) shared between pairs of patients (rows, ordered as in FIG. 3e) and PDX (columns; ordered by hierarchical clustering) modules. Top: origin of each PDX module. (f) Module membership in the top 30 (black) or 50 (grey) of selected genes (rows) from cell cycle (top), immune-related (middle), and other (bottom) modules across all modules (columns), ordered as in (e). All genes included were shared between a corresponding PDX module and patient ascites module. (FIG. 11g) Cytokine and MHC-II programs are only identified in patient samples. Median expression (x axis) and % of outlier highly expressing cells (y axis; average log₂(TPM+1)>5 and more than 2 SD larger than the mean of all cells) of the cytokine (left) and MHC-II (right) programs in each patient (black) and PDX (blue) samples. N=25 (6 patient samples and 19 PDX samples).

FIG. 12A-12C—On-target activity of JSI-124. (FIG. 12a) Prominent expression of JAK-STAT pathway genes. Mean gene expression (x axis, log₂(TPM+1)) and percentage of expressing cells (y axis) across the entire cell's transcriptomes with highlighted signaling genes in patient cancer cells (top left), PDX models (top right), patient-derived CAFs (bottom left) and macrophages (bottom right). Black curve: LOWESS regression curve. Dark and light blue: top 5 and 10 percentiles calculated in a moving average of 200 genes. STAT3 activity induced by Oncostatin M. Relative (mean) luciferase activity (y axis) in Heya8 ovarian cancer cells transfected with a STAT3 responsive reporter that were stimulated with OSM to activate STAT3 for 6 hours (dark blue) or untreated (light blue) with either 1 h pre-treatment with JSI-124 (1 μM) or vehicle (x axis) for 1 hour. p=0.09, t test. Error bars: SD. (FIG. 12c) JSI-124 treatment reduced pSTAT3. Cropped immunoblot (representative of duplicates; uncropped available in Source Data) of STAT3 and phosphorylated (p-)STAT3 from cells treated with 1 μM JSI-124 for the indicated hours (bottom). Par=parental cell line, and R1 and R2 refer to two independently generated platinum-resistant cell lines.

FIG. 13A-13B—Dose response of JSI-124 in 2D cultures or 3D spheroids. Relative (mean) viability (y axis, relative luminescence signal compared to DMSO control) of three ovarian cancer cell lines (labels, top) grown for 4 days in either ultra-low attachment conditions eliciting formation of spheroids (FIG. 13a) or in 2D cultures in regular plastic culture surfaces (FIG. 13b), and treated with JSI-124, carboplatin, paclitaxel, cisplatin or olaparib at indicated doses (x axis, log μM). Error bars: SD. n=4. Representative of biological duplicates.

The figures herein are for illustrative purposes only and are not necessarily drawn to scale.

### DETAILED DESCRIPTION OF THE EXAMPLE EMBODIMENTS

### General Definitions

Unless defined otherwise, technical and scientific terms used herein have the same meaning as commonly understood by one of ordinary skill in the art to which this disclosure pertains. Definitions of common terms and techniques in molecular biology may be found in Molecular Cloning: A Laboratory Manual, 2^{nd} edition (1989) (Sambrook, Fritsch, and Maniatis); Molecular Cloning: A Laboratory Manual, 4^{th} edition (2012) (Green and Sambrook); Current Protocols in Molecular Biology (1987) (F. M. Ausubel et al. eds.); the series Methods in Enzymology (Academic Press, Inc.): PCR 2: A Practical Approach (1995) (M. J. MacPherson, B. D. Hames, and G. R. Taylor eds.): Antibodies, A Laboratory Manual (1988) (Harlow and Lane, eds.): Antibodies A Laboratory Manual, 2^{nd} edition 2013 (E. A. Greenfield ed.); Animal Cell Culture (1987) (R. I. Freshney, ed.); Benjamin Lewin, Genes IX, published by Jones and Bartlet, 2008 (ISBN 0763752223); Kendrew et al. (eds.), The Encyclopedia of Molecular Biology, published by Blackwell Science Ltd., 1994 (ISBN 0632021829); Robert A. Meyers (ed.), Molecular Biology and Biotechnology: a Comprehensive Desk Reference, published by VCH Publishers, Inc., 1995 (ISBN 9780471185710); Singleton et al., Dictionary of Microbiology and Molecular Biology 2nd ed., J. Wiley & Sons (New York, N.Y. 1994), March, Advanced Organic Chemistry Reactions, Mechanisms and Structure 4th ed., John Wiley & Sons (New York, N.Y. 1992); and Marten H. Hofker and Jan van Deursen, Transgenic Mouse Methods and Protocols, 2^{nd} edition (2011).

As used herein, the singular forms “a”, “an”, and “the” include both singular and plural referents unless the context clearly dictates otherwise.

The term “optional” or “optionally” means that the subsequent described event, circumstance or substituent may or may not occur, and that the description includes instances where the event or circumstance occurs and instances where it does not.

The recitation of numerical ranges by endpoints includes all numbers and fractions subsumed within the respective ranges, as well as the recited endpoints.

The terms “about” or “approximately” as used herein when referring to a measurable value such as a parameter, an amount, a temporal duration, and the like, are meant to encompass variations of and from the specified value, such as variations of +/−10% or less, +/−5% or less, +/−1% or less, and +/−0.1% or less of and from the specified value, insofar such variations are appropriate to perform in the disclosed invention. It is to be understood that the value to which the modifier “about” or “approximately” refers is itself also specifically, and preferably, disclosed.

As used herein, a “biological sample” may contain whole cells and/or live cells and/or cell debris. The biological sample may contain (or be derived from) a “bodily fluid”. The present invention encompasses embodiments wherein the bodily fluid is selected from amniotic fluid, aqueous humour, vitreous humour, bile, blood serum, breast milk, cerebrospinal fluid, cerumen (earwax), chyle, chyme, endolymph, perilymph, exudates, feces, female ejaculate, gastric acid, gastric juice, lymph, mucus (including nasal drainage and phlegm), pericardial fluid, peritoneal fluid, pleural fluid, pus, rheum, saliva, sebum (skin oil), semen, sputum, synovial fluid, sweat, tears, urine, vaginal secretion, vomit and mixtures of one or more thereof. Biological samples include cell cultures, bodily fluids, cell cultures from bodily fluids. Bodily fluids may be obtained from a mammal organism, for example by puncture, or other collecting or sampling procedures.

The terms “subject,” “individual,” and “patient” are used interchangeably herein to refer to a vertebrate, preferably a mammal, more preferably a human. Mammals include, but are not limited to, murines, simians, humans, farm animals, sport animals, and pets. Tissues, cells and their progeny of a biological entity obtained in vivo or cultured in vitro are also encompassed.

Various embodiments are described hereinafter. It should be noted that the specific embodiments are not intended as an exhaustive description or as a limitation to the broader aspects discussed herein. One aspect described in conjunction with a particular embodiment is not necessarily limited to that embodiment and can be practiced with any other embodiment(s). Reference throughout this specification to “one embodiment”, “an embodiment,” “an example embodiment,” means that a particular feature, structure or characteristic described in connection with the embodiment is included in at least one embodiment of the present invention. Thus, appearances of the phrases “in one embodiment,” “in an embodiment,” or “an example embodiment” in various places throughout this specification are not necessarily all referring to the same embodiment, but may. Furthermore, the particular features, structures or characteristics may be combined in any suitable manner, as would be apparent to a person skilled in the art from this disclosure, in one or more embodiments. Furthermore, while some embodiments described herein include some but not other features included in other embodiments, combinations of features of different embodiments are meant to be within the scope of the invention. For example, in the appended claims, any of the claimed embodiments can be used in any combination.

Reference is made to International Patent Application PCT/US2018/025491, filed Mar. 30, 2018 and published as WO2018183908. Reference is also made to Izar B, Tirosh I, Stover E H, Wakiro I, Cuoco M S, Alter I, Rodman C, Leeson R, Su M J, Shah P, Iwanicki M, Walker S R, Kanodia A, Melms J C, Mei S, Lin J R, Porter C B M, Slyper M, Waldman J, Jerby-Arnon L, Ashenberg O, Brinker T J, Mills C, Rogava M, Vigneau S, Sorger P K, Garraway L A, Konstantinopoulos P A, Liu J F, Matulonis U, Johnson B E, Rozenblatt-Rosen O, Rotem A, Regev A. A single-cell landscape of high-grade serous ovarian cancer. Nat Med. 2020 August; 26(8):1271-1279. doi: 10.1038/s41591-020-0926-0. Epub 2020 Jun. 22. PMID: 32572264; PMCID: PMC7723336.

All publications, published patent documents, and patent applications cited herein are hereby incorporated by reference to the same extent as though each individual publication, published patent document, or patent application was specifically and individually indicated as being incorporated by reference.

### Overview

Embodiments disclosed herein provide tumor markers and biological programs differentially expressed in malignant cells and the tumor microenvironment (TME). Malignant abdominal fluid (ascites) frequently develops in women with advanced high-grade serous ovarian cancer (HGSOC) and is associated with drug resistance and a poor prognosis. Here, Applicants studied ascites samples from HGSOC patients, primary tumors and patient-derived xenograft (PDX) models by single-cell RNA-seq (scRNA-seq), to resolve the expression profiles of diverse cancer, immune and stromal cells, and their interactions, each of which may contribute to disease development and treatment resistance⁸⁻¹¹. To comprehensively characterize the HGSOC ascites ecosystem, Applicants used single-cell RNA-seq (scRNA-seq) to profile ˜11,000 cells from 22 ascites specimens from 11 HGSOC patients. Applicants found significant inter-patient variability in the composition and functional programs of ascites cells, including immunomodulatory fibroblast sub-populations and dichotomous macrophage populations. Applicants found that the previously described “immunoreactive” and “mesenchymal” subtypes of HGSOC, which have prognostic implications, reflect the abundance of immune infiltrates and fibroblasts rather than distinct subsets of malignant cells. Malignant cell variability was partly explained by heterogeneous copy number alterations (CNA) patterns or expression of a stemness program. Malignant cells shared expression of inflammatory programs that were largely recapitulated in scRNA-seq of ˜35,000 cells from additionally collected samples, including three ascites, two primary HGSOC tumors and three patient-ascites-derived xenograft models. Inhibition of the JAK/STAT-pathway, which was expressed in both malignant cells and cancer-associated fibroblasts (CAFs), had potent anti-tumor activity in primary short-term cultures and PDX models.

Tumor markers and biological programs were identified in the single cells. The markers and biological programs were identified in ovarian cancer, but they may be applicable to other cancers as well. In certain embodiments, the tumor markers are not expressed or expressed significantly lower on non-malignant cells. The tumor markers and biological programs can be used for therapeutic, diagnostic and screening applications. In certain embodiments, tumor markers that are highly expressed on malignant cells can be targeted. In certain embodiments, T cells, in particular CAR T cells, target the tumor markers. In certain embodiments, one or more agents capable of modulating a biological program are used to treat a tumor. In certain embodiments, biological programs are targeted in combination with an additional agent. In certain embodiments, modulation of a biological program can increase the response to an anti-tumor therapy (e.g., checkpoint blockade therapy (CPB), or inhibition of the JAK/STAT pathway). This work also contributes to resolving the HSGOC landscape¹⁻³ and provides a resource for the development of novel therapeutic approaches (e.g., targeting tumor markers not expressed on non-malignant cells).

### Tumor Specific Genes and Expression Programs

Applicants identified genes that were differentially expressed between the malignant and non-malignant single cell clusters. Applicants used dimension reduction to cluster cells based on gene expression in each cell. Table 2 shows the average expression of all genes included in the scRNA-seq for each cluster. In embodiments, tumor specific markers are identified. In embodiments, the tumor specific markers are expressed higher in malignant cells than non-malignant cells. The increased expression in specific clusters of malignant cells was identified using single cell RNA sequencing and may not be apparent using prior methods, such as bulk sequencing techniques. In embodiments, the tumor markers are expressed in one or more malignant cell clusters, such as a specific subset of malignant cells. In certain embodiments, the tumors markers have an average expression in a malignant cell cluster that is at least 1. In certain embodiments, the average expression is greater than 2, greater than 3, or greater than 4. In certain embodiments, the expression of a tumor marker is less than 1 in non-malignant single cell clusters. In certain embodiments, the tumor marker has greater than two-fold higher expression in malignant cells. In certain embodiments, the tumor markers are surface proteins, thus tumor cells can be targeted by an extracellular agent (e.g., CAR T cells). Example tumor markers that were found to be expressed greater than 4 in malignant cells and were expressed greater than 2-fold as compared to non-malignant cells include CLDN3, CLDN7, CLDN4, EPCAM, TACSTD2, MAL2, LCN2, CKB, RBP1, CDKN2A, C19orf33, WFDC2, LSR, CRABP2, S100A13, KRT7, CRIP2, MDK, CD9, SPINT2, SLPI, KRT19, KRT18, KRT8, TM4SF1, NGFRAP1, S100A16, PCBD1, OCIAD2, ZNF428, TMEM205, TSTD1, TNFRSF12A, MARCKSL1, IFI27, CD47, POLR2I, CCDC124, PDCD5 and DPY30. Surface markers, which may be targeted by an extracellular agent, include CLDN3, CLDN7, CLDN4, EPCAM, TACSTD2, MAL2, LSR, CD9, SPINT2, TM4SF1, TMEM205, TNFRSF12A and CD47. The tumor markers included known and unknown tumor markers (see, e.g., Epithelial cell adhesion molecule (EpCAM); and Mohtar, et al., Revisiting the Roles of Pro-Metastatic EpCAM in Cancer, Biomolecules. 2020 February; 10(2): 255). Example tumor markers that were found to be expressed greater than 4 in malignant cells, less than 1 in non-malignant cells and were expressed greater than 2 fold as compared to non-malignant cells include CLDN3, CLDN7, CLDN4, EPCAM, TACSTD2, MAL2, LCN2, CKB, RBP1, CDKN2A, C19orf33, and LSR. Example tumor markers that were found to be expressed greater than 4 in malignant cells and were expressed greater than 5 fold as compared to non-malignant cells include CLDN3, CLDN7, CLDN4, EPCAM, TACSTD2, MAL2, LCN2, CKB, RBP1, CDKN2A, C19orf33, WFDC2 and LSR. Example tumor markers that were found to be expressed greater than 4 in malignant cells and were expressed greater than 10 fold as compared to non-malignant cells include CLDN3, CLDN7, CLDN4, EPCAM, TACSTD2, MAL2 and LCN2. Example tumor markers that were found to be expressed greater than 4 in malignant cells and were expressed greater than 15 fold as compared to non-malignant cells include CLDN3, CLDN7 and CLDN4.

CLDN3, CLDN7 and CLDN4 are claudins. Claudins are major integral proteins of tight junctions (TJs), the apical cell-cell adhesions that enable maintaining polarity of epithelial cells, their differentiation, and cell signaling. Claudins are a group of transmembrane proteins which play a critical role, along with other tight junction (TJ) proteins, in the proper functioning of epithelial tight junctions (TJs) (see, e.g., Bhat, et al., Tight Junction Proteins and Signaling Pathways in Cancer and Inflammation: A Functional Crosstalk, Front Physiol. 2019 Jan. 23; 9:1942). In certain embodiments, overexpression of claudin-3 and -4 in ovarian cancer cells promotes cancer progression (Agarwal et al., 2005, Claudin-3 and claudin-4 expression in ovarian epithelial cells enhances invasion and is associated with increased matrix metalloproteinase-2 activity. Cancer Res. 65 7378-7385) in both mouse and human ovarian cancer xenografts model (Shang et al., 2012, Tight junction proteins claudin-3 and claudin-4 control tumor growth and metastases. Neoplasia 14 974-985). The role of claudin-4 in pro-angiogenic and enhanced motility in ovarian cancer was also demonstrated (Li et al., 2009, Possible angiogenic roles for claudin-4 in ovarian cancer. Cancer Biol. Ther. 8 1806-1814). In certain embodiments, claudins-3 and -4 have been shown to be tumor suppressors. Their overexpression decreases Wnt signaling, affects E-cadherin expression, and decreases in vitro cell migration and invasion. In ovarian cancer, downregulation of claudin-3/-4 promotes tumor growth and metastasis, while less expression of claudin-3/-4 along with claudin-7 results in high malignancy in breast cancer (Prat et al., 2010, Phenotypic and molecular characterization of the claudin-low intrinsic subtype of breast cancer. Breast Cancer Res. 12:R68). In certain embodiments, high expression of claudin-4 suppresses invasion and metastasis in pancreatic cancer (Michl et al., 2003, Claudin-4 expression decreases invasiveness and metastatic potential of pancreatic cancer. Cancer Res. 63 6265-6271) while in gastric cancer cells similar inhibition is seen without affecting the cell growth (Kwon et al., 2011, Claudin-4 overexpression is associated with epigenetic derepression in gastric carcinoma. Lab. Invest. 91 1652-1667). In certain embodiments, claudin-7 functions both as tumor suppressor and promoter. In esophageal squamous cell carcinoma claudin-7 has been shown to enhance cell growth and metastasis (Lioni et al., 2007, Dysregulation of claudin-7 leads to loss of E-cadherin expression and the increased invasion of esophageal squamous cell carcinoma cells. Am. J. Pathol. 170 709-721). In CRC and ovarian cancer, claudin-7 overexpression promotes tumor formation and invasiveness (Johnson et al., 2005, Expression of tight-junction protein claudin-7 is an early event in gastric tumorigenesis. Am. J. Pathol. 167 577-584; Dahiya et al., 2011, Claudin-7 is frequently overexpressed in ovarian cancer and promotes invasion. PLoS One 6:e22119). In certain embodiments, in colon cancer, claudin-7 was shown to have a tumor suppressor effect (Bhat et al., 2015, Claudin-7 expression induces mesenchymal to epithelial transformation (MET) to inhibit colon tumorigenesis. Oncogene 34 4570-4580).

As used herein CLDN3 refers to claudin 3 (also known as: C7orf1, CPE-R2, CPETR2, HRVP1, RVP1) in all organisms, in particular human. Reference sequences include NM_001306.4, NM_009902.4, and NM_031700.2.

As used herein CLDN7 refers to claudin 7 (also known as: CEPTRL2, CLDN-7, CPETRL2, Hs.84359, claudin-1) in all organisms, in particular human. Reference sequences include NM_001307.6, NM_001185023.1, NM_001185022.1, NM_016887.6, NM_001193619.1, and NM_031702.1.

As used herein CLDN4 refers to claudin 4 (also known as: CPE-R, CPER, CPETR, CPETR1, WBSCR8, hCPE-R) in all organisms, in particular human. Reference sequences include NM_001305.4, NM_009903.2 and NM_001012022.1.

Applicants also identified expression programs that vary among malignant cells. The programs have co-varying gene expression. As used herein, co-varying refers to genes that go up and down together. As used herein the term “modules” refers to sets of genes that co-vary across subjects or single cells. Modules may be characterized as an expression program based on the genes expressed in the modules (e.g., cell cycle, inflammatory cytokines, MHC-class II antigen presentation, or interferon-response modules). For ease of discussion, when discussing gene expression, any of gene or genes, protein or proteins, or epigenetic element(s) may be substituted. Tables 3 and 4 show exemplary gene modules comprising co-varying genes. As used herein the term “expression program” can be used interchangeably with “biological program” or “transcriptional program” and may refer to a set of genes that share a role in a biological function (e.g., cell cycle, inflammatory cytokines, MHC-class II antigen presentation, or interferon-response programs). Biological programs can include a pattern of gene expression that result in a corresponding physiological event or phenotypic trait. Biological programs can include up to several hundred genes that are expressed in a spatially and temporally controlled fashion. Expression of individual genes can be shared between biological programs. Expression of individual genes can be shared among different single cell types; however, expression of a biological program may be cell type specific or temporally specific (e.g., the biological program is expressed in a cell type at a specific time). Multiple biological programs may include the same gene, reflecting the gene's roles in different processes. Expression of a biological program may be regulated by a master switch, such as a nuclear receptor or transcription factor. As used herein, the term “topic” refers to a biological program. The biological program can be modeled as a distribution over expressed genes.

Expression programs can be identified using the single cell RNA sequencing of the present invention. One method to identify cell programs is non-negative matrix factorization (NMF) (see, e.g., Lee D D and Seung H S, Learning the parts of objects by non-negative matrix factorization, Nature. 1999 Oct. 21; 401(6755):788-91). As an alternative, a generative model based on latent Dirichlet allocation (LDA) (Blei, D. M., Ng, A. Y., and Jordan, M. I. (2003). Latent Dirichlet allocation. J Mach Learn Res 3, 993-1022), or “topic modeling” may be created. Topic modeling is a statistical data mining approach for discovering the abstract topics that explain the words occurring in a collection of text documents. Originally developed to discover key semantic topics reflected by the words used in a corpus of documents (Dumais, S. T., Furnas, G. W., Landauer, T. K., and Harshman, R. (1990). Indexing by Latent Semantic Analysis. Journal of the American Society for Information Science 41, 391-407), topic modeling can be used to explore gene programs (“topics”) in each cell (“document”) based on the distribution of genes (“words”) expressed in the cell. A gene can belong to multiple programs, and its relative relevance in the topic is reflected by a weight. A cell is then represented as a weighted mixture of topics, where the weights reflect the importance of the corresponding gene program in the cell. Topic modeling using LDA has recently been applied to scRNA-seq data (see, e.g., Bielecki, Riesenfeld, Kowalczyk, et al., 2018 Skin inflammation driven by differentiation of quiescent tissue-resident ILCs into a spectrum of pathogenic effectors. bioRxiv 461228; and duVerle, D. A., Yotsukura, S., Nomura, S., Aburatani, H., and Tsuda, K. (2016). CellTree: an R/bioconductor package to infer the hierarchical structure of cell populations from single-cell RNA-seq data. BMC Bioinformatics 17, 363). Other approaches include word embeddings. Identifying cell programs can recover cell states and bridge differences between cells. Single cell types may span a range of continuous cell states (see, e.g., Shekhar et al., Comprehensive Classification of Retinal Bipolar Neurons by Single-Cell Transcriptomics Cell. 2016 Aug. 25; 166(5):1308-1323.e30; and Bielecki, et al., 2018).

Applicants identified programs that were shared between malignant cells across patients. Thus, the shared programs can be used to shift tumor cells or to diagnose a subject across subjects suffering from cancer. The programs can indicate response to therapies or can be shifted to increase a response to a therapy. Exemplary programs identified in single cells according to the present invention include inflammatory cytokines programs, MHC-class II antigen presentation programs, and interferon-response programs.

Inflammatory cytokines programs may include one or more genes in Pt8_cluster8 (SOD2, CXCL3, TNFAIP3, CXCL1, TUBA1A, TPM1, EDN1, TAGLN, CLDN1, CXCL2, TNFAIP2, NEDD9, ADAMTS9, FLNA, ARHGAP29, CTHRC1, RGS10, UBD, THBS1, CALD1, PTX3, RELB, CYR61, NFKBIA, CCL2, TNF, ICAM1, CCL20, IL8 and IL32) or Pt9_cluster8 (UBD, TAP1, TNFAIP2, CRYAB, MARCO, LGALS14, PSMB9, CD74, TUBB2B, KRT23, IL4I1, TAPBP, C10orf10, CLDN1, SOD2, SELM, JAK3, TNFSF10, B2M, HLA-H, COL4A2, HLA-B, IL8, IL23A, ICAM1, TNF, CCL20, CCL2, IL32 and CD40).

MHC-class II antigen presentation programs may include one or more genes in Pt7_cluster2 (LYNX1, LYPD2, CLU, TRIM29, MMP7, MUC4, TSPAN1, ATP6V1B1, SERPINA1, SLPI, KCNN4, SYT8, RARRES3, PTGES, RASAL1, CP, FOLR1, UNC5B, ALOX5, MUC20, FOS, SLC4A11, FXYD3, C3, UNC5B-AS1, DEFB1, COL12A1, CD74, HLA-DRB1 and HLA-DRA) or Pt8_cluster3 (RARRES1, CRYAB, NNMT, PLA2G16, MGST1, HLA-DRB6, MT1F, CLU, CD14, NFIB, C3, BNIP3L, OAT, SLC34A2, NUPR1, ANXA1, MT1X, CDC42EP2, TUBB6, SAA1, CSTB, VIM, GPX3, MT1E, HLA-DRB1, HLA-DRA, HLA-DRB5, HLA-DMA, CD74 and HLA-DPA1).

Interferon-response programs may include one or more genes in Pt9_cluster1 (CLU, TACSTD2, MFAP2, LGALS3BP, FBLN2, LY6E, VTCN1, CLIC5, PTPRS, RAB25, SH3BGRL, BST2, SERPING1, MFGE8, ANKRD65, UCA1, THEM6, HSPG2, MSLN, NDRG2, TAPBP, CDH6, PLD3, RBMS3, COMP, SEPP1, RNF213, CD74, IFIT3 and IFI6) or Pt10_cluster7 (CCBP2, TACSTD2, OAS1, MX1, GPNMB, OAS2, KRT23, MSLN, TXNIP, C15orf48, UPK3BL, PTGES, LCN2, CD82, SAT1, VTCN1, ITGB2, NCCRP1, CEACAM6, AGR2, PSCA, PARP14, HERC6, GABRP, IFI44L, IFIT3, IFI6, IFI44, ISG15 and IFIT1) or DF68_cluster3 (XAF1, MX1, PARP14, DDX60, C19orf66, OAS3, STAT1, OAS1, PARP9, PLSCR1, IFI44, IFIT3, OASL, TRIM22, DTX3L, IFITM1, PSMB8, RTP4, RSAD2, DDX58, IFITM3, NMI, UBE2L6, UBA7, SP100, OAS2, ISG15, IFIT1, IFI44L and IFI6) or DF101_cluster2 (TACSTD2, LOC100505633, CLU, HLA-H, CLDN1, HLA-C, CD24, TSPAN15, HLA-B, CCL28, FAM107A, RARRES3, TNC, GPX1, KLK5, IFI27, SEPP1, CCDC3, PLA2G16, MAL, CNN3, CMBL, PSMB10, CRIP1, KRT5, C3orf55, HLA-A, ISG15, IFIT1 and IFI6) or DF101_cluster3 (IFIT3, ISG20, IFIT2, MX1, OAS1, HLA-H, RSAD2, C19orf66, PARP14, STAT1, HLA-A, EPSTI1, CMPK2, KRT6A, IFI35, HLA-F, HLA-B, OAS3, B2M, DDX58, CFB, ANXA3, TIMP1, DTX3L, PARP9, GPRC5A, ISG15, IFIT1, IFI44L and OAS2).

Applicants found that the previously described “immunoreactive” and “mesenchymal” subtypes of HGSOC, which have prognostic implications, reflect the abundance of immune infiltrates and fibroblasts rather than distinct subsets of malignant cells. The mesenchymal signature defining the mesenchymal phenotype is expressed mainly in cancer-associated fibroblasts (CAFs). Thus, in certain embodiments, a mesenchymal phenotype can be detected by detecting the gene signature in CAFs. In certain embodiments, the mesenchymal gene signature detected in CAFs includes one or more genes selected from the group consisting of: PDPN, C1QA/B/C, CFB, CXCL12, CXCL1, CXCL2, CXCL10, IL6, IL10, ALDH1A2, ACTA2, COL1A2, LUM, COL3A1, DCN and COL1A1; or clusters 6-9 of Table 2. The immunoreactive signature defining the immunoreactive phenotype is expressed mainly in macrophages. Thus, in certain embodiments, an immunoreactive phenotype can be detected by detecting the gene signature in macrophages. In certain embodiments, the immunoreactive gene signature detected in macrophages includes one or more genes selected from the group consisting of: CD52, CD14, AIF1, CSF1R, C1QB, C1QA, CD163, CD36, FCGR3A, CCL3, CCL4, IFNGR1, CD1D, C2, APOE, APOC1, CTSD, CTSZ, LYZ, FCN1, DDX5, MNDA, C3AR1, VISG4, SERPINA1, HLA-DPA1, HLA-DRA, HLA-DPB1 and HLA-DRB5; or clusters 10-13 of Table 2.

In certain embodiments, the tumor markers or expression programs are part of a gene signature. In certain embodiments, a signature is characterized as being specific for a particular tumor cell or tumor cell (sub)population if it is upregulated or only present, detected or detectable in that particular tumor cell or tumor cell (sub)population, or alternatively is downregulated or only absent, or undetectable in that particular tumor cell or tumor cell (sub)population. In this context, a signature consists of one or more differentially expressed genes/proteins or differential epigenetic elements when comparing different cells or cell (sub)populations, including comparing different tumor cells or tumor cell (sub)populations, as well as comparing tumor cells or tumor cell (sub)populations with non-tumor cells or non-tumor cell (sub)populations. It is to be understood that “differentially expressed” genes/proteins include genes/proteins which are up- or down-regulated as well as genes/proteins which are turned on or off. When referring to up- or down-regulation, in certain embodiments, such up- or down-regulation is preferably at least two-fold, such as two-fold, three-fold, four-fold, five-fold, or more, such as for instance at least ten-fold, at least 20-fold, at least 30-fold, at least 40-fold, at least 50-fold, or more. Alternatively, or in addition, differential expression may be determined based on common statistical tests, as is known in the art.

When referring to induction, or alternatively suppression of a particular signature, preferable is meant induction or alternatively suppression (or upregulation or downregulation) of at least one gene/protein and/or epigenetic element of the signature, such as for instance at least two, at least three, at least four, at least five, at least six, or all genes/proteins and/or epigenetic elements of the signature.

All gene name symbols refer to the gene as commonly known in the art. The examples described herein that refer to the human gene names are to be understood to also encompasses genes in any other organism (e.g., homologous, orthologous genes). The term, homolog, may apply to the relationship between genes separated by the event of speciation (e.g., ortholog). Orthologs are genes in different species that evolved from a common ancestral gene by speciation. Normally, orthologs retain the same function in the course of evolution. Gene symbols may be those referred to by the HUGO Gene Nomenclature Committee (HGNC) or National Center for Biotechnology Information (NCBI). Any reference to the gene symbol is a reference made to the entire gene or variants of the gene.

### Cancer

The tumor markers and/or expression programs may be expressed in any cancer. Thus, the therapeutic, diagnostic and screening methods are generally applicable to any cancer expressing the tumor markers and/or expression programs. Non-limiting cancers include liquid tumors such as leukemia (e.g., acute leukemia, acute lymphocytic leukemia, acute myelocytic leukemia, acute myeloblastic leukemia, acute promyelocytic leukemia, acute myelomonocytic leukemia, acute monocytic leukemia, acute erythroleukemia, chronic leukemia, chronic myelocytic leukemia, chronic lymphocytic leukemia), polycythemia vera, lymphoma (e.g., Hodgkin's disease, non-Hodgkin's disease), Waldenstrom's macroglobulinemia, heavy chain disease, or multiple myeloma. The cancer may include, without limitation, solid tumors such as sarcomas and carcinomas. Examples of solid tumors include, but are not limited to fibrosarcoma, myxosarcoma, liposarcoma, chondrosarcoma, osteogenic sarcoma, chordoma, angiosarcoma, endotheliosarcoma, lymphangiosarcoma, lymphangioendotheliosarcoma, synovioma, mesothelioma, Ewing's tumor, leiomyosarcoma, rhabdomyosarcoma, squamous cell carcinoma, basal cell carcinoma, adenocarcinoma, sweat gland carcinoma, sebaceous gland carcinoma, papillary carcinoma, papillary adenocarcinomas, cystadenocarcinoma, medullary carcinoma, epithelial carcinoma, bronchogenic carcinoma, hepatoma, colorectal cancer (e.g., colon cancer, rectal cancer), anal cancer, pancreatic cancer (e.g., pancreatic adenocarcinoma, islet cell carcinoma, neuroendocrine tumors), breast cancer (e.g., ductal carcinoma, lobular carcinoma, inflammatory breast cancer, clear cell carcinoma, mucinous carcinoma), ovarian carcinoma (e.g., ovarian epithelial carcinoma or surface epithelial-stromal tumour including serous tumour, endometrioid tumor and mucinous cystadenocarcinoma, sex-cord-stromal tumor), prostate cancer, liver and bile duct carcinoma (e.g., hepatocellular carcinoma, cholangiocarcinoma, hemangioma), choriocarcinoma, seminoma, embryonal carcinoma, kidney cancer (e.g., renal cell carcinoma, clear cell carcinoma, Wilm's tumor, nephroblastoma), cervical cancer, uterine cancer (e.g., endometrial adenocarcinoma, uterine papillary serous carcinoma, uterine clear-cell carcinoma, uterine sarcomas and leiomyosarcomas, mixed mullerian tumors), testicular cancer, germ cell tumor, lung cancer (e.g., lung adenocarcinoma, squamous cell carcinoma, large cell carcinoma, bronchioloalveolar carcinoma, non-small-cell carcinoma, small cell carcinoma, mesothelioma), bladder carcinoma, signet ring cell carcinoma, cancer of the head and neck (e.g., squamous cell carcinomas), esophageal carcinoma (e.g., esophageal adenocarcinoma), tumors of the brain (e.g., glioma, glioblastoma, medullablastoma, astrocytoma, medulloblastoma, craniopharyngioma, ependymoma, pinealoma, hemangioblastoma, acoustic neuroma, oligodenroglioma, schwannoma, meningioma), neuroblastoma, retinoblastoma, neuroendocrine tumor, melanoma, cancer of the stomach (e.g., stomach adenocarcinoma, gastrointestinal stromal tumor), or carcinoids. Lymphoproliferative disorders are also considered to be proliferative diseases.

### Ovarian Cancer

Ovarian cancer is a cancer that forms in or on an ovary. Symptoms may include bloating, pelvic pain, abdominal swelling, and loss of appetite, among others. Common areas to which the cancer may spread include the lining of the abdomen, lymph nodes, lungs, and liver. The most common type of ovarian cancer is ovarian carcinoma (>95% of all cases). There are five main subtypes of ovarian carcinoma, of which high-grade serous carcinoma is the most common. These tumors are believed to start in the cells covering the ovaries, though some may form at the Fallopian tubes. Less common types of ovarian cancer include germ cell tumors and sex cord stromal tumors. A diagnosis of ovarian cancer is confirmed through a biopsy of tissue, usually removed during surgery.

High-grade serous ovarian carcinoma (HGSOC) is a type of tumor that arises from the serous epithelial layer in the abdominopelvic cavity and is mainly found in the ovary. HGSOCs make up the majority of ovarian cancer cases and have the lowest survival rates. HGSOC is distinct from low-grade serous carcinoma (LGSC), which arises from ovarian tissue, is less aggressive and is present in stage I ovarian cancer where tumors are localized to the ovary. Although originally thought to arise from the squamous epithelial cell layer covering the ovary, HGSOC is now thought to originate in the Fallopian tube epithelium. HGSOC is much more invasive than LGSC with a higher fatality rate; although it is more sensitive to platinum-based chemotherapy, possibly due to its rapid growth rate. In rare cases, HGSOCs can develop from LGSCs, but generally the two types arise independently of each other. See, e.g., Hatano Y, Hatano K, Tamada M, et al. A Comprehensive Review of Ovarian Serous Carcinoma. Adv Anat Pathol. 2019; 26(5):329-339.

If caught and treated in an early stage, ovarian cancer is often curable. Treatment usually includes some combination of surgery, radiation therapy, and chemotherapy. Outcomes depend on the extent of the disease, the subtype of cancer present, and other medical conditions. The overall five-year survival rate in the United States is 45%.

If ovarian cancer recurs, it is considered partially platinum-sensitive or platinum-resistant, based on the time since the last recurrence treated with platins: partially platinum-sensitive cancers recurred 6-12 months after last treatment, and platinum-resistant cancers have an interval of less than 6 months.

For platinum-sensitive tumors, platins are utilized for second-line chemotherapy, often in combination with other cytotoxic agents. Regimens include carboplatin combined with pegylated liposomal doxorubicin, gemcitabine, or paclitaxel. If the tumor is determined to be platinum-resistant, vincristine, dactinomycin, and cyclophosphamide (VAC) or some combination of paclitaxel, gemcitabine, and oxaliplatin can be used as a second-line therapy.

### Therapeutic Methods

In one aspect, any of the tumor specific markers or expression programs described herein can be therapeutically targeted to treat a cancer as described herein. In certain embodiments, the therapeutic methods may be used in combination with a standard treatment for the cancer.

In an embodiment, a population of ex vivo T cells is provided which comprises an exogenous nucleic acid sequence encoding a binding protein specific for binding a surface protein. Surface proteins are as described elsewhere herein, and may be chosen based on a higher average expression in one or more clusters, for example, clusters 1 to 5, or cell subpopulations relative to one or more other clusters, for example any one of clusters 6 to 18, or cell subpopulations. In an aspect, T cells specific for a surface protein are selected from CLDN3, CLDN7, CLDN4, EPCAM, TACSTD2, MAL2, LSR, CD9, SPINT2, TM4SF1, TMEM205, TNFRSF12A and CD47, more preferably CLDN3, CLDN7 and/or CLDN4. Methods of treating cancer may comprise administering the population of T cells to a subject in need thereof.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested. As used herein “treating” includes ameliorating, curing, preventing it from becoming worse, slowing the rate of progression, or preventing the disorder from re-occurring (i.e., to prevent a relapse).

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The term also applies to a dose that will provide an image for detection by any one of the imaging methods described herein. The specific dose may vary depending on one or more of: the particular agent chosen, the dosing regimen to be followed, whether it is administered in combination with other compounds, timing of administration, the tissue to be imaged, and the physical delivery system in which it is carried.

### Adoptive Cell Transfer

Using single cell RNA sequencing, Applicants identified tumor specific markers expressed in subpopulations of tumor cells (Table 2). These markers include surface markers. The surface markers include CLDN3, CLDN7, CLDN4, EPCAM, TACSTD2, MAL2, LSR, CD9, SPINT2, TM4SF1, TMEM205, TNFRSF12A and CD47. These markers can be used to target tumor cells with adoptive cell transfer strategies (e.g., CAR T cell therapy). In prior studies, adoptive transfer of EpCAM CAR-T cells significantly delayed tumor growth and formation in xenograft models (Zhang, et al., Preclinical Evaluation of Chimeric Antigen Receptor-Modified T Cells Specific to Epithelial Cell Adhesion Molecule for Treating Colorectal Cancer, Hum Gene Ther. 2019 April; 30(4):402-412). CLDN3, CLDN7 and CLDN4 were found to have more malignant cell specificity in the single cells than any other marker identified. Adoptive cell transfer can also be administered in a combination with one or more agents capable of shifting an expression program described herein or in combination with a JAK/STAT inhibitor described herein.

As used herein, “ACT”, “adoptive cell therapy” and “adoptive cell transfer” may be used interchangeably. In certain embodiments, adoptive cell therapy (ACT) can refer to the transfer of cells to a patient with the goal of transferring the functionality and characteristics into the new host by engraftment of the cells (see, e.g., Mettananda et al., Editing an α-globin enhancer in primary human hematopoietic stem cells as a treatment for β-thalassemia, Nat Commun. 2017 Sep. 4; 8(1):424). As used herein, the term “engraft” or “engraftment” refers to the process of cell incorporation into a tissue of interest in vivo through contact with existing cells of the tissue. Adoptive cell therapy (ACT) can refer to the transfer of cells, most commonly immune-derived cells, back into the same patient or into a new recipient host with the goal of transferring the immunologic functionality and characteristics into the new host. If possible, use of autologous cells helps the recipient by minimizing graft vs host disease (GVHD) issues. The adoptive transfer of autologous tumor infiltrating lymphocytes (TILs) (Zacharakis et al., (2018) Nat Med. 2018 June; 24(6):724-730; Besser et al., (2010) Clin. Cancer Res 16 (9) 2646-55; Dudley et al., (2002) Science 298 (5594): 850-4; and Dudley et al., (2005) Journal of Clinical Oncology 23 (10): 2346-57) or genetically re-directed peripheral blood mononuclear cells (Johnson et al., (2009) Blood 114 (3): 535-46; and Morgan et al., (2006) Science 314(5796) 126-9) has been used to successfully treat patients with advanced solid tumors, including melanoma, metastatic breast cancer and colorectal carcinoma, as well as patients with CD19-expressing hematologic malignancies (Kalos et al., (2011) Science Translational Medicine 3 (95): 95ra73). In certain embodiments, allogenic cells immune cells are transferred (see, e.g., Ren et al., (2017) Clin Cancer Res 23 (9) 2255-2266). As described further herein, allogenic cells can be edited to reduce alloreactivity and prevent graft-versus-host disease. Thus, use of allogenic cells allows for cells to be obtained from healthy donors and prepared for use in patients as opposed to preparing autologous cells from a patient after diagnosis.

Aspects of the invention involve the adoptive transfer of immune system cells, such as T cells, specific for selected antigens, such as tumor associated antigens or tumor specific neoantigens (see, e.g., Maus et al., 2014, Adoptive Immunotherapy for Cancer or Viruses, Annual Review of Immunology, Vol. 32: 189-225; Rosenberg and Restifo, 2015, Adoptive cell transfer as personalized immunotherapy for human cancer, Science Vol. 348 no. 6230 pp. 62-68; Restifo et al., 2015, Adoptive immunotherapy for cancer: harnessing the T cell response. Nat. Rev. Immunol. 12(4): 269-281; and Jenson and Riddell, 2014, Design and implementation of adoptive therapy with chimeric antigen receptor-modified T cells. Immunol Rev. 257(1): 127-144; and Rajasagi et al., 2014, Systematic identification of personal tumor-specific neoantigens in chronic lymphocytic leukemia. Blood. 2014 Jul. 17; 124(3):453-62).

In certain embodiments, an antigen (such as a tumor antigen) to be targeted in adoptive cell therapy (such as particularly CAR or TCR T-cell therapy) of a disease (such as particularly of tumor or cancer) may be selected from a group consisting of: CLDN3, CLDN7, CLDN4, EPCAM, TACSTD2, MAL2, LSR, CD9, SPINT2, TM4SF1, TMEM205, TNFRSF12A and CD47.

Various strategies may for example be employed to genetically modify T cells by altering the specificity of the T cell receptor (TCR) for example by introducing new TCR α and β chains with selected peptide specificity (see U.S. Pat. No. 8,697,854; PCT Patent Publications: WO2003020763, WO2004033685, WO2004044004, WO2005114215, WO2006000830, WO2008038002, WO2008039818, WO2004074322, WO2005113595, WO2006125962, WO2013166321, WO2013039889, WO2014018863, WO2014083173; U.S. Pat. No. 8,088,379).

As an alternative to, or addition to, TCR modifications, chimeric antigen receptors (CARs) may be used in order to generate immunoresponsive cells, such as T cells, specific for selected targets, such as malignant cells, with a wide variety of receptor chimera constructs having been described (see U.S. Pat. Nos. 5,843,728; 5,851,828; 5,912,170; 6,004,811; 6,284,240; 6,392,013; 6,410,014; 6,753,162; 8,211,422; and, PCT Publication WO9215322).

In general, CARs are comprised of an extracellular domain, a transmembrane domain, and an intracellular domain, wherein the extracellular domain comprises an antigen-binding domain that is specific for a predetermined target. While the antigen-binding domain of a CAR is often an antibody or antibody fragment (e.g., a single chain variable fragment, scFv), the binding domain is not particularly limited so long as it results in specific recognition of a target. For example, in some embodiments, the antigen-binding domain may comprise a receptor, such that the CAR is capable of binding to the ligand of the receptor. Alternatively, the antigen-binding domain may comprise a ligand, such that the CAR is capable of binding the endogenous receptor of that ligand.

The antigen-binding domain of a CAR is generally separated from the transmembrane domain by a hinge or spacer. The spacer is also not particularly limited, and it is designed to provide the CAR with flexibility. For example, a spacer domain may comprise a portion of a human Fc domain, including a portion of the CH3 domain, or the hinge region of any immunoglobulin, such as IgA, IgD, IgE, IgG, or IgM, or variants thereof. Furthermore, the hinge region may be modified so as to prevent off-target binding by FcRs or other potential interfering objects. For example, the hinge may comprise an IgG4 Fc domain with or without a S228P, L235E, and/or N297Q mutation (according to Kabat numbering) in order to decrease binding to FcRs. Additional spacers/hinges include, but are not limited to, CD4, CD8, and CD28 hinge regions.

The transmembrane domain of a CAR may be derived either from a natural or from a synthetic source. Where the source is natural, the domain may be derived from any membrane bound or transmembrane protein. Transmembrane regions of particular use in this disclosure may be derived from CD8, CD28, CD3, CD45, CD4, CD5, CDS, CD9, CD 16, CD22, CD33, CD37, CD64, CD80, CD86, CD 134, CD137, CD 154, TCR. Alternatively, the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

Alternative CAR constructs may be characterized as belonging to successive generations. First-generation CARs typically consist of a single-chain variable fragment of an antibody specific for an antigen, for example comprising a VL linked to a VH of a specific antibody, linked by a flexible linker, for example by a CD8α hinge domain and a CD8α transmembrane domain, to the transmembrane and intracellular signaling domains of either CD3ζ or FcRγ (scFv-CD3ζ or scFv-FcRγ; see U.S. Pat. Nos. 7,741,465; 5,912,172; 5,906,936). Second-generation CARs incorporate the intracellular domains of one or more costimulatory molecules, such as CD28, OX40 (CD134), or 4-1BB (CD137) within the endodomain (for example scFv-CD28/OX40/4-1BB-CD3ζ; see U.S. Pat. Nos. 8,911,993; 8,916,381; 8,975,071; 9,101,584; 9,102,760; 9,102,761). Third-generation CARs include a combination of costimulatory endodomains, such a CD3ζ-chain, CD97, GDI 1a-CD18, CD2, ICOS, CD27, CD154, CDS, OX40, 4-1BB, CD2, CD7, LIGHT, LFA-1, NKG2C, B7-H3, CD30, CD40, PD-1, or CD28 signaling domains (for example scFv-CD28-4-1BB-CD3ζ or scFv-CD28-OX40-CD3ζ; see U.S. Pat. Nos. 8,906,682; 8,399,645; 5,686,281; PCT Publication No. WO2014134165; PCT Publication No. WO2012079000). In certain embodiments, the primary signaling domain comprises a functional signaling domain of a protein selected from the group consisting of CD3 zeta, CD3 gamma, CD3 delta, CD3 epsilon, common FcR gamma (FCERIG), FcR beta (Fc Epsilon Rib), CD79a, CD79b, Fc gamma RIIa, DAP10, and DAP12. In certain preferred embodiments, the primary signaling domain comprises a functional signaling domain of CD3ζ or FcRγ. In certain embodiments, the one or more costimulatory signaling domains comprise a functional signaling domain of a protein selected, each independently, from the group consisting of: CD27, CD28, 4-1BB (CD137), OX40, CD30, CD40, PD-1, ICOS, lymphocyte function-associated antigen-1 (LFA-1), CD2, CD7, LIGHT, NKG2C, B7-H3, a ligand that specifically binds with CD83, CDS, ICAM-1, GITR, BAFFR, HVEM (LIGHTR), SLAMF7, NKp80 (KLRF1), CD160, CD19, CD4, CD8 alpha, CD8 beta, IL2R beta, IL2R gamma, IL7R alpha, ITGA4, VLA1, CD49a, ITGA4, IA4, CD49D, ITGA6, VLA-6, CD49f, ITGAD, CD11d, ITGAE, CD103, ITGAL, CD11a, LFA-1, ITGAM, CD11b, ITGAX, CD11c, ITGB1, CD29, ITGB2, CD18, ITGB7, TNFR2, TRANCE/RANKL, DNAM1 (CD226), SLAMF4 (CD244, 2B4), CD84, CD96 (Tactile), CEACAM1, CRTAM, Ly9 (CD229), CD160 (BY55), PSGL1, CD100 (SEMA4D), CD69, SLAMF6 (NTB-A, Lyl08), SLAM (SLAMF1, CD150, IPO-3), BLAME (SLAMF8), SELPLG (CD162), LTBR, LAT, GADS, SLP-76, PAG/Cbp, NKp44, NKp30, NKp46, and NKG2D. In certain embodiments, the one or more costimulatory signaling domains comprise a functional signaling domain of a protein selected, each independently, from the group consisting of: 4-1BB, CD27, and CD28. In certain embodiments, a chimeric antigen receptor may have the design as described in U.S. Pat. No. 7,446,190, comprising an intracellular domain of CD3ζ chain (such as amino acid residues 52-163 of the human CD3 zeta chain, as shown in SEQ ID NO: 14 of U.S. Pat. No. 7,446,190), a signaling region from CD28 and an antigen-binding element (or portion or domain; such as scFv). The CD28 portion, when between the zeta chain portion and the antigen-binding element, may suitably include the transmembrane and signaling domains of CD28 (such as amino acid residues 114-220 of SEQ ID NO: 10, full sequence shown in SEQ ID NO: 6 of U.S. Pat. No. 7,446,190; these can include the following portion of CD28 as set forth in Genbank identifier NM_006139 (sequence version 1, 2 or 3): IEVMYPPPYLDNEKSNGTIIHVKGKHLCPSPLFPGPSKPFWVLVVVGGVLACYSLLVTVA FIIFWVRSKRSRLLHSDYMNMTPRRPGPTRKHYQPYAPPRDFAAYRS)) (SEQ ID No: 1). Alternatively, when the zeta sequence lies between the CD28 sequence and the antigen-binding element, intracellular domain of CD28 can be used alone (such as amino sequence set forth in SEQ ID NO: 9 of U.S. Pat. No. 7,446,190). Hence, certain embodiments employ a CAR comprising (a) a zeta chain portion comprising the intracellular domain of human CD3ζ chain, (b) a costimulatory signaling region, and (c) an antigen-binding element (or portion or domain), wherein the costimulatory signaling region comprises the amino acid sequence encoded by SEQ ID NO: 6 of U.S. Pat. No. 7,446,190.

Alternatively, costimulation may be orchestrated by expressing CARs in antigen-specific T cells, chosen so as to be activated and expanded following engagement of their native αβTCR, for example by antigen on professional antigen-presenting cells, with attendant costimulation. In addition, additional engineered receptors may be provided on the immunoresponsive cells, for example to improve targeting of a T-cell attack and/or minimize side effects

By means of an example and without limitation, Kochenderfer et al., (2009) J Immunother. 32 (7): 689-702 described anti-CD19 chimeric antigen receptors (CAR). FMC63-28Z CAR contained a single chain variable region moiety (scFv) recognizing CD19 derived from the FMC63 mouse hybridoma (described in Nicholson et al., (1997) Molecular Immunology 34: 1157-1165), a portion of the human CD28 molecule, and the intracellular component of the human TCR-ζ molecule. FMC63-CD828BBZ CAR contained the FMC63 scFv, the hinge and transmembrane regions of the CD8 molecule, the cytoplasmic portions of CD28 and 4-1BB, and the cytoplasmic component of the TCR-ζ molecule. The exact sequence of the CD28 molecule included in the FMC63-28Z CAR corresponded to Genbank identifier NM_006139; the sequence included all amino acids starting with the amino acid sequence IEVMYPPPY (SEQ. I.D. No. 2) and continuing all the way to the carboxy-terminus of the protein. To encode the anti-CD19 scFv component of the vector, the authors designed a DNA sequence which was based on a portion of a previously published CAR (Cooper et al., (2003) Blood 101: 1637-1644). This sequence encoded the following components in frame from the 5′ end to the 3′ end: an XhoI site, the human granulocyte-macrophage colony-stimulating factor (GM-CSF) receptor α-chain signal sequence, the FMC63 light chain variable region (as in Nicholson et al., supra), a linker peptide (as in Cooper et al., supra), the FMC63 heavy chain variable region (as in Nicholson et al., supra), and a NotI site. A plasmid encoding this sequence was digested with XhoI and NotI. To form the MSGV-FMC63-28Z retroviral vector, the XhoI and NotI-digested fragment encoding the FMC63 scFv was ligated into a second XhoI and NotI-digested fragment that encoded the MSGV retroviral backbone (as in Hughes et al., (2005) Human Gene Therapy 16: 457-472) as well as part of the extracellular portion of human CD28, the entire transmembrane and cytoplasmic portion of human CD28, and the cytoplasmic portion of the human TCR-ζ molecule (as in Maher et al., 2002) Nature Biotechnology 20: 70-75). The FMC63-28Z CAR is included in the KTE-C19 (axicabtagene ciloleucel) anti-CD19 CAR-T therapy product in development by Kite Pharma, Inc. for the treatment of inter alia patients with relapsed/refractory aggressive B-cell non-Hodgkin lymphoma (NHL). Accordingly, in certain embodiments, cells intended for adoptive cell therapies, more particularly immunoresponsive cells such as T cells, may express the FMC63-28Z CAR as described by Kochenderfer et al. (supra). Hence, in certain embodiments, cells intended for adoptive cell therapies, more particularly immunoresponsive cells such as T cells, may comprise a CAR comprising an extracellular antigen-binding element (or portion or domain; such as scFv) that specifically binds to an antigen, an intracellular signaling domain comprising an intracellular domain of a CD3ζ chain, and a costimulatory signaling region comprising a signaling domain of CD28. Preferably, the CD28 amino acid sequence is as set forth in Genbank identifier NM_006139 (sequence version 1, 2 or 3) starting with the amino acid sequence IEVMYPPPY (SEQ ID NO: 2) and continuing all the way to the carboxy-terminus of the protein. The sequence is reproduced herein:

(SEQ ID NO: 3)
IEVMYPPPYLDNEKSNGTIIHVKGKHLCPSPLFPGPSKPFWVLVVVGGVLA
CYSLLVTVAFIIFWVRSKRSRLLHSDYMNMTPRRPGPTRKHYQPYAPPRDF
AAYRS.

Additional anti-CD19 CARs are further described in WO2015187528. More particularly Example 1 and Table 1 of WO2015187528, incorporated by reference herein, demonstrate the generation of anti-CD19 CARs based on a fully human anti-CD19 monoclonal antibody (47G4, as described in US20100104509) and murine anti-CD19 monoclonal antibody (as described in Nicholson et al. and explained above). Various combinations of a signal sequence (human CD8-alpha or GM-CSF receptor), extracellular and transmembrane regions (human CD8-alpha) and intracellular T-cell signalling domains (CD28-CD3ζ; 4-1BB-CD3ζ; CD27-CD3ζ; CD28-CD27-CD3ζ, 4-1BB-CD27-CD3ζ; CD27-4-1BB-CD3ζ; CD28-CD27-FcεRI gamma chain; or CD28-FcεRI gamma chain) were disclosed. Hence, in certain embodiments, cells intended for adoptive cell therapies, more particularly immunoresponsive cells such as T cells, may comprise a CAR comprising an extracellular antigen-binding element that specifically binds to an antigen, an extracellular and transmembrane region as set forth in Table 1 of WO2015187528 and an intracellular T-cell signaling domain as set forth in Table 1 of WO2015187528.

By means of an example and without limitation, chimeric antigen receptor that recognizes the CD70 antigen is described in WO2012058460A2 (see also, Park et al., CD70 as a target for chimeric antigen receptor T cells in head and neck squamous cell carcinoma, Oral Oncol. 2018 March; 78:145-150; and Jin et al., CD70, a novel target of CAR T-cell therapy for gliomas, Neuro Oncol. 2018 Jan. 10; 20(1):55-65). CD70 is expressed by diffuse large B-cell and follicular lymphoma and also by the malignant cells of Hodgkins lymphoma, Waldenstrom's macroglobulinemia and multiple myeloma, and by HTLV-1- and EBV-associated malignancies. (Agathanggelou et al. Am. J. Pathol. 1995; 147: 1152-1160; Hunter et al., Blood 2004; 104:4881. 26; Lens et al., J Immunol. 2005; 174:6212-6219; Baba et al., J Virol. 2008; 82:3843-3852.) In addition, CD70 is expressed by non-hematological malignancies such as renal cell carcinoma and glioblastoma. (Junker et al., J Urol. 2005; 173:2150-2153; Chahlavi et al., Cancer Res 2005; 65:5428-5438) Physiologically, CD70 expression is transient and restricted to a subset of highly activated T, B, and dendritic cells.

By means of an example and without limitation, chimeric antigen receptor that recognizes BCMA has been described (see, e.g., US20160046724A1; WO2016014789A2; WO2017211900A1; WO2015158671A1; US20180085444A1; WO2018028647A1; US20170283504A1; and WO2013154760A1).

In certain embodiments, the immune cell may, in addition to a CAR or exogenous TCR as described herein, further comprise a chimeric inhibitory receptor (inhibitory CAR) that specifically binds to a second target antigen and is capable of inducing an inhibitory or immunosuppressive or repressive signal to the cell upon recognition of the second target antigen. In certain embodiments, the chimeric inhibitory receptor comprises an extracellular antigen-binding element (or portion or domain) configured to specifically bind to a target antigen, a transmembrane domain, and an intracellular immunosuppressive or repressive signaling domain. In certain embodiments, the second target antigen is an antigen that is not expressed on the surface of a cancer cell or infected cell or the expression of which is downregulated on a cancer cell or an infected cell. In certain embodiments, the second target antigen is an MHC-class I molecule. In certain embodiments, the intracellular signaling domain comprises a functional signaling portion of an immune checkpoint molecule, such as for example PD-1 or CTLA4. Advantageously, the inclusion of such inhibitory CAR reduces the chance of the engineered immune cells attacking non-target (e.g., non-cancer) tissues.

Alternatively, T-cells expressing CARs may be further modified to reduce or eliminate expression of endogenous TCRs in order to reduce off-target effects. Reduction or elimination of endogenous TCRs can reduce off-target effects and increase the effectiveness of the T cells (U.S. Pat. No. 9,181,527). T cells stably lacking expression of a functional TCR may be produced using a variety of approaches. T cells internalize, sort, and degrade the entire T cell receptor as a complex, with a half-life of about 10 hours in resting T cells and 3 hours in stimulated T cells (von Essen, M. et al. 2004. J. Immunol. 173:384-393). Proper functioning of the TCR complex requires the proper stoichiometric ratio of the proteins that compose the TCR complex. TCR function also requires two functioning TCR zeta proteins with ITAM motifs. The activation of the TCR upon engagement of its MHC-peptide ligand requires the engagement of several TCRs on the same T cell, which all must signal properly. Thus, if a TCR complex is destabilized with proteins that do not associate properly or cannot signal optimally, the T cell will not become activated sufficiently to begin a cellular response.

Accordingly, in some embodiments, TCR expression may eliminated using RNA interference (e.g., shRNA, siRNA, miRNA, etc.), CRISPR, or other methods that target the nucleic acids encoding specific TCRs (e.g., TCR-α and TCR-β) and/or CD3 chains in primary T cells. By blocking expression of one or more of these proteins, the T cell will no longer produce one or more of the key components of the TCR complex, thereby destabilizing the TCR complex and preventing cell surface expression of a functional TCR.

In some instances, CAR may also comprise a switch mechanism for controlling expression and/or activation of the CAR. For example, a CAR may comprise an extracellular, transmembrane, and intracellular domain, in which the extracellular domain comprises a target-specific binding element that comprises a label, binding domain, or tag that is specific for a molecule other than the target antigen that is expressed on or by a target cell. In such embodiments, the specificity of the CAR is provided by a second construct that comprises a target antigen binding domain (e.g., an scFv or a bispecific antibody that is specific for both the target antigen and the label or tag on the CAR) and a domain that is recognized by or binds to the label, binding domain, or tag on the CAR. See, e.g., WO 2013/044225, WO 2016/000304, WO 2015/057834, WO 2015/057852, WO 2016/070061, U.S. Pat. No. 9,233,125, US 2016/0129109. In this way, a T-cell that expresses the CAR can be administered to a subject, but the CAR cannot bind its target antigen until the second composition comprising an antigen-specific binding domain is administered.

Alternative switch mechanisms include CARs that require multimerization in order to activate their signaling function (see, e.g., US 2015/0368342, US 2016/0175359, US 2015/0368360) and/or an exogenous signal, such as a small molecule drug (US 2016/0166613, Yung et al., Science, 2015), in order to elicit a T-cell response. Some CARs may also comprise a “suicide switch” to induce cell death of the CAR T-cells following treatment (Buddee et al., PLoS One, 2013) or to downregulate expression of the CAR following binding to the target antigen (WO 2016/011210).

Alternative techniques may be used to transform target immunoresponsive cells, such as protoplast fusion, lipofection, transfection or electroporation. A wide variety of vectors may be used, such as retroviral vectors, lentiviral vectors, adenoviral vectors, adeno-associated viral vectors, plasmids or transposons, such as a Sleeping Beauty transposon (see U.S. Pat. Nos. 6,489,458; 7,148,203; 7,160,682; 7,985,739; 8,227,432), may be used to introduce CARs, for example using 2nd generation antigen-specific CARs signaling through CD3ζ and either CD28 or CD137. Viral vectors may for example include vectors based on HIV, SV40, EBV, HSV or BPV. In certain embodiments, inducible gene switches are used to regulate expression of a CAR or TCR (see, e.g., Chakravarti, Deboki et al. “Inducible Gene Switches with Memory in Human T Cells for Cellular Immunotherapy.” ACS synthetic biology vol. 8, 8 (2019): 1744-1754).

Cells that are targeted for transformation may for example include T cells, Natural Killer (NK) cells, cytotoxic T lymphocytes (CTL), regulatory T cells, human embryonic stem cells, tumor-infiltrating lymphocytes (TIL) or a pluripotent stem cell from which lymphoid cells may be differentiated. T cells expressing a desired CAR may for example be selected through co-culture with γ-irradiated activating and propagating cells (AaPC), which co-express the cancer antigen and co-stimulatory molecules. The engineered CAR T-cells may be expanded, for example by co-culture on AaPC in presence of soluble factors, such as IL-2 and IL-21. This expansion may for example be carried out so as to provide memory CAR+ T cells (which may for example be assayed by non-enzymatic digital array and/or multi-panel flow cytometry). In this way, CAR T cells may be provided that have specific cytotoxic activity against antigen-bearing tumors (optionally in conjunction with production of desired chemokines such as interferon-γ). CAR T cells of this kind may for example be used in animal models, for example to treat tumor xenografts.

In certain embodiments, ACT includes co-transferring CD4+ Th1 cells and CD8+ CTLs to induce a synergistic antitumour response (see, e.g., Li et al., Adoptive cell therapy with CD4+ T helper 1 cells and CD8+ cytotoxic T cells enhances complete rejection of an established tumour, leading to generation of endogenous memory responses to non-targeted tumour epitopes. Clin Transl Immunology. 2017 October; 6(10): e160).

In certain embodiments, Th17 cells are transferred to a subject in need thereof. Th17 cells have been reported to directly eradicate melanoma tumors in mice to a greater extent than Th1 cells (Muranski P, et al., Tumor-specific Th17-polarized cells eradicate large established melanoma. Blood. 2008 Jul. 15; 112(2):362-73; and Martin-Orozco N, et al., T helper 17 cells promote cytotoxic T cell activation in tumor immunity. Immunity. 2009 Nov. 20; 31(5):787-98). Those studies involved an adoptive T cell transfer (ACT) therapy approach, which takes advantage of CD4⁺ T cells that express a TCR recognizing tyrosinase tumor antigen. Exploitation of the TCR leads to rapid expansion of Th17 populations to large numbers ex vivo for reinfusion into the autologous tumor-bearing hosts.

In certain embodiments, ACT may include autologous iPSC-based vaccines, such as irradiated iPSCs in autologous anti-tumor vaccines (see e.g., Kooreman, Nigel G. et al., Autologous iPSC-Based Vaccines Elicit Anti-tumor Responses In Vivo, Cell Stem Cell 22, 1-13, 2018, doi.org/10.1016/j.stem.2018.01.016).

Unlike T-cell receptors (TCRs) that are MHC restricted, CARs can potentially bind any cell surface-expressed antigen and can thus be more universally used to treat patients (see Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267). In certain embodiments, in the absence of endogenous T-cell infiltrate (e.g., due to aberrant antigen processing and presentation), which precludes the use of TIL therapy and immune checkpoint blockade, the transfer of CAR T-cells may be used to treat patients (see, e.g., Hinrichs C S, Rosenberg S A. Exploiting the curative potential of adoptive T-cell therapy for cancer. Immunol Rev (2014) 257(1):56-71. doi:10.1111/imr.12132).

Approaches such as the foregoing may be adapted to provide methods of treating and/or increasing survival of a subject having a disease, such as a neoplasia, for example by administering an effective amount of an immunoresponsive cell comprising an antigen recognizing receptor that binds a selected antigen, wherein the binding activates the immunoresponsive cell, thereby treating or preventing the disease (such as a neoplasia, a pathogen infection, an autoimmune disorder, or an allogeneic transplant reaction).

In certain embodiments, the treatment can be administered after lymphodepleting pretreatment in the form of chemotherapy (typically a combination of cyclophosphamide and fludarabine) or radiation therapy. Initial studies in ACT had short lived responses and the transferred cells did not persist in vivo for very long (Houot et al., T-cell-based immunotherapy: adoptive cell transfer and checkpoint inhibition. Cancer Immunol Res (2015) 3(10):1115-22; and Kamta et al., Advancing Cancer Therapy with Present and Emerging Immuno-Oncology Approaches. Front. Oncol. (2017) 7:64). Immune suppressor cells like Tregs and MDSCs may attenuate the activity of transferred cells by outcompeting them for the necessary cytokines. Not being bound by a theory lymphodepleting pretreatment may eliminate the suppressor cells allowing the TILs to persist.

In one embodiment, the treatment can be administrated into patients undergoing an immunosuppressive treatment (e.g., glucocorticoid treatment). The cells or population of cells, may be made resistant to at least one immunosuppressive agent due to the inactivation of a gene encoding a receptor for such immunosuppressive agent. In certain embodiments, the immunosuppressive treatment provides for the selection and expansion of the immunoresponsive T cells within the patient.

In certain embodiments, the treatment can be administered before primary treatment (e.g., surgery or radiation therapy) to shrink a tumor before the primary treatment. In another embodiment, the treatment can be administered after primary treatment to remove any remaining cancer cells.

In certain embodiments, immunometabolic barriers can be targeted therapeutically prior to and/or during ACT to enhance responses to ACT or CAR T-cell therapy and to support endogenous immunity (see, e.g., Irving et al., Engineering Chimeric Antigen Receptor T-Cells for Racing in Solid Tumors: Don't Forget the Fuel, Front. Immunol., 3 Apr. 2017, doi.org/10.3389/fimmu.2017.00267).

The administration of cells or population of cells, such as immune system cells or cell populations, such as more particularly immunoresponsive cells or cell populations, as disclosed herein may be carried out in any convenient manner, including by aerosol inhalation, injection, ingestion, transfusion, implantation or transplantation. The cells or population of cells may be administered to a patient subcutaneously, intradermally, intratumorally, intranodally, intramedullary, intramuscularly, intrathecally, by intravenous or intralymphatic injection, or intraperitoneally. In some embodiments, the disclosed CARs may be delivered or administered into a cavity formed by the resection of tumor tissue (i.e. intracavity delivery) or directly into a tumor prior to resection (i.e. intratumoral delivery). In one embodiment, the cell compositions of the present invention are preferably administered by intravenous injection.

The administration of the cells or population of cells can consist of the administration of 10⁴-10⁹ cells per kg body weight, preferably 10⁵ to 10⁶ cells/kg body weight including all integer values of cell numbers within those ranges. Dosing in CAR T cell therapies may for example involve administration of from 10⁶ to 10⁹ cells/kg, with or without a course of lymphodepletion, for example with cyclophosphamide. The cells or population of cells can be administrated in one or more doses. In another embodiment, the effective amount of cells are administrated as a single dose. In another embodiment, the effective amount of cells are administrated as more than one dose over a period time. Timing of administration is within the judgment of managing physician and depends on the clinical condition of the patient. The cells or population of cells may be obtained from any source, such as a blood bank or a donor. While individual needs vary, determination of optimal ranges of effective amounts of a given cell type for a particular disease or conditions are within the skill of one in the art. An effective amount means an amount which provides a therapeutic or prophylactic benefit. The dosage administrated will be dependent upon the age, health and weight of the recipient, kind of concurrent treatment, if any, frequency of treatment and the nature of the effect desired.

In another embodiment, the effective amount of cells or composition comprising those cells are administrated parenterally. The administration can be an intravenous administration. The administration can be directly done by injection within a tumor.

To guard against possible adverse reactions, engineered immunoresponsive cells may be equipped with a transgenic safety switch, in the form of a transgene that renders the cells vulnerable to exposure to a specific signal. For example, the herpes simplex viral thymidine kinase (TK) gene may be used in this way, for example by introduction into allogeneic T lymphocytes used as donor lymphocyte infusions following stem cell transplantation (Greco, et al., Improving the safety of cell therapy with the TK-suicide gene. Front. Pharmacol. 2015; 6: 95). In such cells, administration of a nucleoside prodrug such as ganciclovir or acyclovir causes cell death. Alternative safety switch constructs include inducible caspase 9, for example triggered by administration of a small-molecule dimerizer that brings together two nonfunctional icasp9 molecules to form the active enzyme. A wide variety of alternative approaches to implementing cellular proliferation controls have been described (see U.S. Patent Publication No. 20130071414; PCT Patent Publication WO2011146862; PCT Patent Publication WO2014011987; PCT Patent Publication WO2013040371; Zhou et al. BLOOD, 2014, 123/25:3895-3905; Di Stasi et al., The New England Journal of Medicine 2011; 365:1673-1683; Sadelain M, The New England Journal of Medicine 2011; 365:1735-173; Ramos et al., Stem Cells 28(6):1107-15 (2010)).

In a further refinement of adoptive therapies, genome editing may be used to tailor immunoresponsive cells to alternative implementations, for example providing edited CAR T cells (see Poirot et al., 2015, Multiplex genome edited T-cell manufacturing platform for “off-the-shelf” adoptive T-cell immunotherapies, Cancer Res 75 (18): 3853; Ren et al., 2017, Multiplex genome editing to generate universal CAR T cells resistant to PD1 inhibition, Clin Cancer Res. 2017 May 1; 23(9):2255-2266. doi: 10.1158/1078-0432.CCR-16-1300. Epub 2016 Nov. 4; Qasim et al., 2017, Molecular remission of infant B-ALL after infusion of universal TALEN gene-edited CAR T cells, Sci Transl Med. 2017 Jan. 25; 9(374); Legut, et al., 2018, CRISPR-mediated TCR replacement generates superior anticancer transgenic T cells. Blood, 131(3), 311-322; Georgiadis et al., Long Terminal Repeat CRISPR-CAR-Coupled “Universal” T Cells Mediate Potent Anti-leukemic Effects, Molecular Therapy, In Press, Corrected Proof, Available online 6 Mar. 2018; and Roth, T. L. Editing of Endogenous Genes in Cellular Immunotherapies. Curr Hematol Malig Rep 15, 235-240 (2020)). Cells may be edited using any CRISPR system and method of use thereof as described herein. CRISPR systems may be delivered to an immune cell by any method described herein. In preferred embodiments, cells are edited ex vivo and transferred to a subject in need thereof. Immunoresponsive cells, CAR T cells or any cells used for adoptive cell transfer may be edited. Editing may be performed for example to insert or knock-in an exogenous gene, such as an exogenous gene encoding a CAR or a TCR, at a preselected locus in a cell (e.g. TRAC locus); to eliminate potential alloreactive T-cell receptors (TCR) or to prevent inappropriate pairing between endogenous and exogenous TCR chains, such as to knock-out or knock-down expression of an endogenous TCR in a cell; to disrupt the target of a chemotherapeutic agent in a cell; to block an immune checkpoint, such as to knock-out or knock-down expression of an immune checkpoint protein or receptor in a cell; to knock-out or knock-down expression of other gene or genes in a cell, the reduced expression or lack of expression of which can enhance the efficacy of adoptive therapies using the cell; to knock-out or knock-down expression of an endogenous gene in a cell, said endogenous gene encoding an antigen targeted by an exogenous CAR or TCR; to knock-out or knock-down expression of one or more MHC constituent proteins in a cell; to activate a T cell; to modulate cells such that the cells are resistant to exhaustion or dysfunction; and/or increase the differentiation and/or proliferation of functionally exhausted or dysfunctional CD8+ T-cells (see PCT Patent Publications: WO2013176915, WO2014059173, WO2014172606, WO2014184744, and WO2014191128).

In certain embodiments, editing may result in inactivation of a gene. By inactivating a gene, it is intended that the gene of interest is not expressed in a functional protein form. In a particular embodiment, the CRISPR system specifically catalyzes cleavage in one targeted gene thereby inactivating said targeted gene. The nucleic acid strand breaks caused are commonly repaired through the distinct mechanisms of homologous recombination or non-homologous end joining (NHEJ). However, NHEJ is an imperfect repair process that often results in changes to the DNA sequence at the site of the cleavage. Repair via non-homologous end joining (NHEJ) often results in small insertions or deletions (Indel) and can be used for the creation of specific gene knockouts. Cells in which a cleavage induced mutagenesis event has occurred can be identified and/or selected by well-known methods in the art. In certain embodiments, homology directed repair (HDR) is used to concurrently inactivate a gene (e.g., TRAC) and insert an endogenous TCR or CAR into the inactivated locus.

Hence, in certain embodiments, editing of cells (such as by CRISPR/Cas), particularly cells intended for adoptive cell therapies, more particularly immunoresponsive cells such as T cells, may be performed to insert or knock-in an exogenous gene, such as an exogenous gene encoding a CAR or a TCR, at a preselected locus in a cell. Conventionally, nucleic acid molecules encoding CARs or TCRs are transfected or transduced to cells using randomly integrating vectors, which, depending on the site of integration, may lead to clonal expansion, oncogenic transformation, variegated transgene expression and/or transcriptional silencing of the transgene. Directing of transgene(s) to a specific locus in a cell can minimize or avoid such risks and advantageously provide for uniform expression of the transgene(s) by the cells. Without limitation, suitable ‘safe harbor’ loci for directed transgene integration include CCR5 or AAVS1. Homology-directed repair (HDR) strategies are known and described elsewhere in this specification allowing to insert transgenes into desired loci (e.g., TRAC locus).

Further suitable loci for insertion of transgenes, in particular CAR or exogenous TCR transgenes, include without limitation loci comprising genes coding for constituents of endogenous T-cell receptor, such as T-cell receptor alpha locus (TRA) or T-cell receptor beta locus (TRB), for example T-cell receptor alpha constant (TRAC) locus, T-cell receptor beta constant 1 (TRBC1) locus or T-cell receptor beta constant 2 (TRBC1) locus. Advantageously, insertion of a transgene into such locus can simultaneously achieve expression of the transgene, potentially controlled by the endogenous promoter, and knock-out expression of the endogenous TCR. This approach has been exemplified in Eyquem et al., (2017) Nature 543: 113-117, wherein the authors used CRISPR/Cas9 gene editing to knock-in a DNA molecule encoding a CD19-specific CAR into the TRAC locus downstream of the endogenous promoter; the CAR-T cells obtained by CRISPR were significantly superior in terms of reduced tonic CAR signaling and exhaustion.

T cell receptors (TCR) are cell surface receptors that participate in the activation of T cells in response to the presentation of antigen. The TCR is generally made from two chains, α and β, which assemble to form a heterodimer and associates with the CD3-transducing subunits to form the T cell receptor complex present on the cell surface. Each α and β chain of the TCR consists of an immunoglobulin-like N-terminal variable (V) and constant (C) region, a hydrophobic transmembrane domain, and a short cytoplasmic region. As for immunoglobulin molecules, the variable region of the α and β chains are generated by V(D)J recombination, creating a large diversity of antigen specificities within the population of T cells. However, in contrast to immunoglobulins that recognize intact antigen, T cells are activated by processed peptide fragments in association with an MHC molecule, introducing an extra dimension to antigen recognition by T cells, known as MHC restriction. Recognition of MHC disparities between the donor and recipient through the T cell receptor leads to T cell proliferation and the potential development of graft versus host disease (GVHD). The inactivation of TCRα or TCRβ can result in the elimination of the TCR from the surface of T cells preventing recognition of alloantigen and thus GVHD. However, TCR disruption generally results in the elimination of the CD3 signaling component and alters the means of further T cell expansion.

Hence, in certain embodiments, editing of cells (such as by CRISPR/Cas), particularly cells intended for adoptive cell therapies, more particularly immunoresponsive cells such as T cells, may be performed to knock-out or knock-down expression of an endogenous TCR in a cell. For example, NHEJ-based or HDR-based gene editing approaches can be employed to disrupt the endogenous TCR alpha and/or beta chain genes. For example, gene editing system or systems, such as CRISPR/Cas system or systems, can be designed to target a sequence found within the TCR beta chain conserved between the beta 1 and beta 2 constant region genes (TRBC1 and TRBC2) and/or to target the constant region of the TCR alpha chain (TRAC) gene.

Allogeneic cells are rapidly rejected by the host immune system. It has been demonstrated that, allogeneic leukocytes present in non-irradiated blood products will persist for no more than 5 to 6 days (Boni, Muranski et al. 2008 Blood 1; 112(12):4746-54). Thus, to prevent rejection of allogeneic cells, the host's immune system usually has to be suppressed to some extent. However, in the case of adoptive cell transfer the use of immunosuppressive drugs also have a detrimental effect on the introduced therapeutic T cells. Therefore, to effectively use an adoptive immunotherapy approach in these conditions, the introduced cells would need to be resistant to the immunosuppressive treatment. Thus, in a particular embodiment, the present invention further comprises a step of modifying T cells to make them resistant to an immunosuppressive agent, preferably by inactivating at least one gene encoding a target for an immunosuppressive agent. An immunosuppressive agent is an agent that suppresses immune function by one of several mechanisms of action. An immunosuppressive agent can be, but is not limited to a calcineurin inhibitor, a target of rapamycin, an interleukin-2 receptor α-chain blocker, an inhibitor of inosine monophosphate dehydrogenase, an inhibitor of dihydrofolic acid reductase, a corticosteroid or an immunosuppressive antimetabolite. The present invention allows conferring immunosuppressive resistance to T cells for immunotherapy by inactivating the target of the immunosuppressive agent in T cells. As non-limiting examples, targets for an immunosuppressive agent can be a receptor for an immunosuppressive agent such as: CD52, glucocorticoid receptor (GR), a FKBP family gene member and a cyclophilin family gene member.

In certain embodiments, editing of cells (such as by CRISPR/Cas), particularly cells intended for adoptive cell therapies, more particularly immunoresponsive cells such as T cells, may be performed to block an immune checkpoint, such as to knock-out or knock-down expression of an immune checkpoint protein or receptor in a cell. Immune checkpoints are inhibitory pathways that slow down or stop immune reactions and prevent excessive tissue damage from uncontrolled activity of immune cells. In certain embodiments, the immune checkpoint targeted is the programmed death-1 (PD-1 or CD279) gene (PDCD1) (see, e.g., Rupp U, Schumann K, Roybal K T, et al. CRISPR/Cas9-mediated PD-1 disruption enhances anti-tumor efficacy of human chimeric antigen receptor T cells. Sci Rep. 2017; 7(1):737). In other embodiments, the immune checkpoint targeted is cytotoxic T-lymphocyte-associated antigen (CTLA-4). In additional embodiments, the immune checkpoint targeted is another member of the CD28 and CTLA4 Ig superfamily such as BTLA, LAG3, ICOS, PDL1 or KIR. In further additional embodiments, the immune checkpoint targeted is a member of the TNFR superfamily such as CD40, OX40, CD137, GITR, CD27 or TIM-3.

Additional immune checkpoints include Src homology 2 domain-containing protein tyrosine phosphatase 1 (SIP-1) (Watson H A, et al., SHP-1: the next checkpoint target for cancer immunotherapy? Biochem Soc Trans. 2016 Apr. 15; 44(2):356-62). SHP-1 is a widely expressed inhibitory protein tyrosine phosphatase (PTP). In T-cells, it is a negative regulator of antigen-dependent activation and proliferation. It is a cytosolic protein, and therefore not amenable to antibody-mediated therapies, but its role in activation and proliferation makes it an attractive target for genetic manipulation in adoptive transfer strategies, such as chimeric antigen receptor (CAR) T cells. Immune checkpoints may also include T cell immunoreceptor with Ig and ITIM domains (TIGIT/Vstm3/WUCAM/VSIG9) and VISTA (Le Mercier I, et al., (2015) Beyond CTLA-4 and PD-1, the generation Z of negative checkpoint regulators. Front. Immunol. 6:418).

WO2014172606 relates to the use of MT1 and/or MT2 inhibitors to increase proliferation and/or activity of exhausted CD8+ T-cells and to decrease CD8+ T-cell exhaustion (e.g., decrease functionally exhausted or unresponsive CD8+ immune cells). In certain embodiments, metallothioneins are targeted by gene editing in adoptively transferred T cells.

In certain embodiments, targets of gene editing may be at least one targeted locus involved in the expression of an immune checkpoint protein. Such targets may include, but are not limited to CTLA4, PPP2CA, PPP2CB, PTPN6, PTPN22, PDCD1, ICOS (CD278), PDL1, KIR, LAG3, HAVCR2, BTLA, CD160, TIGIT, CD96, CRTAM, LAIR1, SIGLEC7, SIGLEC9, CD244 (2B4), TNFRSF10B, TNFRSF10A, CASP8, CASP10, CASP3, CASP6, CASP7, FADD, FAS, TGFBRII, TGFRBRI, SMAD2, SMAD3, SMAD4, SMAD10, SKI, SKIL, TGIF1, IL10RA, IL10RB, HMOX2, IL6R, IL6ST, EIF2AK4, CSK, PAG1, SIT1, FOXP3, PRDM1, BATF, VISTA, GUCY1A2, GUCY1A3, GUCY1B2, GUCY1B3, MT1, MT2, CD40, OX40, CD137, GITR, CD27, SHP-1, TIM-3, CEACAM-1, CEACAM-3, or CEACAM-5. In preferred embodiments, the gene locus involved in the expression of PD-1 or CTLA-4 genes is targeted. In other preferred embodiments, combinations of genes are targeted, such as but not limited to PD-1 and TIGIT.

By means of an example and without limitation, WO2016196388 concerns an engineered T cell comprising (a) a genetically engineered antigen receptor that specifically binds to an antigen, which receptor may be a CAR; and (b) a disrupted gene encoding a PD-L1, an agent for disruption of a gene encoding a PD-L1, and/or disruption of a gene encoding PD-L1, wherein the disruption of the gene may be mediated by a gene editing nuclease, a zinc finger nuclease (ZFN), CRISPR/Cas9 and/or TALEN. WO2015142675 relates to immune effector cells comprising a CAR in combination with an agent (such as CRISPR, TALEN or ZFN) that increases the efficacy of the immune effector cells in the treatment of cancer, wherein the agent may inhibit an immune inhibitory molecule, such as PD1, PD-L1, CTLA-4, TIM-3, LAG-3, VISTA, BTLA, TIGIT, LAIR1, CD160, 2B4, TGFR beta, CEACAM-1, CEACAM-3, or CEACAM-5. Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In certain embodiments, cells may be engineered to express a CAR, wherein expression and/or function of methylcytosine dioxygenase genes (TET1, TET2 and/or TET3) in the cells has been reduced or eliminated, such as by CRISPR, ZNF or TALEN (for example, as described in WO201704916).

In certain embodiments, editing of cells (such as by CRISPR/Cas), particularly cells intended for adoptive cell therapies, more particularly immunoresponsive cells such as T cells, may be performed to knock-out or knock-down expression of an endogenous gene in a cell, said endogenous gene encoding an antigen targeted by an exogenous CAR or TCR, thereby reducing the likelihood of targeting of the engineered cells. In certain embodiments, the targeted antigen may be one or more antigen selected from the group consisting of CD38, CD138, CS-1, CD33, CD26, CD30, CD53, CD92, CD100, CD148, CD150, CD200, CD261, CD262, CD362, human telomerase reverse transcriptase (hTERT), survivin, mouse double minute 2 homolog (MDM2), cytochrome P450 1B1 (CYP1B), HER2/neu, Wilms' tumor gene 1 (WT1), livin, alphafetoprotein (AFP), carcinoembryonic antigen (CEA), mucin 16 (MUC16), MUC1, prostate-specific membrane antigen (PSMA), p53, cyclin (D1), B cell maturation antigen (BCMA), transmembrane activator and CAML Interactor (TACI), and B-cell activating factor receptor (BAFF-R) (for example, as described in WO2016011210 and WO2017011804).

In certain embodiments, editing of cells (such as by CRISPR/Cas), particularly cells intended for adoptive cell therapies, more particularly immunoresponsive cells such as T cells, may be performed to knock-out or knock-down expression of one or more MHC constituent proteins, such as one or more HLA proteins and/or beta-2 microglobulin (B2M), in a cell, whereby rejection of non-autologous (e.g., allogeneic) cells by the recipient's immune system can be reduced or avoided. In preferred embodiments, one or more HLA class I proteins, such as HLA-A, B and/or C, and/or B2M may be knocked-out or knocked-down. Preferably, B2M may be knocked-out or knocked-down. By means of an example, Ren et al., (2017) Clin Cancer Res 23 (9) 2255-2266 performed lentiviral delivery of CAR and electro-transfer of Cas9 mRNA and gRNAs targeting endogenous TCR, β-2 microglobulin (B2M) and PD1 simultaneously, to generate gene-disrupted allogeneic CAR T cells deficient of TCR, HLA class I molecule and PD1.

In other embodiments, at least two genes are edited. Pairs of genes may include, but are not limited to PD1 and TCRα, PD1 and TCRβ, CTLA-4 and TCRα, CTLA-4 and TCRβ, LAG3 and TCRα, LAG3 and TCRβ, Tim3 and TCRα, Tim3 and TCRβ, BTLA and TCRα, BTLA and TCRβ, BY55 and TCRα, BY55 and TCRβ, TIGIT and TCRα, TIGIT and TCRβ, B7H5 and TCRα, B7H5 and TCRβ, LAIR1 and TCRα, LAIR1 and TCRβ, SIGLEC10 and TCRα, SIGLEC10 and TCRβ, 2B4 and TCRα, 2B4 and TCRβ, B2M and TCRα, B2M and TCRβ.

In certain embodiments, a cell may be multiply edited (multiplex genome editing) as taught herein to (1) knock-out or knock-down expression of an endogenous TCR (for example, TRBC1, TRBC2 and/or TRAC), (2) knock-out or knock-down expression of an immune checkpoint protein or receptor (for example PD1, PD-L1 and/or CTLA4); and (3) knock-out or knock-down expression of one or more MHC constituent proteins (for example, HLA-A, B and/or C, and/or B2M, preferably B2M).

Whether prior to or after genetic modification of the T cells, the T cells can be activated and expanded generally using methods as described, for example, in U.S. Pat. Nos. 6,352,694; 6,534,055; 6,905,680; 5,858,358; 6,887,466; 6,905,681; 7,144,575; 7,232,566; 7,175,843; 5,883,223; 6,905,874; 6,797,514; 6,867,041; and 7,572,631. T cells can be expanded in vitro or in vivo.

Immune cells may be obtained using any method known in the art. In one embodiment, allogenic T cells may be obtained from healthy subjects. In one embodiment T cells that have infiltrated a tumor are isolated. T cells may be removed during surgery. T cells may be isolated after removal of tumor tissue by biopsy. T cells may be isolated by any means known in the art. In one embodiment, T cells are obtained by apheresis. In one embodiment, the method may comprise obtaining a bulk population of T cells from a tumor sample by any suitable method known in the art. For example, a bulk population of T cells can be obtained from a tumor sample by dissociating the tumor sample into a cell suspension from which specific cell populations can be selected. Suitable methods of obtaining a bulk population of T cells may include, but are not limited to, any one or more of mechanically dissociating (e.g., mincing) the tumor, enzymatically dissociating (e.g., digesting) the tumor, and aspiration (e.g., as with a needle).

The bulk population of T cells obtained from a tumor sample may comprise any suitable type of T cell. Preferably, the bulk population of T cells obtained from a tumor sample comprises tumor infiltrating lymphocytes (TILs).

The tumor sample may be obtained from any mammal. Unless stated otherwise, as used herein, the term “mammal” refers to any mammal including, but not limited to, mammals of the order Logomorpha, such as rabbits; the order Carnivora, including Felines (cats) and Canines (dogs); the order Artiodactyla, including Bovines (cows) and Swines (pigs); or of the order Perssodactyla, including Equines (horses). The mammals may be non-human primates, e.g., of the order Primates, Ceboids, or Simoids (monkeys) or of the order Anthropoids (humans and apes). In some embodiments, the mammal may be a mammal of the order Rodentia, such as mice and hamsters. Preferably, the mammal is a non-human primate or a human. An especially preferred mammal is the human.

T cells can be obtained from a number of sources, including peripheral blood mononuclear cells (PBMC), bone marrow, lymph node tissue, spleen tissue, and tumors. In certain embodiments of the present invention, T cells can be obtained from a unit of blood collected from a subject using any number of techniques known to the skilled artisan, such as Ficoll separation. In one preferred embodiment, cells from the circulating blood of an individual are obtained by apheresis or leukapheresis. The apheresis product typically contains lymphocytes, including T cells, monocytes, granulocytes, B cells, other nucleated white blood cells, red blood cells, and platelets. In one embodiment, the cells collected by apheresis may be washed to remove the plasma fraction and to place the cells in an appropriate buffer or media for subsequent processing steps. In one embodiment of the invention, the cells are washed with phosphate buffered saline (PBS). In an alternative embodiment, the wash solution lacks calcium and may lack magnesium or may lack many if not all divalent cations. Initial activation steps in the absence of calcium lead to magnified activation. As those of ordinary skill in the art would readily appreciate a washing step may be accomplished by methods known to those in the art, such as by using a semi-automated “flow-through” centrifuge (for example, the Cobe 2991 cell processor) according to the manufacturer's instructions. After washing, the cells may be resuspended in a variety of biocompatible buffers, such as, for example, Ca-free, Mg-free PBS. Alternatively, the undesirable components of the apheresis sample may be removed and the cells directly resuspended in culture media.

In another embodiment, T cells are isolated from peripheral blood lymphocytes by lysing the red blood cells and depleting the monocytes, for example, by centrifugation through a PERCOLL™ gradient. A specific subpopulation of T cells, such as CD28+, CD4+, CDC, CD45RA+, and CD45RO+ T cells, can be further isolated by positive or negative selection techniques. For example, in one preferred embodiment, T cells are isolated by incubation with anti-CD3/anti-CD28 (i.e., 3×28)-conjugated beads, such as DYNABEADS® M-450 CD3/CD28 T, or XCYTE DYNABEADS™ for a time period sufficient for positive selection of the desired T cells. In one embodiment, the time period is about 30 minutes. In a further embodiment, the time period ranges from 30 minutes to 36 hours or longer and all integer values there between. In a further embodiment, the time period is at least 1, 2, 3, 4, 5, or 6 hours. In yet another preferred embodiment, the time period is 10 to 24 hours. In one preferred embodiment, the incubation time period is 24 hours. For isolation of T cells from patients with leukemia, use of longer incubation times, such as 24 hours, can increase cell yield. Longer incubation times may be used to isolate T cells in any situation where there are few T cells as compared to other cell types, such in isolating tumor infiltrating lymphocytes (TIL) from tumor tissue or from immunocompromised individuals. Further, use of longer incubation times can increase the efficiency of capture of CD8+ T cells.

Enrichment of a T cell population by negative selection can be accomplished with a combination of antibodies directed to surface markers unique to the negatively selected cells. A preferred method is cell sorting and/or selection via negative magnetic immunoadherence or flow cytometry that uses a cocktail of monoclonal antibodies directed to cell surface markers present on the cells negatively selected. For example, to enrich for CD4+ cells by negative selection, a monoclonal antibody cocktail typically includes antibodies to CD14, CD20, CD11b, CD16, HLA-DR, and CD8.

Further, monocyte populations (i.e., CD14+ cells) may be depleted from blood preparations by a variety of methodologies, including anti-CD14 coated beads or columns, or utilization of the phagocytotic activity of these cells to facilitate removal. Accordingly, in one embodiment, the invention uses paramagnetic particles of a size sufficient to be engulfed by phagocytotic monocytes. In certain embodiments, the paramagnetic particles are commercially available beads, for example, those produced by Life Technologies under the trade name Dynabeads™. In one embodiment, other non-specific cells are removed by coating the paramagnetic particles with “irrelevant” proteins (e.g., serum proteins or antibodies). Irrelevant proteins and antibodies include those proteins and antibodies or fragments thereof that do not specifically target the T cells to be isolated. In certain embodiments, the irrelevant beads include beads coated with sheep anti-mouse antibodies, goat anti-mouse antibodies, and human serum albumin.

In brief, such depletion of monocytes is performed by preincubating T cells isolated from whole blood, apheresed peripheral blood, or tumors with one or more varieties of irrelevant or non-antibody coupled paramagnetic particles at any amount that allows for removal of monocytes (approximately a 20:1 bead:cell ratio) for about 30 minutes to 2 hours at 22 to 37 degrees C., followed by magnetic removal of cells which have attached to or engulfed the paramagnetic particles. Such separation can be performed using standard methods available in the art. For example, any magnetic separation methodology may be used including a variety of which are commercially available, (e.g., DYNAL® Magnetic Particle Concentrator (DYNAL MPC®)). Assurance of requisite depletion can be monitored by a variety of methodologies known to those of ordinary skill in the art, including flow cytometric analysis of CD14 positive cells, before and after depletion.

For isolation of a desired population of cells by positive or negative selection, the concentration of cells and surface (e.g., particles such as beads) can be varied. In certain embodiments, it may be desirable to significantly decrease the volume in which beads and cells are mixed together (i.e., increase the concentration of cells), to ensure maximum contact of cells and beads. For example, in one embodiment, a concentration of 2 billion cells/ml is used. In one embodiment, a concentration of 1 billion cells/ml is used. In a further embodiment, greater than 100 million cells/ml is used. In a further embodiment, a concentration of cells of 10, 15, 20, 25, 30, 35, 40, 45, or 50 million cells/ml is used. In yet another embodiment, a concentration of cells from 75, 80, 85, 90, 95, or 100 million cells/ml is used. In further embodiments, concentrations of 125 or 150 million cells/ml can be used. Using high concentrations can result in increased cell yield, cell activation, and cell expansion. Further, use of high cell concentrations allows more efficient capture of cells that may weakly express target antigens of interest, such as CD28-negative T cells, or from samples where there are many tumor cells present (i.e., leukemic blood, tumor tissue, etc). Such populations of cells may have therapeutic value and would be desirable to obtain. For example, using high concentration of cells allows more efficient selection of CD8+ T cells that normally have weaker CD28 expression.

In a related embodiment, it may be desirable to use lower concentrations of cells. By significantly diluting the mixture of T cells and surface (e.g., particles such as beads), interactions between the particles and cells is minimized. This selects for cells that express high amounts of desired antigens to be bound to the particles. For example, CD4+ T cells express higher levels of CD28 and are more efficiently captured than CD8+ T cells in dilute concentrations. In one embodiment, the concentration of cells used is 5×10⁶/ml. In other embodiments, the concentration used can be from about 1×10⁵/ml to 1×10⁶/ml, and any integer value in between.

T cells can also be frozen. Wishing not to be bound by theory, the freeze and subsequent thaw step provides a more uniform product by removing granulocytes and to some extent monocytes in the cell population. After a washing step to remove plasma and platelets, the cells may be suspended in a freezing solution. While many freezing solutions and parameters are known in the art and will be useful in this context, one method involves using PBS containing 20% DMSO and 8% human serum albumin, or other suitable cell freezing media, the cells then are frozen to −80° C. at a rate of 1° per minute and stored in the vapor phase of a liquid nitrogen storage tank. Other methods of controlled freezing may be used as well as uncontrolled freezing immediately at −20° C. or in liquid nitrogen.

T cells for use in the present invention may also be antigen-specific T cells. For example, tumor-specific T cells can be used. In certain embodiments, antigen-specific T cells can be isolated from a patient of interest, such as a patient afflicted with a cancer or an infectious disease. In one embodiment, neoepitopes are determined for a subject and T cells specific to these antigens are isolated. Antigen-specific cells for use in expansion may also be generated in vitro using any number of methods known in the art, for example, as described in U.S. Patent Publication No. US 20040224402 entitled, Generation and Isolation of Antigen-Specific T Cells, or in U.S. Pat. No. 6,040,177. Antigen-specific cells for use in the present invention may also be generated using any number of methods known in the art, for example, as described in Current Protocols in Immunology, or Current Protocols in Cell Biology, both published by John Wiley & Sons, Inc., Boston, Mass.

In a related embodiment, it may be desirable to sort or otherwise positively select (e.g. via magnetic selection) the antigen specific cells prior to or following one or two rounds of expansion. Sorting or positively selecting antigen-specific cells can be carried out using peptide-MHC tetramers (Altman, et al., Science. 1996 Oct. 4; 274(5284):94-6). In another embodiment, the adaptable tetramer technology approach is used (Andersen et al., 2012 Nat Protoc. 7:891-902). Tetramers are limited by the need to utilize predicted binding peptides based on prior hypotheses, and the restriction to specific HLAs. Peptide-MHC tetramers can be generated using techniques known in the art and can be made with any MHC molecule of interest and any antigen of interest as described herein. Specific epitopes to be used in this context can be identified using numerous assays known in the art. For example, the ability of a polypeptide to bind to MHC class I may be evaluated indirectly by monitoring the ability to promote incorporation of ¹²⁵I labeled β2-microglobulin (β2m) into MHC class I/β2m/peptide heterotrimeric complexes (see Parker et al., J. Immunol. 152:163, 1994).

In one embodiment cells are directly labeled with an epitope-specific reagent for isolation by flow cytometry followed by characterization of phenotype and TCRs. In one embodiment, T cells are isolated by contacting with T cell specific antibodies. Sorting of antigen-specific T cells, or generally any cells of the present invention, can be carried out using any of a variety of commercially available cell sorters, including, but not limited to, MoFlo sorter (DakoCytomation, Fort Collins, Colo.), FACSAria™, FACSArray™, FACSVantage™, BD™ LSR II, and FACSCalibur™ (BD Biosciences, San Jose, Calif.).

In a preferred embodiment, the method comprises selecting cells that also express CD3. The method may comprise specifically selecting the cells in any suitable manner. Preferably, the selecting is carried out using flow cytometry. The flow cytometry may be carried out using any suitable method known in the art. The flow cytometry may employ any suitable antibodies and stains. Preferably, the antibody is chosen such that it specifically recognizes and binds to the particular biomarker being selected. For example, the specific selection of CD3, CD8, TIM-3, LAG-3, 4-1BB, or PD-1 may be carried out using anti-CD3, anti-CD8, anti-TIM-3, anti-LAG-3, anti-4-1BB, or anti-PD-1 antibodies, respectively. The antibody or antibodies may be conjugated to a bead (e.g., a magnetic bead) or to a fluorochrome. Preferably, the flow cytometry is fluorescence-activated cell sorting (FACS). TCRs expressed on T cells can be selected based on reactivity to autologous tumors. Additionally, T cells that are reactive to tumors can be selected for based on markers using the methods described in patent publication Nos. WO2014133567 and WO2014133568, herein incorporated by reference in their entirety. Additionally, activated T cells can be selected for based on surface expression of CD107a.

In one embodiment of the invention, the method further comprises expanding the numbers of T cells in the enriched cell population. Such methods are described in U.S. Pat. No. 8,637,307 and is herein incorporated by reference in its entirety. The numbers of T cells may be increased at least about 3-fold (or 4-, 5-, 6-, 7-, 8-, or 9-fold), more preferably at least about 10-fold (or 20-, 30-, 40-, 50-, 60-, 70-, 80-, or 90-fold), more preferably at least about 100-fold, more preferably at least about 1,000 fold, or most preferably at least about 100,000-fold. The numbers of T cells may be expanded using any suitable method known in the art. Exemplary methods of expanding the numbers of cells are described in patent publication No. WO 2003057171, U.S. Pat. No. 8,034,334, and U.S. Patent Application Publication No. 2012/0244133, each of which is incorporated herein by reference.

In one embodiment, ex vivo T cell expansion can be performed by isolation of T cells and subsequent stimulation or activation followed by further expansion. In one embodiment of the invention, the T cells may be stimulated or activated by a single agent. In another embodiment, T cells are stimulated or activated with two agents, one that induces a primary signal and a second that is a co-stimulatory signal. Ligands useful for stimulating a single signal or stimulating a primary signal and an accessory molecule that stimulates a second signal may be used in soluble form. Ligands may be attached to the surface of a cell, to an Engineered Multivalent Signaling Platform (EMSP), or immobilized on a surface. In a preferred embodiment both primary and secondary agents are co-immobilized on a surface, for example a bead or a cell. In one embodiment, the molecule providing the primary activation signal may be a CD3 ligand, and the co-stimulatory molecule may be a CD28 ligand or 4-1BB ligand.

In certain embodiments, T cells comprising a CAR or an exogenous TCR, may be manufactured as described in WO2015120096, by a method comprising: enriching a population of lymphocytes obtained from a donor subject; stimulating the population of lymphocytes with one or more T-cell stimulating agents to produce a population of activated T cells, wherein the stimulation is performed in a closed system using serum-free culture medium; transducing the population of activated T cells with a viral vector comprising a nucleic acid molecule which encodes the CAR or TCR, using a single cycle transduction to produce a population of transduced T cells, wherein the transduction is performed in a closed system using serum-free culture medium; and expanding the population of transduced T cells for a predetermined time to produce a population of engineered T cells, wherein the expansion is performed in a closed system using serum-free culture medium. In certain embodiments, T cells comprising a CAR or an exogenous TCR, may be manufactured as described in WO2015120096, by a method comprising: obtaining a population of lymphocytes; stimulating the population of lymphocytes with one or more stimulating agents to produce a population of activated T cells, wherein the stimulation is performed in a closed system using serum-free culture medium; transducing the population of activated T cells with a viral vector comprising a nucleic acid molecule which encodes the CAR or TCR, using at least one cycle transduction to produce a population of transduced T cells, wherein the transduction is performed in a closed system using serum-free culture medium; and expanding the population of transduced T cells to produce a population of engineered T cells, wherein the expansion is performed in a closed system using serum-free culture medium. The predetermined time for expanding the population of transduced T cells may be 3 days. The time from enriching the population of lymphocytes to producing the engineered T cells may be 6 days. The closed system may be a closed bag system. Further provided is population of T cells comprising a CAR or an exogenous TCR obtainable or obtained by said method, and a pharmaceutical composition comprising such cells.

In certain embodiments, T cell maturation or differentiation in vitro may be delayed or inhibited by the method as described in WO2017070395, comprising contacting one or more T cells from a subject in need of a T cell therapy with an AKT inhibitor (such as, e.g., one or a combination of two or more AKT inhibitors disclosed in claim 8 of WO2017070395) and at least one of exogenous Interleukin-7 (IL-7) and exogenous Interleukin-15 (IL-15), wherein the resulting T cells exhibit delayed maturation or differentiation, and/or wherein the resulting T cells exhibit improved T cell function (such as, e.g., increased T cell proliferation; increased cytokine production; and/or increased cytolytic activity) relative to a T cell function of a T cell cultured in the absence of an AKT inhibitor.

In certain embodiments, a patient in need of a T cell therapy may be conditioned by a method as described in WO2016191756 comprising administering to the patient a dose of cyclophosphamide between 200 mg/m2/day and 2000 mg/m2/day and a dose of fludarabine between 20 mg/m2/day and 900 mg/m²/day.

### Therapeutic Agents

The present invention also provides for therapeutic agents. The present invention provides for therapeutic agents to modulate or shift one or more of the expression programs described herein (Table 3 and Table 4). The invention also provides for combination therapies to reverse drug resistance or to increase the response to a drug followed by treatment with the drug. For example, subsets of malignant cells expressing the MHC Class II program were present in multiple patients and may be associated with increased abundance of tumor-infiltrating lymphocytes (TILs), improved prognosis and response to immunotherapies (e.g., checkpoint blockade therapy and adoptive cell transfer). Thus, shifting one or more programs can be used in combination with immunotherapy. JAK/STAT signaling in cancer cells is associated with poor prognosis and resistance to chemotherapies. Thus, shifting one or more programs can be used in combination with chemotherapy.

The terms “therapeutic agent”, “therapeutic capable agent” or “treatment agent” are used interchangeably and refer to a molecule or compound that confers some beneficial effect upon administration to a subject. The beneficial effect includes enablement of diagnostic determinations; amelioration of a disease, symptom, disorder, or pathological condition; reducing or preventing the onset of a disease, symptom, disorder or condition; and generally counteracting a disease, symptom, disorder or pathological condition.

Applicants identified subpopulations of cancer cells that highly expressed three immune-related expression programs that may be downstream to the JAK/STAT pathway (inflammatory cytokines program, MHC-class II antigen presentation program, and interferon-response program). Applicants have shown that JAK/STAT inhibition had anti-tumor activity at nanomolar doses. Inhibition of the JAK/STAT pathway may have unintended side effects. Inhibition of one or more of the immune-related programs may provide for improved and/or targeted anti-tumor activity. Shared activation of the JAK/STAT-pathway in cancer cells and CAFs suggests that paracrine (and/or autocrine) signaling via this pathway may contribute to the pathogenesis of malignant ascites and drug resistance. Thus, modulation of one or more of the downstream expression programs can be used to reverse drug resistance.

### STAT Molecules

Members of the signal transducer and activator of transcription (STAT) protein family are intracellular transcription factors that mediate many aspects of cellular immunity, proliferation, apoptosis and differentiation. There are seven mammalian STAT family members that have been identified: STAT1, STAT2, STAT3, STAT4, STAT5 (STAT5A and STAT5B), and STAT6. STAT proteins are primarily activated by membrane receptor-associated Janus kinases (JAK). Dysregulation of the JAK/STAT pathway is frequently observed in primary tumors and leads to increased angiogenesis, enhanced survival of tumors, and immunosuppression. STAT proteins are involved in the development and function of the immune system and play a role in maintaining immune tolerance and tumor surveillance.

STAT proteins are present in the cytoplasm of cells under basal conditions. When activated by tyrosine phosphorylation, STAT proteins form dimers and translocate to the nucleus where they can bind specific nine base pair sequences in the regulatory regions of target genes, thereby activating transcription. A variety of tyrosine kinases, including polypeptide growth factor receptors, Src family members, and other kinases can catalyze this phosphorylation. While tyrosine phosphorylation is essential for their activation, STAT proteins can also be phosphorylated on unique serine residues. Although this is not sufficient to induce dimerization and DNA binding, STAT serine phosphorylation modulates the transcriptional response mediated by a tyrosine-phosphorylated STAT dimer, and may mediate distinct biological effects (Zhang X, et al. Science 1995; 267:1990-1994; Wen Z, et al. Cell 1995; 82:241-250; Kumar A, et al. Science 1997; 278:1630-1632). STAT proteins have been found to function inappropriately in many human malignancies (Alvarez J V, et al., Cancer Res 2005; 65(12):5054-62; Frank D A, et al. Cancer Treat. Res. 2003; 115:267-291; Bowman T, et al. Oncogene 2000; 19(21):2474-88).

### JAK/STAT Inhibitors

STAT3 is activated in several human tumors, including common epithelial cancers such as cancer of the breast, prostate, lung, pancreas, and ovary; hematologic cancers such as multiple myeloma, and acute leukemias; and diverse tumors such as melanoma and gliomas (Frank D A, et al. Cancer Treat. Res. 2003; 115:267-291). Many of the target genes of STAT3 code for proteins involved in cell survival, cell cycle progression, differentiation inhibition, invasion, and angiogenesis, all of the essential processes necessary for tumor formation and maintenance (Alvarez J V, et al., Cancer Res 2005; 65(12):5054-62). Inhibition of STAT3 function in cancer cells associated with enhanced STAT3 activity leads to a loss of proliferation and survival of the cancer cells (Frank D A. Curr. Cancer Therapy Reviews 2006; 2:57-65). Despite the central role that STAT3 plays in these diverse processes in tumor cell biology, loss of STAT3 function in normal adult cells has few if any serious consequences, and may in fact decrease the ability of a cell to become transformed.

An exemplary human STAT3 amino acid sequence is set forth below (SEQ ID NO: 4; GenBank Accession No: AAH14482, Version 1, incorporated herein by reference):

| | |
|---|---|
| 1 | maqwnqlqql dtryleqlhq lysdsfpmel rqflapwies qdwayaaske shatlvfhnl |
| 61 | lgeidqqysr flqesnvlyq hnlrrikqfl qsrylekpme iarivarclw eesrllqtaa |
| 121 | taaqqggqan hptaavvtek qqmleqhlqd vrkrvqdleq kmkvvenlqd dfdfnyktlk |
| 181 | sqgdmqdlng nnqsvtrqkm qqlegmltal dqmrrsivse lagllsamey vqktltdeel |
| 241 | adwkrrqqia ciggppnicl drlenwitsl aesqlqtrqq ikkleelqqk vsykgdpivq |
| 301 | hrpmleeriv elfrnlmksa fvverqpcmp mhpdrplvik tgvqfttkvr llvkfpelny |
| 361 | qlkikvcidk dsgdvaalrg srkfnilgtn tkvmnmeesn ngslsaefkh ltlreqrcgn |
| 421 | ggrancdasl ivteelhlit fetevyhqgl kidlethslp vvvisnicqm pnawasilwy |
| 481 | nmltnnpknv nfftkppigt wdqvaevlsw qfssttkrgl sieqlttlae kllgpgvnys |
| 541 | gcqitwakfc kenmagkgfs fwvwldniid lvkkyilalw negyimgfis kererailst |
| 601 | kppgtfllrf sesskeggvt ftwvekdisg ktqiqsvepy tkqqlnnmsf aeiimgykim |
| 661 | datnilvspl vylypdipke eafgkycrpe sqehpeadpg saapylktkf icvtpttcsn |
| 721 | tidlpmsprt ldslmqfgnn gegaepsagg qfesltfdme ltsecatspm |

An exemplary human STAT3 nucleic acid sequence is set forth below (SEQ ID NO: 5; GenBank Accession No: NM_139276, Version 2, incorporated herein by reference):

| | |
|---|---|
| 1 | ggtttccgga gctgcggcgg cgcagactgg gagggggagc cgggggttcc gacgtcgcag |
| 61 | ccgagggaac aagccccaac cggatcctgg acaggcaccc cggcttggcg ctgtctctcc |
| 121 | ccctcggctc ggagaggccc ttcggcctga gggagcctcg ccgcccgtcc ccggcacacg |
| 181 | cgcagccccg gcctctcggc ctctgccgga gaaacagttg ggacccctga ttttagcagg |
| 241 | atggcccaat ggaatcagct acagcagctt gacacacggt acctggagca gctccatcag |
| 301 | ctctacagtg acagcttccc aatggagctg cggcagtttc tggccccttg gattgagagt |
| 361 | caagattggg catatgcggc cagcaaagaa tcacatgcca ctttggtgtt tcataatctc |
| 421 | ctgggagaga ttgaccagca gtatagccgc ttcctgcaag agtcgaatgt tctctatcag |
| 481 | cacaatctac gaagaatcaa gcagtttctt cagagcaggt atcttgagaa gccaatggag |
| 541 | attgcccgga ttgtggcccg gtgcctgtgg gaagaatcac gccttctaca gactgcagcc |
| 601 | actgcggccc agcaaggggg ccaggccaac caccccacag cagccgtggt gacggagaag |
| 661 | cagcagatgc tggagcagca ccttcaggat gtccggaaga gagtgcagga tctagaacag |
| 721 | aaaatgaaag tggtagagaa tctccaggat gactttgatt tcaactataa aaccctcaag |
| 781 | agtcaaggag acatgcaaga tctgaatgga aacaaccagt cagtgaccag gcagaagatg |
| 841 | cagcagctgg aacagatgct cactgcgctg gaccagatgc ggagaagcat cgtgagtgag |
| 901 | ctggcggggc ttttgtcagc gatggagtac gtgcagaaaa ctctcacgga cgaggagctg |
| 961 | gctgactgga agaggcggca acagattgcc tgcattggag gcccgcccaa catctgccta |
| 1021 | gatcggctag aaaactggat aacgtcatta gcagaatctc aacttcagac ccgtcaacaa |
| 1081 | attaagaaac tggaggagtt gcagcaaaaa gtttcctaca aaggggaccc cattgtacag |
| 1141 | caccggccga tgctggagga gagaatcgtg gagctgttta gaaacttaat gaaaagtgcc |
| 1201 | tttgtggtgg agcggcagcc ctgcatgccc atgcatcctg accggcccct cgtcatcaag |
| 1261 | accggcgtcc agttcactac taaagtcagg ttgctggtca aattccctga gttgaattat |
| 1321 | cagcttaaaa ttaaagtgtg cattgacaaa gactctgggg acgttgcagc tctcagagga |
| 1381 | tcccggaaat ttaacattct gggcacaaac acaaaagtga tgaacatgga agaatccaac |
| 1441 | aacggcagcc tctctgcaga attcaaacac ttgaccctga gggagcagag atgtgggaat |
| 1501 | gggggccgag ccaattgtga tgcttccctg attgtgactg aggagctgca cctgatcacc |
| 1561 | tttgagaccg aggtgtatca ccaaggcctc aagattgacc tagagaccca ctccttgcca |
| 1621 | gttgtggtga tctccaacat ctgtcagatg ccaaatgcct gggcgtccat cctgtggtac |
| 1681 | aacatgctga ccaacaatcc caagaatgta aactttttta ccaagccccc aattggaacc |
| 1741 | tgggatcaag tggccgaggt cctgagctgg cagttctcct ccaccaccaa gcgaggactg |
| 1801 | agcatcgagc agctgactac actggcagag aaactcttgg gacctggtgt gaattattca |
| 1861 | gggtgtcaga tcacatgggc taaattttgc aaagaaaaca tggctggcaa gggcttctcc |
| 1921 | ttctgggtct ggctggacaa tatcattgac cttgtgaaaa agtacatcct ggccctttgg |
| 1981 | aacgaagggt acatcatggg ctttatcagt aaggagcggg agcgggccat cttgagcact |
| 2041 | aagcctccag gcaccttcct gctaagattc agtgaaagca gcaaagaagg aggcgtcact |
| 2101 | ttcacttggg tggagaagga catcagcggt aagacccaga tccagtccgt ggaaccatac |
| 2161 | acaaagcagc agctgaacaa catgtcattt gctgaaatca tcatgggcta taagatcatg |
| 2221 | gatgctacca atatcctggt gtctccactg gtctatctct atcctgacat tcccaaggag |
| 2281 | gaggcattcg gaaagtattg tcggccagag agccaggagc atcctgaagc tgacccaggt |
| 2341 | agcgctgccc catacctgaa gaccaagttt atctgtgtga caccaacgac ctgcagcaat |
| 2401 | accattgacc tgccgatgtc cccccgcact ttagattcat tgatgcagtt tggaaataat |
| 2461 | ggtgaaggtg ctgaaccctc agcaggaggg cagtttgagt ccctcacctt tgacatggag |
| 2521 | ttgacctcgg agtgcgctac ctcccccatg tgaggagctg agaacggaag ctgcagaaag |
| 2581 | atacgactga ggcgcctacc tgcattctgc cacccctcac acagccaaac cccagatcat |
| 2641 | ctgaaactac taactttgtg gttccagatt ttttttaatc tcctacttct gctatctttg |
| 2701 | agcaatctgg gcacttttaa aaatagagaa atgagtgaat gtgggtgatc tgcttttatc |
| 2761 | taaatgcaaa taaggatgtg ttctctgaga cccatgatca ggggatgtgg cggggggtgg |
| 2821 | ctagagggag aaaaaggaaa tgtcttgtgt tgttttgttc ccctgccctc ctttctcagc |
| 2881 | agctttttgt tattgttgtt gttgttctta gacaagtgcc tcctggtgcc tgcggcatcc |
| 2941 | ttctgcctgt ttctgtaagc aaatgccaca ggccacctat agctacatac tcctggcatt |
| 3001 | gcacttttta accttgctga catccaaata gaagatagga ctatctaagc cctaggtttc |
| 3061 | tttttaaatt aagaaataat aacaattaaa gggcaaaaaa cactgtatca gcatagcctt |
| 3121 | tctgtattta agaaacttaa gcagccgggc atggtggctc acgcctgtaa tcccagcact |
| 3181 | ttgggaggcc gaggcggatc ataaggtcag gagatcaaga ccatcctggc taacacggtg |
| 3241 | aaaccccgtc tctactaaaa gtacaaaaaa ttagctgggt gtggtggtgg gcgcctgtag |
| 3301 | tcccagctac tcgggaggct gaggcaggag aatcgcttga acctgagagg cggaggttgc |
| 3361 | agtgagccaa aattgcacca ctgcacactg cactccatcc tgggcgacag tctgagactc |
| 3421 | tgtctcaaaa aaaaaaaaaa aaaaaagaaa cttcagttaa cagcctcctt ggtgctttaa |
| 3481 | gcattcagct tccttcaggc tggtaattta tataatccct gaaacgggct tcaggtcaaa |
| 3541 | cccttaagac atctgaagct gcaacctggc ctttggtgtt gaaataggaa ggtttaagga |
| 3601 | gaatctaagc attttagact tttttttata aatagactta ttttcctttg taatgtattg |
| 3661 | gccttttagt gagtaaggct gggcagaggg tgcttacaac cttgactccc tttctccctg |
| 3721 | gacttgatct gctgtttcag aggctaggtt gtttctgtgg gtgccttatc agggctggga |
| 3781 | tacttctgat tctggcttcc ttcctgcccc accctcccga ccccagtccc cctgatcctg |
| 3841 | ctagaggcat gtctccttgc gtgtctaaag gtccctcatc ctgtttgttt taggaatcct |
| 3901 | ggtctcagga cctcatggaa gaagaggggg agagagttac aggttggaca tgatgcacac |
| 3961 | tatggggccc cagcgacgtg tctggttgag ctcagggaat atggttctta gccagtttct |
| 4021 | tggtgatatc cagtggcact tgtaatggcg tcttcattca gttcatgcag ggcaaaggct |
| 4081 | tactgataaa cttgagtctg ccctcgtatg agggtgtata cctggcctcc ctctgaggct |
| 4141 | ggtgactcct ccctgctggg gccccacagg tgaggcagaa cagctagagg gcctccccgc |
| 4201 | ctgcccgcct tggctggcta gctcgcctct cctgtgcgta tgggaacacc tagcacgtgc |
| 4261 | tggatgggct gcctctgact cagaggcatg gccggatttg gcaactcaaa accaccttgc |
| 4321 | ctcagctgat cagagtttct gtggaattct gtttgttaaa tcaaattagc tggtctctga |
| 4381 | attaaggggg agacgacctt ctctaagatg aacagggttc gccccagtcc tcctgcctgg |
| 4441 | agacagttga tgtgtcatgc agagctctta cttctccagc aacactcttc agtacataat |
| 4501 | aagcttaact gataaacaga atatttagaa aggtgagact tgggcttacc attgggttta |
| 4561 | aatcataggg acctagggcg agggttcagg gcttctctgg agcagatatt gtcaagttca |
| 4621 | tggccttagg tagcatgtat ctggtcttaa ctctgattgt agcaaaagtt ctgagaggag |
| 4681 | ctgagccctg ttgtggccca ttaaagaaca gggtcctcag gccctgcccg cttcctgtcc |
| 4741 | actgccccct ccccatcccc agcccagccg agggaatccc gtgggttgct tacctaccta |
| 4801 | taaggtggtt tataagctgc tgtcctggcc actgcattca aattccaatg tgtacttcat |
| 4861 | agtgtaaaaa tttatattat tgtgaggttt tttgtctttt tttttttttt ttttttttgg |
| 4921 | tatattgctg tatctacttt aacttccaga aataaacgtt atataggaac cgtaaaaa |

The following compounds are STAT3 inhibitors: pyrimethamine, atovaquone, pimozide, guanabenz acetate, alprenolol hydrochloride, nifuroxazide, solanine alpha, fluoxetine hydrochloride, ifosfamide, pyrvinium pamoate, moricizine hydrochloride, 3,3′-oxybis[tetrahydrothiophene, 1,1,1′,1′-tetraoxide], 3-(1,3-benzodioxol-5-yl)-1,6-dimethyl-pyrimido[5,4-e]-1,2,4-triazine-5,7(-1H,6H)-dione, 2-(1,8-Naphthyridin-2-yl)phenol, 3-(2-hydroxyphenyl)-3-phenyl-N,N-dipropylpropanamide as well as any derivatives of these compounds or analogues thereof. These compounds are commercially available through various sources.

Another exemplary STAT3 inhibitor includes JSI-124. Cucurbitacin I (JSI-124) is a selective inhibitor of the janus kinase 2/signal transducer and activator of transcription 3 (JAK2/STAT3) signaling pathway with anti-proliferative and anti-tumor properties. The structure of JSI-124 (cucurbitacin I) is set forth below (Blaskovich et al., 2003 Cancer Res., 63(6): 1270-1279; incorporated herein by reference).

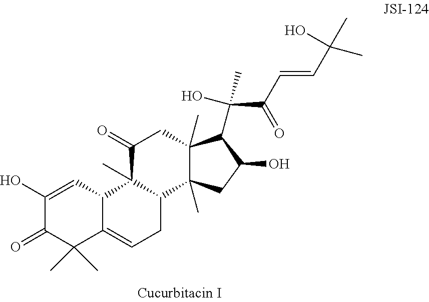 [SMILES: CC(C)(O)/C=C/C(=O)[C@](C)(O)[C@H]1[C@@H](O)C[C@@]2(C)[C@@H]3CC=C4[C@H](C=C(O)C(=O)C4(C)C)[C@@]3(C)C(=O)C[C@@]12C]

In certain embodiments, the STAT3 activity inhibitor is administered intraperitoneally. In certain embodiments, the STAT3 activity inhibitor comprises JSI-124 (cucurbitacin I). In certain embodiments, JSI-124 (cucurbitacin I) is administered at a dose of about 0.01 μM to about 10.0 μM, e.g., 0.01 μM, 0.02 μM, 0.03 μM, 0.04 μM, 0.06 μM, 0.07 μM, 0.08 μM, 0.09 μM, 0.1 μM, 0.2 μM, 0.3 μM, 0.4 μM, 0.5 μM, 0.6 μM, 0.7 μM, 0.8 μM, 0.9 μM, 1.0 μM, 2.0 μM, 3.0 μM, 4.0 μM, 5.0 μM, 6.0 μM, 7.0 μM, 8.0 μM, 9.0 μM, or 10.0 μM. Alternatively, the JSI-124 is administered at a dose of 1 mg/kg/day.

In certain embodiments, the STAT3 inhibitor is administered three times per day, once per day, three times per week, once per week, three times per month, once per month, once every three months, or once every six months. In some cases, the STAT3 inhibitor is administered for one month, three months, six months, one year, or more.

### Chemotherapy

In certain embodiments, the methods further comprise administering a therapeutically effective amount of a chemotherapeutic agent. Chemotherapy (often abbreviated to chemo and sometimes CTX or CTx) is a type of cancer treatment that uses one or more anti-cancer drugs (chemotherapeutic agents) as part of a standardized chemotherapy regimen. Chemotherapy may be given with a curative intent (which almost always involves combinations of drugs), or it may aim to prolong life or to reduce symptoms (palliative chemotherapy). For example, the chemotherapeutic agent comprises a platinum-based chemotherapeutic agent or a taxane-based chemotherapeutic agent. Suitable platinum-based chemotherapeutic agents include cisplatin and carboplatin. In certain embodiments, the chemotherapy is administered according to the standard of care for ovarian cancer or a specific cancer.

### Checkpoint Blockade Therapy

Immunotherapy can include checkpoint blockade (CPB), chimeric antigen receptors (CARs), and adoptive T-cell therapy. Antibodies that block the activity of checkpoint receptors, including CTLA-4, PD-1, Tim-3, Lag-3, and TIGIT, either alone or in combination, have been associated with improved effector CD8⁺ T cell responses in multiple pre-clinical cancer models (Johnston et al., 2014. The immunoreceptor TIGIT regulates antitumor and antiviral CD8(+) T cell effector function. Cancer cell 26, 923-937; Ngiow et al., 2011. Anti-TIM3 antibody promotes T cell IFN-gamma-mediated antitumor immunity and suppresses established tumors. Cancer research 71, 3540-3551; Sakuishi et al., 2010. Targeting Tim-3 and PD-1 pathways to reverse T cell exhaustion and restore anti-tumor immunity. The Journal of experimental medicine 207, 2187-2194; and Woo et al., 2012. Immune inhibitory molecules LAG-3 and PD-1 synergistically regulate T-cell function to promote tumoral immune escape. Cancer research 72, 917-927). Similarly, blockade of CTLA-4 and PD-1 in patients (Brahmer et al., 2012. Safety and activity of anti-PD-L1 antibody in patients with advanced cancer. The New England journal of medicine 366, 2455-2465; Hodi et al., 2010. Improved survival with ipilimumab in patients with metastatic melanoma. The New England journal of medicine 363, 711-723; Schadendorf et al., 2015. Pooled Analysis of Long-Term Survival Data From Phase II and Phase III Trials of Ipilimumab in Unresectable or Metastatic Melanoma. Journal of clinical oncology: official journal of the American Society of Clinical Oncology 33, 1889-1894; Topalian et al., 2012. Safety, activity, and immune correlates of anti-PD-1 antibody in cancer. The New England journal of medicine 366, 2443-2454; and Wolchok et al., 2017. Overall Survival with Combined Nivolumab and Ipilimumab in Advanced Melanoma. The New England journal of medicine 377, 1345-1356) has shown increased frequencies of proliferating T cells, often with specificity for tumor antigens, as well as increased CD8⁺ T cell effector function (Ayers et al., 2017. IFN-gamma-related mRNA profile predicts clinical response to PD-1 blockade. The Journal of clinical investigation 127, 2930-2940; Das et al., 2015. Combination therapy with anti-CTLA-4 and anti-PD-1 leads to distinct immunologic changes in vivo. Journal of immunology 194, 950-959; Gubin et al., 2014. Checkpoint blockade cancer immunotherapy targets tumour-specific mutant antigens. Nature 515, 577-581; Huang et al., 2017. T-cell invigoration to tumour burden ratio associated with anti-PD-1 response. Nature 545, 60-65; Kamphorst et al., 2017. Proliferation of PD-1+ CD8 T cells in peripheral blood after PD-1-targeted therapy in lung cancer patients. Proceedings of the National Academy of Sciences of the United States of America 114, 4993-4998; Kvistborg et al., 2014. Anti-CTLA-4 therapy broadens the melanoma-reactive CD8+ T cell response. Science translational medicine 6, 254ra128; van Rooij et al., 2013. Tumor exome analysis reveals neoantigen-specific T-cell reactivity in an ipilimumab-responsive melanoma. Journal of clinical oncology: official journal of the American Society of Clinical Oncology 31, e439-442; and Yuan et al., 2008. CTLA-4 blockade enhances polyfunctional NY-ESO-1 specific T cell responses in metastatic melanoma patients with clinical benefit. Proceedings of the National Academy of Sciences of the United States of America 105, 20410-20415). Accordingly, the success of checkpoint receptor blockade has been attributed to the binding of blocking antibodies to checkpoint receptors expressed on dysfunctional CD8⁺ T cells and restoring effector function in these cells. The check point blockade therapy may be an inhibitor of any check point protein described herein. The checkpoint blockade therapy may comprise anti-TIM3, anti-CTLA4, anti-PD-L1, anti-PD1, anti-TIGIT, anti-LAG3, or combinations thereof. Anti-PD1 antibodies are disclosed in U.S. Pat. No. 8,735,553. Antibodies to LAG-3 are disclosed in U.S. Pat. No. 9,132,281. Anti-CTLA4 antibodies are disclosed in U.S. Pat. Nos. 9,327,014; 9,320,811; and 9,062,111. Specific check point inhibitors include, but are not limited to anti-CTLA4 antibodies (e.g., Ipilimumab and tremelimumab), anti-PD-1 antibodies (e.g., Nivolumab, Pembrolizumab), and anti-PD-L1 antibodies (e.g., Atezolizumab).

In certain embodiments, therapeutic agents may also be identified by screening methods, discussed further herein.

In an aspect, a method of treating a subject with ovarian cancer is provided wherein when the ovarian tumor exhibits increased macrophages characteristic of an immunoreactive phenotype, a therapeutic regimen comprising a CPB is administered.

In an aspect, a method of treating a subject with ovarian cancer is provided wherein when the ovarian tumor exhibits increased expression or activity of one or more MHC class II biological programs, a therapeutic regimen comprising a CPB is administered.

In an aspect, a method of treating a subject with ovarian cancer is provided wherein when the ovarian tumor does not exhibit increased CAFs characteristic of a mesenchymal phenotype, a therapeutic regimen comprising a CPB is administered.

### CAF Modulators

CAFs are important for survival and response to treatment (see, e.g., Su et al., CD10+GPR77+ Cancer-Associated Fibroblasts Promote Cancer Formation and Chemoresistance by Sustaining Cancer Stemness, 2018, Cell 172, 1-16). “Carcinoma-associated fibroblasts (CAFs) are activated fibroblasts (Orimo and Weinberg, 2006) constituting the major stromal components in many types of malignancies (Kalluri, 2016). Accumulating evidence suggests that CAFs play a crucial role in tumor development and are potential therapeutic targets for cancer. However, recent studies suggest that CAFs are heterogeneous and contain different subpopulations with distinct phenotypes and functions, which hinders their application in diagnosis and targeted therapy. Different CAF populations that secrete distinct profiles of cytokines have been identified in a variety of cancers (Öhlund et al., 2017, Sugimoto et al., 2006). Although significant prognostic impacts of CAFs have been studied in various tumors, including breast and lung cancers, whether CAFs are associated with good or poor prognosis is contradictory in different studies (Paulsson and Micke, 2014). More importantly, although it is generally thought that CAFs promote tumor progression, targeting CAFs leads to disease exacerbation in a cohort of pancreatic cancer patients (Amakye et al., 2013) and in a mouse model of pancreatic cancer (Özdemir et al., 2014, Rhim et al., 2014), suggesting that different fibroblast subsets may exert opposite functions in cancer progression. Therefore, to precisely target the cancer-promoting CAF subsets, it is necessary to identify specific markers to define these subpopulations and understand their functions and mechanisms.” (see, e.g., Su et al., 2018).

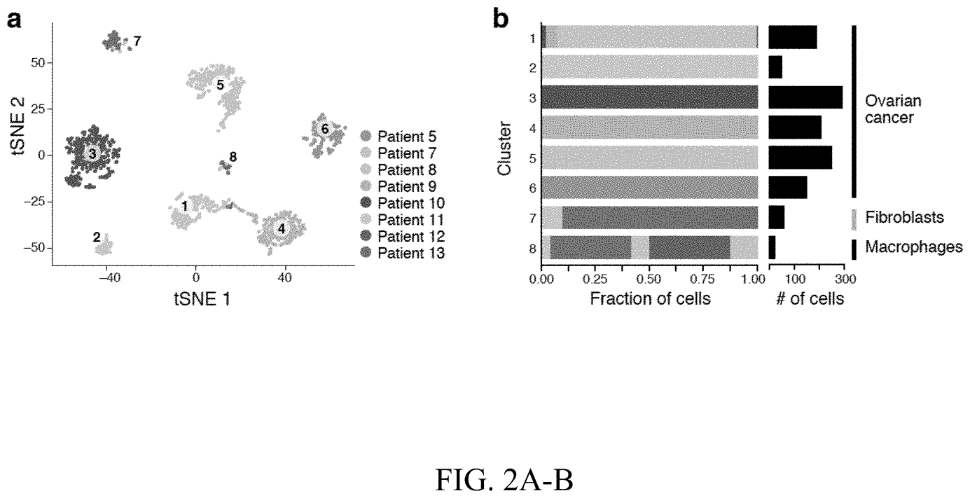

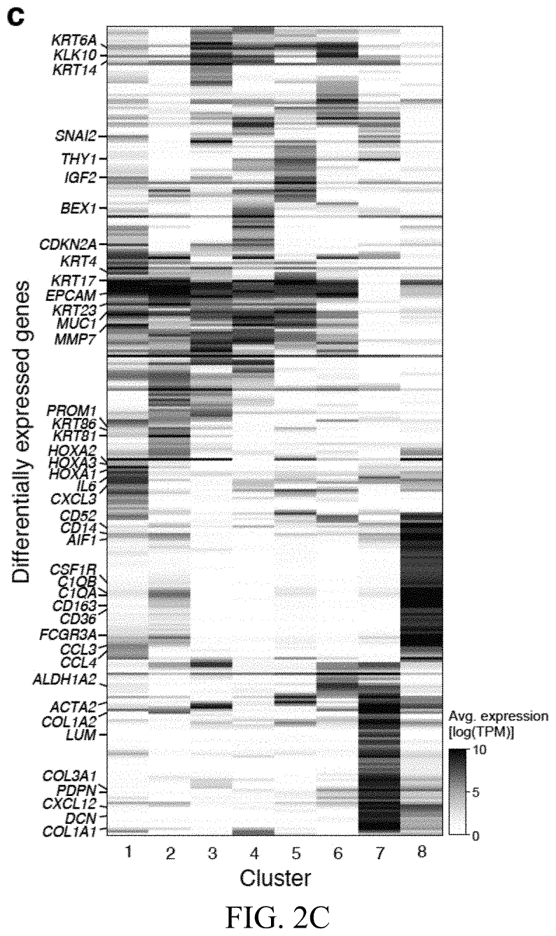

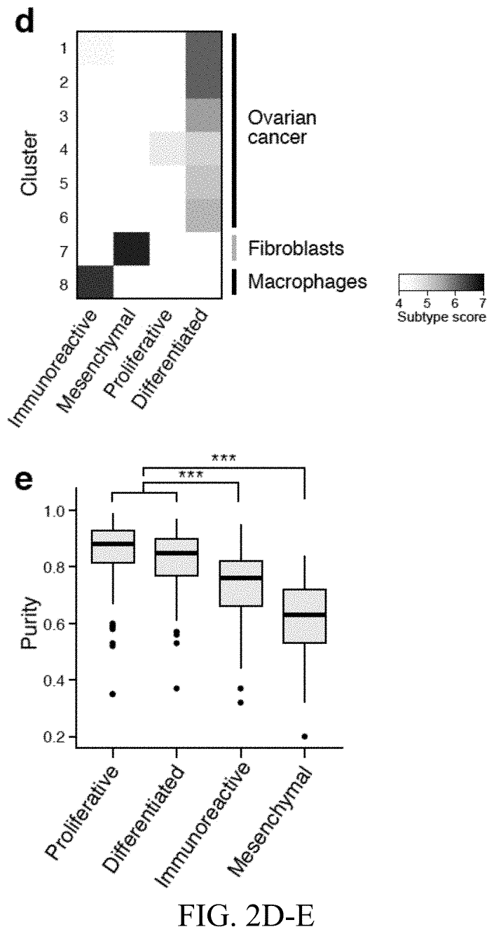

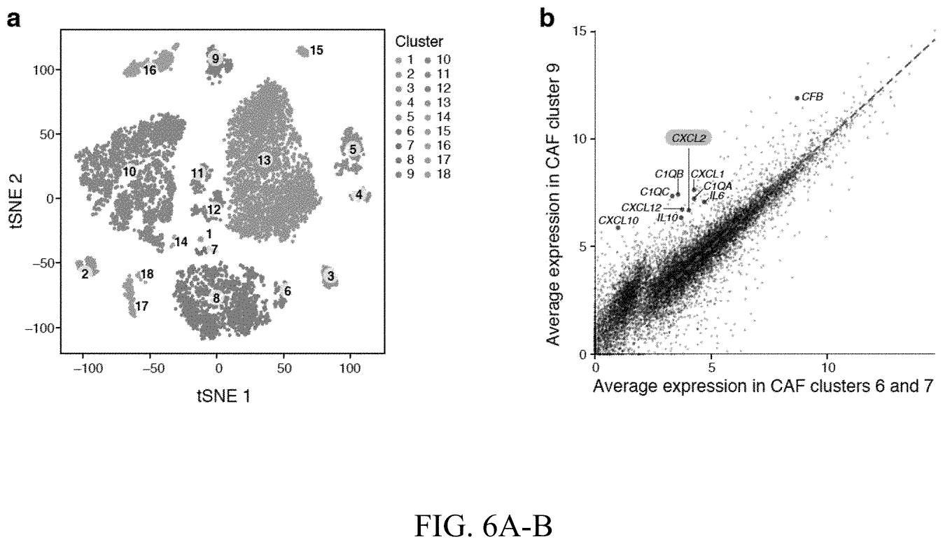

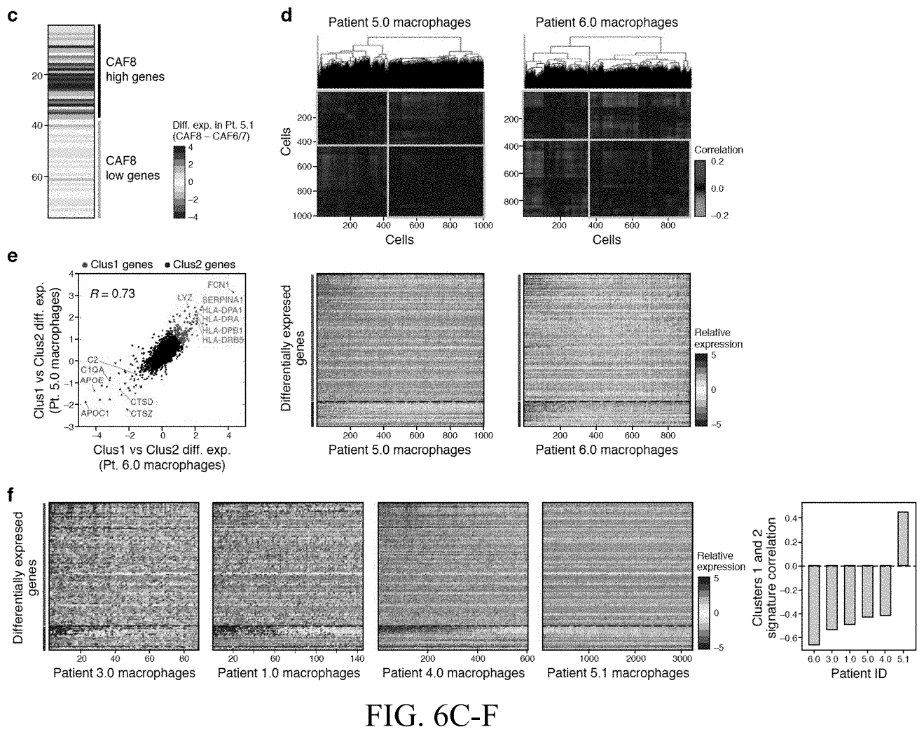

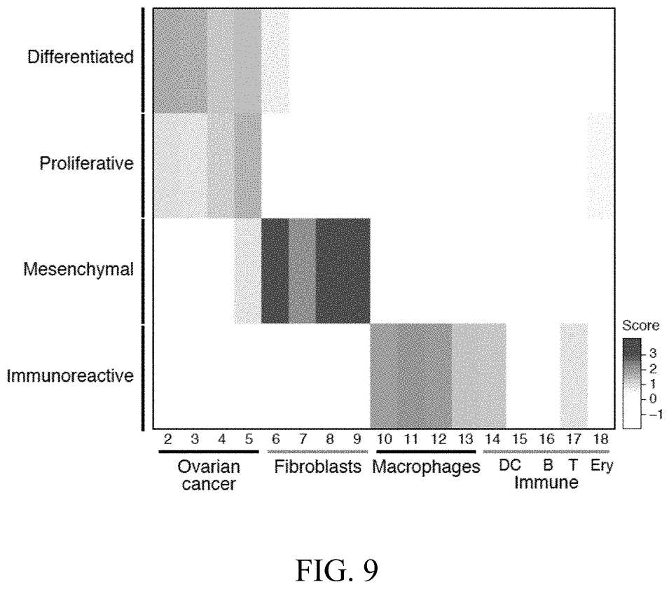

In certain embodiments, different subtypes of ovarian cancer are treated by targeting CAFs. The mesenchymal and immunoreactive subtypes were characterized herein by differential non-malignant cell representation (i.e., CAFs and macrophages). The mesenchymal subtype had increased CAFs. The immunoreactive subtype is associated with superior overall survival (OS), and the mesenchymal subtype is associated with the worst OS (see, e.g., Lheureux et al., Epithelial ovarian cancer: Evolution of management in the era of precision medicine. CA Cancer J Clin. 2019 July; 69(4):280-304). In certain embodiments, CAFs characteristic of a mesenchymal phenotype described herein are modulated in ovarian cancer. In certain embodiments, the agent modulates one or more genes expressed in the CAFs associated with the mesenchymal subtype (see, e.g., Table 2, clusters 6-9; FIG. 2A-E; FIG. 6A-6F; and FIG. 9). In certain embodiments, the one or more genes are selected from the group consisting of PDPN, C1QA/B/C, CFB, CXCL12, CXCL1, CXCL2, CXCL10, IL6, IL10, ALDH1A2, ACTA2, COL1A2, LUM, COL3A1, DCN and COL1A1.

Podoplanin is a protein that in humans is encoded by the “PDPN” gene and is also known as PDPN, AGGRUS, GP36, GP40, Gp38, HT1A-1, OTS8, PA2.26, T1A, T1A-2, T1A2, and TI1A. As used herein, PDPN may refer to nucleotide or protein sequence according to accession numbers NM_001006625.1, NM_001006624.1, NM_198389.2, NM_006474.4, NM_010329.3, NM_001290822.1, NP_001006626.1, NP 001006625.1, NP_938203.2 and NP_006465.3. PDPN has been studied extensively in the cancer field. It is a specific lymphatic vessel marker, and since lymphangiogenesis levels are correlated with poor prognosis in cancer patients, it can be used as a diagnostic marker (Astarita, J L, et al., (2012). “Podoplanin: emerging functions in development, the immune system, and cancer”. Frontiers in Immunology. 3: 283). It is often upregulated in certain types of cancer, including several types of squamous cell carcinomas, malignant mesothelioma and brain tumors (Astarita, et al., 2012). Moreover, it can be upregulated by cancer-associated fibroblasts (CAFs) in the tumor stroma, (Astarita, et al., 2012; and Kitano, H; et al., (2010). “Podoplanin expression in cancerous stroma induces lymphangiogenesis and predicts lymphatic spread and patient survival”. Archives of pathology & laboratory medicine. 134 (10): 1520-7) where it has been associated with poor prognosis (Chuang, W Y, et al., (2014). “Concordant podoplanin expression in cancer-associated fibroblasts and tumor cells is an adverse prognostic factor in esophageal squamous cell carcinoma”. International Journal of Clinical and Experimental Pathology. 7 (8): 4847-56). In squamous cell carcinomas, PDPN is believed to play a role in the cancer cell invasiveness by controlling invadopodia, and thus mediating efficient ECM degradation (Martín-Villar, E, et al., (2015). “Podoplanin mediates ECM degradation by squamous carcinoma cells through control of invadopodia stability”. Oncogene. 34 (34): 4531-44).

In certain embodiments, a therapeutic agent modulates CAF activation, such as a complement factor (see, e.g., US20200023007A1; and WO2018232195A1).

### Combination Therapy

In certain embodiments, the present invention provides for one or more therapeutic agents against combinations of targets identified. Targeting the identified combinations may provide for enhanced or otherwise previously unknown activity in the treatment of disease. In certain embodiments, an agent against one of the targets in a combination may already be known or used clinically. In certain embodiments, targeting the combination may require less of the agent as compared to the current standard of care and provide for less toxicity and improved treatment.

In certain embodiments, the present invention provides for a combination therapy comprising a treatment described herein with a treatment that is part of the standard of care for a cancer (i.e., a therapeutic regime). In certain embodiments, a treatment, such as ACT or a therapeutic agent is used in combination with the standard of care. In certain embodiments, the standard of care for treating ovarian cancer comprises surgery, chemotherapy, and targeted therapy (see, e.g., Lheureux et al., Epithelial ovarian cancer: Evolution of management in the era of precision medicine. CA Cancer J Clin. 2019 July; 69(4):280-304). Systemic therapy can include single to combination chemotherapy approaches alone or in combination with targeted therapy. In certain embodiments, surgery includes surgery for accurate surgical staging, primary debulking surgery, interval debulking surgery, and secondary debulking surgery. In certain embodiments, chemotherapy includes carboplatin, cisplatin and paclitaxel. In certain embodiments, targeted therapy includes Bevacizumab, which is a humanized monoclonal antibody against vascular endothelial growth factor (VEGF), and poly (ADP-ribose) polymerase (PARP) inhibitors (e.g., Olaparib, Niraparib, and Rucaparib). Other therapies that may be used in combination with the present invention include agents targeting the folate receptor (e.g., mirvetuximab soravtansine (IMGN853), which is an ADC consisting of an anti-FRα antibody linked to the tubulin-disrupting maytansinoid DM4 drug, a potent antimitotic agent). In certain embodiments, checkpoint blockade therapy is used in a combination therapy. In certain embodiments, chemotherapy in combination with immunotherapy is used in the treatment of ovarian cancer. In certain embodiments, the combination therapy comprises paclitaxel plus pembrolizumab, preferably in patients with platinum-resistant ovarian cancer. In certain embodiments, the combination therapy comprises immunotherapy combined with PARP inhibitors.

In one example, agents are administered in a combination therapy, i.e., combined with other agents, e.g., therapeutic agents, that are useful for treating pathological conditions or disorders, such as various forms of cancer. The term “in combination” in this context means that the agents are given substantially contemporaneously, either simultaneously or sequentially. If given sequentially, at the onset of administration of the second agent, the first of the two agents is in some cases still detectable at effective concentrations at the site of treatment.

The administration of an agent or a combination of agents for the treatment of a neoplasia may be by any suitable means that results in a concentration of the therapeutic that, combined with other components, is effective in ameliorating, reducing, or stabilizing a neoplasia. The agent may be contained in any appropriate amount in any suitable carrier substance, and is generally present in an amount of 1-95% by weight of the total weight of the composition. The composition may be provided in a dosage form that is suitable for parenteral (e.g., subcutaneously, intravenously, intramuscularly, or intraperitoneally) administration route. The pharmaceutical compositions may be formulated according to conventional pharmaceutical practice (see, e.g., Remington: The Science and Practice of Pharmacy (20th ed.), ed. A. R. Gennaro, Lippincott Williams & Wilkins, 2000 and Encyclopedia of Pharmaceutical Technology, eds. J. Swarbrick and J. C. Boylan, 1988-1999, Marcel Dekker, New York).

Accordingly, in some examples, the prophylactic and/or therapeutic regimen comprises administration of a agent of the invention in combination with one or more additional anticancer therapeutics. In one example, the dosages of the one or more additional anticancer therapeutics used in the combination therapy is lower than those which have been or are currently being used to prevent, treat, and/or manage cancer. The recommended dosages of the one or more additional anticancer therapeutics currently used for the prevention, treatment, and/or management of cancer can be obtained from any reference in the art including, but not limited to, Hardman et al., eds., Goodman & Gilman's The Pharmacological Basis Of Basis Of Therapeutics, 10th ed., McGraw-Hill, New York, 2001; Physician's Desk Reference (60^{th} ed., 2006), which is incorporated herein by reference in its entirety.

The agent of the invention and the one or more additional anticancer therapeutics can be administered separately, simultaneously, or sequentially. In various aspects, the agent of the invention and the additional anticancer therapeutic are administered less than 5 minutes apart, less than 30 minutes apart, less than 1 hour apart, at about 1 hour apart, at about 1 to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, at about 12 hours to 18 hours apart, 18 hours to 24 hours apart, 24 hours to 36 hours apart, 36 hours to 48 hours apart, 48 hours to 52 hours apart, 52 hours to 60 hours apart, 60 hours to 72 hours apart, 72 hours to 84 hours apart, 84 hours to 96 hours apart, or 96 hours to 120 hours part. In another example, two or more anticancer therapeutics are administered within the same patient visit.

In certain aspects, the agent of the invention and the additional anticancer therapeutic are cyclically administered. Cycling therapy involves the administration of one anticancer therapeutic for a period of time, followed by the administration of a second anticancer therapeutic for a period of time and repeating this sequential administration, i.e., the cycle, in order to reduce the development of resistance to one or both of the anticancer therapeutics, to avoid or reduce the side effects of one or both of the anticancer therapeutics, and/or to improve the efficacy of the therapies. In one example, cycling therapy involves the administration of a first anticancer therapeutic for a period of time, followed by the administration of a second anticancer therapeutic for a period of time, optionally, followed by the administration of a third anticancer therapeutic for a period of time and so forth, and repeating this sequential administration, i.e., the cycle in order to reduce the development of resistance to one of the anticancer therapeutics, to avoid or reduce the side effects of one of the anticancer therapeutics, and/or to improve the efficacy of the anticancer therapeutics.

In another example, the anticancer therapeutics are administered concurrently to a subject in separate compositions. The combination anticancer therapeutics of the invention may be administered to a subject by the same or different routes of administration.

When an agent of the invention and the additional anticancer therapeutic are administered to a subject concurrently, the term “concurrently” is not limited to the administration of the anticancer therapeutics at exactly the same time, but rather, it is meant that they are administered to a subject in a sequence and within a time interval such that they can act together (e.g., synergistically to provide an increased benefit than if they were administered otherwise). For example, the anticancer therapeutics may be administered at the same time or sequentially in any order at different points in time; however, if not administered at the same time, they should be administered sufficiently close in time so as to provide the desired therapeutic effect, preferably in a synergistic fashion. The combination anticancer therapeutics of the invention can be administered separately, in any appropriate form and by any suitable route. When the components of the combination anticancer therapeutics are not administered in the same pharmaceutical composition, it is understood that they can be administered in any order to a subject in need thereof. For example, a agent of the invention can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the additional anticancer therapeutic, to a subject in need thereof. In various aspects, the anticancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one example, the anticancer therapeutics are administered within the same office visit. In another example, the combination anticancer therapeutics of the invention are administered at 1 minute to 24 hours apart.

### Small Molecules

In certain embodiments, the one or more agents is a small molecule. The term “small molecule” refers to compounds, preferably organic compounds, with a size comparable to those organic molecules generally used in pharmaceuticals. The term excludes biological macromolecules (e.g., proteins, peptides, nucleic acids, etc.). Preferred small organic molecules range in size up to about 5000 Da, e.g., up to about 4000, preferably up to 3000 Da, more preferably up to 2000 Da, even more preferably up to about 1000 Da, e.g., up to about 900, 800, 700, 600 or up to about 500 Da. In certain embodiments, the small molecule may act as an antagonist or agonist (e.g., blocking an enzyme active site or activating a receptor by binding to a ligand binding site).

One type of small molecule applicable to the present invention is a degrader molecule. Proteolysis Targeting Chimera (PROTAC) technology is a rapidly emerging alternative therapeutic strategy with the potential to address many of the challenges currently faced in modern drug development programs. PROTAC technology employs small molecules that recruit target proteins for ubiquitination and removal by the proteasome (see, e.g., Zhou et al., Discovery of a Small-Molecule Degrader of Bromodomain and Extra-Terminal (BET) Proteins with Picomolar Cellular Potencies and Capable of Achieving Tumor Regression. J. Med. Chem. 2018, 61, 462-481; Bondeson and Crews, Targeted Protein Degradation by Small Molecules, Annu Rev Pharmacol Toxicol. 2017 Jan. 6; 57: 107-123; and Lai et al., Modular PROTAC Design for the Degradation of Oncogenic BCR-ABL Angew Chem Int Ed Engl. 2016 Jan. 11; 55(2): 807-810).

### Genetic Modifying Agents

In certain embodiments, the one or more modulating agents may be a genetic modifying agent. The genetic modifying agent may comprise a CRISPR system, a zinc finger nuclease system, a TALEN, a meganuclease or RNAi system. The genetic modifying agent preferably modulates expression of one or more genes selected from the group consisting of CLDN3, CLDN7, CLDN4, EPCAM, TACSTD2, MAL2, LCN2, CKB, RBP1, CDKN2A, C19orf33, WFDC2, LSR, CRABP2, S100A13, KRT7, CRIP2, MDK, CD9, SPINT2, SLPI, KRT19, KRT18, KRT8, TM4SF1, NGFRAP1, S100A16, PCBD1, OCIAD2, ZNF428, TMEM205, TSTD1, TNFRSF12A, MARCKSL1, IFI27, CD47, POLR2I, CCDC124, PDCD5 and DPY30; or one or more genes selected from the group consisting of CLU, TACSTD2, MFAP2, LGALS3BP, FBLN2, LY6E, VTCN1, CLIC5, PTPRS, RAB25, SH3BGRL, BST2, SERPING1, MFGE8, ANKRD65, UCA1, THEM6, HSPG2, MSLN, NDRG2, TAPBP, CDH6, PLD3, RBMS3, COMP, SEPP1, RNF213, CD74, IFIT3 and IFI6; or CCBP2, TACSTD2, OAS1, MX1, GPNMB, OAS2, KRT23, MSLN, TXNIP, C15orf48, UPK3BL, PTGES, LCN2, CD82, SAT1, VTCN1, ITGB2, NCCRP1, CEACAM6, AGR2, PSCA, PARP14, HERC6, GABRP, IFI44L, IFIT3, IFI6, IFI44, ISG15 and IFIT1; or XAF1, MX1, PARP14, DDX60, C19orf66, OAS3, STAT1, OAS1, PARP9, PLSCR1, IFI44, IFIT3, OASL, TRIM22, DTX3L, IFITM1, PSMB8, RTP4, RSAD2, DDX58, IFITM3, NMI, UBE2L6, UBA7, SP100, OAS2, ISG15, IFIT1, IFI44L and IFI6; or TACSTD2, LOC100505633, CLU, HLA-H, CLDN1, HLA-C, CD24, TSPAN15, HLA-B, CCL28, FAM107A, RARRES3, TNC, GPX1, KLK5, IFI27, SEPP1, CCDC3, PLA2G16, MAL, CNN3, CMBL, PSMB10, CRIP1, KRT5, C3orf55, HLA-A, ISG15, IFIT1 and IFI6; or IFIT3, ISG20, IFIT2, MX1, OAS1, HLA-H, RSAD2, C19orf66, PARP14, STAT1, HLA-A, EPSTI1, CMPK2, KRT6A, IFI35, HLA-F, HLA-B, OAS3, B2M, DDX58, CFB, ANXA3, TIMP1, DTX3L, PARP9, GPRC5A, ISG15, IFIT1, IFI44L and OAS2; or LYNX1, LYPD2, CLU, TRIM29, MMP7, MUC4, TSPAN1, ATP6V1B1, SERPINA1, SLPI, KCNN4, SYT8, RARRES3, PTGES, RASAL1, CP, FOLR1, UNC5B, ALOX5, MUC20, FOS, SLC4A11, FXYD3, C3, UNC5B-AS1, DEFB1, COL12A1, CD74, HLA-DRB1 and HLA-DRA; or RARRES1, CRYAB, NNMT, PLA2G16, MGST1, HLA-DRB6, MT1F, CLU, CD14, NFIB, C3, BNIP3L, OAT, SLC34A2, NUPR1, ANXA1, MT1X, CDC42EP2, TUBB6, SAA1, CSTB, VIM, GPX3, MT1E, HLA-DRB1, HLA-DRA, HLA-DRB5, HLA-DMA, CD74 and HLA-DPA1; or GPX3, LCN2, CLDN4, GPR56, DHRS3, SLC44A4, LYPD2, MUC20, TMPRSS4, MUC1, ELF3, SRGAP1, FOLR1, B2M, MDK, WFDC2, CMTM7, FTH1, CP, ESR1, CLDN3, RBP1, TNFAIP2, RNF213, MUC4, TACSTD2, CD74, HLA-DRA, HLA-DRB1 and HLA-DPA1; or SOD2, CXCL3, TNFAIP3, CXCL1, TUBA1A, TPM1, EDN1, TAGLN, CLDN1, CXCL2, TNFAIP2, NEDD9, ADAMTS9, FLNA, ARHGAP29, CTHRC1, RGS10, UBD, THBS1, CALD1, PTX3, RELB, CYR61, NFKBIA, CCL2, TNF, ICAM1, CCL20, IL8 and IL32; or UBD, TAP1, TNFAIP2, CRYAB, MARCO, LGALS14, PSMB9, CD74, TUBB2B, KRT23, IL4I1, TAPBP, C10orf10, CLDN1, SOD2, SELM, JAK3, TNFSF10, B2M, HLA-H, COL4A2, HLA-B, IL8, IL23A, ICAM1, TNF, CCL20, CCL2, IL32 and CD40.

### CRISPR-Cas Modification

In some embodiments, a polynucleotide of the present invention described elsewhere herein can be modified using a CRISPR-Cas and/or Cas-based system.

In general, a CRISPR-Cas or CRISPR system as used herein and in other documents, such as WO 2014/093622 (PCT/US2013/074667), refers collectively to transcripts and other elements involved in the expression of or directing the activity of CRISPR-associated (“Cas”) genes, including sequences encoding a Cas gene, a tracr (trans-activating CRISPR) sequence (e.g., tracrRNA or an active partial tracrRNA), a tracr-mate sequence (encompassing a “direct repeat” and a tracrRNA-processed partial direct repeat in the context of an endogenous CRISPR system), a guide sequence (also referred to as a “spacer” in the context of an endogenous CRISPR system), or “RNA(s)” as that term is herein used (e.g., RNA(s) to guide Cas, such as Cas9, e.g., CRISPR RNA and transactivating (tracr) RNA or a single guide RNA (sgRNA) (chimeric RNA)) or other sequences and transcripts from a CRISPR locus. In general, a CRISPR system is characterized by elements that promote the formation of a CRISPR complex at the site of a target sequence (also referred to as a protospacer in the context of an endogenous CRISPR system). See, e.g, Shmakov et al. (2015) “Discovery and Functional Characterization of Diverse Class 2 CRISPR-Cas Systems”, Molecular Cell, DOI: dx.doi.org/10.1016/j.molcel.2015.10.008.

CRISPR-Cas systems can generally fall into two classes based on their architectures of their effector molecules, which are each further subdivided by type and subtype. The two class are Class 1 and Class 2. Class 1 CRISPR-Cas systems have effector modules composed of multiple Cas proteins, some of which form crRNA-binding complexes, while Class 2 CRISPR-Cas systems include a single, multi-domain crRNA-binding protein.

In some embodiments, the CRISPR-Cas system that can be used to modify a polynucleotide of the present invention described herein can be a Class 1 CRISPR-Cas system. In some embodiments, the CRISPR-Cas system that can be used to modify a polynucleotide of the present invention described herein can be a Class 2 CRISPR-Cas system.

### Class 1 CRISPR-Cas Systems

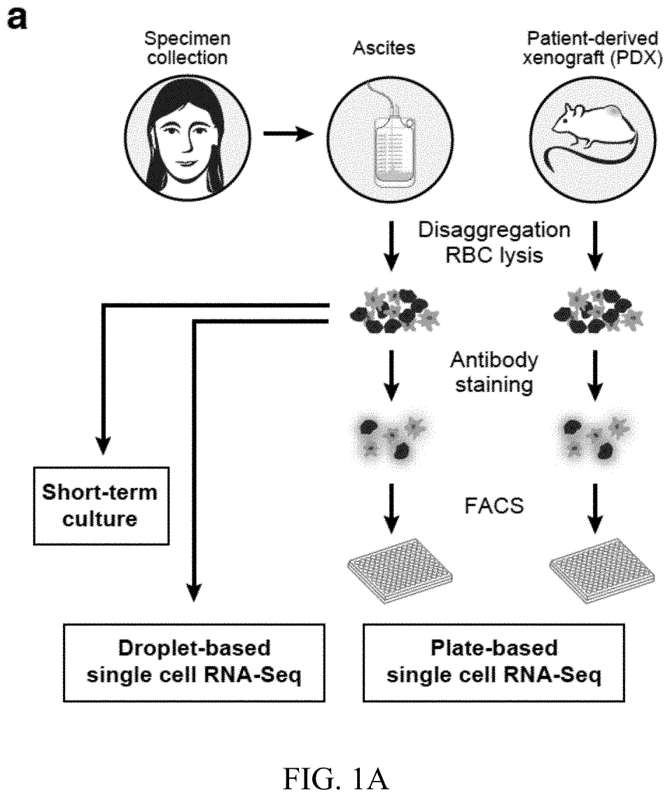

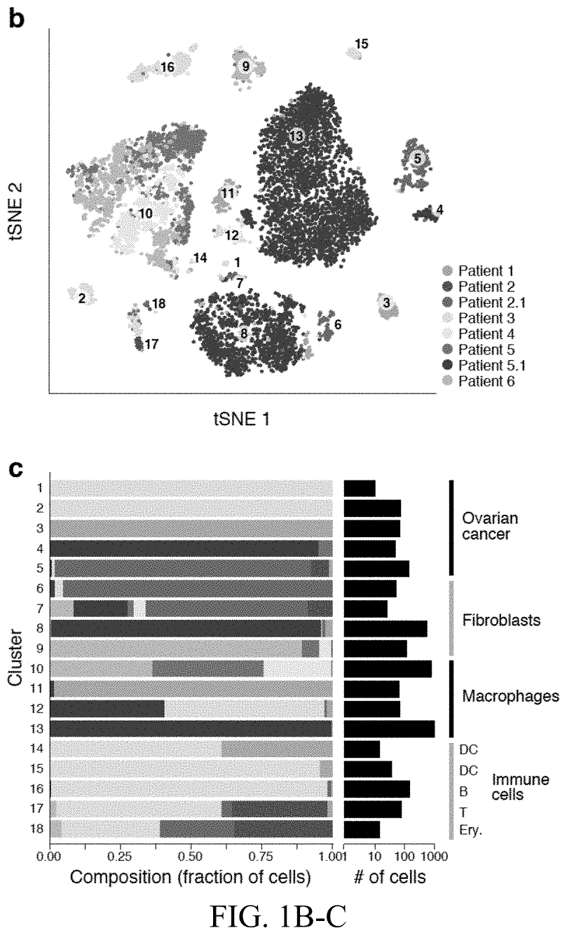

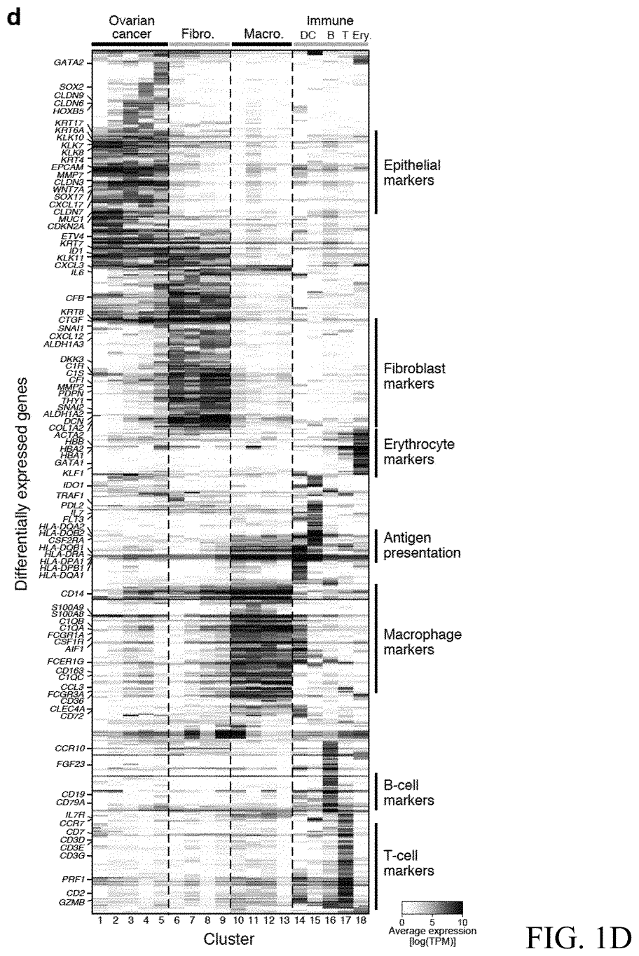

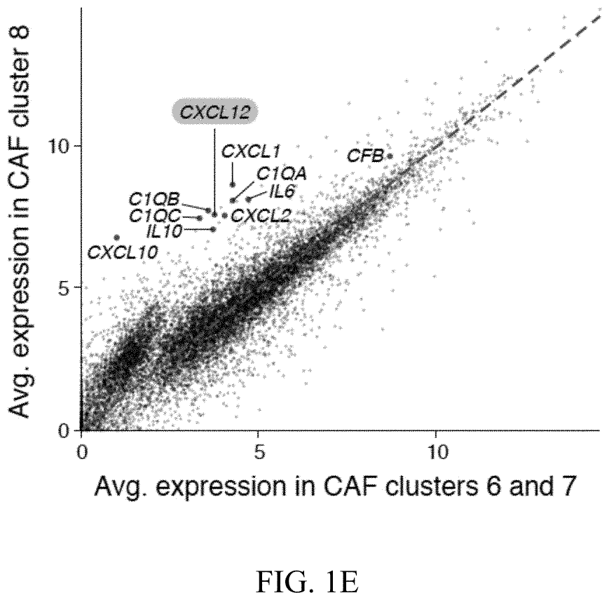

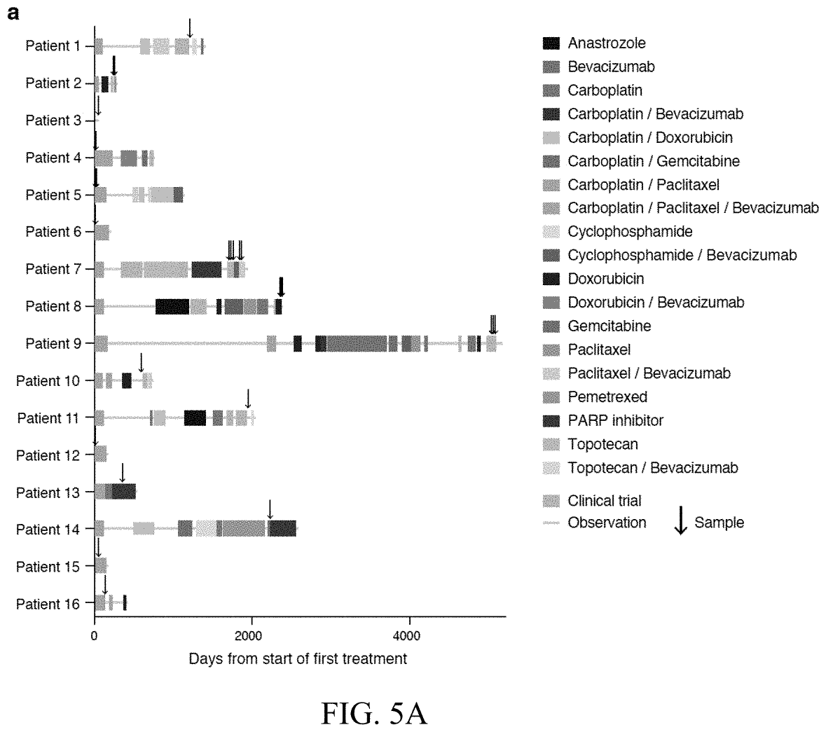

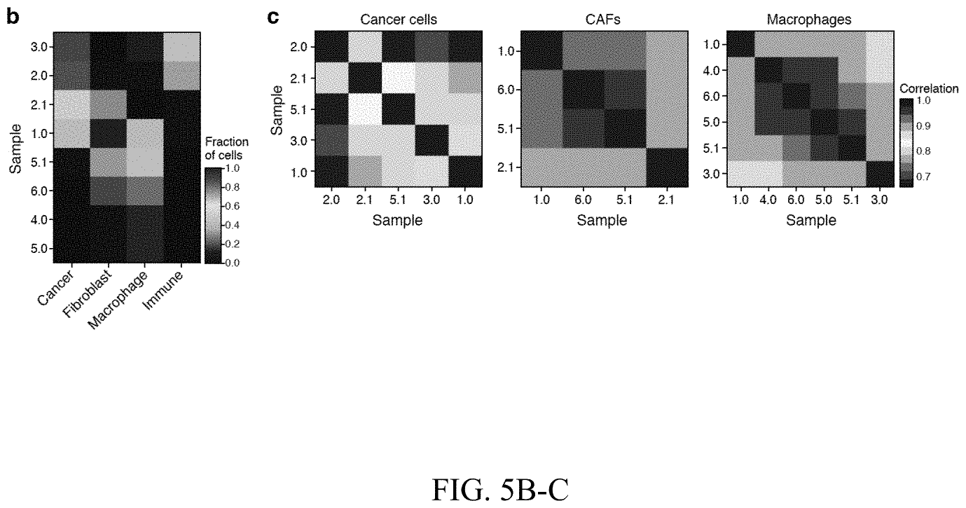

In some embodiments, the CRISPR-Cas system that can be used to modify a polynucleotide of the present invention described herein can be a Class 1 CRISPR-Cas system. Class 1 CRISPR-Cas systems are divided into types I, II, and IV. Makarova et al. 2020. Nat. Rev. 18: 67-83, particularly as described in FIG. 1. Type I CRISPR-Cas systems are divided into 9 subtypes (I-A, I-B, I-C, I-D, I-E, I-F1, I-F2, I-F3, and IG). Makarova et al., 2020. Class 1, Type I CRISPR-Cas systems can contain a Cas3 protein that can have helicase activity. Type III CRISPR-Cas systems are divided into 6 subtypes (III-A, III-B, III-C, III-D, III-E, and III-F). Type III CRISPR-Cas systems can contain a Cas10 that can include an RNA recognition motif called Palm and a cyclase domain that can cleave polynucleotides. Makarova et al., 2020. Type IV CRISPR-Cas systems are divided into 3 subtypes. (IV-A, IV-B, and IV-C). Makarova et al., 2020. Class 1 systems also include CRISPR-Cas variants, including Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems. Peters et al., PNAS 114 (35) (2017); DOI: 10.1073/pnas.1709035114; see also, Makarova et al. 2018. The CRISPR Journal, v. 1, n5, FIG. 5.

The Class 1 systems typically use a multi-protein effector complex, which can, in some embodiments, include ancillary proteins, such as one or more proteins in a complex referred to as a CRISPR-associated complex for antiviral defense (Cascade), one or more adaptation proteins (e.g., Cas1, Cas2, RNA nuclease), and/or one or more accessory proteins (e.g., Cas 4, DNA nuclease), CRISPR associated Rossman fold (CARF) domain containing proteins, and/or RNA transcriptase.

The backbone of the Class 1 CRISPR-Cas system effector complexes can be formed by RNA recognition motif domain-containing protein(s) of the repeat-associated mysterious proteins (RAMPs) family subunits (e.g., Cas 5, Cas6, and/or Cas7). RAMP proteins are characterized by having one or more RNA recognition motif domains. In some embodiments, multiple copies of RAMPs can be present. In some embodiments, the Class I CRISPR-Cas system can include 1, 2, 3, 4, 5, 6, 7, 8, 9, 10, 11, 12 or more Cas5, Cas6, and/or Cas 7 proteins. In some embodiments, the Cas6 protein is an RNAse, which can be responsible for pre-crRNA processing. When present in a Class 1 CRISPR-Cas system, Cas6 can be optionally physically associated with the effector complex.

Class 1 CRISPR-Cas system effector complexes can, in some embodiments, also include a large subunit. The large subunit can be composed of or include a Cas8 and/or Cas10 protein. See, e.g., FIGS. 1 and 2. Koonin E V, Makarova K S. 2019. Phil. Trans. R. Soc. B 374: 20180087, DOI: 10.1098/rstb.2018.0087 and Makarova et al. 2020.

Class 1 CRISPR-Cas system effector complexes can, in some embodiments, include a small subunit (for example, Cas11). See, e.g., FIGS. 1 and 2. Koonin E V, Makarova K S. 2019 Origins and Evolution of CRISPR-Cas systems. Phil. Trans. R. Soc. B 374: 20180087, DOI: 10.1098/rstb.2018.0087.

In some embodiments, the Class 1 CRISPR-Cas system can be a Type I CRISPR-Cas system. In some embodiments, the Type I CRISPR-Cas system can be a subtype I-A CRISPR-Cas system. In some embodiments, the Type I CRISPR-Cas system can be a subtype I-B CRISPR-Cas system. In some embodiments, the Type I CRISPR-Cas system can be a subtype I-C CRISPR-Cas system. In some embodiments, the Type I CRISPR-Cas system can be a subtype I-D CRISPR-Cas system. In some embodiments, the Type I CRISPR-Cas system can be a subtype I-E CRISPR-Cas system. In some embodiments, the Type I CRISPR-Cas system can be a subtype I-F1 CRISPR-Cas system. In some embodiments, the Type I CRISPR-Cas system can be a subtype I-F2 CRISPR-Cas system. In some embodiments, the Type I CRISPR-Cas system can be a subtype I-F3 CRISPR-Cas system. In some embodiments, the Type I CRISPR-Cas system can be a subtype I-G CRISPR-Cas system. In some embodiments, the Type I CRISPR-Cas system can be a CRISPR Cas variant, such as a Type I-A, I-B, I-E, I-F and I-U variants, which can include variants carried by transposons and plasmids, including versions of subtype I-F encoded by a large family of Tn7-like transposon and smaller groups of Tn7-like transposons that encode similarly degraded subtype I-B systems as previously described.

In some embodiments, the Class 1 CRISPR-Cas system can be a Type III CRISPR-Cas system. In some embodiments, the Type III CRISPR-Cas system can be a subtype III-A CRISPR-Cas system. In some embodiments, the Type III CRISPR-Cas system can be a subtype III-B CRISPR-Cas system. In some embodiments, the Type III CRISPR-Cas system can be a subtype III-C CRISPR-Cas system. In some embodiments, the Type III CRISPR-Cas system can be a subtype III-D CRISPR-Cas system. In some embodiments, the Type III CRISPR-Cas system can be a subtype III-E CRISPR-Cas system. In some embodiments, the Type III CRISPR-Cas system can be a subtype III-F CRISPR-Cas system.

In some embodiments, the Class 1 CRISPR-Cas system can be a Type IV CRISPR-Cas-system. In some embodiments, the Type IV CRISPR-Cas system can be a subtype IV-A CRISPR-Cas system. In some embodiments, the Type IV CRISPR-Cas system can be a subtype IV-B CRISPR-Cas system. In some embodiments, the Type IV CRISPR-Cas system can be a subtype IV-C CRISPR-Cas system.

The effector complex of a Class 1 CRISPR-Cas system can, in some embodiments, include a Cas3 protein that is optionally fused to a Cas2 protein, a Cas4, a Cas5, a Cas6, a Cas7, a Cas8, a Cas10, a Cas11, or a combination thereof. In some embodiments, the effector complex of a Class 1 CRISPR-Cas system can have multiple copies, such as 1, 2, 3, 4, 5, 6, 7, 8, 9, 10, 11, 12, 13, or 14, of any one or more Cas proteins.

### Class 2 CRISPR-Cas Systems

The compositions, systems, and methods described in greater detail elsewhere herein can be designed and adapted for use with Class 2 CRISPR-Cas systems. Thus, in some embodiments, the CRISPR-Cas system is a Class 2 CRISPR-Cas system. Class 2 systems are distinguished from Class 1 systems in that they have a single, large, multi-domain effector protein. In certain example embodiments, the Class 2 system can be a Type II, Type V, or Type VI system, which are described in Makarova et al. “Evolutionary classification of CRISPR-Cas systems: a burst of class 2 and derived variants” Nature Reviews Microbiology, 18:67-81 (February 2020), incorporated herein by reference. Each type of Class 2 system is further divided into subtypes. See Markova et al. 2020, particularly at Figure. 2. Class 2, Type II systems can be divided into 4 subtypes: II-A, II-B, II-C1, and II-C2. Class 2, Type V systems can be divided into 17 subtypes: V-A, V-B1, V-B2, V-C, V-D, V-E, V-F1, V-F1(V-U3), V-F2, V-F3, V-G, V-H, V-I, V-K (V-U5), V-U1, V-U2, and V-U4. Class 2, Type IV systems can be divided into 5 subtypes: VI-A, VI-B1, VI-B2, VI-C, and VI-D.

The distinguishing feature of these types is that their effector complexes consist of a single, large, multi-domain protein. Type V systems differ from Type II effectors (e.g., Cas9), which contain two nuclear domains that are each responsible for the cleavage of one strand of the target DNA, with the HNH nuclease inserted inside the Ruv-C like nuclease domain sequence. The Type V systems (e.g., Cas12) only contain a RuvC-like nuclease domain that cleaves both strands. Type VI (Cas13) are unrelated to the effectors of Type II and V systems and contain two HEPN domains and target RNA. Cas13 proteins also display collateral activity that is triggered by target recognition. Some Type V systems have also been found to possess this collateral activity with two single-stranded DNA in in vitro contexts.

In some embodiments, the Class 2 system is a Type II system. In some embodiments, the Type II CRISPR-Cas system is a II-A CRISPR-Cas system. In some embodiments, the Type II CRISPR-Cas system is a II-B CRISPR-Cas system. In some embodiments, the Type II CRISPR-Cas system is a II-C1 CRISPR-Cas system. In some embodiments, the Type II CRISPR-Cas system is a II-C2 CRISPR-Cas system. In some embodiments, the Type II system is a Cas9 system. In some embodiments, the Type II system includes a Cas9.

In some embodiments, the Class 2 system is a Type V system. In some embodiments, the Type V CRISPR-Cas system is a V-A CRISPR-Cas system. In some embodiments, the Type V CRISPR-Cas system is a V-B1 CRISPR-Cas system. In some embodiments, the Type V CRISPR-Cas system is a V-B2 CRISPR-Cas system. In some embodiments, the Type V CRISPR-Cas system is a V-C CRISPR-Cas system. In some embodiments, the Type V CRISPR-Cas system is a V-D CRISPR-Cas system. In some embodiments, the Type V CRISPR-Cas system is a V-E CRISPR-Cas system. In some embodiments, the Type V CRISPR-Cas system is a V-F1 CRISPR-Cas system. In some embodiments, the Type V CRISPR-Cas system is a V-F1 (V-U3) CRISPR-Cas system. In some embodiments, the Type V CRISPR-Cas system is a V-F2 CRISPR-Cas system. In some embodiments, the Type V CRISPR-Cas system is a V-F3 CRISPR-Cas system. In some embodiments, the Type V CRISPR-Cas system is a V-G CRISPR-Cas system. In some embodiments, the Type V CRISPR-Cas system is a V-H CRISPR-Cas system. In some embodiments, the Type V CRISPR-Cas system is a V-I CRISPR-Cas system. In some embodiments, the Type V CRISPR-Cas system is a V-K (V-U5) CRISPR-Cas system. In some embodiments, the Type V CRISPR-Cas system is a V-U1 CRISPR-Cas system. In some embodiments, the Type V CRISPR-Cas system is a V-U2 CRISPR-Cas system. In some embodiments, the Type V CRISPR-Cas system is a V-U4 CRISPR-Cas system. In some embodiments, the Type V CRISPR-Cas system includes a Cas12a (Cpf1), Cas12b (C2c1), Cas12c (C2c3), CasX, and/or Cas14.

In some embodiments the Class 2 system is a Type VI system. In some embodiments, the Type VI CRISPR-Cas system is a VI-A CRISPR-Cas system. In some embodiments, the Type VI CRISPR-Cas system is a VI-B1 CRISPR-Cas system. In some embodiments, the Type VI CRISPR-Cas system is a VI-B2 CRISPR-Cas system. In some embodiments, the Type VI CRISPR-Cas system is a VI-C CRISPR-Cas system. In some embodiments, the Type VI CRISPR-Cas system is a VI-D CRISPR-Cas system. In some embodiments, the Type VI CRISPR-Cas system includes a Cas13a (C2c2), Cas13b (Group 29/30), Cas13c, and/or Cas13d.

### Specialized Cas-Based Systems

In some embodiments, the system is a Cas-based system that is capable of performing a specialized function or activity. For example, the Cas protein may be fused, operably coupled to, or otherwise associated with one or more functionals domains. In certain example embodiments, the Cas protein may be a catalytically dead Cas protein (“dCas”) and/or have nickase activity. A nickase is a Cas protein that cuts only one strand of a double stranded target. In such embodiments, the dCas or nickase provide a sequence specific targeting functionality that delivers the functional domain to or proximate a target sequence. Example functional domains that may be fused to, operably coupled to, or otherwise associated with a Cas protein can be or include, but are not limited to a nuclear localization signal (NLS) domain, a nuclear export signal (NES) domain, a translational activation domain, a transcriptional activation domain (e.g. VP64, p65, MyoD1, HSF1, RTA, and SET7/9), a translation initiation domain, a transcriptional repression domain (e.g., a KRAB domain, NuE domain, NcoR domain, and a SID domain such as a SID4X domain), a nuclease domain (e.g., FokI), a histone modification domain (e.g., a histone acetyltransferase), a light inducible/controllable domain, a chemically inducible/controllable domain, a transposase domain, a homologous recombination machinery domain, a recombinase domain, an integrase domain, and combinations thereof. Methods for generating catalytically dead Cas9 or a nickase Cas9 (WO 2014/204725, Ran et al. Cell. 2013 Sep. 12; 154(6):1380-1389), Cas12 (Liu et al. Nature Communications, 8, 2095 (2017), and Cas13 (WO 2019/005884, WO2019/060746) are known in the art and incorporated herein by reference.

In some embodiments, the functional domains can have one or more of the following activities: methylase activity, demethylase activity, translation activation activity, translation initiation activity, translation repression activity, transcription activation activity, transcription repression activity, transcription release factor activity, histone modification activity, nuclease activity, single-strand RNA cleavage activity, double-strand RNA cleavage activity, single-strand DNA cleavage activity, double-strand DNA cleavage activity, molecular switch activity, chemical inducibility, light inducibility, and nucleic acid binding activity. In some embodiments, the one or more functional domains may comprise epitope tags or reporters. Non-limiting examples of epitope tags include histidine (His) tags, V5 tags, FLAG tags, influenza hemagglutinin (HA) tags, Myc tags, VSV-G tags, and thioredoxin (Trx) tags. Examples of reporters include, but are not limited to, glutathione-S-transferase (GST), horseradish peroxidase (HRP), chloramphenicol acetyltransferase (CAT) beta-galactosidase, beta-glucuronidase, luciferase, green fluorescent protein (GFP), HcRed, DsRed, cyan fluorescent protein (CFP), yellow fluorescent protein (YFP), and auto-fluorescent proteins including blue fluorescent protein (BFP).

The one or more functional domain(s) may be positioned at, near, and/or in proximity to a terminus of the effector protein (e.g., a Cas protein). In embodiments having two or more functional domains, each of the two can be positioned at or near or in proximity to a terminus of the effector protein (e.g., a Cas protein). In some embodiments, such as those where the functional domain is operably coupled to the effector protein, the one or more functional domains can be tethered or linked via a suitable linker (including, but not limited to, GlySer linkers) to the effector protein (e.g., a Cas protein). When there is more than one functional domain, the functional domains can be same or different. In some embodiments, all the functional domains are the same. In some embodiments, all of the functional domains are different from each other. In some embodiments, at least two of the functional domains are different from each other. In some embodiments, at least two of the functional domains are the same as each other.

Other suitable functional domains can be found, for example, in International Application Publication No. WO 2019/018423.

### Split CRISPR-Cas Systems

In some embodiments, the CRISPR-Cas system is a split CRISPR-Cas system. See e.g., Zetche et al., 2015. Nat. Biotechnol. 33(2): 139-142 and WO 2019/018423, the compositions and techniques of which can be used in and/or adapted for use with the present invention. Split CRISPR-Cas proteins are set forth herein and in documents incorporated herein by reference in further detail herein. In certain embodiments, each part of a split CRISPR protein are attached to a member of a specific binding pair, and when bound with each other, the members of the specific binding pair maintain the parts of the CRISPR protein in proximity. In certain embodiments, each part of a split CRISPR protein is associated with an inducible binding pair. An inducible binding pair is one which is capable of being switched “on” or “off” by a protein or small molecule that binds to both members of the inducible binding pair. In some embodiments, CRISPR proteins may preferably split between domains, leaving domains intact. In particular embodiments, said Cas split domains (e.g., RuvC and HNH domains in the case of Cas9) can be simultaneously or sequentially introduced into the cell such that said split Cas domain(s) process the target nucleic acid sequence in the algae cell. The reduced size of the split Cas compared to the wild type Cas allows other methods of delivery of the systems to the cells, such as the use of cell penetrating peptides as described herein.

### DNA and RNA Base Editing

In some embodiments, a polynucleotide of the present invention described elsewhere herein can be modified using a base editing system. In some embodiments, a Cas protein is connected or fused to a nucleotide deaminase. Thus, in some embodiments the Cas-based system can be a base editing system. As used herein “base editing” refers generally to the process of polynucleotide modification via a CRISPR-Cas-based or Cas-based system that does not include excising nucleotides to make the modification. Base editing can convert base pairs at precise locations without generating excess undesired editing byproducts that can be made using traditional CRISPR-Cas systems.

In certain example embodiments, the nucleotide deaminase may be a DNA base editor used in combination with a DNA binding Cas protein such as, but not limited to, Class 2 Type II and Type V systems. Two classes of DNA base editors are generally known: cytosine base editors (CBEs) and adenine base editors (ABEs). CBEs convert a C⋅G base pair into a T⋅A base pair (Komor et al. 2016. Nature. 533:420-424; Nishida et al. 2016. Science. 353; and Li et al. Nat. Biotech. 36:324-327) and ABEs convert an A⋅T base pair to a G⋅C base pair. Collectively, CBEs and ABEs can mediate all four possible transition mutations (C to T, A to G, T to C, and G to A). Rees and Liu. 2018. Nat. Rev. Genet. 19(12): 770-788, particularly at FIGS. 1b, 2a-2c, 3a-3f, and Table 1. In some embodiments, the base editing system includes a CBE and/or an ABE. In some embodiments, a polynucleotide of the present invention described elsewhere herein can be modified using a base editing system. Rees and Liu. 2018. Nat. Rev. Gent. 19(12):770-788. Base editors also generally do not need a DNA donor template and/or rely on homology-directed repair. Komor et al. 2016. Nature. 533:420-424; Nishida et al. 2016. Science. 353; and Gaudeli et al. 2017. Nature. 551:464-471. Upon binding to a target locus in the DNA, base pairing between the guide RNA of the system and the target DNA strand leads to displacement of a small segment of ssDNA in an “R-loop”. Nishimasu et al. Cell. 156:935-949. DNA bases within the ssDNA bubble are modified by the enzyme component, such as a deaminase. In some systems, the catalytically disabled Cas protein can be a variant or modified Cas can have nickase functionality and can generate a nick in the non-edited DNA strand to induce cells to repair the non-edited strand using the edited strand as a template. Komor et al. 2016. Nature. 533:420-424; Nishida et al. 2016. Science. 353; and Gaudeli et al. 2017. Nature. 551:464-471. Base editors may be further engineered to optimize conversion of nucleotides (e.g. A:T to G:C). Richter et al. 2020. Nature Biotechnology. doi.org/10.1038/s41587-020-0453-z.

Other Example Type V base editing systems are described in WO 2018/213708, WO 2018/213726, PCT/US2018/067207, PCT/US2018/067225, and PCT/US2018/067307 which are incorporated by referenced herein.

In certain example embodiments, the base editing system may be a RNA base editing system. As with DNA base editors, a nucleotide deaminase capable of converting nucleotide bases may be fused to a Cas protein. However, in these embodiments, the Cas protein will need to be capable of binding RNA. Example RNA binding Cas proteins include, but are not limited to, RNA-binding Cas9s such as Francisella novicida Cas9 (“FnCas9”), and Class 2 Type VI Cas systems. The nucleotide deaminase may be a cytidine deaminase or an adenosine deaminase, or an adenosine deaminase engineered to have cytidine deaminase activity. In certain example embodiments, the RNA based editor may be used to delete or introduce a post-translation modification site in the expressed mRNA. In contrast to DNA base editors, whose edits are permanent in the modified cell, RNA base editors can provide edits where finer temporal control may be needed, for example in modulating a particular immune response. Example Type VI RNA-base editing systems are described in Cox et al. 2017. Science 358: 1019-1027, WO 2019/005884, WO 2019/005886, WO 2019/071048, PCT/US20018/05179, PCT/US2018/067207, which are incorporated herein by reference. An example FnCas9 system that may be adapted for RNA base editing purposes is described in WO 2016/106236, which is incorporated herein by reference.

An example method for delivery of base-editing systems, including use of a split-intein approach to divide CBE and ABE into reconstituble halves, is described in Levy et al. Nature Biomedical Engineering doi.org/10.1038/s41441-019-0505-5 (2019), which is incorporated herein by reference.

### Prime Editors

In some embodiments, a polynucleotide of the present invention described elsewhere herein can be modified using a prime editing system See e.g. Anzalone et al. 2019. Nature. 576: 149-157. Like base editing systems, prime editing systems can be capable of targeted modification of a polynucleotide without generating double stranded breaks and does not require donor templates. Further prime editing systems can be capable of all 12 possible combination swaps. Prime editing can operate via a “search-and-replace” methodology and can mediate targeted insertions, deletions, all 12 possible base-to-base conversion, and combinations thereof. Generally, a prime editing system, as exemplified by PE1, PE2, and PE3 (Id.), can include a reverse transcriptase fused or otherwise coupled or associated with an RNA-programmable nickase, and a prime-editing extended guide RNA (pegRNA) to facility direct copying of genetic information from the extension on the pegRNA into the target polynucleotide. Embodiments that can be used with the present invention include these and variants thereof. Prime editing can have the advantage of lower off-target activity than traditional CRIPSR-Cas systems along with few byproducts and greater or similar efficiency as compared to traditional CRISPR-Cas systems.

In some embodiments, the prime editing guide molecule can specify both the target polynucleotide information (e.g. sequence) and contain a new polynucleotide cargo that replaces target polynucleotides. To initiate transfer from the guide molecule to the target polynucleotide, the PE system can nick the target polynucleotide at a target side to expose a 3′hydroxyl group, which can prime reverse transcription of an edit-encoding extension region of the guide molecule (e.g. a prime editing guide molecule or peg guide molecule) directly into the target site in the target polynucleotide. See e.g. Anzalone et al. 2019. Nature. 576: 149-157, particularly at FIGS. 1b, 1c, related discussion, and Supplementary discussion.

In some embodiments, a prime editing system can be composed of a Cas polypeptide having nickase activity, a reverse transcriptase, and a guide molecule. The Cas polypeptide can lack nuclease activity. The guide molecule can include a target binding sequence as well as a primer binding sequence and a template containing the edited polynucleotide sequence. The guide molecule, Cas polypeptide, and/or reverse transcriptase can be coupled together or otherwise associate with each other to form an effector complex and edit a target sequence. In some embodiments, the Cas polypeptide is a Class 2, Type V Cas polypeptide. In some embodiments, the Cas polypeptide is a Cas9 polypeptide (e.g. is a Cas9 nickase). In some embodiments, the Cas polypeptide is fused to the reverse transcriptase. In some embodiments, the Cas polypeptide is linked to the reverse transcriptase.

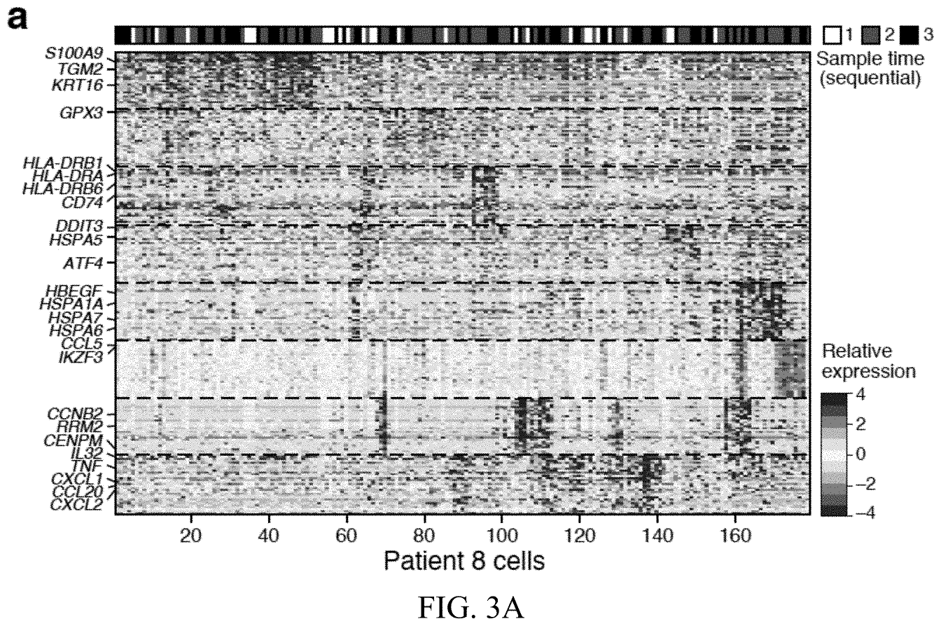

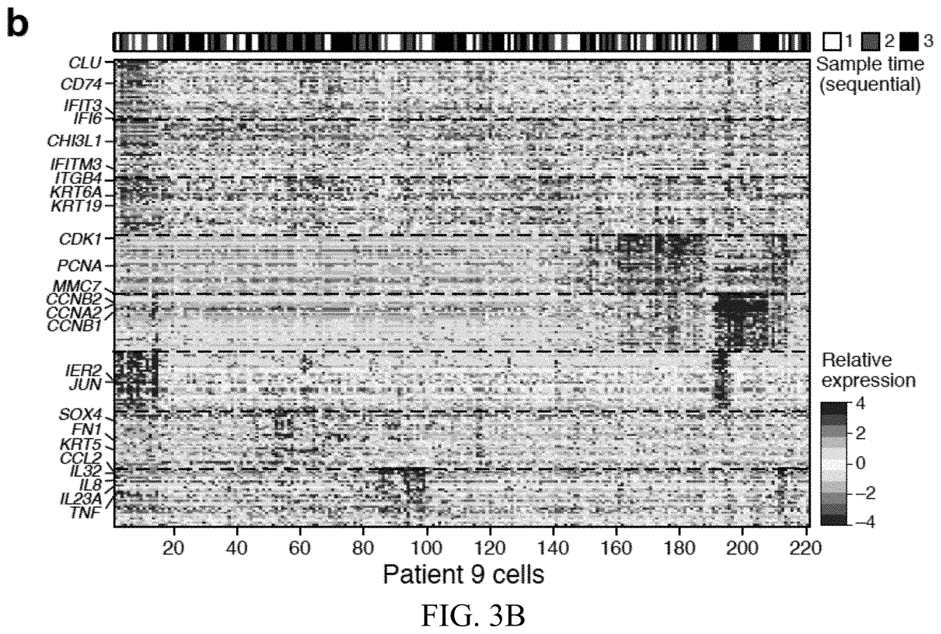

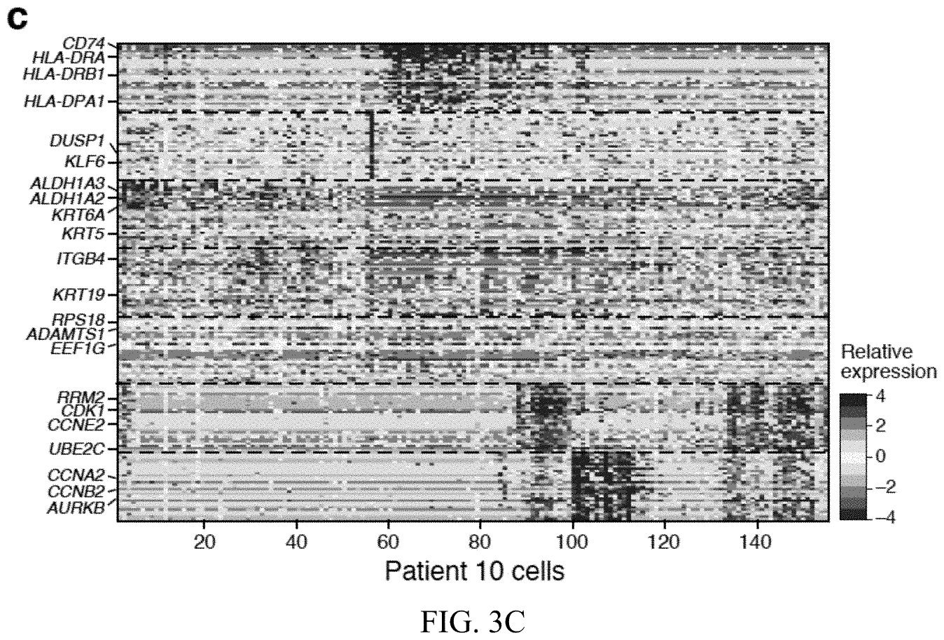

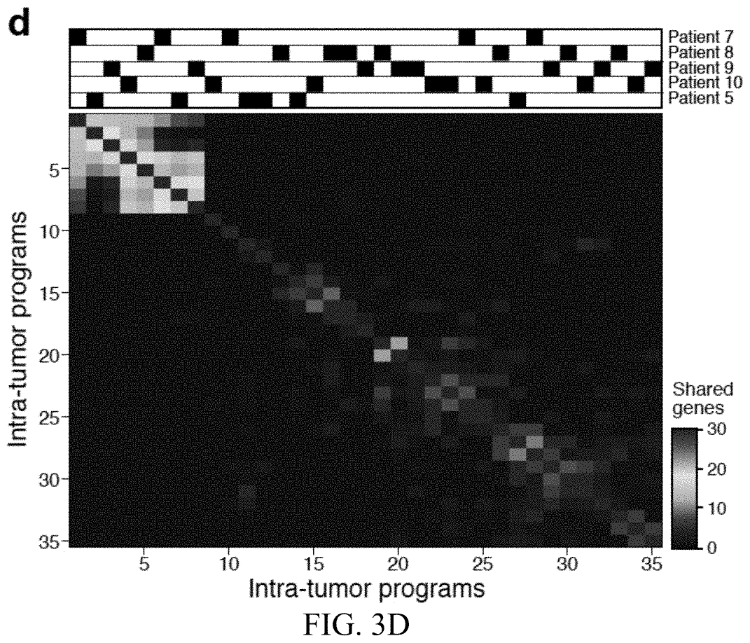

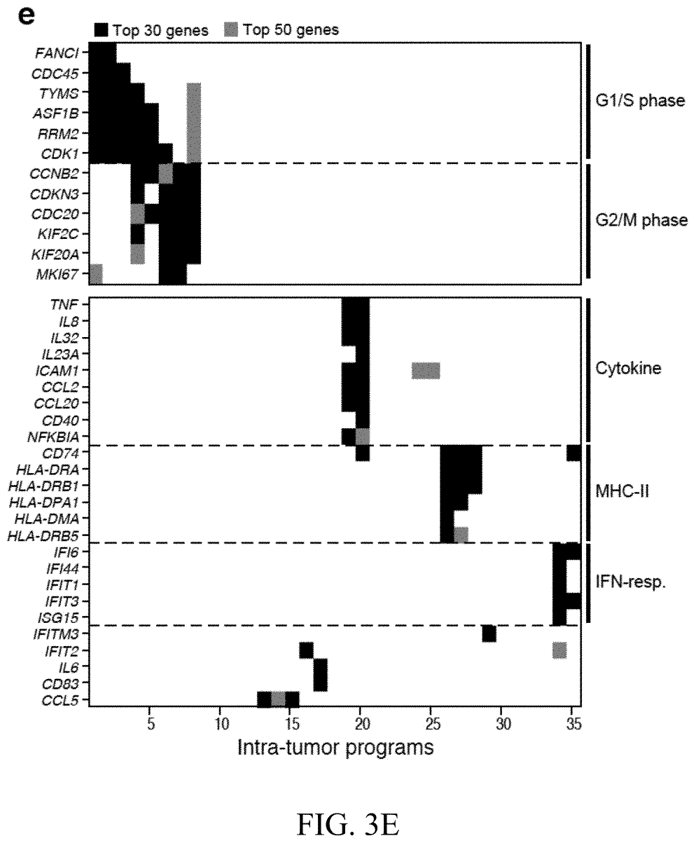

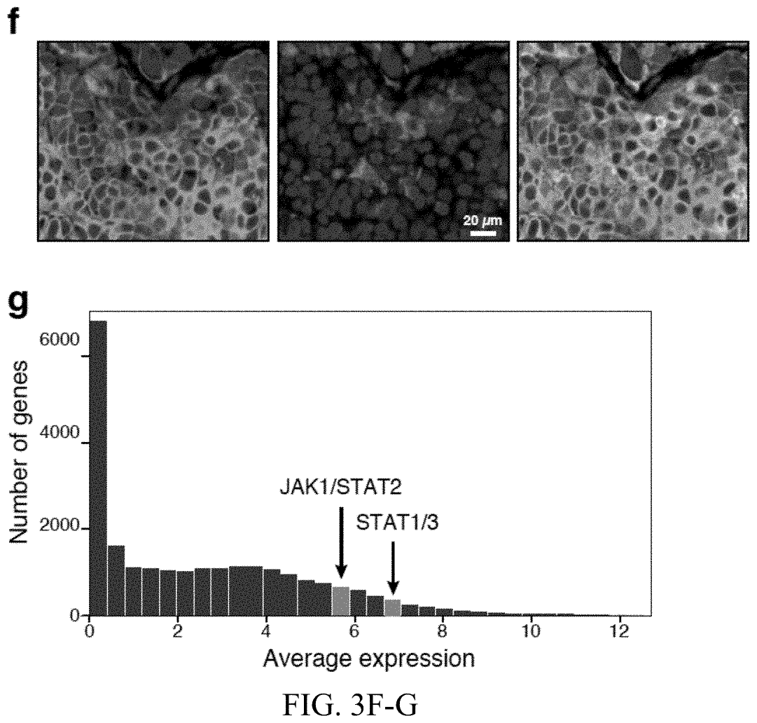

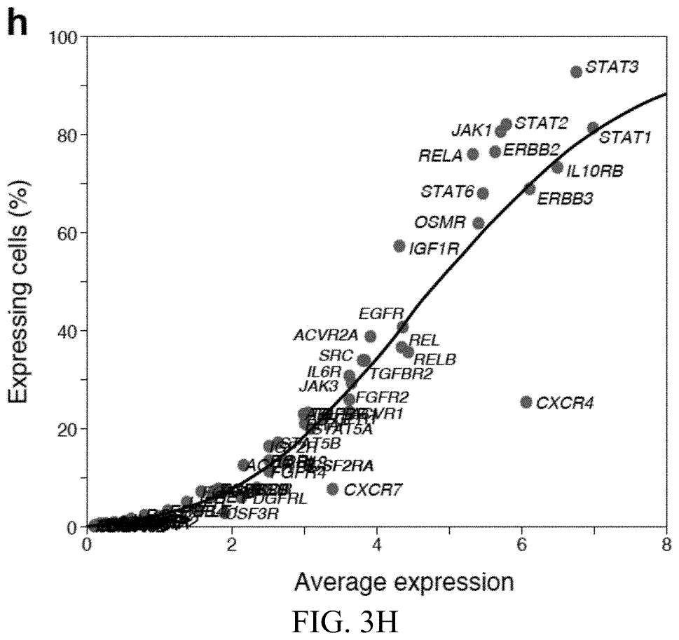

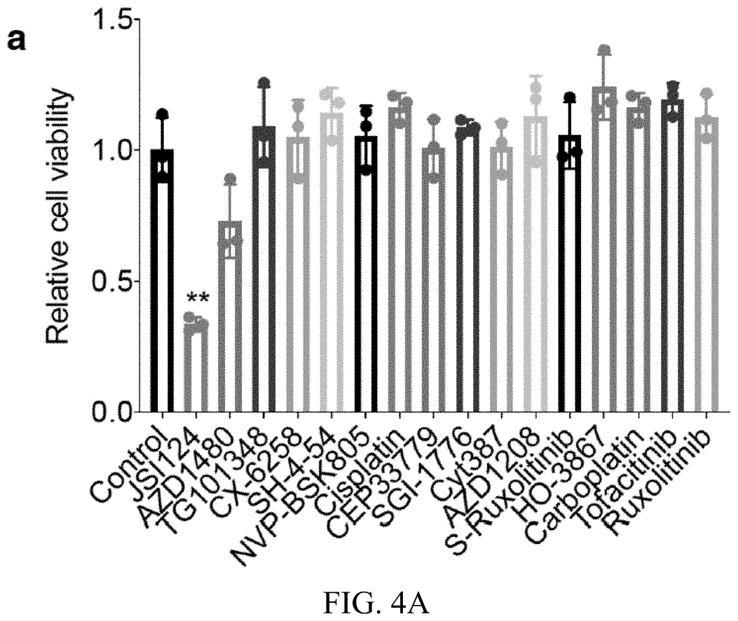

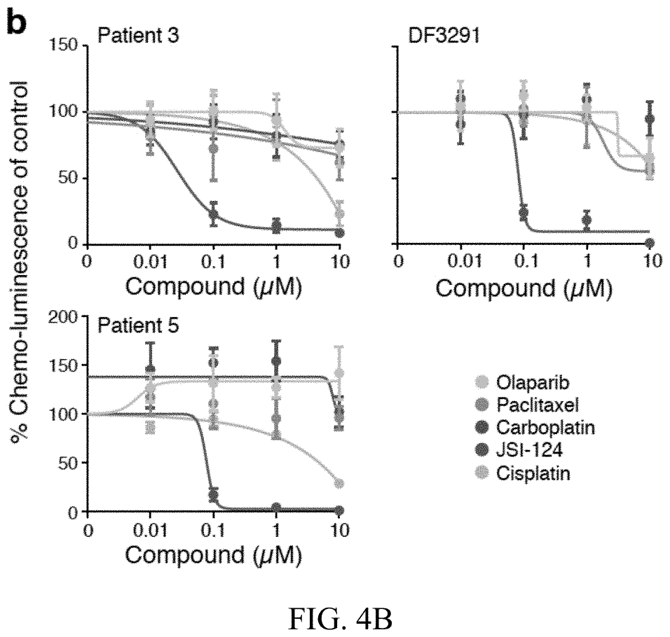

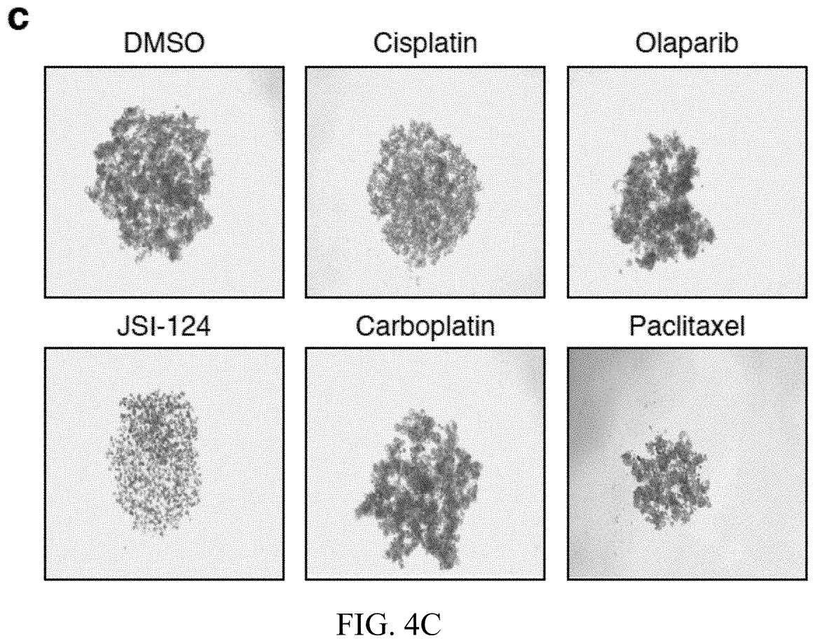

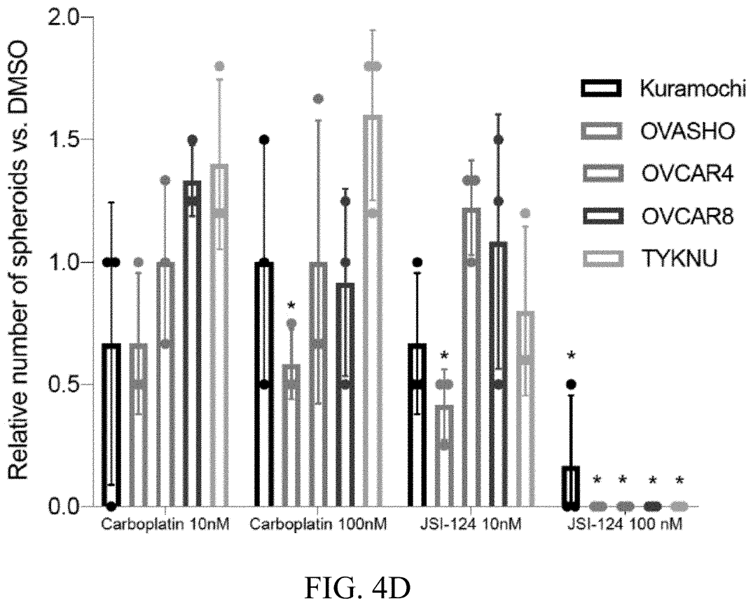

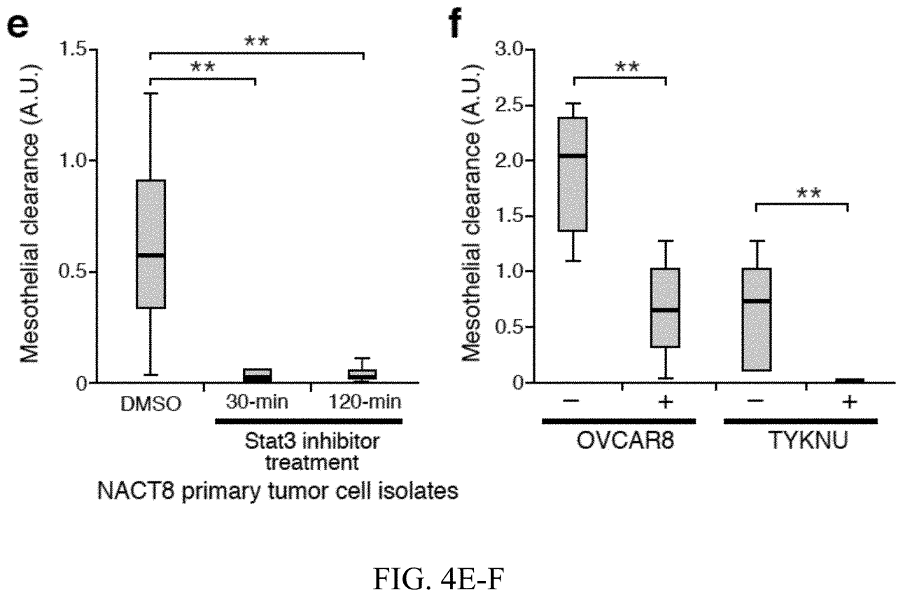

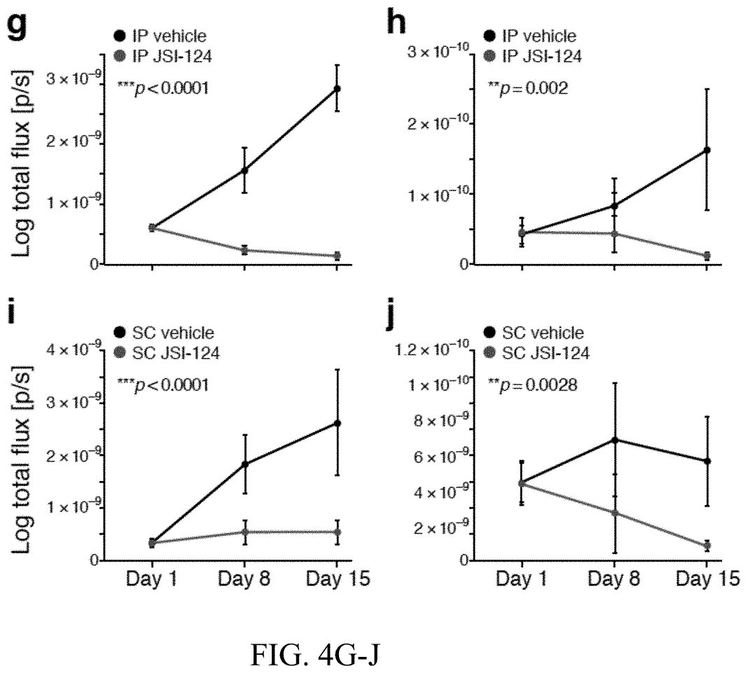

In some embodiments, the prime editing system can be a PE1 system or variant thereof, a PE2 system or variant thereof, or a PE3 (e.g. PE3, PE3b) system. See e.g., Anzalone et al. 2019. Nature. 576: 149-157, particularly at pgs. 2-3, FIGS. 2a, 3a-3f, 4a-4b, Extended data FIGS. 3a-3b, 4,

The peg guide molecule can be about 10 to about 200 or more nucleotides in length, such as 10 to/or 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149, 150, 151, 152, 153, 154, 155, 156, 157, 158, 159, 160, 161, 162, 163, 164, 165, 166, 167, 168, 169, 170, 171, 172, 173, 174, 175, 176, 177, 178, 179, 180, 181, 182, 183, 184, 185, 186, 187, 188, 189, 190, 191, 192, 193, 194, 195, 196, 197, 198, 199, or 200 or more nucleotides in length. Optimization of the peg guide molecule can be accomplished as described in Anzalone et al. 2019. Nature. 576: 149-157, particularly at pg. 3, FIG. 2a-2b, and Extended Data FIGS. 5a-c.

### CRISPR Associated Transposase (CAST) Systems

In some embodiments, a polynucleotide of the present invention described elsewhere herein can be modified using a CRISPR Associated Transposase (“CAST”) system. CAST system can include a Cas protein that is catalytically inactive, or engineered to be catalytically active, and further comprises a transposase (or subunits thereof) that catalyze RNA-guided DNA transposition. Such systems are able to insert DNA sequences at a target site in a DNA molecule without relying on host cell repair machinery. CAST systems can be Class1 or Class 2 CAST systems. An example Class 1 system is described in Klompe et al. Nature, doi:10.1038/s41586-019-1323, which is in incorporated herein by reference. An example Class 2 system is described in Strecker et al. Science. 10/1126/science. aax9181 (2019), and PCT/US2019/066835 which are incorporated herein by reference.

### Guide Molecules

The CRISPR-Cas or Cas-Based system described herein can, in some embodiments, include one or more guide molecules. The terms guide molecule, guide sequence and guide polynucleotide, refer to polynucleotides capable of guiding Cas to a target genomic locus and are used interchangeably as in foregoing cited documents such as WO 2014/093622 (PCT/US2013/074667). In general, a guide sequence is any polynucleotide sequence having sufficient complementarity with a target polynucleotide sequence to hybridize with the target sequence and direct sequence-specific binding of a CRISPR complex to the target sequence. The guide molecule can be a polynucleotide.

The ability of a guide sequence (within a nucleic acid-targeting guide RNA) to direct sequence-specific binding of a nucleic acid-targeting complex to a target nucleic acid sequence may be assessed by any suitable assay. For example, the components of a nucleic acid-targeting CRISPR system sufficient to form a nucleic acid-targeting complex, including the guide sequence to be tested, may be provided to a host cell having the corresponding target nucleic acid sequence, such as by transfection with vectors encoding the components of the nucleic acid-targeting complex, followed by an assessment of preferential targeting (e.g., cleavage) within the target nucleic acid sequence, such as by Surveyor assay (Qui et al. 2004. BioTechniques. 36(4)702-707). Similarly, cleavage of a target nucleic acid sequence may be evaluated in a test tube by providing the target nucleic acid sequence, components of a nucleic acid-targeting complex, including the guide sequence to be tested and a control guide sequence different from the test guide sequence, and comparing binding or rate of cleavage at the target sequence between the test and control guide sequence reactions. Other assays are possible and will occur to those skilled in the art.

In some embodiments, the guide molecule is an RNA. The guide molecule(s) (also referred to interchangeably herein as guide polynucleotide and guide sequence) that are included in the CRISPR-Cas or Cas based system can be any polynucleotide sequence having sufficient complementarity with a target nucleic acid sequence to hybridize with the target nucleic acid sequence and direct sequence-specific binding of a nucleic acid-targeting complex to the target nucleic acid sequence. In some embodiments, the degree of complementarity, when optimally aligned using a suitable alignment algorithm, can be about or more than about 50%, 60%, 75%, 80%, 85%, 90%, 95%, 97.5%, 99%, or more. Optimal alignment may be determined with the use of any suitable algorithm for aligning sequences, non-limiting examples of which include the Smith-Waterman algorithm, the Needleman-Wunsch algorithm, algorithms based on the Burrows-Wheeler Transform (e.g., the Burrows Wheeler Aligner), ClustalW, Clustal X, BLAT, Novoalign (Novocraft Technologies; available at www.novocraft.com), ELAND (Illumina, San Diego, Calif.), SOAP (available at soap.genomics.org.cn), and Maq (available at maq.sourceforge.net).

A guide sequence, and hence a nucleic acid-targeting guide may be selected to target any target nucleic acid sequence. The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

In some embodiments, a nucleic acid-targeting guide is selected to reduce the degree secondary structure within the nucleic acid-targeting guide. In some embodiments, about or less than about 75%, 50%, 40%, 30%, 25%, 20%, 15%, 10%, 5%, 1%, or fewer of the nucleotides of the nucleic acid-targeting guide participate in self-complementary base pairing when optimally folded. Optimal folding may be determined by any suitable polynucleotide folding algorithm. Some programs are based on calculating the minimal Gibbs free energy. An example of one such algorithm is mFold, as described by Zuker and Stiegler (Nucleic Acids Res. 9 (1981), 133-148). Another example folding algorithm is the online webserver RNAfold, developed at Institute for Theoretical Chemistry at the University of Vienna, using the centroid structure prediction algorithm (see e.g., A. R. Gruber et al., 2008, Cell 106(1): 23-24; and P A Carr and G M Church, 2009, Nature Biotechnology 27(12): 1151-62).

In certain embodiments, a guide RNA or crRNA may comprise, consist essentially of, or consist of a direct repeat (DR) sequence and a guide sequence or spacer sequence. In certain embodiments, the guide RNA or crRNA may comprise, consist essentially of, or consist of a direct repeat sequence fused or linked to a guide sequence or spacer sequence. In certain embodiments, the direct repeat sequence may be located upstream (i.e., 5′) from the guide sequence or spacer sequence. In other embodiments, the direct repeat sequence may be located downstream (i.e., 3′) from the guide sequence or spacer sequence.

In certain embodiments, the crRNA comprises a stem loop, preferably a single stem loop. In certain embodiments, the direct repeat sequence forms a stem loop, preferably a single stem loop.

In certain embodiments, the spacer length of the guide RNA is from 15 to 35 nt. In certain embodiments, the spacer length of the guide RNA is at least 15 nucleotides. In certain embodiments, the spacer length is from 15 to 17 nt, e.g., 15, 16, or 17 nt, from 17 to 20 nt, e.g., 17, 18, 19, or 20 nt, from 20 to 24 nt, e.g., 20, 21, 22, 23, or 24 nt, from 23 to 25 nt, e.g., 23, 24, or 25 nt, from 24 to 27 nt, e.g., 24, 25, 26, or 27 nt, from 27 to 30 nt, e.g., 27, 28, 29, or 30 nt, from 30 to 35 nt, e.g., 30, 31, 32, 33, 34, or 35 nt, or 35 nt or longer.

The “tracrRNA” sequence or analogous terms includes any polynucleotide sequence that has sufficient complementarity with a crRNA sequence to hybridize. In some embodiments, the degree of complementarity between the tracrRNA sequence and crRNA sequence along the length of the shorter of the two when optimally aligned is about or more than about 25%, 30%, 40%, 50%, 60%, 70%, 80%, 90%, 95%, 97.5%, 99%, or higher. In some embodiments, the tracr sequence is about or more than about 5, 6, 7, 8, 9, 10, 11, 12, 13, 14, 15, 16, 17, 18, 19, 20, 25, 30, 40, 50, or more nucleotides in length. In some embodiments, the tracr sequence and crRNA sequence are contained within a single transcript, such that hybridization between the two produces a transcript having a secondary structure, such as a hairpin.

In general, degree of complementarity is with reference to the optimal alignment of the sca sequence and tracr sequence, along the length of the shorter of the two sequences. Optimal alignment may be determined by any suitable alignment algorithm, and may further account for secondary structures, such as self-complementarity within either the sca sequence or tracr sequence. In some embodiments, the degree of complementarity between the tracr sequence and sca sequence along the length of the shorter of the two when optimally aligned is about or more than about 25%, 30%, 40%, 50%, 60%, 70%, 80%, 90%, 95%, 97.5%, 99%, or higher.

In some embodiments, the degree of complementarity between a guide sequence and its corresponding target sequence can be about or more than about 50%, 60%, 75%, 80%, 85%, 90%, 95%, 97.5%, 99%, or 100%; a guide or RNA or sgRNA can be about or more than about 5, 10, 11, 12, 13, 14, 15, 16, 17, 18, 19, 20, 21, 22, 23, 24, 25, 26, 27, 28, 29, 30, 35, 40, 45, 50, 75, or more nucleotides in length; or guide or RNA or sgRNA can be less than about 75, 50, 45, 40, 35, 30, 25, 20, 15, 12, or fewer nucleotides in length; and tracr RNA can be 30 or 50 nucleotides in length. In some embodiments, the degree of complementarity between a guide sequence and its corresponding target sequence is greater than 94.5% or 95% or 95.5% or 96% or 96.5% or 97% or 97.5% or 98% or 98.5% or 99% or 99.5% or 99.9%, or 100%. Off target is less than 100% or 99.9% or 99.5% or 99% or 99% or 98.5% or 98% or 97.5% or 97% or 96.5% or 96% or 95.5% or 95% or 94.5% or 94% or 93% or 92% or 91% or 90% or 89% or 88% or 87% or 86% or 85% or 84% or 83% or 82% or 81% or 80% complementarity between the sequence and the guide, with it advantageous that off target is 100% or 99.9% or 99.5% or 99% or 99% or 98.5% or 98% or 97.5% or 97% or 96.5% or 96% or 95.5% or 95% or 94.5% complementarity between the sequence and the guide.

In some embodiments according to the invention, the guide RNA (capable of guiding Cas to a target locus) may comprise (1) a guide sequence capable of hybridizing to a genomic target locus in the eukaryotic cell; (2) a tracr sequence; and (3) a tracr mate sequence. All (1) to (3) may reside in a single RNA, i.e., an sgRNA (arranged in a 5′ to 3′ orientation), or the tracr RNA may be a different RNA than the RNA containing the guide and tracr sequence. The tracr hybridizes to the tracr mate sequence and directs the CRISPR/Cas complex to the target sequence. Where the tracr RNA is on a different RNA than the RNA containing the guide and tracr sequence, the length of each RNA may be optimized to be shortened from their respective native lengths, and each may be independently chemically modified to protect from degradation by cellular RNase or otherwise increase stability.

Many modifications to guide sequences are known in the art and are further contemplated within the context of this invention. Various modifications may be used to increase the specificity of binding to the target sequence and/or increase the activity of the Cas protein and/or reduce off-target effects. Example guide sequence modifications are described in PCT US2019/045582, specifically paragraphs [0178]-[0333]. which is incorporated herein by reference.

### Target Sequences, PAMs, and PFSs

### Target Sequences

In the context of formation of a CRISPR complex, “target sequence” refers to a sequence to which a guide sequence is designed to have complementarity, where hybridization between a target sequence and a guide sequence promotes the formation of a CRISPR complex. A target sequence may comprise RNA polynucleotides. The term “target RNA” refers to an RNA polynucleotide being or comprising the target sequence. In other words, the target polynucleotide can be a polynucleotide or a part of a polynucleotide to which a part of the guide sequence is designed to have complementarity with and to which the effector function mediated by the complex comprising the CRISPR effector protein and a guide molecule is to be directed. In some embodiments, a target sequence is located in the nucleus or cytoplasm of a cell.

The guide sequence can specifically bind a target sequence in a target polynucleotide. The target polynucleotide may be DNA. The target polynucleotide may be RNA. The target polynucleotide can have one or more (e.g., 1, 2, 3, 4, 5, 6, 7, 8, 9, 10, etc. or more) target sequences. The target polynucleotide can be on a vector. The target polynucleotide can be genomic DNA. The target polynucleotide can be episomal. Other forms of the target polynucleotide are described elsewhere herein.

The target sequence may be DNA. The target sequence may be any RNA sequence. In some embodiments, the target sequence may be a sequence within an RNA molecule selected from the group consisting of messenger RNA (mRNA), pre-mRNA, ribosomal RNA (rRNA), transfer RNA (tRNA), micro-RNA (miRNA), small interfering RNA (siRNA), small nuclear RNA (snRNA), small nucleolar RNA (snoRNA), double stranded RNA (dsRNA), non-coding RNA (ncRNA), long non-coding RNA (lncRNA), and small cytoplasmatic RNA (scRNA). In some preferred embodiments, the target sequence (also referred to herein as a target polynucleotide) may be a sequence within an RNA molecule selected from the group consisting of mRNA, pre-mRNA, and rRNA. In some preferred embodiments, the target sequence may be a sequence within an RNA molecule selected from the group consisting of ncRNA, and lncRNA. In some more preferred embodiments, the target sequence may be a sequence within an mRNA molecule or a pre-mRNA molecule.

### PAM and PFS Elements

PAM elements are sequences that can be recognized and bound by Cas proteins. Cas proteins/effector complexes can then unwind the dsDNA at a position adjacent to the PAM element. It will be appreciated that Cas proteins and systems that include them that target RNA do not require PAM sequences (Marraffini et al. 2010. Nature. 463:568-571). Instead, many rely on PFSs, which are discussed elsewhere herein. In certain embodiments, the target sequence should be associated with a PAM (protospacer adjacent motif) or PFS (protospacer flanking sequence or site), that is, a short sequence recognized by the CRISPR complex. Depending on the nature of the CRISPR-Cas protein, the target sequence should be selected, such that its complementary sequence in the DNA duplex (also referred to herein as the non-target sequence) is upstream or downstream of the PAM. In the embodiments, the complementary sequence of the target sequence is downstream or 3′ of the PAM or upstream or 5′ of the PAM. The precise sequence and length requirements for the PAM differ depending on the Cas protein used, but PAMs are typically 2-5 base pair sequences adjacent the protospacer (that is, the target sequence). Examples of the natural PAM sequences for different Cas proteins are provided herein below and the skilled person will be able to identify further PAM sequences for use with a given Cas protein.

The ability to recognize different PAM sequences depends on the Cas polypeptide(s) included in the system. See e.g., Gleditzsch et al. 2019. RNA Biology. 16(4):504-517. Table 3 below shows several Cas polypeptides and the PAM sequence they recognize.

TABLE 3
Example PAM Sequences

| Cas Protein | PAM Sequence |
|---|---|
| SpCas9 | NGG/NRG |
| SaCas9 | NGRRT or NGRRN |
| NmeCas9 | NNNNGATT |
| CjCas9 | NNNNRYAC |
| StCas9 | NNAGAAW |
| Cas12a(Cpf1)(including LbCpf1 | TTTV |
| and AsCpf1) | |
| Cas12b (C2c1) | TTT, TTA, and TTC |
| Cas12c (C2c3) | TA |
| Cas12d (CasY) | TA |
| Cas12e (CasX) | 5′-TTCN-3′ |

In a preferred embodiment, the CRISPR effector protein may recognize a 3′ PAM. In certain embodiments, the CRISPR effector protein may recognize a 3′ PAM which is 5′H, wherein H is A, C or U.

Further, engineering of the PAM Interacting (PI) domain on the Cas protein may allow programing of PAM specificity, improve target site recognition fidelity, and increase the versatility of the CRISPR-Cas protein, for example as described for Cas9 in Kleinstiver B P et al. Engineered CRISPR-Cas9 nucleases with altered PAM specificities. Nature. 2015 Jul. 23; 523(7561):481-5. doi: 10.1038/nature14592. As further detailed herein, the skilled person will understand that Cas13 proteins may be modified analogously. Gao et al, “Engineered Cpf1 Enzymes with Altered PAM Specificities,” bioRxiv 091611; doi: http://dx.doi.org/10.1101/091611 (Dec. 4, 2016). Doench et al. created a pool of sgRNAs, tiling across all possible target sites of a panel of six endogenous mouse and three endogenous human genes and quantitatively assessed their ability to produce null alleles of their target gene by antibody staining and flow cytometry. The authors showed that optimization of the PAM improved activity and also provided an on-line tool for designing sgRNAs.

PAM sequences can be identified in a polynucleotide using an appropriate design tool, which are commercially available as well as online. Such freely available tools include, but are not limited to, CRISPRFinder and CRISPRTarget. Mojica et al. 2009. Microbiol. 155(Pt. 3):733-740; Atschul et al. 1990. J. Mol. Biol. 215:403-410; Biswass et al. 2013 RNA Biol. 10:817-827; and Grissa et al. 2007. Nucleic Acid Res. 35:W52-57. Experimental approaches to PAM identification can include, but are not limited to, plasmid depletion assays (Jiang et al. 2013. Nat. Biotechnol. 31:233-239; Esvelt et al. 2013. Nat. Methods. 10:1116-1121; Kleinstiver et al. 2015. Nature. 523:481-485), screened by a high-throughput in vivo model called PAM-SCNAR (Pattanayak et al. 2013. Nat. Biotechnol. 31:839-843 and Leenay et al. 2016. Mol. Cell. 16:253), and negative screening (Zetsche et al. 2015. Cell. 163:759-771).

As previously mentioned, CRISPR-Cas systems that target RNA do not typically rely on PAM sequences. Instead such systems typically recognize protospacer flanking sites (PFSs) instead of PAMs Thus, Type VI CRISPR-Cas systems typically recognize protospacer flanking sites (PFSs) instead of PAMs. PFSs represents an analogue to PAMs for RNA targets. Type VI CRISPR-Cas systems employ a Cas13. Some Cas13 proteins analyzed to date, such as Cas13a (C2c2) identified from Leptotrichia shahii (LShCAs13a) have a specific discrimination against G at the 3′end of the target RNA. The presence of a C at the corresponding crRNA repeat site can indicate that nucleotide pairing at this position is rejected. However, some Cas13 proteins (e.g., LwaCAs13a and PspCas13b) do not seem to have a PFS preference. See e.g., Gleditzsch et al. 2019. RNA Biology. 16(4):504-517.

Some Type VI proteins, such as subtype B, have 5′-recognition of D (G, T, A) and a 3′-motif requirement of NAN or NNA. One example is the Cas13b protein identified in Bergeyella zoohelcum (BzCas13b). See e.g., Gleditzsch et al. 2019. RNA Biology. 16(4):504-517.

Overall Type VI CRISPR-Cas systems appear to have less restrictive rules for substrate (e.g., target sequence) recognition than those that target DNA (e.g., Type V and type II).

### Zinc Finger Nucleases

In some embodiments, the polynucleotide is modified using a Zinc Finger nuclease or system thereof. One type of programmable DNA-binding domain is provided by artificial zinc-finger (ZF) technology, which involves arrays of ZF modules to target new DNA-binding sites in the genome. Each finger module in a ZF array targets three DNA bases. A customized array of individual zinc finger domains is assembled into a ZF protein (ZFP).

ZFPs can comprise a functional domain. The first synthetic zinc finger nucleases (ZFNs) were developed by fusing a ZF protein to the catalytic domain of the Type IIS restriction enzyme FokI. (Kim, Y. G. et al., 1994, Chimeric restriction endonuclease, Proc. Natl. Acad. Sci. U.S.A. 91, 883-887; Kim, Y. G. et al., 1996, Hybrid restriction enzymes: zinc finger fusions to Fok I cleavage domain. Proc. Natl. Acad. Sci. U.S.A. 93, 1156-1160). Increased cleavage specificity can be attained with decreased off target activity by use of paired ZFN heterodimers, each targeting different nucleotide sequences separated by a short spacer. (Doyon, Y. et al., 2011, Enhancing zinc-finger-nuclease activity with improved obligate heterodimeric architectures. Nat. Methods 8, 74-79). ZFPs can also be designed as transcription activators and repressors and have been used to target many genes in a wide variety of organisms. Exemplary methods of genome editing using ZFNs can be found for example in U.S. Pat. Nos. 6,534,261, 6,607,882, 6,746,838, 6,794,136, 6,824,978, 6,866,997, 6,933,113, 6,979,539, 7,013,219, 7,030,215, 7,220,719, 7,241,573, 7,241,574, 7,585,849, 7,595,376, 6,903,185, and 6,479,626, all of which are specifically incorporated by reference.

### Sequences Related to Nucleus Targeting and Transportation

In some embodiments, one or more components (e.g., the Cas protein and/or deaminase) in the composition for engineering cells may comprise one or more sequences related to nucleus targeting and transportation. Such sequence may facilitate the one or more components in the composition for targeting a sequence within a cell. In order to improve targeting of the CRISPR-Cas protein and/or the nucleotide deaminase protein or catalytic domain thereof used in the methods of the present disclosure to the nucleus, it may be advantageous to provide one or both of these components with one or more nuclear localization sequences (NLSs).

In some embodiments, the NLSs used in the context of the present disclosure are heterologous to the proteins. Non-limiting examples of NLSs include an NLS sequence derived from: the NLS of the SV40 virus large T-antigen, having the amino acid sequence PKKKRKV (SEQ ID No. 6) or PKKKRKVEAS (SEQ ID No. 7); the NLS from nucleoplasmin (e.g., the nucleoplasmin bipartite NLS with the sequence KRPAATKKAGQAKKKK (SEQ ID No. 8)); the c-myc NLS having the amino acid sequence PAAKRVKLD (SEQ ID No. 9) or RQRRNELKRSP (SEQ ID No. 10); the hRNPA1 M9 NLS having the sequence NQSSNFGPMKGGNFGGRSSGPYGGGGQYFAKPRNQGGY (SEQ ID No. 11); the sequence RMRIZFKNKGKDTAELRRRRVEVSVELRKAKKDEQILKRRNV (SEQ ID No. 12) of the IBB domain from importin-alpha; the sequences VSRKRPRP (SEQ ID No. 13) and PPKKARED (SEQ ID No. 14) of the myoma T protein; the sequence PQPKKKPL (SEQ ID No. 15) of human p53; the sequence SALIKKKKKMAP (SEQ ID No. 16) of mouse c-abl IV; the sequences DRLRR (SEQ ID No. 17) and PKQKKRK (SEQ ID No. 18) of the influenza virus NS1; the sequence RKLKKKIKKL (SEQ ID No. 19) of the Hepatitis virus delta antigen; the sequence REKKKFLKRR (SEQ ID No. 20) of the mouse Mx1 protein; the sequence KRKGDEVDGVDEVAKKKSKK (SEQ ID No. 21) of the human poly(ADP-ribose) polymerase; and the sequence RKCLQAGMNLEARKTKK (SEQ ID No. 22) of the steroid hormone receptors (human) glucocorticoid. In general, the one or more NLSs are of sufficient strength to drive accumulation of the DNA-targeting Cas protein in a detectable amount in the nucleus of a eukaryotic cell. In general, strength of nuclear localization activity may derive from the number of NLSs in the CRISPR-Cas protein, the particular NLS(s) used, or a combination of these factors. Detection of accumulation in the nucleus may be performed by any suitable technique. For example, a detectable marker may be fused to the nucleic acid-targeting protein, such that location within a cell may be visualized, such as in combination with a means for detecting the location of the nucleus (e.g., a stain specific for the nucleus such as DAPI). Cell nuclei may also be isolated from cells, the contents of which may then be analyzed by any suitable process for detecting protein, such as immunohistochemistry, Western blot, or enzyme activity assay. Accumulation in the nucleus may also be determined indirectly, such as by an assay for the effect of nucleic acid-targeting complex formation (e.g., assay for deaminase activity) at the target sequence, or assay for altered gene expression activity affected by DNA-targeting complex formation and/or DNA-targeting), as compared to a control not exposed to the CRISPR-Cas protein and deaminase protein, or exposed to a CRISPR-Cas and/or deaminase protein lacking the one or more NLSs.

The CRISPR-Cas and/or nucleotide deaminase proteins may be provided with 1 or more, such as with, 2, 3, 4, 5, 6, 7, 8, 9, 10, or more heterologous NLSs. In some embodiments, the proteins comprises about or more than about 1, 2, 3, 4, 5, 6, 7, 8, 9, 10, or more NLSs at or near the amino-terminus, about or more than about 1, 2, 3, 4, 5, 6, 7, 8, 9, 10, or more NLSs at or near the carboxy-terminus, or a combination of these (e.g., zero or at least one or more NLS at the amino-terminus and zero or at one or more NLS at the carboxy terminus). When more than one NLS is present, each may be selected independently of the others, such that a single NLS may be present in more than one copy and/or in combination with one or more other NLSs present in one or more copies. In some embodiments, an NLS is considered near the N- or C-terminus when the nearest amino acid of the NLS is within about 1, 2, 3, 4, 5, 10, 15, 20, 25, 30, 40, 50, or more amino acids along the polypeptide chain from the N- or C-terminus. In preferred embodiments of the CRISPR-Cas proteins, an NLS attached to the C-terminal of the protein.

In certain embodiments, the CRISPR-Cas protein and the deaminase protein are delivered to the cell or expressed within the cell as separate proteins. In these embodiments, each of the CRISPR-Cas and deaminase protein can be provided with one or more NLSs as described herein. In certain embodiments, the CRISPR-Cas and deaminase proteins are delivered to the cell or expressed with the cell as a fusion protein. In these embodiments one or both of the CRISPR-Cas and deaminase protein is provided with one or more NLSs. Where the nucleotide deaminase is fused to an adaptor protein (such as MS2) as described above, the one or more NLS can be provided on the adaptor protein, provided that this does not interfere with aptamer binding. In particular embodiments, the one or more NLS sequences may also function as linker sequences between the nucleotide deaminase and the CRISPR-Cas protein.

In certain embodiments, guides of the disclosure comprise specific binding sites (e.g. aptamers) for adapter proteins, which may be linked to or fused to an nucleotide deaminase or catalytic domain thereof. When such a guide forms a CRISPR complex (e.g., CRISPR-Cas protein binding to guide and target) the adapter proteins bind and, the nucleotide deaminase or catalytic domain thereof associated with the adapter protein is positioned in a spatial orientation which is advantageous for the attributed function to be effective.

The skilled person will understand that modifications to the guide which allow for binding of the adapter+nucleotide deaminase, but not proper positioning of the adapter+nucleotide deaminase (e.g. due to steric hindrance within the three dimensional structure of the CRISPR complex) are modifications which are not intended. The one or more modified guide may be modified at the tetra loop, the stem loop 1, stem loop 2, or stem loop 3, as described herein, preferably at either the tetra loop or stem loop 2, and in some cases at both the tetra loop and stem loop 2.

In some embodiments, a component (e.g., the dead Cas protein, the nucleotide deaminase protein or catalytic domain thereof, or a combination thereof) in the systems may comprise one or more nuclear export signals (NES), one or more nuclear localization signals (NLS), or any combinations thereof. In some cases, the NES may be an HIV Rev NES. In certain cases, the NES may be MAPK NES. When the component is a protein, the NES or NLS may be at the C terminus of component. Alternatively or additionally, the NES or NLS may be at the N terminus of component. In some examples, the Cas protein and optionally said nucleotide deaminase protein or catalytic domain thereof comprise one or more heterologous nuclear export signal(s) (NES(s)) or nuclear localization signal(s) (NLS(s)), preferably an HIV Rev NES or MAPK NES, preferably C-terminal.

### Templates

In some embodiments, the composition for engineering cells comprise a template, e.g., a recombination template. A template may be a component of another vector as described herein, contained in a separate vector, or provided as a separate polynucleotide. In some embodiments, a recombination template is designed to serve as a template in homologous recombination, such as within or near a target sequence nicked or cleaved by a nucleic acid-targeting effector protein as a part of a nucleic acid-targeting complex.

In an embodiment, the template nucleic acid alters the sequence of the target position. In an embodiment, the template nucleic acid results in the incorporation of a modified, or non-naturally occurring base into the target nucleic acid.

The template sequence may undergo a breakage mediated or catalyzed recombination with the target sequence. In an embodiment, the template nucleic acid may include sequence that corresponds to a site on the target sequence that is cleaved by a Cas protein mediated cleavage event. In an embodiment, the template nucleic acid may include sequence that corresponds to both, a first site on the target sequence that is cleaved in a first Cas protein mediated event, and a second site on the target sequence that is cleaved in a second Cas protein mediated event.

In certain embodiments, the template nucleic acid can include sequence which results in an alteration in the coding sequence of a translated sequence, e.g., one which results in the substitution of one amino acid for another in a protein product, e.g., transforming a mutant allele into a wild type allele, transforming a wild type allele into a mutant allele, and/or introducing a stop codon, insertion of an amino acid residue, deletion of an amino acid residue, or a nonsense mutation. In certain embodiments, the template nucleic acid can include sequence which results in an alteration in a non-coding sequence, e.g., an alteration in an exon or in a 5′ or 3′ non-translated or non-transcribed region. Such alterations include an alteration in a control element, e.g., a promoter, enhancer, and an alteration in a cis-acting or trans-acting control element.

A template nucleic acid having homology with a target position in a target gene may be used to alter the structure of a target sequence. The template sequence may be used to alter an unwanted structure, e.g., an unwanted or mutant nucleotide. The template nucleic acid may include sequence which, when integrated, results in: decreasing the activity of a positive control element; increasing the activity of a positive control element; decreasing the activity of a negative control element; increasing the activity of a negative control element; decreasing the expression of a gene; increasing the expression of a gene; increasing resistance to a disorder or disease; increasing resistance to viral entry; correcting a mutation or altering an unwanted amino acid residue conferring, increasing, abolishing or decreasing a biological property of a gene product, e.g., increasing the enzymatic activity of an enzyme, or increasing the ability of a gene product to interact with another molecule.

The template nucleic acid may include sequence which results in: a change in sequence of 1, 2, 3, 4, 5, 6, 7, 8, 9, 10, 11, 12 or more nucleotides of the target sequence.

A template polynucleotide may be of any suitable length, such as about or more than about 10, 15, 20, 25, 50, 75, 100, 150, 200, 500, 1000, or more nucleotides in length. In an embodiment, the template nucleic acid may be 20+/−10, 30+/−10, 40+/−10, 50+/−10, 60+/−10, 70+/−10, 80+/−10, 90+/−10, 100+/−10, 110+/−10, 120+/−10, 130+/−10, 140+/−10, 150+/−10, 160+/−10, 170+/−10, 180+/−10, 190+/−10, 200+/−10, 210+/−10, of 220+/−10 nucleotides in length. In an embodiment, the template nucleic acid may be 30+/−20, 40+/−20, 50+/−20, 60+/−20, 70+/−20, 80+/−20, 90+/−20, 100+/−20, 110+/−20, 120+/−20, 130+/−20, 140+/−20, 150+/−20, 160+/−20, 170+/−20, 180+/−20, 190+/−20, 200+/−20, 210+/−20, of 220+/−20 nucleotides in length. In an embodiment, the template nucleic acid is 10 to 1,000, 20 to 900, 30 to 800, 40 to 700, 50 to 600, 50 to 500, 50 to 400, 50 to 300, 50 to 200, or 50 to 100 nucleotides in length.

In some embodiments, the template polynucleotide is complementary to a portion of a polynucleotide comprising the target sequence. When optimally aligned, a template polynucleotide might overlap with one or more nucleotides of a target sequences (e.g. about or more than about 1, 5, 10, 15, 20, 25, 30, 35, 40, 45, 50, 60, 70, 80, 90, 100 or more nucleotides). In some embodiments, when a template sequence and a polynucleotide comprising a target sequence are optimally aligned, the nearest nucleotide of the template polynucleotide is within about 1, 5, 10, 15, 20, 25, 50, 75, 100, 200, 300, 400, 500, 1000, 5000, 10000, or more nucleotides from the target sequence.

The exogenous polynucleotide template comprises a sequence to be integrated (e.g., a mutated gene). The sequence for integration may be a sequence endogenous or exogenous to the cell. Examples of a sequence to be integrated include polynucleotides encoding a protein or a non-coding RNA (e.g., a microRNA). Thus, the sequence for integration may be operably linked to an appropriate control sequence or sequences. Alternatively, the sequence to be integrated may provide a regulatory function.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000.

An upstream or downstream sequence may comprise from about 20 bp to about 2500 bp, for example, about 50, 100, 200, 300, 400, 500, 600, 700, 800, 900, 1000, 1100, 1200, 1300, 1400, 1500, 1600, 1700, 1800, 1900, 2000, 2100, 2200, 2300, 2400, or 2500 bp. In some methods, the exemplary upstream or downstream sequence have about 200 bp to about 2000 bp, about 600 bp to about 1000 bp, or more particularly about 700 bp to about 1000

In certain embodiments, one or both homology arms may be shortened to avoid including certain sequence repeat elements. For example, a 5′ homology arm may be shortened to avoid a sequence repeat element. In other embodiments, a 3′ homology arm may be shortened to avoid a sequence repeat element. In some embodiments, both the 5′ and the 3′ homology arms may be shortened to avoid including certain sequence repeat elements.

In some methods, the exogenous polynucleotide template may further comprise a marker. Such a marker may make it easy to screen for targeted integrations. Examples of suitable markers include restriction sites, fluorescent proteins, or selectable markers. The exogenous polynucleotide template of the disclosure can be constructed using recombinant techniques (see, for example, Sambrook et al., 2001 and Ausubel et al., 1996).

In certain embodiments, a template nucleic acid for correcting a mutation may designed for use as a single-stranded oligonucleotide. When using a single-stranded oligonucleotide, 5′ and 3′ homology arms may range up to about 200 base pairs (bp) in length, e.g., at least 25, 50, 75, 100, 125, 150, 175, or 200 bp in length.

Suzuki et al. describe in vivo genome editing via CRISPR/Cas9 mediated homology-independent targeted integration (2016, Nature 540:144-149).

### TALE Nucleases

In some embodiments, a TALE nuclease or TALE nuclease system can be used to modify a polynucleotide. In some embodiments, the methods provided herein use isolated, non-naturally occurring, recombinant or engineered DNA binding proteins that comprise TALE monomers or TALE monomers or half monomers as a part of their organizational structure that enable the targeting of nucleic acid sequences with improved efficiency and expanded specificity.

Naturally occurring TALEs or “wild type TALEs” are nucleic acid binding proteins secreted by numerous species of proteobacteria. TALE polypeptides contain a nucleic acid binding domain composed of tandem repeats of highly conserved monomer polypeptides that are predominantly 33, 34 or 35 amino acids in length and that differ from each other mainly in amino acid positions 12 and 13. In advantageous embodiments the nucleic acid is DNA. As used herein, the term “polypeptide monomers”, “TALE monomers” or “monomers” will be used to refer to the highly conserved repetitive polypeptide sequences within the TALE nucleic acid binding domain and the term “repeat variable di-residues” or “RVD” will be used to refer to the highly variable amino acids at positions 12 and 13 of the polypeptide monomers. As provided throughout the disclosure, the amino acid residues of the RVD are depicted using the IUPAC single letter code for amino acids. A general representation of a TALE monomer which is comprised within the DNA binding domain is X₁₋₁₁-(X₁₂X₁₃)-X₁₄₋₃₃ or 34 or 35, where the subscript indicates the amino acid position and X represents any amino acid. X₁₂X₁₃ indicate the RVDs. In some polypeptide monomers, the variable amino acid at position 13 is missing or absent and in such monomers, the RVD consists of a single amino acid. In such cases the RVD may be alternatively represented as X*, where X represents X₁₂ and (*) indicates that X₁₃ is absent. The DNA binding domain comprises several repeats of TALE monomers and this may be represented as (X₁₋₁₁-(X₁₂X₁₃)-X₁₄₋₃₃ or 34 or 35)_{z}, where in an advantageous embodiment, z is at least 5 to 40. In a further advantageous embodiment, z is at least 10 to 26.

The TALE monomers can have a nucleotide binding affinity that is determined by the identity of the amino acids in its RVD. For example, polypeptide monomers with an RVD of NI can preferentially bind to adenine (A), monomers with an RVD of NG can preferentially bind to thymine (T), monomers with an RVD of HD can preferentially bind to cytosine (C) and monomers with an RVD of NN can preferentially bind to both adenine (A) and guanine (G). In some embodiments, monomers with an RVD of IG can preferentially bind to T. Thus, the number and order of the polypeptide monomer repeats in the nucleic acid binding domain of a TALE determines its nucleic acid target specificity. In some embodiments, monomers with an RVD of NS can recognize all four base pairs and can bind to A, T, G or C. The structure and function of TALEs is further described in, for example, Moscou et al., Science 326:1501 (2009); Boch et al., Science 326:1509-1512 (2009); and Zhang et al., Nature Biotechnology 29:149-153 (2011).

The polypeptides used in methods of the invention can be isolated, non-naturally occurring, recombinant or engineered nucleic acid-binding proteins that have nucleic acid or DNA binding regions containing polypeptide monomer repeats that are designed to target specific nucleic acid sequences.

As described herein, polypeptide monomers having an RVD of HN or NH preferentially bind to guanine and thereby allow the generation of TALE polypeptides with high binding specificity for guanine containing target nucleic acid sequences. In some embodiments, polypeptide monomers having RVDs RN, NN, NK, SN, NH, KN, HN, NQ, HH, RG, KH, RH and SS can preferentially bind to guanine. In some embodiments, polypeptide monomers having RVDs RN, NK, NQ, HH, KH, RH, SS and SN can preferentially bind to guanine and can thus allow the generation of TALE polypeptides with high binding specificity for guanine containing target nucleic acid sequences. In some embodiments, polypeptide monomers having RVDs HH, KH, NH, NK, NQ, RH, RN and SS can preferentially bind to guanine and thereby allow the generation of TALE polypeptides with high binding specificity for guanine containing target nucleic acid sequences. In some embodiments, the RVDs that have high binding specificity for guanine are RN, NH RH and KH. Furthermore, polypeptide monomers having an RVD of NV can preferentially bind to adenine and guanine. In some embodiments, monomers having RVDs of H*, HA, KA, N*, NA, NC, NS, RA, and S* bind to adenine, guanine, cytosine and thymine with comparable affinity.

The predetermined N-terminal to C-terminal order of the one or more polypeptide monomers of the nucleic acid or DNA binding domain determines the corresponding predetermined target nucleic acid sequence to which the polypeptides of the invention will bind. As used herein the monomers and at least one or more half monomers are “specifically ordered to target” the genomic locus or gene of interest. In plant genomes, the natural TALE-binding sites always begin with a thymine (T), which may be specified by a cryptic signal within the non-repetitive N-terminus of the TALE polypeptide; in some cases, this region may be referred to as repeat 0. In animal genomes, TALE binding sites do not necessarily have to begin with a thymine (T) and polypeptides of the invention may target DNA sequences that begin with T, A, G or C. The tandem repeat of TALE monomers always ends with a half-length repeat or a stretch of sequence that may share identity with only the first 20 amino acids of a repetitive full-length TALE monomer and this half repeat may be referred to as a half-monomer. Therefore, it follows that the length of the nucleic acid or DNA being targeted is equal to the number of full monomers plus two.

As described in Zhang et al., Nature Biotechnology 29:149-153 (2011), TALE polypeptide binding efficiency may be increased by including amino acid sequences from the “capping regions” that are directly N-terminal or C-terminal of the DNA binding region of naturally occurring TALEs into the engineered TALEs at positions N-terminal or C-terminal of the engineered TALE DNA binding region. Thus, in certain embodiments, the TALE polypeptides described herein further comprise an N-terminal capping region and/or a C-terminal capping region.

An exemplary amino acid sequence of a N-terminal capping region is:

(SEQ ID NO: 23)
MDPIRSRTPSPARELLSGPQPDGVQPTADRGVSPPAGGPLDGLPARRTMS
RTRLPSPPAPSPAFSADSFSDLLRQFDPSLFNTSLFDSLPPFGAHHTEAA
TGEWDEVQSGLRAADAPPPTMRVAVTAARPPRAKPAPRRRAAQPSDASPA
AQVDLRTLGYSQQQQEKIKPKVRSTVAQHHEALVGHGFTHAHIVALSQHP
AALGTVAVKYQDMIAALPEATHEAIVGVGKQWSGARALEALLTVAGELRG
PPLQLDTGQLLKIAKRGGVTAVEAVHAWRNALTGAPLN

An exemplary amino acid sequence of a C-terminal capping region is:

(SEQ ID NO: 24)
RPALESIVAQLSRPDPALAALTNDHLVALACLGGRPALDAVKKGLPHAPA
LIKRTNRRIPERTSHRVADHAQVVRVLGFFQCHSHPAQAFDDAMTQFGMS
RHGLLQLFRRVGVTELEARSGTLPPASQRWDRILQASGMKRAKPSPTSTQ
TPDQASLHAFADSLERDLDAPSPMHEGDQTRAS

As used herein the predetermined “N-terminus” to “C terminus” orientation of the N-terminal capping region, the DNA binding domain comprising the repeat TALE monomers and the C-terminal capping region provide structural basis for the organization of different domains in the d-TALEs or polypeptides of the invention.

The entire N-terminal and/or C-terminal capping regions are not necessary to enhance the binding activity of the DNA binding region. Therefore, in certain embodiments, fragments of the N-terminal and/or C-terminal capping regions are included in the TALE polypeptides described herein.

In certain embodiments, the TALE polypeptides described herein contain a N-terminal capping region fragment that included at least 10, 20, 30, 40, 50, 54, 60, 70, 80, 87, 90, 94, 100, 102, 110, 117, 120, 130, 140, 147, 150, 160, 170, 180, 190, 200, 210, 220, 230, 240, 250, 260 or 270 amino acids of an N-terminal capping region. In certain embodiments, the N-terminal capping region fragment amino acids are of the C-terminus (the DNA-binding region proximal end) of an N-terminal capping region. As described in Zhang et al., Nature Biotechnology 29:149-153 (2011), N-terminal capping region fragments that include the C-terminal 240 amino acids enhance binding activity equal to the full length capping region, while fragments that include the C-terminal 147 amino acids retain greater than 80% of the efficacy of the full length capping region, and fragments that include the C-terminal 117 amino acids retain greater than 50% of the activity of the full-length capping region.

In some embodiments, the TALE polypeptides described herein contain a C-terminal capping region fragment that included at least 6, 10, 20, 30, 37, 40, 50, 60, 68, 70, 80, 90, 100, 110, 120, 127, 130, 140, 150, 155, 160, 170, 180 amino acids of a C-terminal capping region. In certain embodiments, the C-terminal capping region fragment amino acids are of the N-terminus (the DNA-binding region proximal end) of a C-terminal capping region. As described in Zhang et al., Nature Biotechnology 29:149-153 (2011), C-terminal capping region fragments that include the C-terminal 68 amino acids enhance binding activity equal to the full-length capping region, while fragments that include the C-terminal 20 amino acids retain greater than 50% of the efficacy of the full-length capping region.

In certain embodiments, the capping regions of the TALE polypeptides described herein do not need to have identical sequences to the capping region sequences provided herein. Thus, in some embodiments, the capping region of the TALE polypeptides described herein have sequences that are at least 50%, 60%, 70%, 80%, 85%, 90%, 91%, 92%, 93%, 94%, 95%, 96%, 97%, 98% or 99% identical or share identity to the capping region amino acid sequences provided herein. Sequence identity is related to sequence homology. Homology comparisons may be conducted by eye, or more usually, with the aid of readily available sequence comparison programs. These commercially available computer programs may calculate percent (%) homology between two or more sequences and may also calculate the sequence identity shared by two or more amino acid or nucleic acid sequences. In some preferred embodiments, the capping region of the TALE polypeptides described herein have sequences that are at least 95% identical or share identity to the capping region amino acid sequences provided herein.

Sequence homologies can be generated by any of a number of computer programs known in the art, which include but are not limited to BLAST or FASTA. Suitable computer programs for carrying out alignments like the GCG Wisconsin Bestfit package may also be used. Once the software has produced an optimal alignment, it is possible to calculate % homology, preferably % sequence identity. The software typically does this as part of the sequence comparison and generates a numerical result.

In some embodiments described herein, the TALE polypeptides of the invention include a nucleic acid binding domain linked to the one or more effector domains. The terms “effector domain” or “regulatory and functional domain” refer to a polypeptide sequence that has an activity other than binding to the nucleic acid sequence recognized by the nucleic acid binding domain. By combining a nucleic acid binding domain with one or more effector domains, the polypeptides of the invention may be used to target the one or more functions or activities mediated by the effector domain to a particular target DNA sequence to which the nucleic acid binding domain specifically binds.

In some embodiments of the TALE polypeptides described herein, the activity mediated by the effector domain is a biological activity. For example, in some embodiments the effector domain is a transcriptional inhibitor (i.e., a repressor domain), such as an mSin interaction domain (SID). SID4X domain or a Kruppel-associated box (KRAB) or fragments of the KRAB domain. In some embodiments the effector domain is an enhancer of transcription (i.e. an activation domain), such as the VP16, VP64 or p65 activation domain. In some embodiments, the nucleic acid binding is linked, for example, with an effector domain that includes but is not limited to a transposase, integrase, recombinase, resolvase, invertase, protease, DNA methyltransferase, DNA demethylase, histone acetylase, histone deacetylase, nuclease, transcriptional repressor, transcriptional activator, transcription factor recruiting, protein nuclear-localization signal or cellular uptake signal.

In some embodiments, the effector domain is a protein domain which exhibits activities which include but are not limited to transposase activity, integrase activity, recombinase activity, resolvase activity, invertase activity, protease activity, DNA methyltransferase activity, DNA demethylase activity, histone acetylase activity, histone deacetylase activity, nuclease activity, nuclear-localization signaling activity, transcriptional repressor activity, transcriptional activator activity, transcription factor recruiting activity, or cellular uptake signaling activity. Other preferred embodiments of the invention may include any combination of the activities described herein.

### Meganucleases

In some embodiments, a meganuclease or system thereof can be used to modify a polynucleotide. Meganucleases, which are endodeoxyribonucleases characterized by a large recognition site (double-stranded DNA sequences of 12 to 40 base pairs). Exemplary methods for using meganucleases can be found in U.S. Pat. Nos. 8,163,514, 8,133,697, 8,021,867, 8,119,361, 8,119,381, 8,124,369, and 8,129,134, which are specifically incorporated by reference.

### RNAi

In certain embodiments, the genetic modifying agent is RNAi (e.g., shRNA). As used herein, “gene silencing” or “gene silenced” in reference to an activity of an RNAi molecule, for example a siRNA or miRNA refers to a decrease in the mRNA level in a cell for a target gene by at least about 5%, about 10%, about 20%, about 30%, about 40%, about 50%, about 60%, about 70%, about 80%, about 90%, about 95%, about 99%, about 100% of the mRNA level found in the cell without the presence of the miRNA or RNA interference molecule. In one preferred embodiment, the mRNA levels are decreased by at least about 70%, about 80%, about 90%, about 95%, about 99%, about 100%.

As used herein, the term “RNAi” refers to any type of interfering RNA, including but not limited to, siRNAi, shRNAi, endogenous microRNA and artificial microRNA. For instance, it includes sequences previously identified as siRNA, regardless of the mechanism of down-stream processing of the RNA (i.e. although siRNAs are believed to have a specific method of in vivo processing resulting in the cleavage of mRNA, such sequences can be incorporated into the vectors in the context of the flanking sequences described herein). The term “RNAi” can include both gene silencing RNAi molecules, and also RNAi effector molecules which activate the expression of a gene.

As used herein, a “siRNA” refers to a nucleic acid that forms a double stranded RNA, which double stranded RNA has the ability to reduce or inhibit expression of a gene or target gene when the siRNA is present or expressed in the same cell as the target gene. The double stranded RNA siRNA can be formed by the complementary strands. In one embodiment, a siRNA refers to a nucleic acid that can form a double stranded siRNA. The sequence of the siRNA can correspond to the full-length target gene, or a subsequence thereof. Typically, the siRNA is at least about 15-50 nucleotides in length (e.g., each complementary sequence of the double stranded siRNA is about 15-50 nucleotides in length, and the double stranded siRNA is about 15-50 base pairs in length, preferably about 19-30 base nucleotides, preferably about 20-25 nucleotides in length, e.g., 20, 21, 22, 23, 24, 25, 26, 27, 28, 29, or 30 nucleotides in length).

As used herein “shRNA” or “small hairpin RNA” (also called stem loop) is a type of siRNA. In one embodiment, these shRNAs are composed of a short, e.g. about 19 to about 25 nucleotide, antisense strand, followed by a nucleotide loop of about 5 to about 9 nucleotides, and the analogous sense strand. Alternatively, the sense strand can precede the nucleotide loop structure and the antisense strand can follow.

The terms “microRNA” or “miRNA” are used interchangeably herein are endogenous RNAs, some of which are known to regulate the expression of protein-coding genes at the posttranscriptional level. Endogenous microRNAs are small RNAs naturally present in the genome that are capable of modulating the productive utilization of mRNA. The term artificial microRNA includes any type of RNA sequence, other than endogenous microRNA, which is capable of modulating the productive utilization of mRNA. MicroRNA sequences have been described in publications such as Lim, et al., Genes & Development, 17, p. 991-1008 (2003), Lim et al Science 299, 1540 (2003), Lee and Ambros Science, 294, 862 (2001), Lau et al., Science 294, 858-861 (2001), Lagos-Quintana et al, Current Biology, 12, 735-739 (2002), Lagos Quintana et al, Science 294, 853-857 (2001), and Lagos-Quintana et al, RNA, 9, 175-179 (2003), which are incorporated by reference. Multiple microRNAs can also be incorporated into a precursor molecule. Furthermore, miRNA-like stem-loops can be expressed in cells as a vehicle to deliver artificial miRNAs and short interfering RNAs (siRNAs) for the purpose of modulating the expression of endogenous genes through the miRNA and or RNAi pathways.

As used herein, “double stranded RNA” or “dsRNA” refers to RNA molecules that are comprised of two strands. Double-stranded molecules include those comprised of a single RNA molecule that doubles back on itself to form a two-stranded structure. For example, the stem loop structure of the progenitor molecules from which the single-stranded miRNA is derived, called the pre-miRNA (Bartel et al. 2004. Cell 1 16:281-297), comprises a dsRNA molecule.

### Antibodies

In certain embodiments, the one or more agents is an antibody. In certain embodiments, the antibody specific for a surface protein. The surface protein may be selected from CLDN3, CLDN7, CLDN4, EPCAM, TACSTD2, MAL2, LSR, CD9, SPINT2, TM4SF1, TMEM205, TNFRSF12A and CD47. The antibodies or CARs herein (e.g., antibodies, bi-specific antibody, antibody-drug conjugate) can include any known antibody, antibody fragment or CDRs specific for the surface proteins. Antibodies for CLDN3 or CLDN3/4 may include or be derived from those previously disclosed (see, e.g., Yang, et al., Development of Human Monoclonal Antibody for Claudin-3 Overexpressing Carcinoma Targeting. Biomolecules. 2020 January; 10(1): 51; Kato-Nakano, et al., Characterization and evaluation of the antitumour activity of a dual-targeting monoclonal antibody against claudin-3 and claudin-4. Anticancer Res. 2010 November; 30(11):4555-62; Li, et al., Development of an anti-claudin-3 and -4 bispecific monoclonal antibody for cancer diagnosis and therapy. J Pharmacol Exp Ther. 2014 October; 351(1):206-13; and Ando, et al., Generation of specific monoclonal antibodies against the extracellular loops of human claudin-3 by immunizing mice with target-expressing cells. Bioscience, Biotechnology, and Biochemistry, 79:8, 1272-1279). Antibodies for CLDN4 may include or be derived from those previously disclosed (see, e.g., U.S. Pat. No. 8,076,458B2; Suzuki, et al., Therapeutic antitumor efficacy of monoclonal antibody against Claudin-4 for pancreatic and ovarian cancers. Cancer Sci. 2009 September; 100(9):1623-30; and Fujiwara-Tani, et al., Anti-claudin-4 extracellular domain antibody enhances the antitumoral effects of chemotherapeutic and antibody drugs in colorectal cancer. Oncotarget. 2018 Dec. 21; 9(100): 37367-37378).

The term “antibody” is used interchangeably with the term “immunoglobulin” herein, and includes intact antibodies, fragments of antibodies, e.g., Fab, F(ab′)2 fragments, and intact antibodies and fragments that have been mutated either in their constant and/or variable region (e.g., mutations to produce chimeric, partially humanized, or fully humanized antibodies, as well as to produce antibodies with a desired trait, e.g., enhanced binding and/or reduced FcR binding). The term “fragment” refers to a part or portion of an antibody or antibody chain comprising fewer amino acid residues than an intact or complete antibody or antibody chain. Fragments can be obtained via chemical or enzymatic treatment of an intact or complete antibody or antibody chain. Fragments can also be obtained by recombinant means. Exemplary fragments include a nanobody, Fab, Fab′, (Fab′)2, Fv, ScFv, diabody, triabody, tetrabody, Bis-scFv, minibody, Fab2, or Fab3 fragment, Fabc, Fd, dAb, V_{HH} and scFv and/or Fv fragments.

As used herein, a preparation of antibody protein having less than about 50% of non-antibody protein (also referred to herein as a “contaminating protein”), or of chemical precursors, is considered to be “substantially free.” 40%, 30%, 20%, 10% and more preferably 5% (by dry weight), of non-antibody protein, or of chemical precursors is considered to be substantially free. When the antibody protein or biologically active portion thereof is recombinantly produced, it is also preferably substantially free of culture medium, i.e., culture medium represents less than about 30%, preferably less than about 20%, more preferably less than about 10%, and most preferably less than about 5% of the volume or mass of the protein preparation.

The term “antigen-binding fragment” refers to a polypeptide fragment of an immunoglobulin or antibody that binds antigen or competes with intact antibody (i.e., with the intact antibody from which they were derived) for antigen binding (i.e., specific binding). As such these antibodies or fragments thereof are included in the scope of the invention, provided that the antibody or fragment binds specifically to a target molecule.

It is intended that the term “antibody” encompass any Ig class or any Ig subclass (e.g. the IgG1, IgG2, IgG3, and IgG4 subclasses of IgG) obtained from any source (e.g., humans and non-human primates, and in rodents, lagomorphs, caprines, bovines, equines, ovines, etc.).

The term “Ig class” or “immunoglobulin class”, as used herein, refers to the five classes of immunoglobulin that have been identified in humans and higher mammals, IgG, IgM, IgA, IgD, and IgE. The term “Ig subclass” refers to the two subclasses of IgM (H and L), three subclasses of IgA (IgA1, IgA2, and secretory IgA), and four subclasses of IgG (IgG1, IgG2, IgG3, and IgG4) that have been identified in humans and higher mammals. The antibodies can exist in monomeric or polymeric form; for example, lgM antibodies exist in pentameric form, and IgA antibodies exist in monomeric, dimeric or multimeric form.

The term “IgG subclass” refers to the four subclasses of immunoglobulin class IgG—IgG1, IgG2, IgG3, and IgG4 that have been identified in humans and higher mammals by the heavy chains of the immunoglobulins, V1-γ4, respectively. The term “single-chain immunoglobulin” or “single-chain antibody” (used interchangeably herein) refers to a protein having a two-polypeptide chain structure consisting of a heavy and a light chain, said chains being stabilized, for example, by interchain peptide linkers, which has the ability to specifically bind antigen. The term “domain” refers to a globular region of a heavy or light chain polypeptide comprising peptide loops (e.g., comprising 3 to 4 peptide loops) stabilized, for example, by β pleated sheet and/or intrachain disulfide bond. Domains are further referred to herein as “constant” or “variable”, based on the relative lack of sequence variation within the domains of various class members in the case of a “constant” domain, or the significant variation within the domains of various class members in the case of a “variable” domain. Antibody or polypeptide “domains” are often referred to interchangeably in the art as antibody or polypeptide “regions”. The “constant” domains of an antibody light chain are referred to interchangeably as “light chain constant regions”, “light chain constant domains”, “CL” regions or “CL” domains. The “constant” domains of an antibody heavy chain are referred to interchangeably as “heavy chain constant regions”, “heavy chain constant domains”, “CH” regions or “CH” domains). The “variable” domains of an antibody light chain are referred to interchangeably as “light chain variable regions”, “light chain variable domains”, “VL” regions or “VL” domains). The “variable” domains of an antibody heavy chain are referred to interchangeably as “heavy chain constant regions”, “heavy chain constant domains”, “VH” regions or “VH” domains).

The term “region” can also refer to a part or portion of an antibody chain or antibody chain domain (e.g., a part or portion of a heavy or light chain or a part or portion of a constant or variable domain, as defined herein), as well as more discrete parts or portions of said chains or domains. For example, light and heavy chains or light and heavy chain variable domains include “complementarity determining regions” or “CDRs” interspersed among “framework regions” or “FRs”, as defined herein.

The term “conformation” refers to the tertiary structure of a protein or polypeptide (e.g., an antibody, antibody chain, domain or region thereof). For example, the phrase “light (or heavy) chain conformation” refers to the tertiary structure of a light (or heavy) chain variable region, and the phrase “antibody conformation” or “antibody fragment conformation” refers to the tertiary structure of an antibody or fragment thereof.

The term “antibody-like protein scaffolds” or “engineered protein scaffolds” broadly encompasses proteinaceous non-immunoglobulin specific-binding agents, typically obtained by combinatorial engineering (such as site-directed random mutagenesis in combination with phage display or other molecular selection techniques). Usually, such scaffolds are derived from robust and small soluble monomeric proteins (such as Kunitz inhibitors or lipocalins) or from a stably folded extra-membrane domain of a cell surface receptor (such as protein A, fibronectin or the ankyrin repeat).

Such scaffolds have been extensively reviewed in Binz et al. (Engineering novel binding proteins from nonimmunoglobulin domains. Nat Biotechnol 2005, 23:1257-1268), Gebauer and Skerra (Engineered protein scaffolds as next-generation antibody therapeutics. Curr Opin Chem Biol. 2009, 13:245-55), Gill and Damle (Biopharmaceutical drug discovery using novel protein scaffolds. Curr Opin Biotechnol 2006, 17:653-658), Skerra (Engineered protein scaffolds for molecular recognition. J Mol Recognit 2000, 13:167-187), and Skerra (Alternative non-antibody scaffolds for molecular recognition. Curr Opin Biotechnol 2007, 18:295-304), and include without limitation affibodies, based on the Z-domain of staphylococcal protein A, a three-helix bundle of 58 residues providing an interface on two of its alpha-helices (Nygren, Alternative binding proteins: Affibody binding proteins developed from a small three-helix bundle scaffold. FEBS J 2008, 275:2668-2676); engineered Kunitz domains based on a small (ca. 58 residues) and robust, disulphide-crosslinked serine protease inhibitor, typically of human origin (e.g. LACI-D1), which can be engineered for different protease specificities (Nixon and Wood, Engineered protein inhibitors of proteases. Curr Opin Drug Discov Dev 2006, 9:261-268); monobodies or adnectins based on the 10th extracellular domain of human fibronectin III (10Fn3), which adopts an Ig-like beta-sandwich fold (94 residues) with 2-3 exposed loops, but lacks the central disulphide bridge (Koide and Koide, Monobodies: antibody mimics based on the scaffold of the fibronectin type III domain. Methods Mol Biol 2007, 352:95-109); anticalins derived from the lipocalins, a diverse family of eight-stranded beta-barrel proteins (ca. 180 residues) that naturally form binding sites for small ligands by means of four structurally variable loops at the open end, which are abundant in humans, insects, and many other organisms (Skerra, Alternative binding proteins: Anticalins harnessing the structural plasticity of the lipocalin ligand pocket to engineer novel binding activities. FEBS J 2008, 275:2677-2683); DARPins, designed ankyrin repeat domains (166 residues), which provide a rigid interface arising from typically three repeated beta-turns (Stumpp et al., DARPins: a new generation of protein therapeutics. Drug Discov Today 2008, 13:695-701); avimers (multimerized LDLR-A module) (Silverman et al., Multivalent avimer proteins evolved by exon shuffling of a family of human receptor domains. Nat Biotechnol 2005, 23:1556-1561); and cysteine-rich knottin peptides (Kolmar, Alternative binding proteins: biological activity and therapeutic potential of cystine-knot miniproteins. FEBS J 2008, 275:2684-2690).

“Specific binding” of an antibody means that the antibody exhibits appreciable affinity for a particular antigen or epitope and, generally, does not exhibit significant cross reactivity. “Appreciable” binding includes binding with an affinity of at least 25 μM. Antibodies with affinities greater than 1×10⁷ M⁻¹ (or a dissociation coefficient of 1 μM or less or a dissociation coefficient of 1 nm or less) typically bind with correspondingly greater specificity. Values intermediate of those set forth herein are also intended to be within the scope of the present invention and antibodies of the invention bind with a range of affinities, for example, 100 nM or less, 75 nM or less, 50 nM or less, 25 nM or less, for example 10 nM or less, 5 nM or less, 1 nM or less, or in embodiments 500 pM or less, 100 pM or less, 50 pM or less or 25 pM or less. An antibody that “does not exhibit significant crossreactivity” is one that will not appreciably bind to an entity other than its target (e.g., a different epitope or a different molecule). For example, an antibody that specifically binds to a target molecule will appreciably bind the target molecule but will not significantly react with non-target molecules or peptides. An antibody specific for a particular epitope will, for example, not significantly crossreact with remote epitopes on the same protein or peptide. Specific binding can be determined according to any art-recognized means for determining such binding. Preferably, specific binding is determined according to Scatchard analysis and/or competitive binding assays.

As used herein, the term “affinity” refers to the strength of the binding of a single antigen-combining site with an antigenic determinant. Affinity depends on the closeness of stereochemical fit between antibody combining sites and antigen determinants, on the size of the area of contact between them, on the distribution of charged and hydrophobic groups, etc. Antibody affinity can be measured by equilibrium dialysis or by the kinetic BIACORE™ method. The dissociation constant, Kd, and the association constant, Ka, are quantitative measures of affinity.

As used herein, the term “monoclonal antibody” refers to an antibody derived from a clonal population of antibody-producing cells (e.g., B lymphocytes or B cells) which is homogeneous in structure and antigen specificity. The term “polyclonal antibody” refers to a plurality of antibodies originating from different clonal populations of antibody-producing cells which are heterogeneous in their structure and epitope specificity but which recognize a common antigen. Monoclonal and polyclonal antibodies may exist within bodily fluids, as crude preparations, or may be purified, as described herein.

The term “binding portion” of an antibody (or “antibody portion”) includes one or more complete domains, e.g., a pair of complete domains, as well as fragments of an antibody that retain the ability to specifically bind to a target molecule. It has been shown that the binding function of an antibody can be performed by fragments of a full-length antibody. Binding fragments are produced by recombinant DNA techniques, or by enzymatic or chemical cleavage of intact immunoglobulins. Binding fragments include Fab, Fab′, F(ab′)2, Fabc, Fd, dAb, Fv, single chains, single-chain antibodies, e.g., scFv, and single domain antibodies.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, FR residues of the human immunoglobulin are replaced by corresponding non-human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FR regions are those of a human immunoglobulin sequence. The humanized antibody optionally also will comprise at least a portion of an immunoglobulin constant region (Fc), typically that of a human immunoglobulin.

Examples of portions of antibodies or epitope-binding proteins encompassed by the present definition include: (i) the Fab fragment, having V_{L}, C_{L}, V_{H} and C_{H}1 domains; (ii) the Fab′ fragment, which is a Fab fragment having one or more cysteine residues at the C-terminus of the C_{H}1 domain; (iii) the Fd fragment having V_{H} and C_{H}1 domains; (iv) the Fd′ fragment having V_{H} and C_{H}1 domains and one or more cysteine residues at the C-terminus of the CHI domain; (v) the Fv fragment having the V_{L} and V_{H} domains of a single arm of an antibody; (vi) the dAb fragment (Ward et al., 341 Nature 544 (1989)) which consists of a V_{H} domain or a V_{L} domain that binds antigen; (vii) isolated CDR regions or isolated CDR regions presented in a functional framework; (viii) F(ab′)₂ fragments which are bivalent fragments including two Fab′ fragments linked by a disulphide bridge at the hinge region; (ix) single chain antibody molecules (e.g., single chain Fv; scFv) (Bird et al., 242 Science 423 (1988); and Huston et al., 85 PNAS 5879 (1988)); (x) “diabodies” with two antigen binding sites, comprising a heavy chain variable domain (V_{H}) connected to a light chain variable domain (V_{L}) in the same polypeptide chain (see, e.g., EP 404,097; WO 93/11161; Hollinger et al., 90 PNAS 6444 (1993)); (xi) “linear antibodies” comprising a pair of tandem Fd segments (V_{H}-Cₕ1-V_{H}-Cₕ1) which, together with complementary light chain polypeptides, form a pair of antigen binding regions (Zapata et al., Protein Eng. 8(10):1057-62 (1995); and U.S. Pat. No. 5,641,870).

As used herein, a “blocking” antibody or an antibody “antagonist” is one which inhibits or reduces biological activity of the antigen(s) it binds. In certain embodiments, the blocking antibodies or antagonist antibodies or portions thereof described herein completely inhibit the biological activity of the antigen(s).

Antibodies may act as agonists or antagonists of the recognized polypeptides. For example, the present invention includes antibodies which disrupt receptor/ligand interactions either partially or fully. The invention features both receptor-specific antibodies and ligand-specific antibodies. The invention also features receptor-specific antibodies which do not prevent ligand binding but prevent receptor activation. Receptor activation (i.e., signaling) may be determined by techniques described herein or otherwise known in the art. For example, receptor activation can be determined by detecting the phosphorylation (e.g., tyrosine or serine/threonine) of the receptor or of one of its down-stream substrates by immunoprecipitation followed by western blot analysis. In specific embodiments, antibodies are provided that inhibit ligand activity or receptor activity by at least 95%, at least 90%, at least 85%, at least 80%, at least 75%, at least 70%, at least 60%, or at least 50% of the activity in absence of the antibody.

The invention also features receptor-specific antibodies which both prevent ligand binding and receptor activation as well as antibodies that recognize the receptor-ligand complex. Likewise, encompassed by the invention are neutralizing antibodies which bind the ligand and prevent binding of the ligand to the receptor, as well as antibodies which bind the ligand, thereby preventing receptor activation, but do not prevent the ligand from binding the receptor. Further included in the invention are antibodies which activate the receptor. These antibodies may act as receptor agonists, i.e., potentiate or activate either all or a subset of the biological activities of the ligand-mediated receptor activation, for example, by inducing dimerization of the receptor. The antibodies may be specified as agonists, antagonists or inverse agonists for biological activities comprising the specific biological activities of the peptides disclosed herein. The antibody agonists and antagonists can be made using methods known in the art. See, e.g., PCT publication WO 96/40281; U.S. Pat. No. 5,811,097; Deng et al., Blood 92(6):1981-1988 (1998); Chen et al., Cancer Res. 58(16):3668-3678 (1998); Harrop et al., J. Immunol. 161(4):1786-1794 (1998); Zhu et al., Cancer Res. 58(15):3209-3214 (1998); Yoon et al., J. Immunol. 160(7):3170-3179 (1998); Prat et al., J. Cell. Sci. III (Pt2):237-247 (1998); Pitard et al., J. Immunol. Methods 205(2):177-190 (1997); Liautard et al., Cytokine 9(4):233-241 (1997); Carlson et al., J. Biol. Chem. 272(17):11295-11301 (1997); Taryman et al., Neuron 14(4):755-762 (1995); Muller et al., Structure 6(9):1153-1167 (1998); Bartunek et al., Cytokine 8(1):14-20 (1996).

The antibodies as defined for the present invention include derivatives that are modified, i.e., by the covalent attachment of any type of molecule to the antibody such that covalent attachment does not prevent the antibody from generating an anti-idiotypic response. For example, but not by way of limitation, the antibody derivatives include antibodies that have been modified, e.g., by glycosylation, acetylation, pegylation, phosphylation, amidation, derivatization by known protecting/blocking groups, proteolytic cleavage, linkage to a cellular ligand or other protein, etc. Any of numerous chemical modifications may be carried out by known techniques, including, but not limited to specific chemical cleavage, acetylation, formylation, metabolic synthesis of tunicamycin, etc. Additionally, the derivative may contain one or more non-classical amino acids.

Simple binding assays can be used to screen for or detect agents that bind to a target protein, or disrupt the interaction between proteins (e.g., a receptor and a ligand). Because certain targets of the present invention are transmembrane proteins, assays that use the soluble forms of these proteins rather than full-length protein can be used, in some embodiments. Soluble forms include, for example, those lacking the transmembrane domain and/or those comprising the IgV domain or fragments thereof which retain their ability to bind their cognate binding partners. Further, agents that inhibit or enhance protein interactions for use in the compositions and methods described herein, can include recombinant peptido-mimetics.

Detection methods useful in screening assays include antibody-based methods, detection of a reporter moiety, detection of cytokines as described herein, and detection of a gene signature as described herein.

Another variation of assays to determine binding of a receptor protein to a ligand protein is through the use of affinity biosensor methods. Such methods may be based on the piezoelectric effect, electrochemistry, or optical methods, such as ellipsometry, optical wave guidance, and surface plasmon resonance (SPR).

### Aptamers

In certain embodiments, the one or more agents is an aptamer. Nucleic acid aptamers are nucleic acid species that have been engineered through repeated rounds of in vitro selection or equivalently, SELEX (systematic evolution of ligands by exponential enrichment) to bind to various molecular targets such as small molecules, proteins, nucleic acids, cells, tissues and organisms. Nucleic acid aptamers have specific binding affinity to molecules through interactions other than classic Watson-Crick base pairing. Aptamers are useful in biotechnological and therapeutic applications as they offer molecular recognition properties similar to antibodies. In addition to their discriminate recognition, aptamers offer advantages over antibodies as they can be engineered completely in a test tube, are readily produced by chemical synthesis, possess desirable storage properties, and elicit little or no immunogenicity in therapeutic applications. In certain embodiments, RNA aptamers may be expressed from a DNA construct. In other embodiments, a nucleic acid aptamer may be linked to another polynucleotide sequence. The polynucleotide sequence may be a double stranded DNA polynucleotide sequence. The aptamer may be covalently linked to one strand of the polynucleotide sequence. The aptamer may be ligated to the polynucleotide sequence. The polynucleotide sequence may be configured, such that the polynucleotide sequence may be linked to a solid support or ligated to another polynucleotide sequence.

Aptamers, like peptides generated by phage display or monoclonal antibodies (“mAbs”), are capable of specifically binding to selected targets and modulating the target's activity, e.g., through binding, aptamers may block their target's ability to function. A typical aptamer is 10-15 kDa in size (30-45 nucleotides), binds its target with sub-nanomolar affinity, and discriminates against closely related targets (e.g., aptamers will typically not bind other proteins from the same gene family). Structural studies have shown that aptamers are capable of using the same types of binding interactions (e.g., hydrogen bonding, electrostatic complementarity, hydrophobic contacts, steric exclusion) that drives affinity and specificity in antibody-antigen complexes.

Aptamers have a number of desirable characteristics for use in research and as therapeutics and diagnostics including high specificity and affinity, biological efficacy, and excellent pharmacokinetic properties. In addition, they offer specific competitive advantages over antibodies and other protein biologics. Aptamers are chemically synthesized and are readily scaled as needed to meet production demand for research, diagnostic or therapeutic applications. Aptamers are chemically robust. They are intrinsically adapted to regain activity following exposure to factors such as heat and denaturants and can be stored for extended periods (>1 yr) at room temperature as lyophilized powders. Not being bound by a theory, aptamers bound to a solid support or beads may be stored for extended periods.

Oligonucleotides in their phosphodiester form may be quickly degraded by intracellular and extracellular enzymes such as endonucleases and exonucleases. Aptamers can include modified nucleotides conferring improved characteristics on the ligand, such as improved in vivo stability or improved delivery characteristics. Examples of such modifications include chemical substitutions at the ribose and/or phosphate and/or base positions. SELEX identified nucleic acid ligands containing modified nucleotides are described, e.g., in U.S. Pat. No. 5,660,985, which describes oligonucleotides containing nucleotide derivatives chemically modified at the 2′ position of ribose, 5 position of pyrimidines, and 8 position of purines, U.S. Pat. No. 5,756,703 which describes oligonucleotides containing various 2′-modified pyrimidines, and U.S. Pat. No. 5,580,737 which describes highly specific nucleic acid ligands containing one or more nucleotides modified with 2′-amino (2′-NH₂), 2′-fluoro (2′-F), and/or 2′-0-methyl (2′-OMe) substituents. Modifications of aptamers may also include, modifications at exocyclic amines, substitution of 4-thiouridine, substitution of 5-bromo or 5-iodo-uracil; backbone modifications, phosphorothioate or allyl phosphate modifications, methylations, and unusual base-pairing combinations such as the isobases isocytidine and isoguanosine. Modifications can also include 3′ and 5′ modifications such as capping. As used herein, the term phosphorothioate encompasses one or more non-bridging oxygen atoms in a phosphodiester bond replaced by one or more sulfur atoms. In further embodiments, the oligonucleotides comprise modified sugar groups, for example, one or more of the hydroxyl groups is replaced with halogen, aliphatic groups, or functionalized as ethers or amines. In one embodiment, the 2′-position of the furanose residue is substituted by any of an O-methyl, O-alkyl, O-allyl, S-alkyl, S-allyl, or halo group. Methods of synthesis of 2′-modified sugars are described, e.g., in Sproat, et al., Nucl. Acid Res. 19:733-738 (1991); Cotten, et al, Nucl. Acid Res. 19:2629-2635 (1991); and Hobbs, et al, Biochemistry 12:5138-5145 (1973). Other modifications are known to one of ordinary skill in the art. In certain embodiments, aptamers include aptamers with improved off-rates as described in International Patent Publication No. WO 2009012418, “Method for generating aptamers with improved off-rates,” incorporated herein by reference in its entirety. In certain embodiments aptamers are chosen from a library of aptamers. Such libraries include, but are not limited to those described in Rohloff et al., “Nucleic Acid Ligands With Protein-like Side Chains: Modified Aptamers and Their Use as Diagnostic and Therapeutic Agents,” Molecular Therapy Nucleic Acids (2014) 3, e201. Aptamers are also commercially available (see, e.g., SomaLogic, Inc., Boulder, Colo.). In certain embodiments, the present invention may utilize any aptamer containing any modification as described herein.

### Antibody Drug Conjugates

The term “antibody-drug-conjugate” or “ADC” refers to a binding protein, such as an antibody or antigen binding fragment thereof, chemically linked to one or more chemical drug(s) (also referred to herein as agent(s)) that may optionally be therapeutic or cytotoxic agents. In a preferred embodiment, an ADC includes an antibody, a cytotoxic or therapeutic drug, and a linker that enables attachment or conjugation of the drug to the antibody. An ADC typically has anywhere from 1 to 8 drugs conjugated to the antibody, including drug loaded species of 2, 4, 6, or 8.

In certain embodiments, the ADC specifically binds to a gene product expressed on the cell surface of a tumor cell. By means of an example, an agent, such as an antibody, capable of specifically binding to a gene product expressed on the cell surface of the tumor cells may be conjugated with a therapeutic or effector agent for targeted delivery of the therapeutic or effector agent to the immune cells.

Examples of such therapeutic or effector agents include immunomodulatory classes as discussed herein, such as without limitation a toxin, drug, radionuclide, cytokine, lymphokine, chemokine, growth factor, tumor necrosis factor, hormone, hormone antagonist, enzyme, oligonucleotide, siRNA, RNAi, photoactive therapeutic agent, anti-angiogenic agent and pro-apoptotic agent.

Non-limiting examples of drugs that may be included in the ADCs are mitotic inhibitors (e.g., maytansinoid DM4), antitumor antibiotics, immunomodulating agents, vectors for gene therapy, alkylating agents, antiangiogenic agents, antimetabolites, boron-containing agents, chemoprotective agents, hormones, antihormone agents, corticosteroids, photoactive therapeutic agents, oligonucleotides, radionuclide agents, topoisomerase inhibitors, tyrosine kinase inhibitors, and radiosensitizers.

Example toxins include ricin, abrin, alpha toxin, saporin, ribonuclease (RNase), DNase I, Staphylococcal enterotoxin-A, pokeweed antiviral protein, gelonin, diphtheria toxin, Pseudomonas exotoxin, or Pseudomonas endotoxin.

Example radionuclides include ^{103m}Rh, ¹⁰³Ru, ¹⁰⁵Rh, ¹⁰⁵Ru, ¹⁰⁷Hg, ¹⁰⁹Pd, ¹⁰⁹Pt, ¹¹¹Ag, ¹¹¹In, ^{113m}In, ¹¹⁹Sb, ¹¹C, ^{121m}Te, ^{122m}Te, ¹²⁵I, ^{125m}Te, ¹²⁶I, ¹³¹I, ¹³³I, ¹³N, ¹⁴²Pr, ¹⁴³Pr, ¹⁴⁹Pm, ¹⁵²Dy, ¹⁵³Sm, ¹⁵O, ¹⁶¹Ho, ¹⁶¹Tb, ¹⁶⁵Tm, ¹⁶⁶Dy, ¹⁶⁶Ho, ¹⁶⁷Tm, ¹⁶⁸Tm, ¹⁶⁹Er, ¹⁶⁹Yb, ¹⁷⁷Lu, ¹⁸⁶Re, ¹⁸⁸Re, ^{189m}Os, ¹⁸⁹Re, ¹⁹²Ir, ¹⁹⁴Ir, ¹⁹⁷Pt, ¹⁹⁸Au, ¹⁹⁹Au, ²⁰¹Tl, ²⁰³Hg, ²¹¹At, ²¹¹Bi, ²¹¹Pb, ²¹²Bi, ²¹²Pb, ²¹³Bi, ²¹⁵Po, ²¹⁷At, ²¹⁹Rn, ²²¹Fr, ²²³Ra, ²²⁴Ac, ²²⁵Ac, ²²⁵Fm, ³²P, ³³P, ⁴⁷Sc, ⁵¹Cr, ⁵⁷Co, ⁵⁸Co, ⁵⁹Fe, ⁶²Cu, ⁶⁷Cu, ⁶⁷Ga, ⁷⁵Br, ⁷⁵Se, ⁷⁶Br, ⁷⁷As, ⁷⁷Br, ^{80m}Br, ⁸⁹Sr, ⁹⁰Y, ⁹⁵Ru, ⁹⁷Ru, ⁹⁹Mo or ^{99m}Tc. Preferably, the radionuclide may be an alpha-particle-emitting radionuclide.

Example enzymes include malate dehydrogenase, staphylococcal nuclease, delta-V-steroid isomerase, yeast alcohol dehydrogenase, alpha-glycerophosphate dehydrogenase, triose phosphate isomerase, horseradish peroxidase, alkaline phosphatase, asparaginase, glucose oxidase, beta-galactosidase, ribonuclease, urease, catalase, glucose-6-phosphate dehydrogenase, glucoamylase or acetylcholinesterase. Such enzymes may be used, for example, in combination with prodrugs that are administered in relatively non-toxic form and converted at the target site by the enzyme into a cytotoxic agent. In other alternatives, a drug may be converted into less toxic form by endogenous enzymes in the subject but may be reconverted into a cytotoxic form by the therapeutic enzyme.

### Bi-Specific Antibodies

In certain embodiments, the one or more therapeutic agents can be bi-specific antigen-binding constructs, e.g., bi-specific antibodies (bsAb), that bind two antigens (see, e.g., Suurs et al., A review of bispecific antibodies and antibody constructs in oncology and clinical challenges. Pharmacol Ther. 2019 September; 201:103-119). The bi-specific antigen-binding construct includes two antigen-binding polypeptide constructs, e.g., antigen binding domains, wherein at least one polypeptide construct specifically binds to a tumor surface protein. In some embodiments, the antigen-binding construct is derived from known antibodies or antigen-binding constructs. In some embodiments, the antigen-binding polypeptide constructs comprise two antigen binding domains that comprise antibody fragments. In some embodiments, the first antigen binding domain and second antigen binding domain each independently comprises an antibody fragment selected from the group of: an scFv, a Fab, and an Fc domain. The antibody fragments may be the same format or different formats from each other. For example, in some embodiments, the antigen-binding polypeptide constructs comprise a first antigen binding domain comprising an scFv and a second antigen binding domain comprising a Fab. In some embodiments, the antigen-binding polypeptide constructs comprise a first antigen binding domain and a second antigen binding domain, wherein both antigen binding domains comprise an scFv. In some embodiments, the first and second antigen binding domains each comprise a Fab. In some embodiments, the first and second antigen binding domains each comprise an Fc domain. Any combination of antibody formats is suitable for the bi-specific antibody constructs disclosed herein.

In certain embodiments, immune cells can be engaged to tumor cells. In certain embodiments, tumor cells are targeted with a bsAb having affinity for both the tumor and a payload. In certain embodiments, two targets are disrupted on a tumor cell by the bsAb (e.g., any two of CLDN3, CLDN7, CLDN4, EPCAM, TACSTD2, MAL2, LSR, CD9, SPINT2, TM4SF1, TMEM205, TNFRSF12A and CD47). By means of an example, an agent, such as a bi-specific antibody, capable of specifically binding to a gene product expressed on the cell surface of the immune cells (e.g., CD3, CD8, CD28, CD16) and a tumor cell (e.g., CLDN3, CLDN7, and/or CLDN4) may be used for targeting polyfunctional immune cells to tumor cells. Immune cells targeted to a tumor may include T cells or Natural Killer cells.

### Administration

For therapeutic uses, the compositions or agents described herein may be administered systemically, for example, formulated in a pharmaceutically-acceptable buffer such as physiological saline. Preferable routes of administration include, for example, subcutaneous, intravenous, interperitoneal, intramuscular, or intradermal injections that provide continuous, sustained levels of the drug in the patient. Treatment of human patients or other animals will be carried out using a therapeutically effective amount of a therapeutic identified herein in a physiologically-acceptable carrier. Suitable carriers and their formulation are described, for example, in Remington's Pharmaceutical Sciences by E. W. Martin. The amount of the therapeutic agent to be administered varies depending upon the manner of administration, the age and body weight of the patient, and with the clinical symptoms of the neoplasia. Generally, amounts will be in the range of those used for other agents used in the treatment of other diseases associated with neoplasia, although in certain instances lower amounts will be needed because of the increased specificity of the compound. For example, a therapeutic compound is administered at a dosage that is cytotoxic to a neoplastic cell.

Human dosage amounts can initially be determined by extrapolating from the amount of compound used in mice, as a skilled artisan recognizes it is routine in the art to modify the dosage for humans compared to animal models. In certain embodiments, it is envisioned that the dosage may vary from between about 1 μg compound/Kg body weight to about 5000 mg compound/Kg body weight; or from about 5 mg/Kg body weight to about 4000 mg/Kg body weight or from about 10 mg/Kg body weight to about 3000 mg/Kg body weight; or from about 50 mg/Kg body weight to about 2000 mg/Kg body weight; or from about 100 mg/Kg body weight to about 1000 mg/Kg body weight; or from about 150 mg/Kg body weight to about 500 mg/Kg body weight. In other cases, this dose may be about 1, 5, 10, 25, 50, 75, 100, 150, 200, 250, 300, 350, 400, 450, 500, 550, 600, 650, 700, 750, 800, 850, 900, 950, 1000, 1050, 1100, 1150, 1200, 1250, 1300, 1350, 1400, 1450, 1500, 1600, 1700, 1800, 1900, 2000, 2500, 3000, 3500, 4000, 4500, or 5000 mg/Kg body weight. In other aspects, it is envisaged that doses may be in the range of about 5 mg compound/Kg body to about 20 mg compound/Kg body. In other embodiments, the doses may be about 8, 10, 12, 14, 16 or 18 mg/Kg body weight. Of course, this dosage amount may be adjusted upward or downward, as is routinely done in such treatment protocols, depending on the results of the initial clinical trials and the needs of a particular patient.

In some cases, the compound or composition of the invention is administered at a dose that is lower than the human equivalent dosage (HED) of the no observed adverse effect level (NOAEL) over a period of three months, four months, six months, nine months, 1 year, 2 years, 3 years, 4 years or more. The NOAEL, as determined in animal studies, is useful in determining the maximum recommended starting dose for human clinical trials. For instance, the NOAELs can be extrapolated to determine human equivalent dosages. Typically, such extrapolations between species are conducted based on the doses that are normalized to body surface area (i.e., mg/m²). In specific embodiments, the NOAELs are determined in mice, hamsters, rats, ferrets, guinea pigs, rabbits, dogs, primates, primates (monkeys, marmosets, squirrel monkeys, baboons), micropigs or minipigs. For a discussion on the use of NOAELs and their extrapolation to determine human equivalent doses, see Guidance for Industry Estimating the Maximum Safe Starting Dose in Initial Clinical Trials for Therapeutics in Adult Healthy Volunteers, U.S. Department of Health and Human Services Food and Drug Administration Center for Drug Evaluation and Research (CDER), Pharmacology and Toxicology, July 2005, incorporated herein by reference.

The amount of an agent of the invention used in the prophylactic and/or therapeutic regimens which will be effective in the prevention, treatment, and/or management of cancer can be based on the currently prescribed dosage of the agent as well as assessed by methods disclosed herein and known in the art. The frequency and dosage will vary also according to factors specific for each patient depending on the specific compounds administered, the severity of the cancerous condition, the route of administration, as well as age, body, weight, response, and the past medical history of the patient. For example, the dosage of an agent of the invention which will be effective in the treatment, prevention, and/or management of cancer can be determined by administering the compound to an animal model such as, e.g., the animal models disclosed herein or known to those skilled in the art. In addition, in vitro assays may optionally be employed to help identify optimal dosage ranges.

In some aspects, the prophylactic and/or therapeutic regimens comprise titrating the dosages administered to the patient so as to achieve a specified measure of therapeutic efficacy. Such measures include a reduction in the cancer cell population in the patient.

In certain cases, the dosage of the compound of the invention in the prophylactic and/or therapeutic regimen is adjusted so as to achieve a reduction in the number or amount of cancer cells found in a test specimen extracted from a patient after undergoing the prophylactic and/or therapeutic regimen, as compared with a reference sample. Here, the reference sample is a specimen extracted from the patient undergoing therapy, wherein the specimen is extracted from the patient at an earlier time point. In one aspect, the reference sample is a specimen extracted from the same patient, prior to receiving the prophylactic and/or therapeutic regimen. For example, the number or amount of cancer cells in the test specimen is at least 2%, 5%, 10%, 15%, 20%, 30%, 40%, 50%, 60%, 70%, 80%, 90%, 95% or 99% lower than in the reference sample.

In some cases, the dosage of the compound of the invention in the prophylactic and/or therapeutic regimen is adjusted so as to achieve a number or amount of cancer cells that falls within a predetermined reference range. In these embodiments, the number or amount of cancer cells in a test specimen is compared with a predetermined reference range.

In other embodiments, the dosage of the compound of the invention in prophylactic and/or therapeutic regimen is adjusted so as to achieve a reduction in the number or amount of cancer cells found in a test specimen extracted from a patient after undergoing the prophylactic and/or therapeutic regimen, as compared with a reference sample, wherein the reference sample is a specimen is extracted from a healthy, noncancer-afflicted patient. For example, the number or amount of cancer cells in the test specimen is at least within 60%, 50%, 40%, 30%, 20%, 15%, 10%, 5%, or 2% of the number or amount of cancer cells in the reference sample.

In treating certain human patients having solid tumors, extracting multiple tissue specimens from a suspected tumor site may prove impracticable. In these cases, the dosage of the compounds of the invention in the prophylactic and/or therapeutic regimen for a human patient is extrapolated from doses in animal models that are effective to reduce the cancer population in those animal models. In the animal models, the prophylactic and/or therapeutic regimens are adjusted so as to achieve a reduction in the number or amount of cancer cells found in a test specimen extracted from an animal after undergoing the prophylactic and/or therapeutic regimen, as compared with a reference sample. The reference sample can be a specimen extracted from the same animal, prior to receiving the prophylactic and/or therapeutic regimen. In specific embodiments, the number or amount of cancer cells in the test specimen is at least 2%, 5%, 10%, 15%, 20%, 30%, 40%, 50% or 60% lower than in the reference sample. The doses effective in reducing the number or amount of cancer cells in the animals can be normalized to body surface area (e.g., mg/m²) to provide an equivalent human dose.

The prophylactic and/or therapeutic regimens disclosed herein comprise administration of compounds of the invention or pharmaceutical compositions thereof to the patient in a single dose or in multiple doses (e.g., 1, 2, 3, 4, 5, 6, 7, 8, 10, 15, 20, or more doses).

In one aspect, the prophylactic and/or therapeutic regimens comprise administration of the compounds of the invention or pharmaceutical compositions thereof in multiple doses. When administered in multiple doses, the compounds or pharmaceutical compositions are administered with a frequency and in an amount sufficient to prevent, treat, and/or manage the condition. For example, the frequency of administration ranges from once a day up to about once every eight weeks. In another example, the frequency of administration ranges from about once a week up to about once every six weeks. In another example, the frequency of administration ranges from about once every three weeks up to about once every four weeks.

Generally, the dosage of a compound of the invention administered to a subject to prevent, treat, and/or manage cancer is in the range of 0.01 to 500 mg/kg, e.g., in the range of 0.1 mg/kg to 100 mg/kg, of the subject's body weight. For example, the dosage administered to a subject is in the range of 0.1 mg/kg to 50 mg/kg, or 1 mg/kg to 50 mg/kg, of the subject's body weight, more preferably in the range of 0.1 mg/kg to 25 mg/kg, or 1 mg/kg to 25 mg/kg, of the patient's body weight. In another example, the dosage of a compound of the invention administered to a subject to prevent, treat, and/or manage cancer in a patient is 500 mg/kg or less, preferably 250 mg/kg or less, 100 mg/kg or less, 95 mg/kg or less, 90 mg/kg or less, 85 mg/kg or less, 80 mg/kg or less, 75 mg/kg or less, 70 mg/kg or less, 65 mg/kg or less, 60 mg/kg or less, 55 mg/kg or less, 50 mg/kg or less, 45 mg/kg or less, 40 mg/kg or less, 35 mg/kg or less, 30 mg/kg or less, 25 mg/kg or less, 20 mg/kg or less, 15 mg/kg or less, 10 mg/kg or less, 5 mg/kg or less, 2.5 mg/kg or less, 2 mg/kg or less, 1.5 mg/kg or less, or 1 mg/kg or less of a patient's body weight.

In another example, the dosage of a compound of the invention administered to a subject to prevent, treat, and/or manage cancer in a patient is a unit dose of 0.1 to 50 mg, 0.1 mg to 20 mg, 0.1 mg to 15 mg, 0.1 mg to 12 mg, 0.1 mg to 10 mg, 0.1 mg to 8 mg, 0.1 mg to 7 mg, 0.1 mg to 5 mg, 0.1 to 2.5 mg, 0.25 mg to 20 mg, 0.25 to 15 mg, 0.25 to 12 mg, 0.25 to 10 mg, 0.25 to 8 mg, 0.25 mg to 7 mg, 0.25 mg to 5 mg, 0.5 mg to 2.5 mg, 1 mg to 20 mg, 1 mg to 15 mg, 1 mg to 12 mg, 1 mg to 10 mg, 1 mg to 8 mg, 1 mg to 7 mg, 1 mg to 5 mg, or 1 mg to 2.5 mg.

In another example, the dosage of a compound of the invention administered to a subject to prevent, treat, and/or manage cancer in a patient is in the range of 0.01 to 10 g/m², and more typically, in the range of 0.1 g/m² to 7.5 g/m², of the subject's body weight. For example, the dosage administered to a subject is in the range of 0.5 g/m² to 5 g/m², or 1 g/m² to 5 g/m² of the subject's body's surface area.

In another example, the prophylactic and/or therapeutic regimen comprises administering to a patient one or more doses of an effective amount of a compound of the invention, wherein the dose of an effective amount achieves a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention.

In another example, the prophylactic and/or therapeutic regimen comprises administering to a patient a plurality of doses of an effective amount of a compound of the invention, wherein the plurality of doses maintains a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention for at least 1 day, 1 month, 2 months, 3 months, 4 months, 5 months, 6 months, 7 months, 8 months, 9 months, 10 months, 11 months, 12 months, 15 months, 18 months, 24 months or 36 months.

In other embodiments, the prophylactic and/or therapeutic regimen comprises administering to a patient a plurality of doses of an effective amount of a compound of the invention, wherein the plurality of doses maintains a plasma level of at least 0.1 μg/mL, at least 0.5 μg/mL, at least 1 μg/mL, at least 2 μg/mL, at least 5 μg/mL, at least 6 μg/mL, at least 10 μg/mL, at least 15 μg/mL, at least 20 μg/mL, at least 25 μg/mL, at least 50 μg/mL, at least 100 μg/mL, at least 125 μg/mL, at least 150 μg/mL, at least 175 μg/mL, at least 200 μg/mL, at least 225 μg/mL, at least 250 μg/mL, at least 275 μg/mL, at least 300 μg/mL, at least 325 μg/mL, at least 350 μg/mL, at least 375 μg/mL, or at least 400 μg/mL of the compound of the invention for at least 1 day, 1 month, 2 months, 3 months, 4 months, 5 months, 6 months, 7 months, 8 months, 9 months, 10 months, 11 months, 12 months, 15 months, 18 months, 24 months or 36 months.

### Release of Pharmaceutical Compositions

Pharmaceutical compositions according to the invention may be formulated to release the active compound substantially immediately upon administration or at any predetermined time or time period after administration. The latter types of compositions are generally known as controlled release formulations, which include (i) formulations that create a substantially constant concentration of the drug within the body over an extended period of time; (ii) formulations that after a predetermined lag time create a substantially constant concentration of the drug within the body over an extended period of time; (iii) formulations that sustain action during a predetermined time period by maintaining a relatively, constant, effective level in the body with concomitant minimization of undesirable side effects associated with fluctuations in the plasma level of the active substance (sawtooth kinetic pattern); (iv) formulations that localize action by, e.g., spatial placement of a controlled release composition adjacent to or in contact with the thymus; (v) formulations that allow for convenient dosing, such that doses are administered, for example, once every one or two weeks; and (vi) formulations that target a neoplasia by using carriers or chemical derivatives to deliver the therapeutic agent to a particular cell type (e.g., neoplastic cell). For some applications, controlled release formulations obviate the need for frequent dosing during the day to sustain the plasma level at a therapeutic level.

Any of a number of strategies can be pursued in order to obtain controlled release in which the rate of release outweighs the rate of metabolism of the compound in question. In one example, controlled release is obtained by appropriate selection of various formulation parameters and ingredients, including, e.g., various types of controlled release compositions and coatings. Thus, the therapeutic is formulated with appropriate excipients into a pharmaceutical composition that, upon administration, releases the therapeutic in a controlled manner. Examples include single or multiple unit tablet or capsule compositions, oil solutions, suspensions, emulsions, microcapsules, microspheres, molecular complexes, nanoparticles, patches, and liposomes.

### Parenteral Compositions

The pharmaceutical composition may be administered parenterally by injection, infusion or implantation (subcutaneous, intravenous, intramuscular, intraperitoneal, or the like) in dosage forms, formulations, or via suitable delivery devices or implants containing conventional, non-toxic pharmaceutically acceptable carriers and adjuvants. The formulation and preparation of such compositions are well known to those skilled in the art of pharmaceutical formulation. Formulations can be found in Remington: The Science and Practice of Pharmacy, supra.

Compositions for parenteral use may be provided in unit dosage forms (e.g., in single-dose ampoules), or in vials containing several doses and in which a suitable preservative may be added (see below). The composition may be in the form of a solution, a suspension, an emulsion, an infusion device, or a delivery device for implantation, or it may be presented as a dry powder to be reconstituted with water or another suitable vehicle before use. Apart from the active agent that reduces or ameliorates a neoplasia, the composition may include suitable parenterally acceptable carriers and/or excipients. The active therapeutic agent(s) may be incorporated into microspheres, microcapsules, nanoparticles, liposomes, or the like for controlled release. Furthermore, the composition may include suspending, solubilizing, stabilizing, pH-adjusting agents, tonicity adjusting agents, and/or dispersing, agents.

As indicated above, the pharmaceutical compositions according to the invention may be in the form suitable for sterile injection. To prepare such a composition, the suitable active antineoplastic therapeutic(s) are dissolved or suspended in a parenterally acceptable liquid vehicle. Among acceptable vehicles and solvents that may be employed are water, water adjusted to a suitable pH by addition of an appropriate amount of hydrochloric acid, sodium hydroxide or a suitable buffer, 1,3-butanediol, Ringer's solution, and isotonic sodium chloride solution and dextrose solution. The aqueous formulation may also contain one or more preservatives (e.g., methyl, ethyl or n-propyl p-hydroxybenzoate). In cases where one of the compounds is only sparingly or slightly soluble in water, a dissolution enhancing or solubilizing agent can be added, or the solvent may include 10-60% w/w of propylene glycol.

### Controlled Release Parenteral Compositions

Controlled release parenteral compositions may be in form of aqueous suspensions, microspheres, microcapsules, magnetic microspheres, oil solutions, oil suspensions, or emulsions. Alternatively, the active drug may be incorporated in biocompatible carriers, liposomes, nanoparticles, implants, or infusion devices.

Materials for use in the preparation of microspheres and/or microcapsules are, e.g., biodegradable/bioerodible polymers such as polygalactin, poly-(isobutyl cyanoacrylate), poly(2-hydroxyethyl-L-glutam-nine) and, poly(lactic acid). Biocompatible carriers that may be used when formulating a controlled release parenteral formulation are carbohydrates (e.g., dextrans), proteins (e.g., albumin), lipoproteins, or antibodies. Materials for use in implants can be non-biodegradable (e.g., polydimethyl siloxane) or biodegradable (e.g., poly(caprolactone), poly(lactic acid), poly(glycolic acid) or poly(ortho esters) or combinations thereof).

### Diagnostic Methods

The invention provides biomarkers (e.g., malignant cell specific markers and expression programs) for the identification, diagnosis, prognosis and manipulation of cell properties, for use in a variety of diagnostic and/or therapeutic indications. In certain embodiments, detecting a tumor marker or expression program may indicate that a subject has cancer. In certain embodiments, detecting a tumor marker or expression program may indicate that a subject suffering from a cancer may respond to JAK/STAT inhibition. In certain embodiments, detecting a tumor marker or expression program may indicate prognosis for a subject suffering from cancer. In certain embodiments, detection one or more tumor markers or biological programs indicates a treatment as described herein. For example, if the ovarian tumor expresses one or more of the MHC class II biological programs, administering a therapeutic regimen that comprises an immunotherapy. In another example, if the ovarian tumor exhibits increased macrophages characteristic of an immunoreactive phenotype, administering a therapeutic regimen that comprises an immunotherapy. In another example, if the ovarian tumor does not exhibit increased CAFs characteristic of a mesenchymal phenotype, administering a therapeutic regimen that comprises an immunotherapy or if the ovarian tumor exhibits increased CAFs characteristic of a mesenchymal phenotype, administering a therapeutic regimen that comprises a JAK/STAT inhibitor.

Biomarkers in the context of the present invention encompasses, without limitation nucleic acids, proteins, reaction products, and metabolites, together with their polymorphisms, mutations, variants, modifications, subunits, fragments, and other analytes or sample-derived measures. In certain embodiments, biomarkers include the signature genes or signature gene products, and/or cells as described herein.

Biomarkers are useful in methods of diagnosing, prognosing and/or staging an immune response in a subject by detecting a first level of expression, activity and/or function of one or more biomarker and comparing the detected level to a control of level wherein a difference in the detected level and the control level indicates that the presence of an immune response in the subject.

The terms “diagnosis” and “monitoring” are commonplace and well-understood in medical practice. By means of further explanation and without limitation the term “diagnosis” generally refers to the process or act of recognising, deciding on or concluding on a disease or condition in a subject on the basis of symptoms and signs and/or from results of various diagnostic procedures (such as, for example, from knowing the presence, absence and/or quantity of one or more biomarkers characteristic of the diagnosed disease or condition).

The terms “prognosing” or “prognosis” generally refer to an anticipation on the progression of a disease or condition and the prospect (e.g., the probability, duration, and/or extent) of recovery. A good prognosis of the diseases or conditions taught herein may generally encompass anticipation of a satisfactory partial or complete recovery from the diseases or conditions, preferably within an acceptable time period. A good prognosis of such may more commonly encompass anticipation of not further worsening or aggravating of such, preferably within a given time period. A poor prognosis of the diseases or conditions as taught herein may generally encompass anticipation of a substandard recovery and/or unsatisfactorily slow recovery, or to substantially no recovery or even further worsening of such.

The biomarkers of the present invention are useful in methods of identifying patient populations at risk or suffering from cancer or for identifying patients that will respond to specific treatments based on a detected level of expression, activity and/or function of one or more biomarkers. These biomarkers are also useful in monitoring subjects undergoing treatments and therapies for suitable or aberrant response(s) to determine efficaciousness of the treatment or therapy and for selecting or modifying therapies and treatments that would be efficacious in treating, delaying the progression of or otherwise ameliorating a symptom. The biomarkers provided herein are useful for selecting a group of patients at a specific state of a disease with accuracy that facilitates selection of treatments.

The term “monitoring” generally refers to the follow-up of a disease or a condition in a subject for any changes which may occur over time.

The terms also encompass prediction of a disease. The terms “predicting” or “prediction” generally refer to an advance declaration, indication or foretelling of a disease or condition in a subject not (yet) having said disease or condition. For example, a prediction of a disease or condition in a subject may indicate a probability, chance or risk that the subject will develop said disease or condition, for example within a certain time period or by a certain age. Said probability, chance or risk may be indicated inter alia as an absolute value, range or statistics, or may be indicated relative to a suitable control subject or subject population (such as, e.g., relative to a general, normal or healthy subject or subject population). Hence, the probability, chance or risk that a subject will develop a disease or condition may be advantageously indicated as increased or decreased, or as fold-increased or fold-decreased relative to a suitable control subject or subject population. As used herein, the term “prediction” of the conditions or diseases as taught herein in a subject may also particularly mean that the subject has a ‘positive’ prediction of such, i.e., that the subject is at risk of having such (e.g., the risk is significantly increased vis-à-vis a control subject or subject population). The term “prediction of no” diseases or conditions as taught herein as described herein in a subject may particularly mean that the subject has a ‘negative’ prediction of such, i.e., that the subject's risk of having such is not significantly increased vis-à-vis a control subject or subject population.

Suitably, an altered quantity or phenotype of the immune cells in the subject compared to a control subject having normal immune status or not having a disease comprising an immune component indicates that the subject has an impaired immune status or has a disease comprising an immune component or would benefit from an immune therapy.

Hence, the methods may rely on comparing the quantity of immune cell populations, biomarkers, or gene or gene product signatures measured in samples from patients with reference values, wherein said reference values represent known predictions, diagnoses and/or prognoses of diseases or conditions as taught herein.

For example, distinct reference values may represent the prediction of a risk (e.g., an abnormally elevated risk) of having a given disease or condition as taught herein vs. the prediction of no or normal risk of having said disease or condition. In another example, distinct reference values may represent predictions of differing degrees of risk of having such disease or condition.

In a further example, distinct reference values can represent the diagnosis of a given disease or condition as taught herein vs. the diagnosis of no such disease or condition (such as, e.g., the diagnosis of healthy, or recovered from said disease or condition, etc.). In another example, distinct reference values may represent the diagnosis of such disease or condition of varying severity.

In yet another example, distinct reference values may represent a good prognosis for a given disease or condition as taught herein vs. a poor prognosis for said disease or condition. In a further example, distinct reference values may represent varyingly favourable or unfavourable prognoses for such disease or condition.

Such comparison may generally include any means to determine the presence or absence of at least one difference and optionally of the size of such difference between values being compared. A comparison may include a visual inspection, an arithmetical or statistical comparison of measurements. Such statistical comparisons include, but are not limited to, applying a rule.

Reference values may be established according to known procedures previously employed for other cell populations, biomarkers and gene or gene product signatures. For example, a reference value may be established in an individual or a population of individuals characterised by a particular diagnosis, prediction and/or prognosis of said disease or condition (i.e., for whom said diagnosis, prediction and/or prognosis of the disease or condition holds true). Such population may comprise without limitation 2 or more, 10 or more, 100 or more, or even several hundred or more individuals.

A “deviation” of a first value from a second value may generally encompass any direction (e.g., increase: first value>second value; or decrease: first value<second value) and any extent of alteration.

For example, a deviation may encompass a decrease in a first value by, without limitation, at least about 10% (about 0.9-fold or less), or by at least about 20% (about 0.8-fold or less), or by at least about 30% (about 0.7-fold or less), or by at least about 40% (about 0.6-fold or less), or by at least about 50% (about 0.5-fold or less), or by at least about 60% (about 0.4-fold or less), or by at least about 70% (about 0.3-fold or less), or by at least about 80% (about 0.2-fold or less), or by at least about 90% (about 0.1-fold or less), relative to a second value with which a comparison is being made.

For example, a deviation may encompass an increase of a first value by, without limitation, at least about 10% (about 1.1-fold or more), or by at least about 20% (about 1.2-fold or more), or by at least about 30% (about 1.3-fold or more), or by at least about 40% (about 1.4-fold or more), or by at least about 50% (about 1.5-fold or more), or by at least about 60% (about 1.6-fold or more), or by at least about 70% (about 1.7-fold or more), or by at least about 80% (about 1.8-fold or more), or by at least about 90% (about 1.9-fold or more), or by at least about 100% (about 2-fold or more), or by at least about 150% (about 2.5-fold or more), or by at least about 200% (about 3-fold or more), or by at least about 500% (about 6-fold or more), or by at least about 700% (about 8-fold or more), or like, relative to a second value with which a comparison is being made.

Preferably, a deviation may refer to a statistically significant observed alteration. For example, a deviation may refer to an observed alteration which falls outside of error margins of reference values in a given population (as expressed, for example, by standard deviation or standard error, or by a predetermined multiple thereof, e.g., ±1×SD or ±2×SD or ±3×SD, or 1×SE or ±2×SE or ±3×SE). Deviation may also refer to a value falling outside of a reference range defined by values in a given population (for example, outside of a range which comprises ≥40%, ≥50%, ≥60%, ≥70%, ≥75% or ≥80% or ≥85% or ≥90% or ≥95% or even ≥100% of values in said population).

In a further embodiment, a deviation may be concluded if an observed alteration is beyond a given threshold or cut-off. Such threshold or cut-off may be selected as generally known in the art to provide for a chosen sensitivity and/or specificity of the prediction methods, e.g., sensitivity and/or specificity of at least 50%, or at least 60%, or at least 70%, or at least 80%, or at least 85%, or at least 90%, or at least 95%.

For example, receiver-operating characteristic (ROC) curve analysis can be used to select an optimal cut-off value of the quantity of a given immune cell population, biomarker or gene or gene product signatures, for clinical use of the present diagnostic tests, based on acceptable sensitivity and specificity, or related performance measures which are well-known per se, such as positive predictive value (PPV), negative predictive value (NPV), positive likelihood ratio (LR+), negative likelihood ratio (LR−), Youden index, or similar.

In one embodiment, the signature genes, biomarkers, and/or cells may be detected or isolated by immunofluorescence, immunohistochemistry (IHC), fluorescence activated cell sorting (FACS), mass spectrometry (MS), mass cytometry (CyTOF), RNA-seq, single cell RNA-seq (described further herein), quantitative RT-PCR, single cell qPCR, FISH, RNA-FISH, MERFISH (multiplex (in situ) RNA FISH) and/or by in situ hybridization. Other methods including absorbance assays and colorimetric assays are known in the art and may be used herein. detection may comprise primers and/or probes or fluorescently bar-coded oligonucleotide probes for hybridization to RNA (see e.g., Geiss G K, et al., Direct multiplexed measurement of gene expression with color-coded probe pairs. Nat Biotechnol. 2008 March; 26(3):317-25).

### MS Methods

Biomarker detection may also be evaluated using mass spectrometry methods. A variety of configurations of mass spectrometers can be used to detect biomarker values. Several types of mass spectrometers are available or can be produced with various configurations. In general, a mass spectrometer has the following major components: a sample inlet, an ion source, a mass analyzer, a detector, a vacuum system, and instrument-control system, and a data system. Difference in the sample inlet, ion source, and mass analyzer generally define the type of instrument and its capabilities. For example, an inlet can be a capillary-column liquid chromatography source or can be a direct probe or stage such as used in matrix-assisted laser desorption. Common ion sources are, for example, electrospray, including nanospray and microspray or matrix-assisted laser desorption. Common mass analyzers include a quadrupole mass filter, ion trap mass analyzer and time-of-flight mass analyzer. Additional mass spectrometry methods are well known in the art (see Burlingame et al., Anal. Chem. 70:647 R-716R (1998); Kinter and Sherman, New York (2000)).

Protein biomarkers and biomarker values can be detected and measured by any of the following: electrospray ionization mass spectrometry (ESI-MS), ESI-MS/MS, ESI-MS/(MS)n, matrix-assisted laser desorption ionization time-of-flight mass spectrometry (MALDI-TOF-MS), surface-enhanced laser desorption/ionization time-of-flight mass spectrometry (SELDI-TOF-MS), desorption/ionization on silicon (DIOS), secondary ion mass spectrometry (SIMS), quadrupole time-of-flight (Q-TOF), tandem time-of-flight (TOF/TOF) technology, called ultraflex III TOF/TOF, atmospheric pressure chemical ionization mass spectrometry (APCI-MS), APCI-MS/MS, APCI-(MS).sup.N, atmospheric pressure photoionization mass spectrometry (APPI-MS), APPI-MS/MS, and APPI-(MS).sup.N, quadrupole mass spectrometry, Fourier transform mass spectrometry (FTMS), quantitative mass spectrometry, and ion trap mass spectrometry.

Sample preparation strategies are used to label and enrich samples before mass spectroscopic characterization of protein biomarkers and determination biomarker values. Labeling methods include but are not limited to isobaric tag for relative and absolute quantitation (iTRAQ) and stable isotope labeling with amino acids in cell culture (SILAC). Capture reagents used to selectively enrich samples for candidate biomarker proteins prior to mass spectroscopic analysis include but are not limited to aptamers, antibodies, nucleic acid probes, chimeras, small molecules, an F(ab′)₂ fragment, a single chain antibody fragment, an Fv fragment, a single chain Fv fragment, a nucleic acid, a lectin, a ligand-binding receptor, affybodies, nanobodies, ankyrins, domain antibodies, alternative antibody scaffolds (e.g. diabodies etc) imprinted polymers, avimers, peptidomimetics, peptoids, peptide nucleic acids, threose nucleic acid, a hormone receptor, a cytokine receptor, and synthetic receptors, and modifications and fragments of these.

### Immunoassays

Immunoassay methods are based on the reaction of an antibody to its corresponding target or analyte and can detect the analyte in a sample depending on the specific assay format. To improve specificity and sensitivity of an assay method based on immunoreactivity, monoclonal antibodies are often used because of their specific epitope recognition. Polyclonal antibodies have also been successfully used in various immunoassays because of their increased affinity for the target as compared to monoclonal antibodies Immunoassays have been designed for use with a wide range of biological sample matrices Immunoassay formats have been designed to provide qualitative, semi-quantitative, and quantitative results.

Quantitative results may be generated through the use of a standard curve created with known concentrations of the specific analyte to be detected. The response or signal from an unknown sample is plotted onto the standard curve, and a quantity or value corresponding to the target in the unknown sample is established.

Numerous immunoassay formats have been designed. ELISA or EIA can be quantitative for the detection of an analyte/biomarker. This method relies on attachment of a label to either the analyte or the antibody and the label component includes, either directly or indirectly, an enzyme. ELISA tests may be formatted for direct, indirect, competitive, or sandwich detection of the analyte. Other methods rely on labels such as, for example, radioisotopes (I¹²⁵) or fluorescence. Additional techniques include, for example, agglutination, nephelometry, turbidimetry, Western blot, immunoprecipitation, immunocytochemistry, immunohistochemistry, flow cytometry, Luminex assay, and others (see ImmunoAssay: A Practical Guide, edited by Brian Law, published by Taylor & Francis, Ltd., 2005 edition).

Exemplary assay formats include enzyme-linked immunosorbent assay (ELISA), radioimmunoassay, fluorescent, chemiluminescence, and fluorescence resonance energy transfer (FRET) or time resolved-FRET (TR-FRET) immunoassays. Examples of procedures for detecting biomarkers include biomarker immunoprecipitation followed by quantitative methods that allow size and peptide level discrimination, such as gel electrophoresis, capillary electrophoresis, planar electrochromatography, and the like.

Methods of detecting and/or quantifying a detectable label or signal generating material depend on the nature of the label. The products of reactions catalyzed by appropriate enzymes (where the detectable label is an enzyme; see above) can be, without limitation, fluorescent, luminescent, or radioactive or they may absorb visible or ultraviolet light. Examples of detectors suitable for detecting such detectable labels include, without limitation, x-ray film, radioactivity counters, scintillation counters, spectrophotometers, colorimeters, fluorometers, luminometers, and densitometers.

Any of the methods for detection can be performed in any format that allows for any suitable preparation, processing, and analysis of the reactions. This can be, for example, in multiwell assay plates (e.g., 96 wells or 384 wells) or using any suitable array or microarray. Stock solutions for various agents can be made manually or robotically, and all subsequent pipetting, diluting, mixing, distribution, washing, incubating, sample readout, data collection and analysis can be done robotically using commercially available analysis software, robotics, and detection instrumentation capable of detecting a detectable label.

### Hybridization Assays

Such applications are hybridization assays in which a nucleic acid that displays “probe” nucleic acids for each of the genes to be assayed/profiled in the profile to be generated is employed. In these assays, a sample of target nucleic acids is first prepared from the initial nucleic acid sample being assayed, where preparation may include labeling of the target nucleic acids with a label, e.g., a member of a signal producing system. Following target nucleic acid sample preparation, the sample is contacted with the array under hybridization conditions, whereby complexes are formed between target nucleic acids that are complementary to probe sequences attached to the array surface. The presence of hybridized complexes is then detected, either qualitatively or quantitatively. Specific hybridization technology which may be practiced to generate the expression profiles employed in the subject methods includes the technology described in U.S. Pat. Nos. 5,143,854; 5,288,644; 5,324,633; 5,432,049; 5,470,710; 5,492,806; 5,503,980; 5,510,270; 5,525,464; 5,547,839; 5,580,732; 5,661,028; 5,800,992; the disclosures of which are herein incorporated by reference; as well as WO 95/21265; WO 96/31622; WO 97/10365; WO 97/27317; EP 373 203; and EP 785 280. In these methods, an array of “probe” nucleic acids that includes a probe for each of the biomarkers whose expression is being assayed is contacted with target nucleic acids as described above. Contact is carried out under hybridization conditions, e.g., stringent hybridization conditions as described above, and unbound nucleic acid is then removed. The resultant pattern of hybridized nucleic acids provides information regarding expression for each of the biomarkers that have been probed, where the expression information is in terms of whether or not the gene is expressed and, typically, at what level, where the expression data, i.e., expression profile, may be both qualitative and quantitative.

Optimal hybridization conditions will depend on the length (e.g., oligomer vs. polynucleotide greater than 200 bases) and type (e.g., RNA, DNA, PNA) of labeled probe and immobilized polynucleotide or oligonucleotide. General parameters for specific (i.e., stringent) hybridization conditions for nucleic acids are described in Sambrook et al., supra, and in Ausubel et al., “Current Protocols in Molecular Biology”, Greene Publishing and Wiley-interscience, NY (1987), which is incorporated in its entirety for all purposes. When the cDNA microarrays are used, typical hybridization conditions are hybridization in 5×SSC plus 0.2% SDS at 65 C for 4 hours followed by washes at 25° C. in low stringency wash buffer (1×SSC plus 0.2% SDS) followed by 10 minutes at 25° C. in high stringency wash buffer (0.1SSC plus 0.2% SDS) (see Shena et al., Proc. Natl. Acad. Sci. USA, Vol. 93, p. 10614 (1996)). Useful hybridization conditions are also provided in, e.g., Tijessen, Hybridization With Nucleic Acid Probes”, Elsevier Science Publishers B.V. (1993) and Kricka, “Nonisotopic DNA Probe Techniques”, Academic Press, San Diego, Calif. (1992).

### Sequencing and Nucleic Acid Profiling

In certain embodiments, the invention involves targeted nucleic acid profiling (e.g., sequencing, quantitative reverse transcription polymerase chain reaction, and the like) (see e.g., Geiss G K, et al., Direct multiplexed measurement of gene expression with color-coded probe pairs. Nat Biotechnol. 2008 March; 26(3):317-25). In certain embodiments, a target nucleic acid molecule (e.g., RNA molecule), may be sequenced by any method known in the art, for example, methods of high-throughput sequencing, also known as next generation sequencing or deep sequencing. A nucleic acid target molecule labeled with a barcode (for example, an origin-specific barcode) can be sequenced with the barcode to produce a single read and/or contig containing the sequence, or portions thereof, of both the target molecule and the barcode. Exemplary next generation sequencing technologies include, for example, Illumina sequencing, Ion Torrent sequencing, 454 sequencing, SOLiD sequencing, and nanopore sequencing amongst others.

In certain embodiments, the invention involves single cell RNA sequencing (see, e.g., Kalisky, T., Blainey, P. & Quake, S. R. Genomic Analysis at the Single-Cell Level. Annual review of genetics 45, 431-445, (2011); Kalisky, T. & Quake, S. R. Single-cell genomics. Nature Methods 8, 311-314 (2011); Islam, S. et al. Characterization of the single-cell transcriptional landscape by highly multiplex RNA-seq. Genome Research, (2011); Tang, F. et al. RNA-Seq analysis to capture the transcriptome landscape of a single cell. Nature Protocols 5, 516-535, (2010); Tang, F. et al. mRNA-Seq whole-transcriptome analysis of a single cell. Nature Methods 6, 377-382, (2009); Ramskold, D. et al. Full-length mRNA-Seq from single-cell levels of RNA and individual circulating tumor cells. Nature Biotechnology 30, 777-782, (2012); and Hashimshony, T., Wagner, F., Sher, N. & Yanai, I. CEL-Seq: Single-Cell RNA-Seq by Multiplexed Linear Amplification. Cell Reports, Cell Reports, Volume 2, Issue 3, p 666-673, 2012).

In certain embodiments, the invention involves plate based single cell RNA sequencing (see, e.g., Picelli, S. et al., 2014, “Full-length RNA-seq from single cells using Smart-seq2” Nature protocols 9, 171-181, doi:10.1038/nprot.2014.006).

In certain embodiments, the invention involves high-throughput single-cell RNA-seq. In this regard reference is made to Macosko et al., 2015, “Highly Parallel Genome-wide Expression Profiling of Individual Cells Using Nanoliter Droplets” Cell 161, 1202-1214; International patent application number PCT/US2015/049178, published as WO2016/040476 on Mar. 17, 2016; Klein et al., 2015, “Droplet Barcoding for Single-Cell Transcriptomics Applied to Embryonic Stem Cells” Cell 161, 1187-1201; International patent application number PCT/US2016/027734, published as WO2016168584A1 on Oct. 20, 2016; Zheng, et al., 2016, “Haplotyping germline and cancer genomes with high-throughput linked-read sequencing” Nature Biotechnology 34, 303-311; Zheng, et al., 2017, “Massively parallel digital transcriptional profiling of single cells” Nat. Commun. 8, 14049 doi: 10.1038/ncomms14049; International patent publication number WO2014210353A2; Zilionis, et al., 2017, “Single-cell barcoding and sequencing using droplet microfluidics” Nat Protoc. January; 12(1):44-73; Cao et al., 2017, “Comprehensive single cell transcriptional profiling of a multicellular organism by combinatorial indexing” bioRxiv preprint first posted online Feb. 2, 2017, doi: dx.doi.org/10.1101/104844; Rosenberg et al., 2017, “Scaling single cell transcriptomics through split pool barcoding” bioRxiv preprint first posted online Feb. 2, 2017, doi: dx.doi.org/10.1101/105163; Rosenberg et al., “Single-cell profiling of the developing mouse brain and spinal cord with split-pool barcoding” Science 15 Mar. 2018; Vitak, et al., “Sequencing thousands of single-cell genomes with combinatorial indexing” Nature Methods, 14(3):302-308, 2017; Cao, et al., Comprehensive single-cell transcriptional profiling of a multicellular organism. Science, 357(6352):661-667, 2017; Gierahn et al., “Seq-Well: portable, low-cost RNA sequencing of single cells at high throughput” Nature Methods 14, 395-398 (2017); and Hughes, et al., “Highly Efficient, Massively-Parallel Single-Cell RNA-Seq Reveals Cellular States and Molecular Features of Human Skin Pathology” bioRxiv 689273; doi: doi.org/10.1101/689273, all the contents and disclosure of each of which are herein incorporated by reference in their entirety.

In certain embodiments, the invention involves single nucleus RNA sequencing. In this regard reference is made to Swiech et al., 2014, “In vivo interrogation of gene function in the mammalian brain using CRISPR-Cas9” Nature Biotechnology Vol. 33, pp. 102-106; Habib et al., 2016, “Div-Seq: Single-nucleus RNA-Seq reveals dynamics of rare adult newborn neurons” Science, Vol. 353, Issue 6302, pp. 925-928; Habib et al., 2017, “Massively parallel single-nucleus RNA-seq with DroNc-seq” Nat Methods. 2017 October; 14(10):955-958; International patent application number PCT/US2016/059239, published as WO2017164936 on Sep. 28, 2017; and Drokhlyansky, et al., “The enteric nervous system of the human and mouse colon at a single-cell resolution,” bioRxiv 746743; doi: doi.org/10.1101/746743, which are herein incorporated by reference in their entirety.

In certain embodiments, the invention involves the Assay for Transposase Accessible Chromatin using sequencing (ATAC-seq) as described. (see, e.g., Buenrostro, et al., Transposition of native chromatin for fast and sensitive epigenomic profiling of open chromatin, DNA-binding proteins and nucleosome position. Nature methods 2013; 10 (12): 1213-1218; Buenrostro et al., Single-cell chromatin accessibility reveals principles of regulatory variation. Nature 523, 486-490 (2015); Cusanovich, D. A., Daza, R., Adey, A., Pliner, H., Christiansen, L., Gunderson, K. L., Steemers, F. J., Trapnell, C. & Shendure, J. Multiplex single-cell profiling of chromatin accessibility by combinatorial cellular indexing. Science. 2015 May 22; 348(6237):910-4. doi: 10.1126/science.aab1601. Epub 2015 May 7; US20160208323A1; US20160060691A1; and WO2017156336A1).

### Screening Methods

In certain embodiments, therapeutic agents that are capable of shifting an expression program as described herein are screened. Screening may be performed in vitro or in vivo. For example, agents that modulate an immune program that is dependent on a tumor microenvironment may be screened in vivo.

In certain embodiments, the methods described herein can be used to identify agents that can complement treatment with STAT3 inhibitors or can identify more specific or targeted agents. For example, although JSI-124 may show potential antitumor effects through inhibition of STAT3, other off-target proinflammatory pathways are activated, emphasizing that more careful and thorough preclinical investigations must be implemented to prevent potential harmful effects (see, e.g., McFarland et al., Activation of the NF-κB pathway by the STAT3 inhibitor JSI-124 in human glioblastoma cells, Mol Cancer Res. 2013 May; 11(5):494-505). Thus, agents that provide a comparable or enhanced therapeutic effect can be identified. Agents providing less side effects may also be identified. In certain embodiments, the agents are identified from a small molecule library as known in the art.

In certain embodiments, screening methods employ microfluidic devices, such as described in WO 2017/075549, “High-Throughput Dynamic Reagent Delivery System.” For example, the microfluidic device can be used to establish a gradient of one agent (e.g., JSI-124) and another agent can be tested for synergistic effects. Such an assay may be used to identify combination treatments that require lower doses and thus less potential side effects.

In certain embodiments, one or more agents targeting one or more genes as described herein are used in a combination treatment (e.g., STAT3 inhibitor and another agent targeting a gene as described herein).

In certain embodiments, screening methods employ PDX mouse models as described herein. In certain embodiments, PDX models can be treated with a chemotherapeutic agent (e.g., platinum based therapy). Agents can be screened for the ability to sensitize or over-come platinum resistance. In certain embodiments, tumor cells grown in culture can be screened as described herein for JSI-124. In certain embodiments 2D or 3D cultures are screened. In certain embodiments, OVACR4, OVACR8, OVASHO or TYKNU cells are screened. In certain embodiments, combination therapies are screened. In certain embodiments, ex vivo cultures of platinum resistant patients are screened. In certain embodiments, reporter cell lines expressing a reporter (e.g., luciferase) are used to screen for agents capable of modulating a gene signature as described herein. The reporter can be specific for a gene as described herein. One or more reporters for one or more genes may also be used. In certain embodiments, spheroid cultures are screened.

A further aspect of the invention relates to a method for identifying an agent capable of modulating one or more phenotypic aspects of a cell or cell population as disclosed herein, comprising: a) applying a candidate agent to the cell or cell population; b) detecting modulation of one or more expression programs in the cell or cell population by the candidate agent, thereby identifying the agent. In certain embodiments, steps can include administering candidate modulating agents to cells, detecting identified cell (sub)populations for changes in signatures, or identifying relative changes in cell (sub)populations which may comprise detecting relative abundance of particular gene signatures.

The term “modulate” broadly denotes a qualitative and/or quantitative alteration, change or variation in that which is being modulated. Where modulation can be assessed quantitatively—for example, where modulation comprises or consists of a change in a quantifiable variable such as a quantifiable property of a cell or where a quantifiable variable provides a suitable surrogate for the modulation—modulation specifically encompasses both increase (e.g., activation) or decrease (e.g., inhibition) in the measured variable. The term encompasses any extent of such modulation, e.g., any extent of such increase or decrease, and may more particularly refer to statistically significant increase or decrease in the measured variable. By means of example, modulation may encompass an increase in the value of the measured variable by at least about 10%, e.g., by at least about 20%, preferably by at least about 30%, e.g., by at least about 40%, more preferably by at least about 50%, e.g., by at least about 75%, even more preferably by at least about 100%, e.g., by at least about 150%, 200%, 250%, 300%, 400% or by at least about 500%, compared to a reference situation without said modulation; or modulation may encompass a decrease or reduction in the value of the measured variable by at least about 10%, e.g., by at least about 20%, by at least about 30%, e.g., by at least about 40%, by at least about 50%, e.g., by at least about 60%, by at least about 70%, e.g., by at least about 80%, by at least about 90%, e.g., by at least about 95%, such as by at least about 96%, 97%, 98%, 99% or even by 100%, compared to a reference situation without said modulation. Preferably, modulation may be specific or selective, hence, one or more desired phenotypic aspects of an immune cell or immune cell population may be modulated without substantially altering other (unintended, undesired) phenotypic aspect(s).

The term “agent” broadly encompasses any condition, substance or agent capable of modulating one or more phenotypic aspects of a cell or cell population as disclosed herein. Such conditions, substances or agents may be of physical, chemical, biochemical and/or biological nature. The term “candidate agent” refers to any condition, substance or agent that is being examined for the ability to modulate one or more phenotypic aspects of a cell or cell population as disclosed herein in a method comprising applying the candidate agent to the cell or cell population (e.g., exposing the cell or cell population to the candidate agent or contacting the cell or cell population with the candidate agent) and observing whether the desired modulation takes place.

Agents may include any potential class of biologically active conditions, substances or agents, such as for instance antibodies, proteins, peptides, nucleic acids, oligonucleotides, small molecules, or combinations thereof, as described herein.

The methods of phenotypic analysis can be utilized for evaluating environmental stress and/or state, for screening of chemical libraries, and to screen or identify structural, syntenic, genomic, and/or organism and species variations. For example, a culture of cells, can be exposed to an environmental stress, such as but not limited to heat shock, osmolarity, hypoxia, cold, oxidative stress, radiation, starvation, a chemical (for example a therapeutic agent or potential therapeutic agent) and the like. After the stress is applied, a representative sample can be subjected to analysis, for example at various time points, and compared to a control, such as a sample from an organism or cell, for example a cell from an organism, or a standard value. By exposing cells, or fractions thereof, tissues, or even whole animals, to different members of the chemical libraries, and performing the methods described herein, different members of a chemical library can be screened for their effect on phenotypes thereof simultaneously in a relatively short amount of time, for example using a high throughput method.

Aspects of the present disclosure relate to the correlation of an agent with the spatial proximity and/or epigenetic profile of the nucleic acids in a sample of cells (e.g., at genes in an expression program). In some embodiments, the disclosed methods can be used to screen chemical libraries for agents that modulate chromatin architecture epigenetic profiles, and/or relationships thereof.

In some embodiments, screening of test agents involves testing a combinatorial library containing a large number of potential modulator agents. A combinatorial chemical library may be a collection of diverse chemical agents generated by either chemical synthesis or biological synthesis, by combining a number of chemical “building blocks” such as reagents. For example, a linear combinatorial chemical library, such as a polypeptide library, is formed by combining a set of chemical building blocks (amino acids) in every possible way for a given agent length (for example the number of amino acids in a polypeptide agent). Millions of chemical agents can be synthesized through such combinatorial mixing of chemical building blocks.

In certain embodiments, the present invention provides for gene signature screening. The concept of signature screening was introduced by Stegmaier et al. (Gene expression-based high-throughput screening (GE-HTS) and application to leukemia differentiation. Nature Genet. 36, 257-263 (2004)), who realized that if a gene-expression signature was the proxy for a phenotype of interest, it could be used to find small molecules that effect that phenotype without knowledge of a validated drug target. The signatures or expression programs of the present invention may be used to screen for drugs that reduce the signature or expression program in cells as described herein. The signature or expression program may be used for GE-HTS. In certain embodiments, pharmacological screens may be used to identify drugs that are selectively toxic to cells having a signature.

The Connectivity Map (cmap) is a collection of genome-wide transcriptional expression data from cultured human cells treated with bioactive small molecules and simple pattern-matching algorithms that together enable the discovery of functional connections between drugs, genes and diseases through the transitory feature of common gene-expression changes (see, Lamb et al., The Connectivity Map: Using Gene-Expression Signatures to Connect Small Molecules, Genes, and Disease. Science 29 Sep. 2006: Vol. 313, Issue 5795, pp. 1929-1935, DOI: 10.1126/science.1132939; and Lamb, J., The Connectivity Map: a new tool for biomedical research. Nature Reviews Cancer January 2007: Vol. 7, pp. 54-60). In certain embodiments, Cmap can be used to screen for small molecules capable of modulating a signature or expression program of the present invention in silico.

### Kits or Pharmaceutical Systems

The present compositions may be assembled into kits or pharmaceutical systems for use in ameliorating a neoplasia. Kits or pharmaceutical systems according to this aspect of the invention comprise a carrier means, such as a box, carton, tube or the like, having in close confinement therein one or more container means, such as vials, tubes, ampoules, or bottles. The kits or pharmaceutical systems of the invention may also comprise associated instructions for using the agents of the invention. The present invention also may comprise a kit with a detection reagent that binds to one or more biomarkers or can be used to detect one or more biomarkers.

Further embodiments are illustrated in the following Examples which are given for illustrative purposes only and are not intended to limit the scope of the invention.

### EXAMPLES

### Example 1—scRNA-Seq of the Ascites Ecosystem of High-Grade Serous Ovarian Cancer

Applicants collected scRNA-seq data from three high-grade serous ovarian cancer (HGSOC) cohorts totaling 35,957 high-quality cell profiles from a set of 22 ascites samples from 11 patients and a validation set (gathered separately) of three additional ascites samples and two primary tumors (Table 1), collected through a translational workflow^{8,12}. Together, this data compendium spans 4 treatment naïve, 2 on-treatment during initial chemotherapy, 18 on-treatment during disease recurrence, and 2 post-neo-adjuvant chemotherapy specimens that reflects the substantial, real-life diversity among HGSOC patients. Applicants used three complementary profiling strategies (FIG. 1a, FIG. 5a, Table 1). First, to obtain a broad view of the different cell types in the ascites ecosystem, Applicants analyzed 8 specimens (that were partly depleted of CD45+ immune cells, Methods) from 6 patients by massively parallel droplet scRNA-seq (Cohort 1) (Methods). Second, because even after CD45⁺ cell depletion, droplet-based profiling had a relatively low proportion of malignant cells (7.9%; see below), Applicants complemented it by isolating 1,297 viable malignant EPCAM⁺CD24⁺ cells, which identify cancer cells with high sensitivity and specificity¹³, from 14 ascites specimens from 6 individuals by fluorophore-activated cell sorting (FACS) into 96-well plates, followed by full length scRNA-seq using a modified SMART-seq2 protocol^{14,15} (Cohort 2) (Methods). Third, as a validation set to examine the generalizability of the results, Applicants assembled three additional ascites specimens (10,688 cells) and two primary tumors (14,505 cells) from the Human Tumor Atlas Pilot Project (HTAPP) (Cohort 3)¹⁶.

Applicants first used the droplet based scRNA-seq (Cohort 1) to identify and annotate 18 distinct cell clusters spanning epithelial cells (5 clusters marked by EPCAM, cytokeratins, kallikreins), macrophages (4 clusters marked by CD14, AIF1, CSF1R, CD163), cancer-associated fibroblasts (CAFs) (4 clusters marked by PDPN, DCN and THY1), dendritic cells (2 clusters marked by CD1C, CD1E, CCR7, CD83), B cells (CD19, CD79A/B), T cells (CD2, CD3D/E/G) and erythrocytes (GATA1, hemoglobin) (FIG. 1b-d, FIG. 6a, Table 2, Methods). Although Applicants depleted CD45⁺ cells, immune cells remained the most abundant component, comprising on average 67% of the cells in each sample (ranging 4-98%), highlighting a unique challenge of analyzing tumor cells from malignant effusions not usually encountered with solid tumor preparations¹⁶. The overall proportion of immune cells in each sample may reflect the differential efficiency of immune depletion (Methods), while other differences between samples may also be of biological origin (FIG. 1c). Applicants examined whether different treatment histories explained differences in cellular ascites composition. Among cancer cells, there was significant inter-patient heterogeneity (FIG. 1b-d) which was not associated with prior treatment history (FIG. 5b). Among CAFs and macrophages, expression profiles were similar across patients, indicating shared phenotypes, while the differences between patients were not clearly correlated with patient's treatment histories (FIG. 5c). Determining whether expression patterns are linked to prior treatment exposures will require larger patient cohorts.

### Example 2—Variation in CAFs and Macrophage Subsets within and Across Patients

Macrophages and CAFs each comprised four clusters (FIG. 1c,d), driven by both inter- and intra-patient variability. While some clusters were comprised almost entirely of cells from a single patient, most had cells from multiple patients (FIG. 1c), including fibroblast clusters composed of cells from three or more patients.

Among CAFs, Applicants identified distinct cell states, including sub-populations with expression of immune-related genes, such as complement factors (C1QA/B/C, CFB), chemokines (CXCL1/2/10/12), and cytokines (IL6 and IL10) in clusters 8 and 9 compared to CAFs in clusters 6 and 7 (FIG. 1e, FIG. 6b), suggesting a role as immunomodulatory CAFs^{17,18}. This variation is also observed within a single patient: repeating the same analysis only with CAFs from Patient sample 5.1, recapitulates CAF sub-populations based on the differential expression of 80 genes (FIG. 6c). Among other cytokines expressed by these fibroblast subsets, CXCL12 and IL6 activate JAK/STAT signaling across cancers¹⁹.

Among macrophages, Clusters 10 and 13 comprised the majority of cells. While most cells from Cluster 13 derived from one patient sample (5.1), Cluster 10 had cells from multiple patients. To examine intra-patient heterogeneity of macrophages, Applicants focused on cells in cluster 10 from Patient samples 6 and 5.0, each separating into two sub-populations (FIG. 6d, Groups 1 and 2), and characterized by consistent transcriptional programs. Group 1 cells co-expressed HLAs encoding for MHC Class II, IFNGR1 CD1D, CD36, and CD52, whereas Group 2 cells expressed complement factor components, cathepsins and APO genes (FIG. 6e). Moreover, Group 1 cells expressed several genes identified as markers for M1 macrophages (IFNGR1, CD36, DDX5, MNDA) and as suppressors of M2 differentiation (C3AR1), while Group 2 cells expressed genes described in M2 macrophages, including those regulating M2 differentiation (e.g., AIF1²⁰, VISG4²¹). While the M1/M2 dichotomy is currently being revised, this separation may indeed be associated with functional pro-/anti-tumor macrophage states. These findings generalized to samples with macrophages from all other patients (except Patient sample 5.1) (FIG. 6f).

### Example 3—Inter- and Intra-Patient Heterogeneity of Malignant Cells in HGSOC

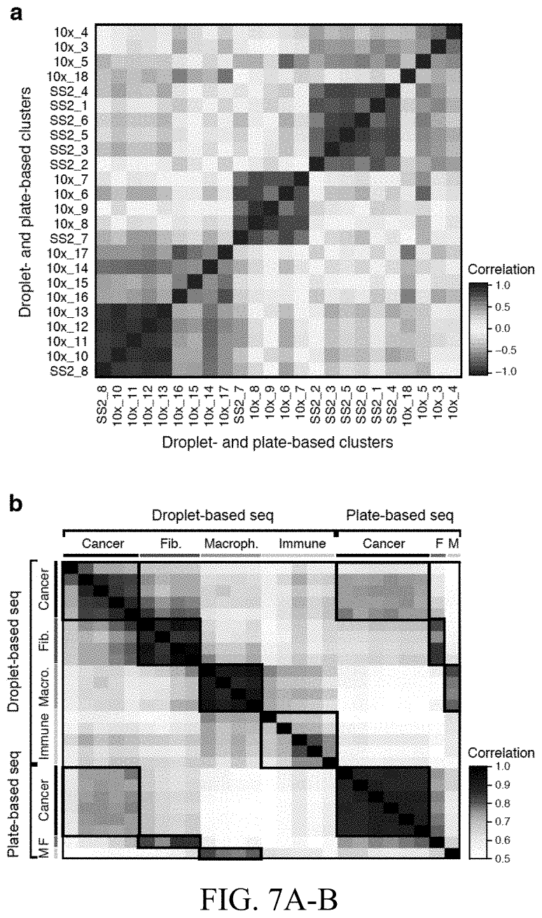

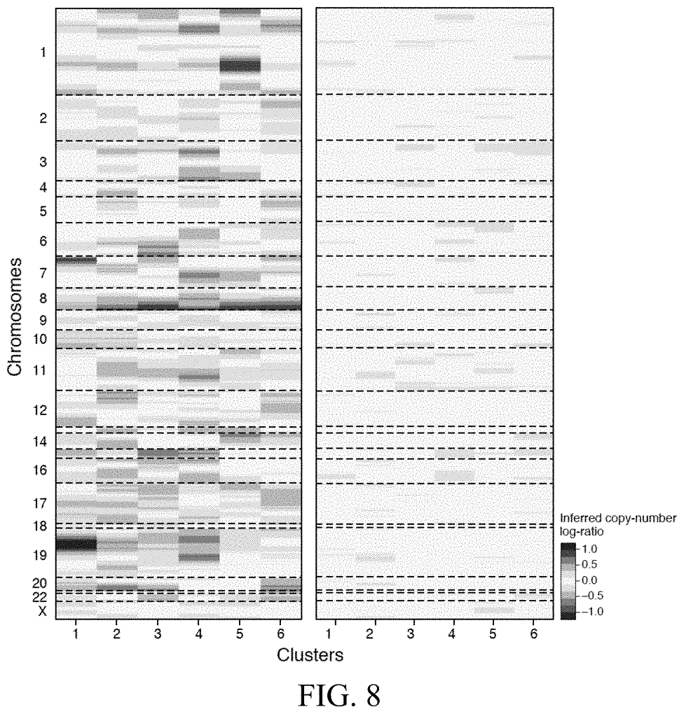

To characterize the variation within the malignant compartment, Applicants focused on the FACS-enriched, deeper-coverage full length scRNA-seq profiles (Cohort 2), and identified eight clusters (FIG. 2a,b), including six of epithelial cells, one of CAFs (cluster 7), and one of macrophages (cluster 8) (FIG. 2b,c, FIG. 7A-7B). Applicants confirmed that the epithelial cells were malignant by inferring chromosomal copy number alterations (CNAs)⁸ (FIG. 8, Methods). In contrast to macrophages and CAFs, the malignant cells clustered by their patient of origin (FIG. 2a,b), highlighting inter-individual variation. Some of this variation reflects the distinct CNA profile of each tumor (FIG. 8), but additional genetic and epigenetic effects likely contribute to inter-patient variability among malignant cells.

### Example 4—the Cancer Genome Atlas (TCGA) Mesenchymal and Immunoreactive Subtypes Reflect Non-Malignant Contributions

Applicants next asked whether inter-patient variability among malignant cells is consistent with the previously described TCGA subtypes—differentiated, proliferative, mesenchymal and immunoreactive—that have been derived from RNA expression profiling of bulk solid tumors from untreated HGSOC patients and have been associated with varying prognoses⁵. Applicants thus scored each cluster for the expression of TCGA-derived subtype signatures (Methods).

All six malignant cell clusters highly expressed the “differentiated” signature and only one (cluster 4) strongly expressed the “proliferative” signature (FIG. 2d, FIG. 9), whereas the “mesenchymal” and “immunoreactive” signatures showed weak or no expression in cancer cell clusters, but were highly expressed by the CAF and macrophage clusters, respectively. Thus, the mesenchymal and immunoreactive subtypes may represent the intra-tumoral abundance of CAFs and macrophages, respectively. This is consistent with previous reports that TCGA tumors with these classifications had a significantly lower purity²² (FIG. 2e), but the single-cell analysis shows directly that the immunoreactive and mesenchymal gene programs are derived from the non-malignant immune cells and CAFs as opposed to expression signatures in malignant cells (such as epithelial-mesenchymal transition) delineates epithelial to mesenchymal transition in cancer cell from CAF gene expression. Thus, subtype classifications based on bulk RNA profiles likely predominantly reflect tumor ecosystem composition rather than cancer cell-derived HGSOC subtypes.

### Example 5—Intra-Patient Variation in Expression Programs of Malignant Cells

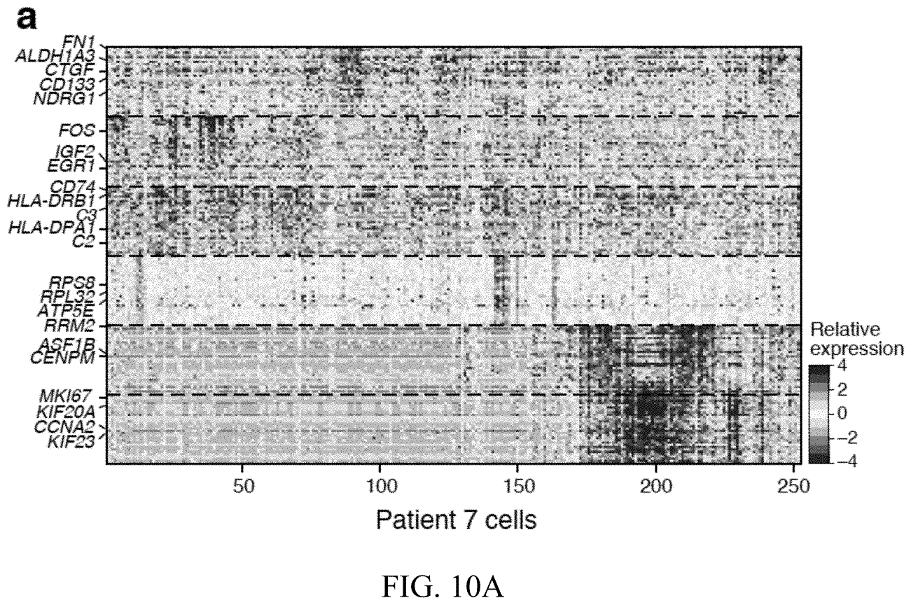

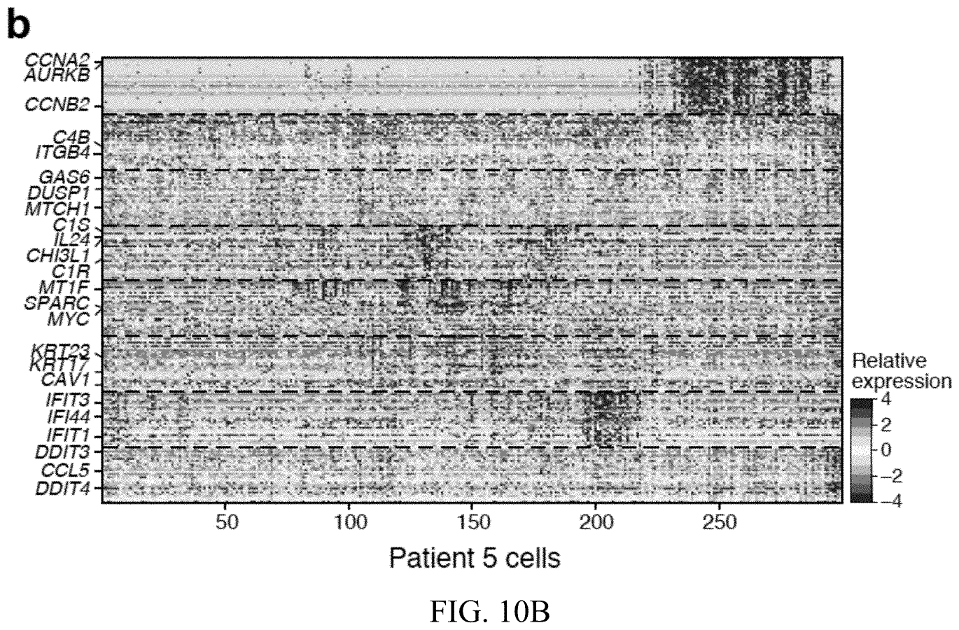

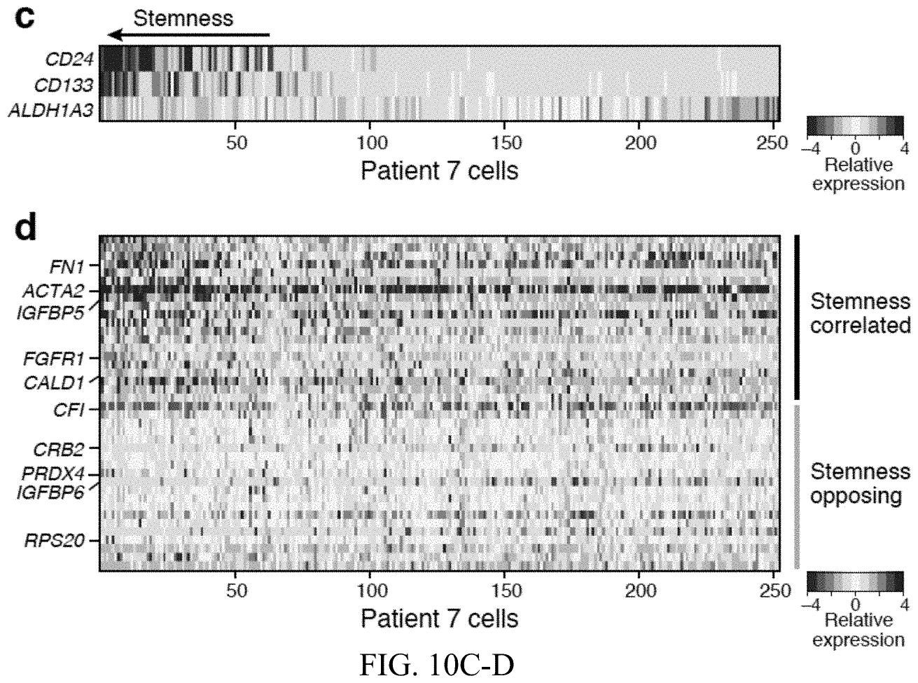

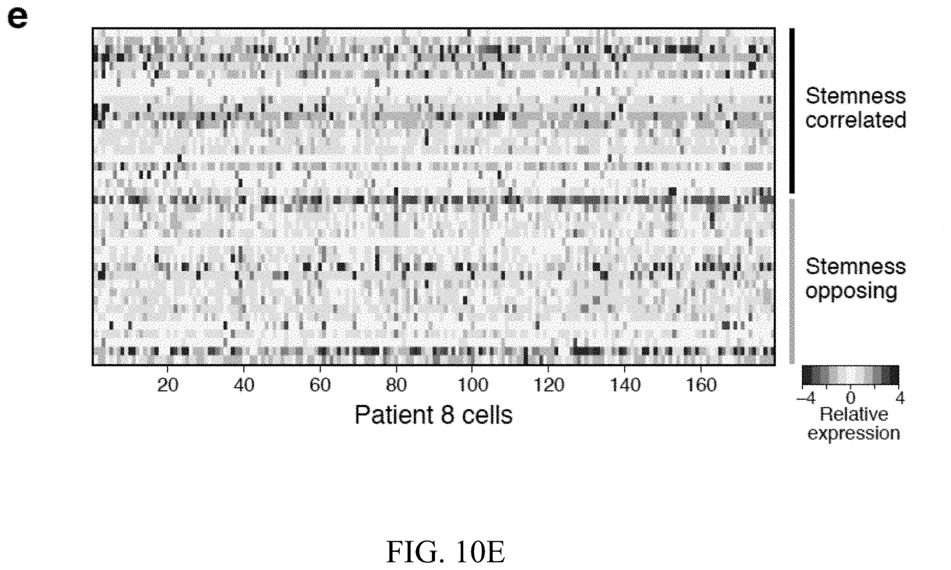

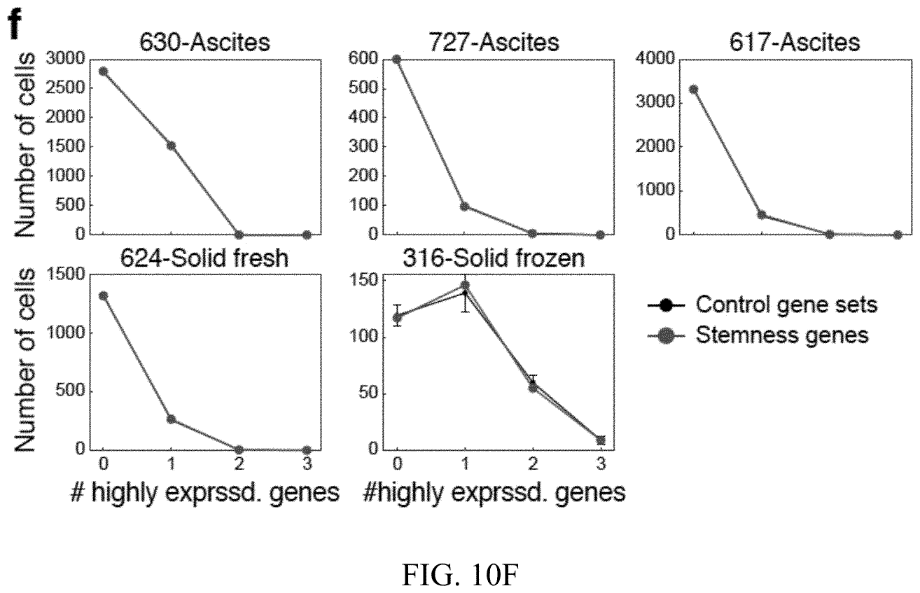

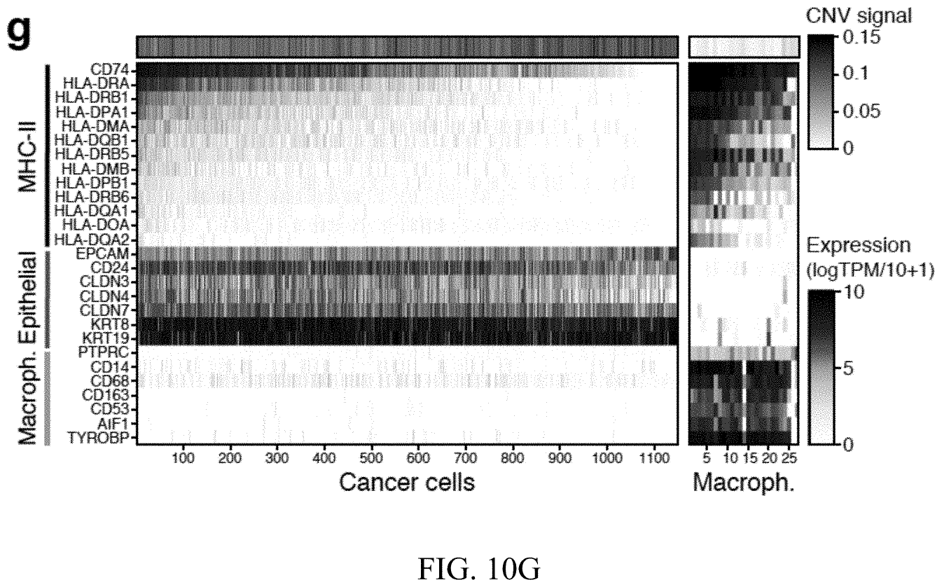

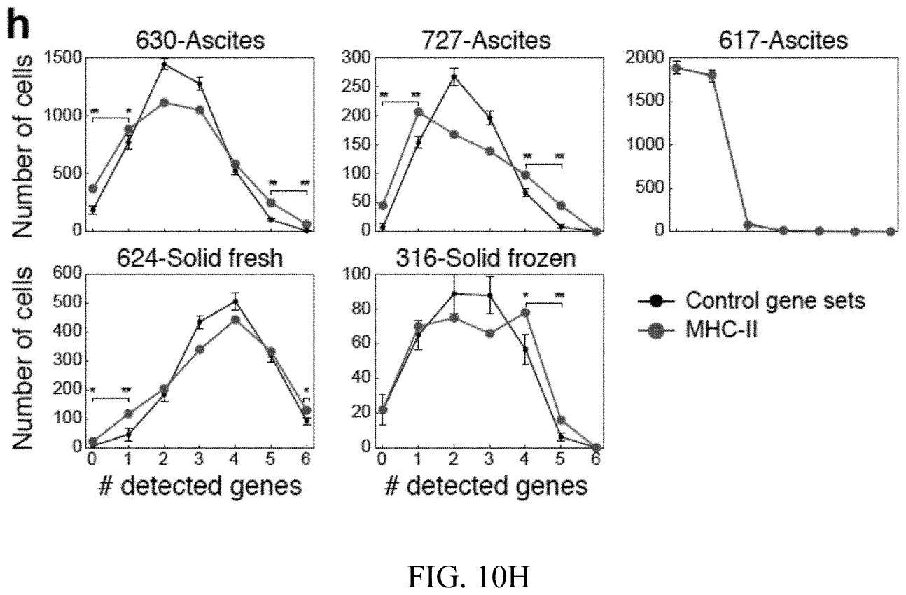

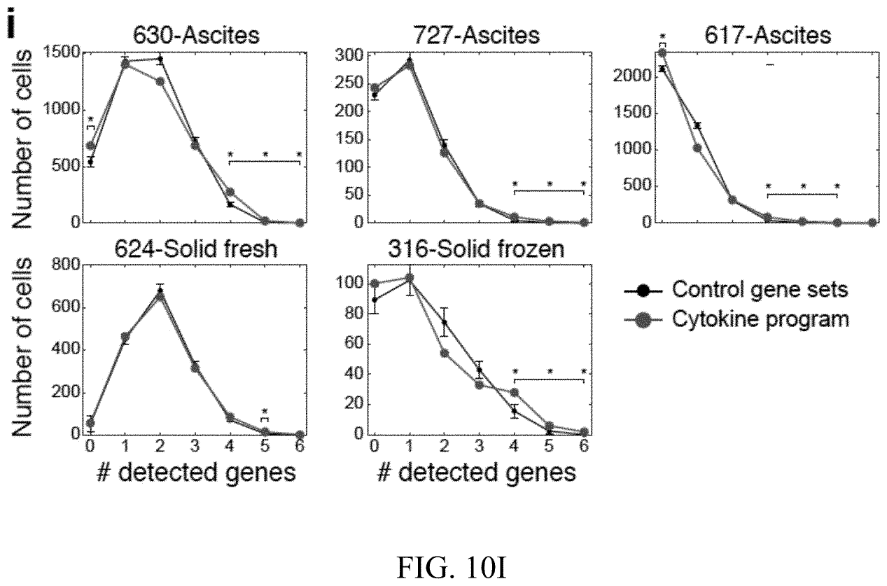

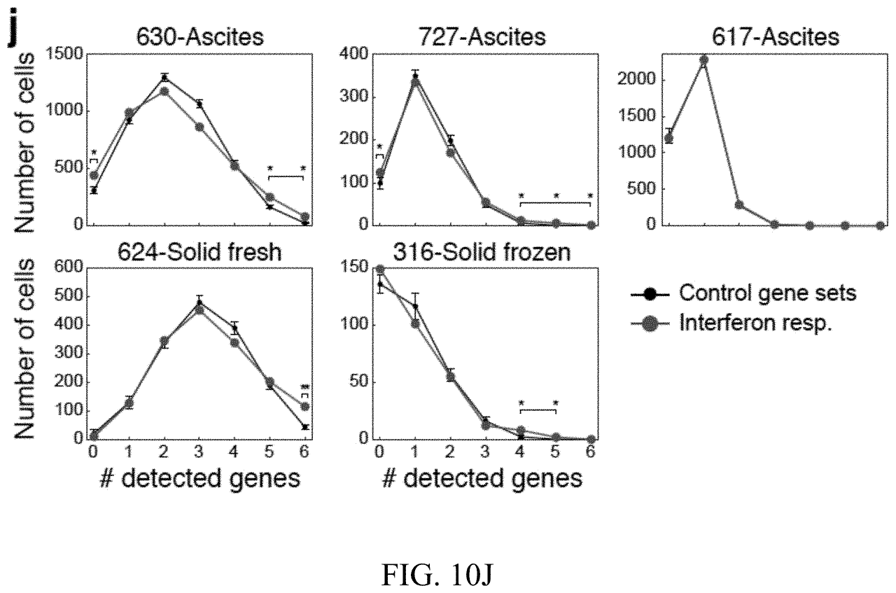

Applicants next identified programs that vary among each patient's malignant cells. Applicants used non-negative matrix factorization (NMF) and identified a total of 35 modules across malignant cells with coherently co-varying gene expression (FIG. 3a-c, FIG. 10a,b and Table 3). The modules spanned diverse functions, reflected by their top-scoring genes, including cell cycle (e.g., CCNA2, CCNB2, AURKB), inflammation (e.g., IL6, IL32, TNF, IFI6), and stress or activation (e.g., HSPA5-7, ATF4, JUN, DDIT3). One intriguing module consisted of prominent stemness²³ (ALDH1A3 and CD133/PROM1) and mesenchymal (FN1, ACTA2 and MYL9) markers, and AXL and its only known ligand GAS6, which is implicated in drug resistance²¹ (FIG. 10c-e); however, this program was unique to patient 7 and was not detected in cancer cells across cohorts examined here (FIG. 10c-f). Profiling of larger patient cohorts will be required to examine whether this is a recurrent stemness signature in HGSOC. Together, and in line with previous work^{8-10,2,1}, Applicants find inter-patient variation across cancer cells and intra- and inter-individual heterogeneity of non-malignant cells, suggesting different functional subpopulations that contribute to shaping the HGSOC ecosystem.

### Example 6—Shared Expression of Cancer Cell-Autonomous Inflammatory Programs Across Patients and PDX Models

To identify cancer cell programs shared across patients, Applicants next compared the modules across different patients (FIG. 3d). As expected, there was strong overlap of cell cycle modules, indicating the presence of proliferating subpopulations of cells in all specimens. In addition, three programs dominated by immune- or inflammation-associated genes were shared among patients (FIG. 3d,e): an inflammatory cytokines module (e.g., IL6, TNF, IL8, IL32), an MHC-class II antigen presentation module (e.g., CD74, HLA-DRA), and an interferon-response module (e.g., IFI6, IFIT1, ISG15). These immune-related programs were also detected in cells from Cohort 3, including three additional ascites samples and two primary tumors (FIG. 10h-j). Cancer cell-intrinsic expression of MHC-Class II protein has recently been described in other epithelial cells^{25,26}, tumor-initiating cells²⁷, and cancers, such as melanoma²⁸, and can be associated with response to immunotherapies even in the absence of MHC Class I expression²⁸. Applicants confirmed the expression of the MHC Class II module in CNV-bearing malignant cells (FIG. 10g) and showed by immunofluorescence staining of independent primary HGSOC tumors the presence of a subpopulation of MHC Class II expressing cancer cells (among other cells that express MHC Class II) (FIG. 3f).

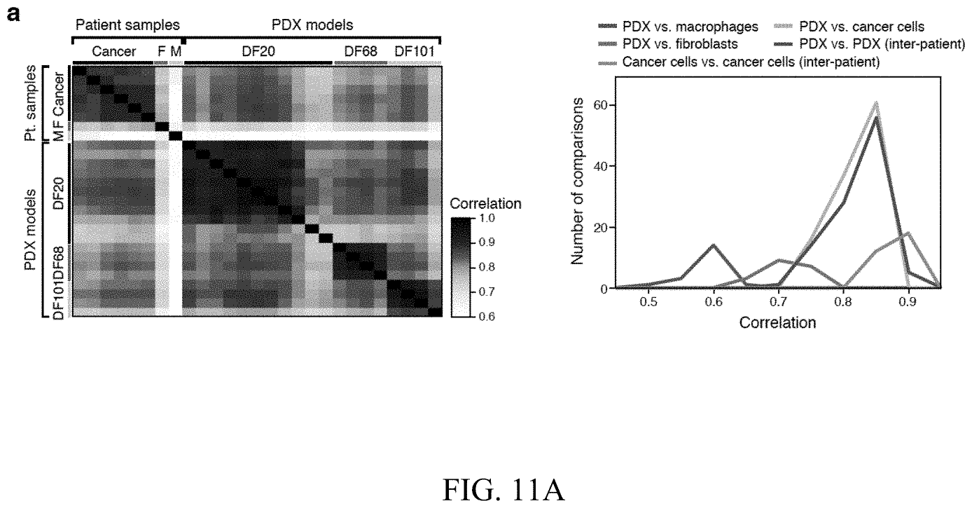

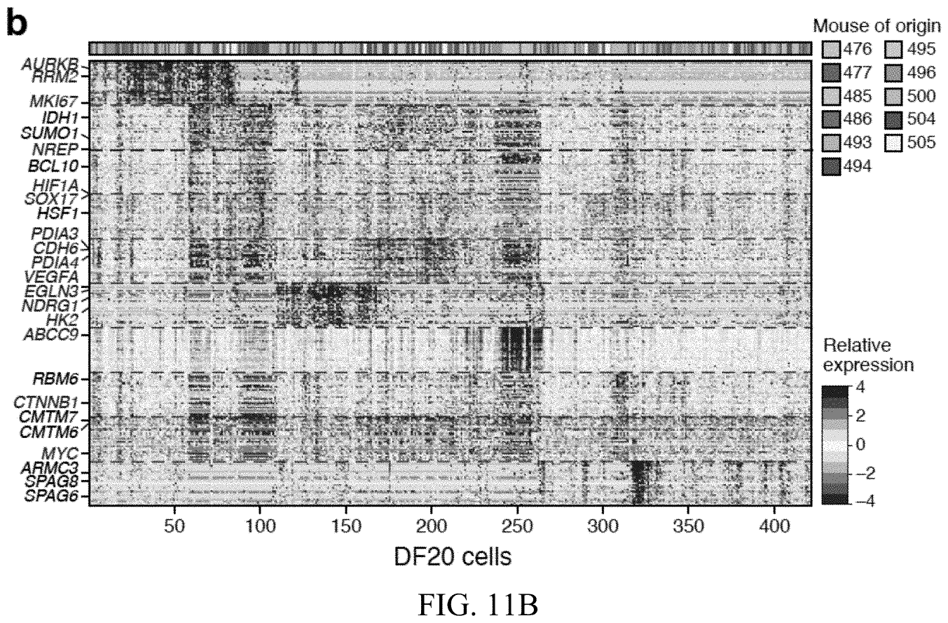

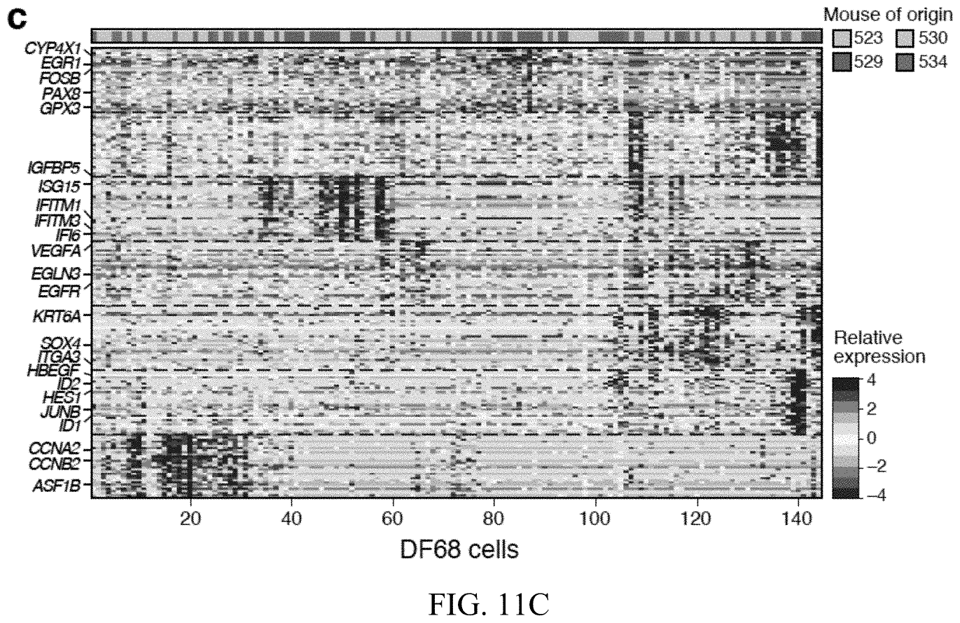

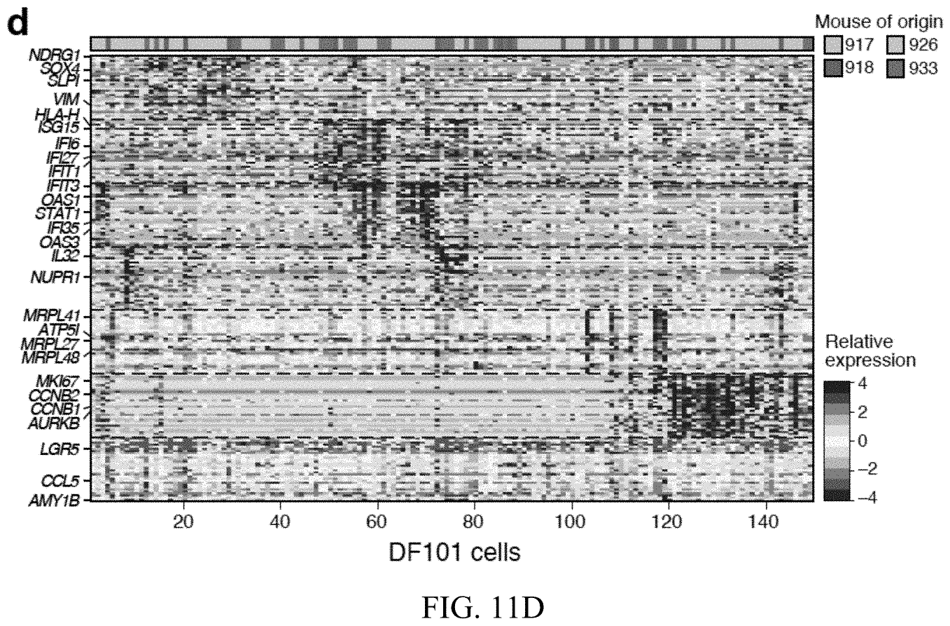

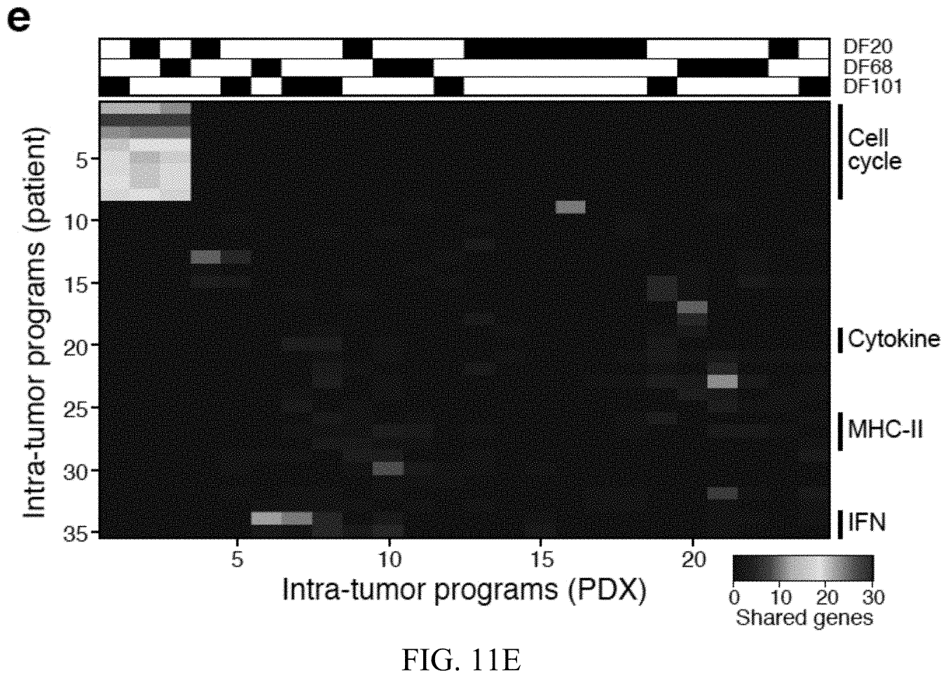

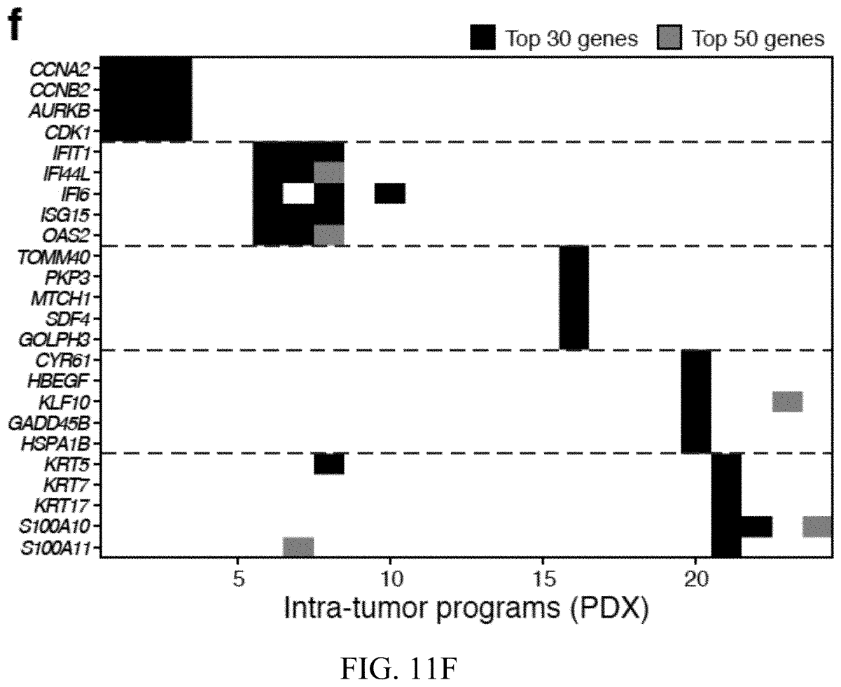

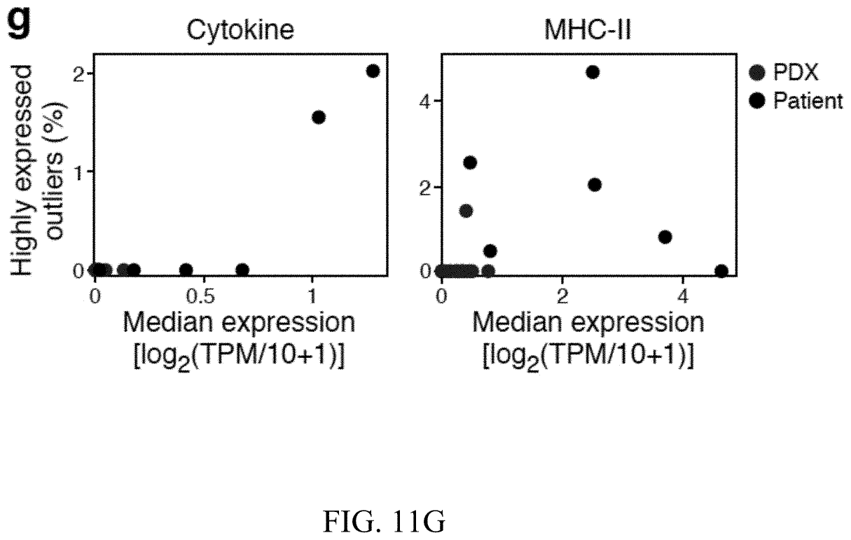

Inflammatory cancer-cell programs may be induced by the ascites microenvironment or reflect an endogenous property of the cancer cells. To evaluate these possibilities, Applicants profiled 795 cells by scRNA-seq of three previously established PDX models (DF20, DF68 and DF101) (Methods) grown as subcutaneous (s.c.) tumors in immunocompromised animals²⁹ collected pre-, on- and post-platinum therapy (Methods). Globally, PDX and patient cancer cells were as strongly correlated with each other (Pearson r=0.819 on average) as cancer cells of different PDX models (r=0.822 on average) or cancer cells across patients (“inter-patient” comparison; r=0.882 on average) (FIG. 11a). Applicants next identified 24 programs in the PDX models by NMF (FIG. 11b-d, Table 4), and compared them to those identified in patient ascites (FIG. 11e,f).

Nine PDX modules were analogous to those from patient samples, including three reflecting cell cycle (PDX modules 1-3) and three interferon-response programs (PDX modules 6-8) (FIG. 11f), highlighting this as the most significant similarity (apart from the cell cycle) between patterns of heterogeneity in patients and PDX models. The other two inflammation-related programs found in patients (cytokine and antigen presentation modules) were not detected in PDX models (FIG. 11g), suggesting that their expression in cancer cells may depend on an intact immune system (mostly absent in nod scid gamma mouse (NSG) mice) or other microenvironmental cues.

### Example 7—JAK/STAT Activation May Represent a Shared and Targetable Vulnerability in HGSOC

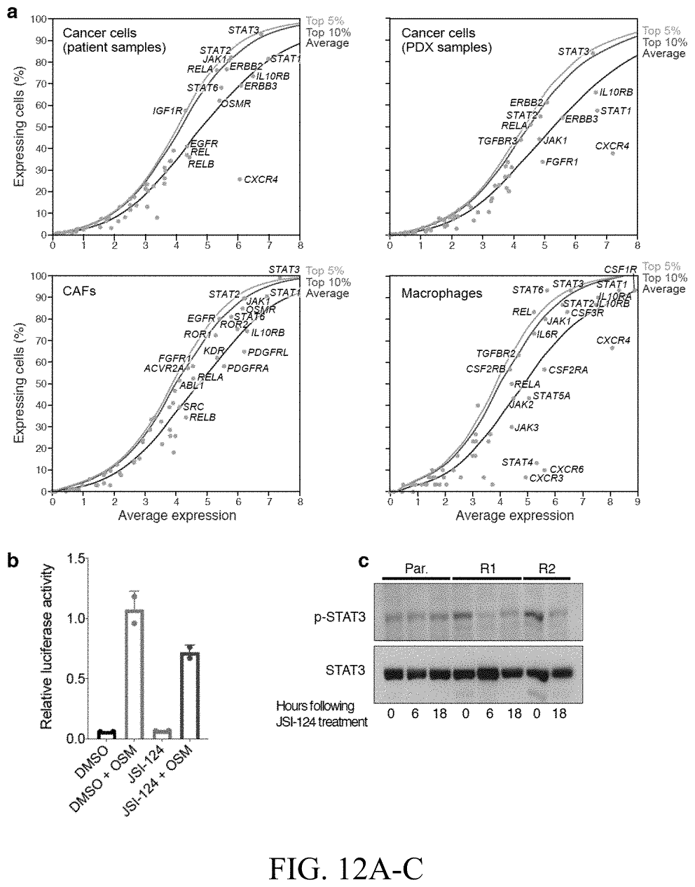

Multiple observations led us to consider the JAK/STAT-pathway as a potential vulnerability. First, as described above, subpopulations of cancer cells highly expressed three immune-related programs that may be downstream to the JAK/STAT pathway. Second, cells in the ascites microenvironment, such as CAFs, highly expressed genes of secreted ligands (e.g. IL6, CXCL12) that activate the JAK/STAT-pathway. Third, analysis of a large set of signaling genes highlighted a particularly high and ubiquitous expression of JAK/STAT-pathway components both in malignant and in non-malignant cells (FIG. 3g,h, FIG. 12a).

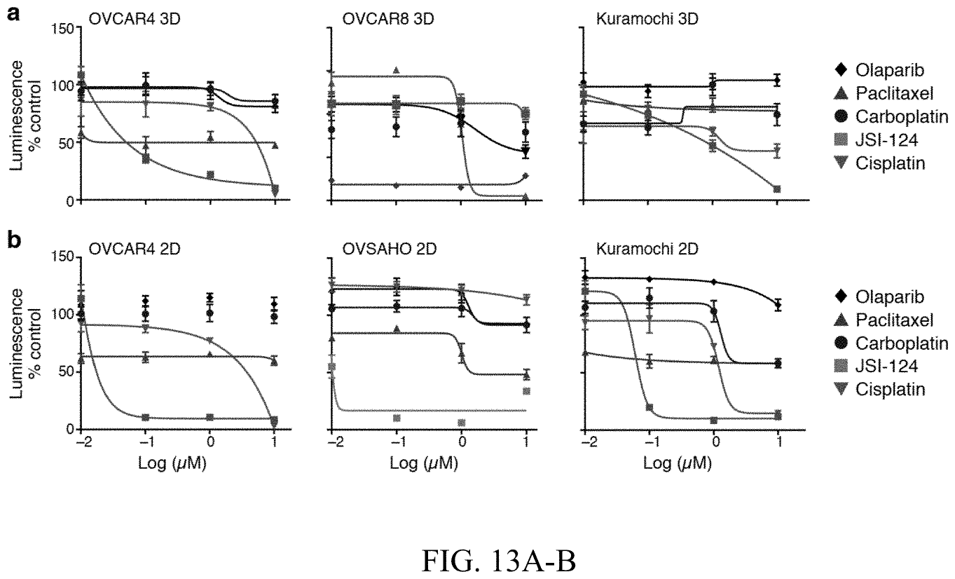

To determine the impact of JAK/STAT inhibition, Applicants performed a drug screen using 15 compounds targeting different nodes of the pathway or its effectors and platinum-chemotherapies (Table 5) in HGSOC cell line OVCAR4, and identified JSI-124³⁰ as a potent inhibitor of cell viability (FIG. 4a; FIG. 12b,c). JSI-124 showed anti-tumor activity at nanomolar doses in three different patient-derived cell culture models and additional HGSOC cell lines³¹, while other drugs frequently used for the treatment of HGSOC patients had little to no activity (FIG. 4b,c; FIG. 13A-13B). JSI-124 also reduced the formation of three-dimensional clusters (spheroids) and their invasion through a mesothelial monolayer (modeling the abdominal peritoneum, which represents an important barrier for metastatic disease) (FIG. 4d-f). In the PDX model DF20, which has substantial transcriptional similarity to patient ascites cancer cells (FIG. 11a), early initiation of JSI-124 treatment (7 days after intraperitoneal [I.P.] or subcutaneous [S.C.] injection of tumor cells) abrogated the development of malignant ascites and tumor growth, respectively (FIG. 4g,i). Furthermore, JS-124 significantly reduced disease burden in models where I.P. ascites and S.C. tumors were grown for 21 days prior to treatment initiation (FIG. 4h,j). Together, these results suggest that JAK/STAT inhibition may be a potent therapeutic option for patients with HGSOC, through action on malignant cells, non-malignant cells or both.

### Example 8—Discussion

This scRNA-seq study of malignant ascites from patients with advanced HGSOC reveals significant variability in cellular states and programs among malignant and non-malignant cells. Among non-malignant cells Applicants observed diversity among CAFs, with a subset expressing immunomodulatory programs, as recently proposed in other cancer types, such as pancreatic ductal adenocarcinoma¹⁸, where “inflammatory” CAFs strongly express IL6 and other cytokines and may promote tumor growth and drug resistance. Shared activation of the JAK/STAT-pathway in cancer cells and CAFs suggests that paracrine (and/or autocrine) signaling via this pathway may contribute to the pathogenesis of malignant ascites and drug resistance, and provide one example of how cell-to-cell interactions shape the ascites ecosystem. Macrophage diversity primarily involved one major axis of variation driven by two gene programs: one including MHC Class II, IFNGR1 and M1-associated genes, and the other by complement factors, and M2-related genes, such as AIF1²⁰ and VSIG4²¹, suggesting that a balance of these phenotypes exists within the ascites ecosystem. Previous reports indicate that platinum-therapy may further push this balance towards M2 macrophages by altering monocyte differentiation³². Such changes may occur rapidly, as indicated in a shift from M1-like to M2-like macrophage programs in one patient pair (5.0 and 5.1), where Applicants examined pre-treatment and on-treatment with platinum chemotherapy.

Variation across cancer cells was driven primarily by inter-patient variation, including CNA patterns, but additional subtler intra-patient variation is also present, such as a putative stemness program unique to a subpopulation of cells in patient 7. Some of the intra-patient patterns of heterogeneity were consistent across multiple patients. For example, subsets of malignant cells expressing the MHC Class II program were present in multiple patients and may be associated with increased abundance of tumor-infiltrating lymphocytes (TILs), improved prognosis and response to immunotherapies³³. MHC Class II expressing subsets were not identified in PDXs, suggesting that they may depend on immune cell interactions. An interferon-response and a cytokine program also co-varied across malignant cells in multiple patients. Thus, significant cellular and transcriptional forces within the ascites ecosystem, pro-tumorigenic or pro-immunogenic, may balance disease progression and responses to therapies. Therapeutically shifting this balance may be one avenue for reshaping the drug resistant milieu.

Previous work focused on intra-patient and intra-lesion genetic variability showed that development and response to therapy of primary tumors or metastatic lesions emerge as a result of co-evolution of malignant and non-malignant compartments^{34,35}. Similarly, Applicants hypothesize that interactions between CAFs and macrophages in the ascites ecosystem regulate or enhance cancer cell-autonomous programs. One example is the putative interaction between CAFs secreting IL6 to stimulate JAK/STAT signaling in cancer cells, which is associated with poor prognosis and resistance to chemotherapies³⁶. Consistently, JAK/STAT inhibition promoted anti-tumor activity in several pre-clinical models. Clinical trials, such as phase I/II study using combination therapies with JAK/STAT inhibitor ruxolitinib (NCT02713386) will help clarifying the role of such therapies in HGSOC.

HGSOC subtypes defined by TCGA have been associated with prognosis and drug response^{5,37}. In this study, the vast majority of cancer cells across patients strongly expressed the “differentiated” subtype program, and a minority of cells from one patient also expressed the “proliferative” subtype program. In contrast, the “mesenchymal” and “immunoreactive” subtype programs were not expressed by cancer cells, but reflected programs expressed by CAFs and macrophages, respectively, and therefore represent tumor composition rather than salient cancer cell programs. While previous work could not evaluate the relative contribution of CAFs and cancer cells to the mesenchymal subtype (which also includes EMT genes), these results suggest that most, if not all, of this subtype can be explained by CAFs. This is consistent with findings in colorectal^{38,39} and head and neck cancer¹¹. Increased CAF infiltration may contribute to the low response rates to certain therapies, such as immune checkpoint inhibitors, whose efficacy is impacted by the tumor microenvironment⁴⁰.

Future studies should enhance this work in two main ways. First, profiling a larger number of patient samples would allow for testing the generality of programs identified only in one patient (e.g., stemness program in patient 7) in this study. Second, single cell profiling of well-stratified clinical cohorts—rather than the diverse broad ranging and heterogenous patient population in this analysis (included to recapitulate true-to-life clinical heterogeneity)—should enhance inter-patient comparisons and identify converging aspects of tumor biology and drug resistance, to improve our understanding of HGSOC.

### Example 9—Materials and Methods

Collection of patient specimens. Specimens were collected from patients with ovarian cancer at Brigham and Women's Hospital and Dana-Farber Cancer Institute under IRB approved protocols 02-051 and 11-104. Ascites fluid was drained by an interventional radiologist and transferred for further processing in closed vacuum bottles. De-identified patient information, including their ovarian cancer histology, stage, treatment history, and BRCA mutation status were collected.

Sample handling, flow cytometry and single cell isolation. Immediately following drainage, malignant ascites was transported on ice, aliquoted into 50 mL conical tubes (BD Falcon) and spun for 5 min at 580×g at 4° C. The supernatant was aspirated and the remaining pellet was resuspended with 5 mL of hypotonic lysis buffer ACK (Life technologies) and incubated on ice for 5 min. 20 ml of PBS was used to quench the lysis buffer. Cell suspensions were pooled and pipetted into a new 50 mL conical tube through a 100 μm mesh and spun for 5 min at 580×g at 4° C. Hypotonic lysis was repeated until no visible red blood cell component was present, usually for a total of 2-3 times (for the plate-based approach). Following red blood cell lysis, the cell pellet was resuspended in PBS with 2% (v/v) FBS. These cells were then used for both plate-based and droplet-based scRNA-seq. For plate-based scRNA-seq, cells were labeled with the following fluorophore-conjugated flow-cytometry antibodies: live/dead stain with Calcein-AM (LifeTechnologies), 7-AAD (7-Aminoactinomycin D) (LifeTechnologies), CD45-FITC (VWR, 304006, Clone HI30), EPCAM-PE (Miltenyi Biotec, 130-111-116, Clone REA764), and CD24-PE/Cy7 (BioLegend, 311119, Clone ML5). Cells were incubated for 30 min on ice in the dark. Cells were washed twice by resuspending with PBS with 2% (v/v) FBS and spun 5 min at 580×g at 4° C. Flow-cytometry and sorting was performed on a BD Biosciences cell sorter. Following doublet exclusion, Calcein^{high}7AAD⁻CD45⁻EPCAM⁺CD24⁺ single cells were sorted into 96-well microtiter plates (on a plate chiller) that were prepared with 10 μL cell lysis buffer (TCL+1% β-mercaptoethanol). Following completion of cell sorting, plates were covered with aluminum lids, vortexed for 10 seconds, centrifuged for 2 min at 580×g at 4° C. and immediately placed on dry ice prior to storage at −80° C.

Plate-based single-cell RNA-seq. Plate-based scRNA-seq following FACS enrichment was used to strongly enrich for malignant cells. This approach complements efforts using droplet-based method (below) where the non-malignant compartment makes up the vast majority of cells, but also includes a relatively small portion of cancer cells but with more shallow data. To balance both approaches, Applicants used both to complement different types of analyses. For plate-based sc-RNA-seq, Applicants performed Whole Transcriptome Amplification (WTA) with a modified Smart-seq2 protocol, as described previously^{14,15} with Maxima Reverse Transcriptase (Life Technologies) instead of Superscript II. Next, WTA products were cleaned with Agencourt XP DNA beads and 70% ethanol (Beckman Coulter, Brea, Calif.) and Illumina sequencing libraries were prepared using Nextera XT (Illumina, San Diego, Calif.), as previously described¹⁵. The 96 samples of a multiwell plate were pooled, and cleaned with two 0.8×DNA SPRIs (Beckman Coulter). Library quality was assessed with a high sensitivity DNA chip (Agilent) and quantified with a high sensitivity dsDNA Quant Kit (Life Technologies). Barcoded single cell transcriptome libraries were sequenced with 38 bp paired end reads on an Illumina NextSeq 500 Instrument.

Droplet-based single-cell RNA-seq. Single cells were isolated from patient-derived ascites as described above for all but one solid tumor, which was prepared for single nucleus isolation as recently described¹⁶. Upon drainage of ascites, Applicants immediately processed fresh specimens by removal of red blood cells using ACK lysis buffer, filtration and isolation of a single cell suspension. Next, CD45⁺ cells were depleted using the MACS beads and columns per manufacturer's instructions (Miltenyi Biotec). While this approach led to only partial depletion of CD45⁺ immune cells (the main component of ascites), Applicants avoided repeated bead-based depletion, because it results in RNA degradation and compromises subsequent scRNA-seg¹⁶. Next, cells were counted and resuspended in PBS supplemented with 0.04% BSA for loading for single-cell library construction on the 10× Genomics platform. Experiments were performed with the Chromium Single Cell 3′ Library & Gel Bead Kit v2 and Chromium Single Cell 3′ Chip kit v2 according to the manufacturer's instructions in the Chromium Single Cell 3′ Reagents Kits V2 User Guide. Briefly, ˜6,000 cells were loaded to each channel, then partitioned into Gel Beads in Emulsion in the GemCode instrument, where cell lysis and barcoded reverse transcription of RNA occurred, followed by amplification, shearing and 5′ adaptor and sample index attachment. Barcoded single cell transcriptome libraries were sequenced with 38 bp paired end reads on an Illumina NextSeq 500 Instrument. The HTAPP cohort was processed and analyzed as recently described¹⁶.

Cell culture. High-grade serous ovarian cancer cell lines Kuramochi, Ovsaho, Ovcar4, Ovcar8, and Tyknu were provided by the CCLE project at the Broad Institute. All cell lines were cultured in RPMI1640 Medium (Gibco), supplemented with 10% Fetal Bovine Serum (FBS) and 1% penicillin/streptomycin (Invitrogen), and were maintained in an incubator at 37° C. and 5% CO₂. Sub-culturing of cell lines was done by detaching cells using 0.05% trypsin EDTA, quenching, washing and re-suspending the cell pellet in fresh media.

In vitro and ex vivo drug sensitivity testing. The Growth In Ultra-Low Attachment (GILA) Assay⁴² was used to assess drug sensitivity of patient-derived cell lines and established cell lines. Five ovarian cancer cell lines (Kuramochi, Ovsaho, Ovcar4, Ovcar8, Tyknu) were each plated at 5,000 cells/100 μL AR-5 medium (ACL4 Media with 5% FBS)⁴³ per well in both flat-bottom high attachment (HA) (Corning Inc., 353072) and round-bottom ultra-low attachment (ULA) (Sigma-Aldrich Co. LLC CLS7007-24EA) 96-well plates. For the initial screen with JAK/STAT-pathway inhibitors, cells were treated with AZD1480 (AstraZeneca), NVP-BSK805 (Selleck Chemicals), TG101348 (STEMCELL Technologies Inc), CX-6258 (Selleck Chemicals), CEP33779 (Selleck Chemicals), Ruxolitinib (Novartis), Tofacitinib (Pfizer), SGI-1776 (Sigma-Aldrich, Inc), Cyt387 (Selleck Chemicals), S-Ruxolitinib (Selleck Chemicals), AZD1208 (AstraZeneca), HO-3867 (Cayman Chemical), SH-4-54 (Selleck Chemicals), trametinib (GlaxoSmithKline), JSI-124 (Sigma Aldrich), cisplatin (APP Pharmaceuticals, Inc) or carboplatin (Hospira, Inc) using 1 μM in both ULA and HA plates. Cells were collected on Day 0 (control) or after 48 hours of treatment, covered with an aluminum lid, and flash frozen at −80° C. Within 24 hours of freezing, cell viability of the samples was determined using CellTiter-Glo® Luminescent Cell Viability (CTG) Assay (Promega Corporation, G7572). The CTG reagent was thawed and diluted with 1×PBS in a 1:1 ratio prior to use. Plates were thawed and resuspended in equal volumes of CTG, shaken on an orbital shaker at 100 rpm for 2 min at room temperature to mix contents and to induce cell lysis. The plates were incubated at room temperature for 10 min to stabilize luminescent signal. The cell culture and CTG reagent mixture was transferred to a white 96-well plates (Thermo Fisher Scientific, 07200722) and the luminescence signal was read on a 2103 EnVision™ Multilabel Plate Reader (PerkinElmer). Data analysis was performed using Excel and Prism.

Primary cell cultures were generated by removing RBCs and depleting CD45-positive cells using the MACS beads and columns per manufacturer's instructions (Miltenyi Biotec), and 5,000 cells/100 μL AR-5 medium per well were plated in both HA and ULA 96-well plates. For the GILA assay, 5,000 cells/well were seeded, maintained for 24 hours and treated with either JSI-124 (Sigma Aldrich), cisplatin (APP Pharmaceuticals, Inc), carboplatin (Hospira, Inc), olaparib (AstraZeneca), or paclitaxel (Life Technologies) at indicated doses of 10 μM 1 μM, 100 μM, and 10 μM in both ULA and HA plates. After three or five days of treatment, plates were covered with aluminum lids and flash frozen at −80° C. followed by CTG assay as described above. Data analysis was performed using Excel and Prism.

Spheroid formation inhibition assay. Five ovarian cancer cell lines (Kuramochi, Ovsaho, Ovcar4, Ovcar8, Tyknu) were plated in flat bottomed ULA plates with either 0.1% DMSO, JSI-124 or carboplatin, at 10 nM, 100 nM, and 1 μM dosing. After 48 and 72 hours, spheroids with a diameter over 200 μm were counted using a CK40 Culture Microscope (OLYMPUS America, Inc). The relative spheroid count was determined as ratio of spheroids in the treatment conditions compared to DMSO treated cells.

Culturing of patient-derived spheroids. Ovarian primary cells were frozen with 90% FBS and 10% DMSO at −80° C. and transferred to a liquid nitrogen tank for long-term storage. For spheroid assays, cells were thawed, washed in PBS, re-suspended in AR-5 media and divided into flat bottom ultra-low attachment 6-well plates (Sigma-Aldrich Co. LLC, CLS3471-24EA) and maintained in an incubator at 37° C. and 5% CO₂ for four days. Media was replaced every 48 hours. After 96 hours, the cell suspension was passed through a 20 μm filter (Miltenyi μm filter (Miltenyi Biotec, 130-101-812) in order to capture the spheroids larger than 20 μm in diameter. Reverse filtration was performed with AR-5 media to capture the spheroids that remain on the filter. The spheroids were collected and plated in ultra-low attachment 6-well plates for short-term culture.

Mesothelial clearance assay. Primary ovarian cancer cells (NACT8) were cultured in a low-attachment 6-well plate for 96 hours. The culture was treated with 10 μM JSI-124 or DMSO for 30 or 120 min. Spheroids were isolated by passing the suspension through a 20 μm filter. Reverse filtration was performed to collect spheroids larger than 20 μm into a 6-well plate. Cells were washed and spheroids were collected for the mesothelial clearance assay. A mesothelial cells monolayer was prepared by plating mesothelial cells on glass-bottom dishes (Mat-TEK Corporation) coated with 5 μg/mL of fibronectin (Sigma, USA). Cells were maintained in culture until confluent (˜48 hours after plating). Suspended NACT8 cell spheroids were collected and added to a confluent mesothelial monolayer expressing green fluorescent protein (GFP), allowed to attach for 30-60 min, and imaged for up to 16 hours using a Nikon Ti-E Inverted Motorized Widefield Fluorescence Microscope equipped with incubation chamber. Only spheres that remained attached during the experiment were used for quantification. Mesothelial clearance was quantified as previously described^{44,45}.

Protein extraction and Western blot analysis. Cells were lysed in RIPA lysis buffer (150 mM NaCl, 1.0% IGEPAL CA-630, 0.5% sodium deoxycholate, 0.1 SDS, 50 mM Tris pH 8.0, with protease and phosphatase inhibitors) on ice for 30 min. Phosphatase and protease inhibitors added to the RIPA buffer (Sigma-Aldrich) were purchased as phosSTOP and complete EDTA free mini tablets respectively (Roche). Westerns were performed as described¹⁴⁶ using the indicated antibodies: Phospho-Tyr705 STAT3 (9131; Cell Signaling Technology), STAT3 antibody (sc-482, Santa Cruz Biotechnology), and Tubulin (T5168, Sigma).

Luciferase assay. Heya8 cells were transfected with the STAT3 responsive luciferase reporter M67-luc (kindly provided by Jacqueline Bromberg, Memorial Sloan Kettering) and renilla luciferase (Promega) using lipofectamine 2000⁴⁶. Cells were pretreated with 1 μM JSI-124 for 1 hour and then stimulated with 10 ng/mL oncostatin M (OSM; Peprotech) for 6 hours. Luciferase activity was measured using a dual luciferase kit (Promega) on a luminoskan luminometer. Firefly luciferase activity is normalized to renilla and expressed relative to media controls.

Immunofluorescence. Formalin-fixed, paraffin-embedded (FFPE) tissues were cut at a thickness of 5 μm and mounted on glass slides. Direct immunofluorescence was performed as previously described⁴⁷ using the following antibodies (manufacturer, clone, dilution): anti-HLA-DPB1-Alexa 647 (Abcam, Clone EPR11226, 1:50) and anti-Pan-Cytokeratin-eFluor 570 (ThermoFisher Science, AE1/AE3, 1:100). Images were acquired on CyteFinder slide scanning fluorescence microscope (RareCyte Inc. Seattle Wash.) using a 10× objective.

Patient-derived xenograft model experiments. HGSOC PDX models derived from patients with different treatment histories were selected for implantation: DF20 (BRCAWT treatment-naïve, clinically platinum sensitive); DF101 (BRCA1 mutant, 2 lines of prior therapy, clinically platinum resistant); DF68 (BRCA1 mutant, 6 lines of prior therapy, clinically platinum resistant)²⁹. For all experiments, Applicants used NOD-SCID IL2Rγnull mice (NSG, Jackson Laboratory). For the carboplatin treatment study, to facilitate detecting minimal residual disease, Applicants used a subcutaneous (SC) model instead of an intraperitoneal (IP) model.

Frozen stocks of tumor cells were briefly thawed and 5×10⁶ cells were injected subcutaneously into the flanks of NOD-SCID IL2Rγnull mice (NSG, Jackson Laboratory). After tumors were established (150-300 mm³), animals were divided into two groups per model: vehicle (n=5) and carboplatin (n=30). Animals were treated with DMSO or with IP carboplatin at 70 mg/kg weekly for 3 total doses. Mice were monitored by weight, clinical appearance, and tumor burden by measurements by calipers and BLI. For BLI imaging, the mice were anesthetized and imaged every 1-2 weeks. Vehicle mice were harvested at endpoint using predefined criteria, 40% weight gain, 20% weight loss, ascites, or humane reasons. Carboplatin-treated mice for the minimal residual disease (MRD) group were harvested at the nadir of BLI signal and size. The remaining carboplatin-treated mice continued weekly to biweekly BLI monitoring and were harvested at endpoint using the same criteria as the vehicle mice. For the MRD cohort, the entirety of the tumor was harvested for scRNA-seq. For the remaining carboplatin cohort, at final harvest the majority of the tumor was harvested for scRNA-seq with one fragment to 10% neutral buffered formalin and 1 fragment snap-frozen. Solid organs were also placed in formalin. Tumors/tissues were disaggregated as previously described⁸. Tumor cells expressing mCherry were isolated and flow-sorted into 96-well plates as described above and subjected to plate-based scRNA-seq and analyzed in a pooled fashion.

For the JSI-124 treatment, PDX model DF20 was selected and 5×10⁶ tumor cells were injected into two cohorts of mice, one cohort SC to induce tumors and one cohort IP to induce ascites (n=5 per group). For the first experiment (tumor formation) at 7 days post-injection of the tumor cells, the animals were treated with DMSO vs. IP JSI-124 at 1 mg/kg daily for 14 days. For the second experiment (established tumors), tumors were allowed to grow for 3 weeks after cell injection, and then DMSO vs. IP JSI-124 dosing proceeded daily for 14 days. In each experiment animals were monitored by BLI weekly and sacrificed at study endpoint at day 16.

Plate-based scRNA-seq data processing. Expression levels were quantified as E_{i,j}=log₂(TPM_{i,j}/10+1) where TPM_{i,j} refers to transcripts per million for gene i in cell j, as calculated by RSEM for Smart-seq2 samples⁴⁸. TPM values were divided by 10 since Applicants estimate the complexity of single-cell libraries to be about 100,000 transcripts and would like to avoid counting each transcript ˜10 times. This would be the case with TPM, which may inflate the difference between the expression level of a gene in cells in which the gene is detected and those in which it is not detected. This modification has a minimal influence on the expression values, but decreases the difference between the expression values of undetected genes (i.e., zero) and that of detected genes (data not shown), thereby reducing the impact of dropouts on downstream analysis.

For each cell, Applicants quantified two quality measures:
(i) The number of genes for which at least one read was mapped, which is indicative of library complexity;
(ii) The average expression level (E) of a curated list of housekeeping genes, which is meant to verify that genes which are expected to be expressed highly, regardless of cell type, are indeed detected as highly expressed.

Scatterplot analyses of all profiled cells separated low and high quality cells based on the these two measures (data not shown), and Applicants therefore conservatively excluded all cells with either fewer than 2,000 detected genes or an average housekeeping expression level (E) below 2.5, as done in previous studies^{8,49}. In each sample, Applicants further excluded cells with ad-hoc thresholds in case there was a subset of cells with fewer detected genes that appeared to be of low quality by manual inspection. For cells passing these quality controls, the median number of detected genes was 7892.

Applicants used the remaining cells (k=1297 for human samples and k=795 for mouse samples) to identify genes that are expressed at sufficient levels by calculating the aggregate expression of each gene i across the k cells, as Eₐ(i)=log₂(Average(TPMᵢ)_{1, . . . , k}+1), and excluded genes with Eₐ<4. For the remaining cells and genes, Applicants defined relative expression by centering the expression levels, Er_{i,j}=E_{i,j}−Average(Eᵢ)_{1, . . . , k}. The relative expression levels, across the remaining subset of cells and genes, were used for downstream analysis.

Droplet based scRNA-seq data processing. The droplet-based data processing followed similar lines to that of the plate-based data, with the necessary changes to accommodate the change in platform:
1. TPM values were obtained from CellRanger.
2. Modified threshold values for the number of detected genes in order to accommodate for the lower detection rate of the droplet-based platform. The minimal number of detected genes was set to 1,000.
3. In addition, the droplet-based platform also enabled quantification of the number of transcripts (i.e. UMI), and therefore Applicants used a second filtering parameter of at least 4,000 transcripts. Applicants did not use a threshold for housekeeping genes.
4. Genes were chosen for downstream analyses if they were detected with more than 5 transcripts by more than 5 cells.

Dimensionality reduction and clustering. Following the initial processing steps described above, Applicants clustered the cells using tSNE (with perplexity of 30 and default parameters of the Matlab's tsne function) followed by density clustering using DBscan (with parameters epsilon=5 and min-points=10). In the case of smartseq2 clusters, cluster 1, which was dominated by cells of patient Patient 8, was also assigned several outlier cells from patients Patient 9 and Patient 10, which were manually excluded from the downstream analysis. Clusters were annotated based on expression of marker genes (as described above) and based on the top 30 most upregulated genes in each cluster (defined by fold change between the average expression in the corresponding cluster compared to average expression in all other clusters) (FIG. 1D, 2C). The malignant cell clusters were further supported by CNAs which were estimated as described previously^{8,49} (code is available at github.com/broadinstitute/inferCNV).

Expression programs of intra-tumoral heterogeneity. For each of five patients and each of the 3 PDX models for which Applicants had profiles of >100 malignant cells, Applicants used non-negative matrix factorization (NMF) to identify 6-9 expression modules of genes coherently co-varying across the cells within each tumor separately. For this purpose, Applicants used non-negative matrix factorization (as implemented by the MATLAB nnmf function, with the number of factors set to 10) to identify variable expression programs. NNMF was applied to the relative expression values (Er), by transforming all negative values to zero. Notably, undetected genes include many drop-out events (genes that are expressed but are not detected in particular cells due to the incomplete transcriptome coverage), which introduce challenges for normalization of single-cell RNA-seq; since NNMF avoids the exact normalized values of undetected genes (as they are all zero), it may be beneficial in analysis of single-cell RNA-seq (data not shown). Applicants retained only programs for which the standard deviation in cell scores within the respective tumor was larger than 0.8, which resulted in a total of 35 programs across the 5 human samples and 24 across the mouse models. The programs were compared by hierarchical clustering, using the number of overlapping genes (among the 50 top-scoring genes of each program) as a similarity metric. Five clusters of programs (two cell cycle and three inflammatory programs) were identified in the human samples based on a minimal overlap of 10 genes between programs and used to define meta-signatures. For each cluster, NNMF gene scores were log 2-transformed and then averaged across the programs in the cluster, genes were ranked by their average scores, and the top 30 genes were defined as the meta-signature.

To evaluate if similar programs of intra-tumor heterogeneity recur in the test dataset, Applicants defined a small set of n core genes for each meta-signature, consisting of those genes that were identified in multiple tumors and/or are established as related to the programs inferred function. Applicants then examined if there is an enrichment of cells in which a large number of those genes are detected as expressed. Applicants counted the number of cells in which expression of X of those genes is detected, for X=[1 . . . n]. To assess the significance of the observed counts, Applicants repeated the analysis 10,000 times with other sets of n genes referred to as control gene-sets. Each control gene-set was chosen such that it has a similar distribution of expression levels to that of the signature's core genes. To that end, Applicants first partitioned all analyzed genes into 50 bins based on their average expression across all cancer cells. Next, Applicants defined each control gene-set by randomly sampling from each bin the same number of genes that are in that bin among the signature's core genes. The fraction of simulations (out of 10,000) in which an equal number of the core signature genes were detected as expressed, was used to define the p-value of observed counts.

TCGA subtype scores and purity estimate. Bulk RNA-seq data of samples, as well as NNMF clustering and differential expression analysis were downloaded from the Broad Firehose website (https://gdac.broadinstitute.org/), along with additional tumor and clinical annotations. Classification of tumors to predefined molecular subtypes was done based on the NNMF clustering with four factors, and the average expression of top 100 differentially expressed genes for each cluster was defined as the subtype signatures, for which single cell clusters were scored. Purity data for defined by including ABSOLUTE⁴¹.

Data Availability. Processed data is available at the Gene Expression Omnibus (GSE146026) and raw data will be available via the Broad Institute Data Use Oversight System (DUOS, duos.broadinstitute.org/#/home).

### REFERENCES

1. Winterhoff, B. J. et al. Single cell sequencing reveals heterogeneity within ovarian cancer epithelium and cancer associated stromal cells. Gynecol. Oncol. 144, 598-606 (2017).
2. Shih, A. J. et al. Identification of grade and origin specific cell populations in serous epithelial ovarian cancer by single cell RNA-seq. PloS One 13, e0206785 (2018).
3. Hu, Z. et al. The Repertoire of Serous Ovarian Cancer Non-genetic Heterogeneity Revealed by Single-Cell Sequencing of Normal Fallopian Tube Epithelial Cells. Cancer Cell 37, 226-242.e7 (2020).
4. Siegel, R. L., Miller, K. D. & Jemal, A. Cancer statistics, 2016. CA. Cancer J. Clin. 66, 7-30 (2016).
5. Network, T. C. G. A. R. Integrated genomic analyses of ovarian carcinoma. Nature 474, 609-615 (2011).
6. Patch, A.-M. et al. Whole-genome characterization of chemoresistant ovarian cancer. Nature 521, 489-494 (2015).
7. Ahmed, N. & Stenvers, K. L. Getting to Know Ovarian Cancer Ascites: Opportunities for Targeted Therapy-Based Translational Research. Front. Oncol. 3, (2013).
8. Tirosh, I. et al. Dissecting the multicellular ecosystem of metastatic melanoma by single-cell RNA-seq. Science 352, 189-196 (2016).
9. Tirosh, I. et al. Single-cell RNA-seq supports a developmental hierarchy in human oligodendroglioma. Nature 539, 309-313 (2016).
10. Venteicher, A. S. et al. Decoupling genetics, lineages, and microenvironment in IDH-mutant gliomas by single-cell RNA-seq. Science 355, (2017).
11. Puram, S. V. et al. Single-Cell Transcriptomic Analysis of Primary and Metastatic Tumor Ecosystems in Head and Neck Cancer. Cell 171, 1611-1624.e24 (2017).
12. Izar, B. & Rotem, A. GILA, a Replacement for the Soft-Agar Assay that Permits High-Throughput Drug and Genetic Screens for Cellular Transformation. Curr. Protoc. Mol. Biol. 116, 28.8.1-28.8.12 (2016).
13. Peterson, V. M. et al. Ascites analysis by a microfluidic chip allows tumor-cell profiling. Proc. Natl. Acad. Sci. U.S.A. 110, E4978-4986 (2013).
14. Picelli, S. et al. Smart-seq2 for sensitive full-length transcriptome profiling in single cells. Nat. Methods 10, 1096-1098 (2013).
15. Trombetta, J. J. et al. Preparation of Single-Cell RNA-Seq Libraries for Next Generation Sequencing. Curr. Protoc. Mol. Biol. Ed. Frederick M Ausubel Al 107, 4.22.1-4.22.17 (2014).
16. Slyper, M. et al. A single-cell and single-nucleus RNA-seq toolbox for fresh and frozen human tumors. bioRxiv 761429 (2019) doi:10.1101/761429.
17. Cirri, P. & Chiarugi, P. Cancer associated fibroblasts: the dark side of the coin. Am. J. Cancer Res. 1, 482-497 (2011).
18. Öhlund, D. et al. Distinct populations of inflammatory fibroblasts and myofibroblasts in pancreatic cancer. J. Exp. Med. 214, 579-596 (2017).
19. Iliopoulos, D., Hirsch, H. A. & Struhl, K. An epigenetic switch involving NF-kappaB, Lin28, Let-7 MicroRNA, and IL6 links inflammation to cell transformation. Cell 139, 693-706 (2009).
20. Cai, H. et al. Colony-stimulating factor-1-induced AIF1 expression in tumor-associated macrophages enhances the progression of hepatocellular carcinoma. Oncoimmunology 6, e1333213 (2017).
21. Li, J. et al. VSIG4 inhibits proinflammatory macrophage activation by reprogramming mitochondrial pyruvate metabolism. Nat. Commun. 8, 1-14 (2017).
22. Aran, D., Sirota, M. & Butte, A. J. Systematic pan-cancer analysis of tumour purity. Nat. Commun. 6, 8971 (2015).
23. Silva, I. A. et al. Aldehyde dehydrogenase in combination with CD133 defines angiogenic ovarian cancer stem cells that portend poor patient survival. Cancer Res. 71, 3991-4001 (2011).
24. Wu, X. et al. AXL kinase as a novel target for cancer therapy. Oncotarget 5, 9546-9563 (2014).
25. Biton, M. et al. T Helper Cell Cytokines Modulate Intestinal Stem Cell Renewal and Differentiation. Cell 175, 1307-1320.e22 (2018).
26. Smillie, C. S. et al. Intra- and Inter-cellular Rewiring of the Human Colon during Ulcerative Colitis. Cell 178, 714-730.e22 (2019).
27. Miao, Y. et al. Adaptive Immune Resistance Emerges from Tumor-Initiating Stem Cells. Cell 177, 1172-1186.e14 (2019).
28. Rodig, S. J. et al. MHC proteins confer differential sensitivity to CTLA-4 and PD-1 blockade in untreated metastatic melanoma. Sci. Transl. Med. 10, (2018).
29. Liu, J. F. et al. Establishment of Patient-Derived Tumor Xenograft Models of Epithelial Ovarian Cancer for Preclinical Evaluation of Novel Therapeutics. Clin. Cancer Res. (2016) doi:10.1158/1078-0432.CCR-16-1237.
30. Blaskovich, M. A. et al. Discovery of JSI-124 (Cucurbitacin I), a Selective Janus Kinase/Signal Transducer and Activator of Transcription 3 Signaling Pathway Inhibitor with Potent Antitumor Activity against Human and Murine Cancer Cells in Mice. Cancer Res. 63, 1270-1279 (2003).
31. Barretina, J. et al. The Cancer Cell Line Encyclopedia enables predictive modelling of anticancer drug sensitivity. Nature 483, 603-607 (2012).
32. Dijkgraaf, E. M. et al. Chemotherapy Alters Monocyte Differentiation to Favor Generation of Cancer-Supporting M2 Macrophages in the Tumor Microenvironment. Cancer Res. 73, 2480-2492 (2013).
33. Axelrod, M. L., Cook, R. S., Johnson, D. B. & Balko, J. M. Biological Consequences of MHC-II Expression by Tumor Cells in Cancer. Clin. Cancer Res. Off. J. Am. Assoc. Cancer Res. 25, 2392-2402 (2019).
34. Jiménez-Sánchez, A. et al. Heterogeneous Tumor-Immune Microenvironments among Differentially Growing Metastases in an Ovarian Cancer Patient. Cell 170, 927-938.e20 (2017).
35. Zhang, A. W. et al. Interfaces of Malignant and Immunologic Clonal Dynamics in Ovarian Cancer. Cell 173, 1755-1769.e22 (2018).
36. Duan, Z. et al. Signal transducers and activators of transcription 3 pathway activation in drug-resistant ovarian cancer. Clin. Cancer Res. Off. J. Am. Assoc. Cancer Res. 12, 5055-5063 (2006).
37. Kommoss, S. et al. Bevacizumab May Differentially Improve Ovarian Cancer Outcome in Patients with Proliferative and Mesenchymal Molecular Subtypes. Clin. Cancer Res. Off. J. Am. Assoc. Cancer Res. 23, 3794-3801 (2017).
38. Isella, C. et al. Stromal contribution to the colorectal cancer transcriptome. Nat. Genet. 47, 312-319 (2015).
39. Calon, A. et al. Stromal gene expression defines poor-prognosis subtypes in colorectal cancer. Nat. Genet. 47, 320-329 (2015).
40. Pitt, J. M. et al. Targeting the tumor microenvironment: removing obstruction to anticancer immune responses and immunotherapy. Ann. Oncol. 27, 1482-1492 (2016).
41. Carter, S. L. et al. Absolute quantification of somatic DNA alterations in human cancer. Nat. Biotechnol. 30, 413-421 (2012).
42. Rotem, A. et al. Alternative to the soft-agar assay that permits high-throughput drug and genetic screens for cellular transformation. Proc. Natl. Acad. Sci. U.S.A. 112, 5708-5713 (2015).
43. Gazdar, A. F. & Oie, H. K. Re: Growth of cell lines and clinical specimens of human non-small cell lung cancer in a serum-free defined medium. Cancer Res. 46, 6011-6012 (1986).
44. Davidowitz, R. A. et al. Mesenchymal gene program-expressing ovarian cancer spheroids exhibit enhanced mesothelial clearance. J. Clin. Invest. 124, 2611-2625 (2014).
45. Iwanicki, M. P. et al. Mutant p53 regulates ovarian cancer transformed phenotypes through autocrine matrix deposition. JCI Insight 1, (2016).
46. Nelson, E. A. et al. Nifuroxazide inhibits survival of multiple myeloma cells by directly inhibiting STAT3. Blood 112, 5095-5102 (2008).
47. Lin, J.-R. et al. Highly multiplexed immunofluorescence imaging of human tissues and tumors using t-CyCIF and conventional optical microscopes. eLife 7, (2018).
48. Li, B. & Dewey, C. N. RSEM: accurate transcript quantification from RNA-Seq data with or without a reference genome. BMC Bioinformatics 12, 323 (2011).
49. Patel, A. P. et al. Single-cell RNA-seq highlights intratumoral heterogeneity in primary glioblastoma. Science 344, 1396-1401 (2014).

Tables

TABLE 1
Clinical and sample data

| | | Number of | | | BRCA | | Upfront surgery or |
|---|---|---|---|---|---|---|---|
| Patient ID | Platform | samples collected | | | status | | neoadjuvant chemotherapy |
| Patient 1 | 10X | 1 | | | WT | | Upfront surgery |
| Patient 2 | 10X | 2 | | | WT | | Neoadjuvant |
| Patient 3 | 10X | 1 | | | N/A | | No surgery |
| Patient 4 | 10X | 1 | | | WT | | Neoadjuvant |
| Patient 5 | 10X, | 2 | | | WT | | Neoadjuvant |
| | Smartseq2 | | | | | | |
| Patient 6 | 10X | 1 | | | N/A | | Neoadjuvant |
| Patient 7 | SmartSeq2 | 5 | | | BRCA2 MUT | | Upfront surgery |
| Patient 8 | SmartSeq2 | 3 | | | WT | | Upfront surgery |
| Patient 9 | SmartSeq2 | 3 | | | WT | | Upfront surgery |
| Patient 10 | SmartSeq2 | 1 | | | WT | | Neoadjuvant |
| Patient 11 | SmartSeq2 | 1 | | | WT | | Upfront surgery |
| Patient 12 | 10X | 1 | | | WT | | Neoadjuvant |
| Patient 13 | 10X | 1 | | | BRCA2 MUT | | Upfront surgery |
| Patient 14 | 10X | 1 | | | WT | | Upfront surgery |
| Patient 15 | 10X | 1 | | | WT | | Neoadjuvant |
| | | | | | | | (carboplatin and paxlitaxel) |
| Patient 16 | sn-RNAseq | 1 | | | N/A | | Neoadjuvant |
| | | | | | | | (carboplatin and paxlitaxel) |
| | Sample | Sample | Sample | Sample | Sample | Treatment | Clinical status at |
| Patient ID | 1 | 2 | 3 | 4 | 5 | status of sample | time of sample |
| Patient 1 | 1212 | | | | | On-treatment | Recurrent |
| Patient 2 | 238 | 257 | | | | On-treatment | Recurrent |
| Patient 3 | 0 | | | | | Treatment-naïve | Diagnosis |
| Patient 4 | 23 | | | | | After 1 cycle | Initial treatment |
| | | | | | | of chemotherapy | |
| Patient 5 | 0 | 8 | | | | Sample 1: | Diagnosis/Initial |
| | (10X and | (10X) | | | | Treatment-naïve; | treatment |
| | SMARTseq2) | | | | | Sample 2: | |
| | | | | | | After 1 cycle of | |
| | | | | | | chemotherapy | |
| Patient 6 | 0 | | | | | Treatment-naïve | Diagnosis |
| Patient 7 | 1701 | 1726 | 1758 | 1844 | 1859 | On-treatment | Recurrent |
| Patient 8 | 2358 | 2367 | 2374 | | | On-treatment | Recurrent |
| Patient 9 | 5040 | 5058 | 5087 | | | On-treatment | Recurrent |
| Patient 10 | 598 | | | | | On-treatment | Recurrent |
| Patient 11 | 1943 | | | | | On-treatment | Recurrent |
| Patient 12 | 0 | | | | | Treatment-naïve | Diagnosis |
| Patient 13 | 352 | | | | | On-treatment | Recurrent |
| Patient 14 | 2213 | | | | | On-treatment | Recurrent |
| Patient 15 | 69 | | | | | Interval | Initial treatment |
| | | | | | | cytoreduction | |
| | | | | | | following | |
| | | | | | | neoadjuvant | |
| | | | | | | chemotherapy | |
| Patient 16 | 122 | | | | | Interval | Initial treatment |
| | | | | | | cytoreduction | |
| | | | | | | following | |
| | | | | | | neoadjuvant | |
| | | | | | | chemotherapy | |

| Cohort | #Samples | #Patients | Method | Enrichment of cancer cells |
|---|---|---|---|---|
| 1 | 8 | 6 | Droplet | CD45 + depletion |
| 2 | 14 | 6 | SMART-Seq2 | Flow-sorting CD24 + |
| | | | | EPCAM + epithelial cells |
| 3 | 5 | 5 | 3 ascites: Droplet | CD45 + depletion in ascites |
| | | | 1 fresh: Droplet | |
| | | | 1 frozen: sn-RNA-seq | |

Table 2. Average expression levels of all genes included in the droplet-based analysis in each of the 18 clusters. (Filed electronically)

TABLE 3
Top 30 genes of 35 NMF-derived expression programs from ovarian cancer ascites
samples. programs are named by the patient sample they were derived from and genes are listed
from most to least significant based on NMF scores.

| Pt7_cluster1 | Pt7_cluster2 | Pt7_cluster3 | Pt7_cluster4 | Pt7_cluster5 | Pt8_cluster1 | Pt8_cluster2 |
|---|---|---|---|---|---|---|
| FN1 | LYNX1 | RPS27 | RRM2 | TOP2A | FAM83A | KLK7 |
| HEG1 | LYPD2 | RPL35 | TK1 | MKI67 | MALL | GPX3 |
| CALD1 | CLU | SERF2 | TYMS | NUSAP1 | VGLL1 | UPK3B |
| TACC1 | TRIM29 | UQCRQ | CDK1 | BIRC5 | KRT4 | DEGS2 |
| ACTA2 | MMP7 | RPL35A | ESCO2 | KIF20A | UPK3BL | FOLR1 |
| ALDH1A3 | MUC4 | RPL41 | KIFC1 | CDCA3 | S100A9 | WFDC2 |
| KRT17 | TSPAN1 | SNRPD2 | DHFR | PLK1 | FUT3 | ATP6V1B1 |
| GPRC5A | ATP6V1B1 | RPL37A | KIAA0101 | TPX2 | PTGES | LAMB2 |
| AKAP2 | SERPINA1 | RPL31 | SHCBP1 | PTTG1 | GPR110 | MDK |
| RARRES1 | SLPI | ROMO1 | ATAD2 | KIF2C | TGM2 | DHCR24 |
| CTGF | KCNN4 | TMA7 | CDCA5 | GTSE1 | TMPRSS13 | SELENBP1 |
| ERRFI1 | SYT8 | RPL37 | NUSAP1 | IQGAP3 | CST6 | KLK8 |
| TAGLN | RARRES3 | RPS8 | ASF1B | TROAP | MUC20 | ABP1 |
| PROM1 | CD74 | RPL34 | CENPM | NUF2 | LCN2 | KLK6 |
| IGFBP5 | PTGES | RPL36A | MYBL2 | CCNA2 | MUC4 | AVPR2 |
| CYR61 | HLA-DRB1 | RPL32 | FANCI | UBE2C | PSCA | SNCG |
| GAS6 | RASAL1 | RPL39 | TOP2A | KIF23 | SMIM5 | MSLN |
| TPM1 | HLA-DRA | C19orf33 | PBK | ANLN | KRT16 | SLC22A18 |
| TNFRSF11B | CP | ATP5E | CDC45 | CDKN3 | GCNT3 | ALDH3B2 |
| NDRG1 | FOLR1 | ATP5I | CENPK | CDCA8 | SCEL | UCP2 |
| PTPRR | UNC5B | RPS21 | RAD51AP1 | PRC1 | LAMA4 | LGALS3BP |
| DPYSL3 | ALOX5 | SNORD47 | RAD51 | BUB1 | TSPAN1 | GRN |
| PDLIM5 | MUC20 | RPS28 | FBXO5 | KIFC1 | GRHL1 | KLK10 |
| CDC42EP3 | FOS | RPS19 | TCF19 | CDC20 | OAS1 | RAB3D |
| KRT7 | SLC4A11 | RPS25 | NDC80 | PBK | MUC16 | TM7SF2 |
| GABRP | FXYD3 | RPS15A | IQGAP3 | KIF11 | PSMB10 | CAPN5 |
| ZFP36L1 | C3 | RPS17L | MAD2L1 | FOXM1 | DHRS9 | ASS1 |
| KLF6 | UNC5B-AS1 | RPS17 | KIF11 | SPAG5 | FLJ23867 | SLC44A2 |
| NNMT | DEFB1 | RPLP1 | UBE2C | TACC3 | GNA15 | RSPO3 |
| GADD45B | COL12A1 | COX6C | CCNA2 | CDK1 | F3 | LRRC41 |
| Pt8_cluster3 | Pt8_cluster4 | Pt8_cluster5 | Pt8_cluster6 | Pt8_cluster7 | Pt8_cluster8 | Pt9_cluster1 |
| RARRES1 | HERPUD1 | NR4A1 | PDDC1 | TOP2A | IL32 | CLU |
| CRYAB | DDIT3 | GADD45B | ZNF793 | BIRC5 | SOD2 | TACSTD2 |
| HLA-DRB1 | TRIB3 | IL6 | CCL5 | NUSAP1 | CXCL3 | MFAP2 |
| HLA-DRA | ZFAND2A | PPP1R15A | IKZF3 | ANLN | NFKBIA | LGALS3BP |
| HLA-DRB5 | DNAJB9 | CYR61 | GPR82 | UBE2C | CCL2 | FBLN2 |
| NNMT | TSPYL2 | HBEGF | TLCD2 | PKMYT1 | TNFAIP3 | LY6E |
| PLA2G16 | HSPA5 | C7orf53 | ZBTB8A | TPX2 | TNF | VTCN1 |
| MGST1 | NUPR1 | KLF4 | LYZ | CDK1 | CXCL1 | CLIC5 |
| HLA-DRB6 | FTL | GEM | LOC100131257 | CCNB2 | TUBA1A | PTPRS |
| MT1F | CRYAB | RGS2 | C21orf62 | TROAP | TPM1 | RAB25 |
| CLU | BRD2 | HSPA1A | CYP20A1 | CDCA5 | EDN1 | SH3BGRL |
| CD14 | KIAA0907 | CD83 | TNFAIP8L1 | CDC20 | TAGLN | CD74 |
| HLA-DMA | ZNF165 | FAM83D | CRX | FAM64A | ICAM1 | BST2 |
| NFIB | IFIT2 | SLC7A5P2 | DDX51 | PLK1 | CCL20 | SERPING1 |
| CD74 | KLHL21 | HSPH1 | LOC100128288 | RRM2 | CLDN1 | MFGE8 |
| C3 | MT2A | HSPA1B | PLCXD1 | PTTG1 | CXCL2 | ANKRD65 |
| BNIP3L | MXD1 | HEXIM1 | SPN | UK | TNFAIP2 | UCA1 |
| OAT | ZFAS1 | MXD1 | ZC3H12D | HMGB2 | NEDD9 | THEM6 |
| HLA-DPA1 | GAS5 | KLF10 | ORAI2 | MAD2L1 | ADAMTS9 | HSPG2 |
| SLC34A2 | ATF4 | HSPA7 | FBLIM1 | TK1 | FLNA | MSLN |
| NUPR1 | BAG3 | BRD2 | NLRP12 | CENPW | ARHGAP29 | NDRG2 |
| ANXA1 | MT1X | C1orf51 | LINC00294 | ZWINT | IL8 | TAPBP |
| MT1X | EPB41L4A-AS1 | ZNF296 | PDE6A | FBXO5 | CTHRC1 | CDH6 |
| CDC42EP2 | FAM46A | HSPA6 | ZNF264 | ASF1B | RGS10 | PLD3 |
| TUBB6 | EIF1 | ING1 | GNE | TACC3 | UBD | RBMS3 |
| SAA1 | RSL1D1 | CDC42EP3 | POLH | NCAPD2 | THBS1 | COMP |
| CSTB | HSP90B1 | FAM46A | SHISA9 | CENPM | CALD1 | SEPP1 |
| VIM | ANKHD1 | TRA2A | LOC646214 | SMC4 | PTX3 | IFIT3 |
| GPX3 | AVPI1 | BAG3 | UGDH-AS1 | ZNF695 | RELB | RNF213 |
| MT1E | MTHFD2 | ATF3 | LOC643406 | CCNB1 | CYR61 | IFI6 |
| Pt9_cluster2 | Pt9_cluster3 | Pt9_cluster4 | Pt9_cluster5 | Pt9_cluster6 | Pt9_cluster7 | Pt9_cluster8 |
| FOLR1 | S100A6 | PKMYT1 | BIRC5 | HIST1H2BG | IGFBP5 | UBD |
| CP | ITGB4 | CDK1 | TPX2 | NR1D1 | NUPR1 | CCL2 |
| ASS1 | UPK1B | KIAA0101 | KIF20A | HIST1H2BD | ST6GAL1 | IL32 |
| SUSD3 | F3 | TYMS | KIF18B | HIST2H2BF | CRYAB | CD40 |
| PSAT1 | KLK10 | CDCA7 | CCNA2 | HIST1H3D | VAT1 | TAP1 |
| CFI | S100A10 | RRM2 | CCNB2 | HIST1H2AC | SOX4 | TNFAIP2 |
| SCGB2A1 | SLPI | ASF1B | PLK1 | HIST1H2BC | MAP1B | IL8 |
| MUC1 | KLK11 | UHRF1 | PTTG1 | ZNF844 | APOL4 | CRYAB |
| ATP6V1B1 | CD9 | TK1 | CDC20 | HIST1H2BK | TUBB2B | MARCO |
| APOA1 | KRT6A | CDC45 | CCNB1 | HIST1H2AE | NREP | IL23A |
| LCN2 | WNT7A | ZWINT | KIF23 | HIST1H4H | TUBA1A | LGALS14 |
| CHI3L1 | S100A4 | PBK | TACC3 | HIST1H2BH | PCMTD1 | PSMB9 |
| GAPDH | RHOF | UBE2C | CDCA8 | ZC3H12A | SLFN5 | ICAM1 |
| DHCR24 | C19orf33 | CDC6 | PRC1 | CCNL1 | ID3 | TNF |
| SLPI | KRT19 | E2F1 | KIF2C | IER2 | WLS | CD74 |
| COQ7 | ANXA2 | PCNA | BUB1 | JUN | FN1 | TUBB2B |
| DEFB1 | B3GNT3 | MCM4 | NUF2 | PPP1R15A | COL1A1 | CCL20 |
| RPS15A | TGM1 | AURKB | CDKN3 | GADD45B | DBN1 | KRT23 |
| PRDX2 | PPL | BIRC5 | HJURP | HIST1H2A1 | GPR56 | IL4I1 |
| CFB | MALL | CDCA5 | TOP2A | HIST2H2AA3 | FTL | TAPBP |
| EEF1G | ITGA3 | ATAD2 | PBK | HIST2H2AA4 | KRT5 | C10orf10 |
| CTSB | FXYD3 | SHCBP1 | NEK2 | ARRDC3 | GDF15 | CLDN1 |
| FOLR3 | KLK13 | MAD2L1 | NDC80 | HIST2H2BE | ERV3-1 | SOD2 |
| MYL9 | APLP1 | GMNN | FAM83D | HIST1H2BJ | CECR1 | SELM |
| KLK10 | FOLR1 | UBE2T | HMGB2 | HIST1H3H | SYT11 | JAK3 |
| IFITM3 | GPX1 | SPC24 | KIF18A | CLK1 | LPP | TNFSF10 |
| KLK13 | TUBB6 | MELK | AURKB | YTHDC1 | KLHL24 | B2M |
| ALDOA | S100A11 | FANCD2 | AURKA | C1orf51 | SCGB2A1 | HLA-H |
| RPS3 | TMPRSS4 | DNMT1 | SHCBP1 | TSC22D2 | PTP4A3 | COL4A2 |
| UPK3B | EZR | MCM7 | TROAP | HIST3H2A | WASF2 | HLA-B |
| Pt10_cluster1 | Pt10_cluster2 | Pt10_cluster3 | Pt10_cluster4 | Pt10_cluster5 | Pt10_cluster6 | Pt10_cluster7 |
| BIRC5 | DAPL1 | RAC1 | NNMT | MT1E | CPA4 | CCBP2 |
| CCNA2 | SCGB2A1 | GOLPH3 | GGT5 | MT1G | KRT7 | TACSTD2 |
| AURKB | CFI | C20orf24 | HP | MT1X | HSPB1 | OAS1 |
| CDK1 | KLK13 | PPP1CC | C1S | MT2A | TAGLN | IF144L |
| TPX2 | SST | GAS6 | MT2A | MT1F | PDLIM5 | MX1 |
| PBK | SLC40A1 | CTSF | RARRES1 | MT1H | S100A10 | GPNMB |
| CDCA5 | DPYSL2 | KCNK15 | IL24 | KLK5 | TNS4 | IFIT3 |
| CDKN3 | ATP6V1B1 | TMEM165 | CHI3L1 | MT1M | TACSTD2 | OAS2 |
| TACC3 | PTGS1 | ZFAND5 | S100A9 | TAGLN | PLK2 | KRT23 |
| MAD2L1 | TSPAN1 | GLTSCR2 | RBP4 | LDOC1 | FHL2 | IEI6 |
| CDCA8 | VTCN1 | DUSP1 | FBXO32 | CADM3 | KRT5 | MSLN |
| KIAA0101 | LYNX1 | TOMM40 | KRT6A | TFPI2 | KRT23 | TXNIP |
| UBE2C | NRP2 | LRP10 | CADM3 | KRT5 | CALD1 | C15orf48 |
| PRC1 | SELENBP1 | PRKCD | SOD2 | CPA4 | FLNA | IFI44 |
| TK1 | UPK3B | XBP1 | TIMP1 | SPARC | MYADM | UPK3BL |
| HMGB2 | WFDC2 | HNRNPU | PI3 | MYC | TPM1 | PTGES |
| PKMYT1 | ST6GALNAC1 | IGFBP7 | PDZK1IP1 | TPM1 | CRYAB | LCN2 |
| KIF2C | C4B | MAGEF1 | TNFRSF11B | FN1 | GPR56 | CD82 |
| PTTG1 | C4B_2 | COTL1 | C1R | CALML5 | KRT80 | SAT1 |
| ASF1B | HSPA12A | GLUD1 | S100A10 | TAGLN2 | KR117 | VTCN1 |
| NUSAP1 | TSPAN3 | MTCH1 | GFPT2 | TGFBI | EDN1 | ITGB2 |
| ZWINT | ITGB4 | SLC44A1 | AQP3 | TPM2 | S100A11 | NCCRP1 |
| TYMS | TRIM17 | EPS8L1 | MT1E | LAMA4 | MYH9 | CEACAM6 |
| MELK | COL13A1 | SLC25A1 | CD44 | MYL9 | CCK | ISG15 |
| RRM2 | SRD5A3 | EBPL | PAPSS2 | KRT7 | TGM2 | IFIT1 |
| CCNB2 | BCAT1 | CCDC85B | CTSB | LGR6 | CNN3 | AGR2 |
| TROAP | KLK11 | PKP3 | FN1 | CRYAB | CAV1 | PSCA |
| TRIP13 | PROS1 | STUB1 | STC1 | EXOSC5 | CLDN4 | PARP14 |
| UBE2T | ST6GAL1 | SDF4 | CTHRC1 | RUVBL1 | PTTG1IP | HERC6 |
| CDCA3 | ADD3 | FAM3C | ACSL4 | CDH5 | AHNAK2 | GABRP |
| Pt10_cluster8 | Pt5_cluster1 | Pt5_cluster2 | Pt5_cluster3 | Pt5_cluster4 | Pt5_cluster5 | Pt5_cluster6 |
| SLC3A2 | ALDH1A2 | CRABP1 | CLSPN | TPX2 | NEDD9 | GPX3 |
| SNHG5 | UPK3B | RMST | KIAA0101 | CENPF | SIRT1 | LCN2 |
| DDIT3 | CALML5 | RP518 | HELLS | NUSAP1 | ZNF57 | CD74 |
| TAF1D | LY6G6C | RPS27A | WDR76 | BIRC5 | ZFAS1 | CLDN4 |
| NUPR1 | LRRN4 | RPS10 | ZWINT | PRC1 | SNORD22 | GPR56 |
| LOC388796 | CLDN15 | ADAMTS1 | FANCD2 | PLK1 | CDKN1A | HLA-DRA |
| SNHG1 | DENND1A | RPL8 | RRM2 | MKI67 | NR4A1 | DHRS3 |
| WDR74 | ADIRF | ZBED2 | CDC45 | KIF20A | NEAT1 | SLC44A4 |
| BRD2 | KRT19 | SORD | TYMS | NCAPG | FABP3 | LYPD2 |
| GAS5 | CYBRD1 | AKR1B1 | CDCA5 | KIF2C | SNHG1 | MUC20 |
| EPB41L4A-AS1 | UPK1B | PAICS | ASF1B | BUB1 | DKK1 | TMPRSS4 |
| IFRD1 | ITGB4 | EEF1G | CDK1 | CCNA2 | TRIB1 | MUC1 |
| ASNS | TM4SF1 | FABP5 | TK1 | TOP2A | SNHG4 | ELF3 |
| CCL5 | ANXA8L2 | RPS3 | UHRF1 | CDC20 | SNHG5 | HLA-DRB1 |
| EIF4EBP1 | TMOD1 | RPL11 | TCF19 | KIF11 | GAS5 | SRGAP1 |
| TRIM28 | LOC100505633 | RRS1 | MLF1IP | KIF4A | LOC284454 | FOLR1 |
| ZFAS1 | CYP2S1 | PEMT | MYBL2 | CCNB2 | RPL12 | B2M |
| FBLIM1 | MYRF | DANCR | CCNE2 | DLGAP5 | DUSP1 | MDK |
| EIF4A2 | ANXA8 | CCDC51 | MCM10 | CDKN3 | LYZ | WFDC2 |
| C6orf48 | SBSPON | ALDH1A2 | GINS2 | ARHGAP11A | EDN1 | CMTM7 |
| NXF1 | LRRC32 | RPL5 | CDCA7 | AURKB | SGK1 | FTH1 |
| HIST2H2BE | SLC34A2 | RTKN | CDCA4 | UBE2C | SNHG3 | CP |
| DDIT4 | AQP9 | PNOC | PKMYT1 | HJURP | KLF6 | ESR1 |
| ILF3-AS1 | CRB2 | FASN | ATAD2 | NUF2 | GATM-AS1 | CLDN3 |
| EFNA1 | DDAH2 | MDH1 | UBE2T | FAM83D | C1orf63 | RBP1 |
| ZNF460 | MYH9 | ALDH2 | MCM4 | TACC3 | OXSR1 | HLA-DPA1 |
| ARID46 | ALDH1A3 | EEF162 | RAD51 | NDC80 | RBM39 | TNFAIP2 |
| LOC643406 | PODXL | PLTP | MCM7 | TTK | TAF1D | RNF213 |
| LOC646214 | C4B | ISOC2 | UBE2C | KIF23 | KIAA0907 | MUC4 |
| ATF4 | C4B_2 | LRRC32 | FANCI | CDCA8 | SIAH1 | TACSTD2 |

TABLE 4
Top 30 genes of 24 NMF-derived expression programs from three PDX models (DF20,
DF68 and DF101). Programs are named by the PDX model they were derived from and genes are
listed from most to least significant based on NMF scores.

| DF20_cluster1 | DF20_cluster2 | DF20_cluster3 | DF20_cluster4 | DF20_cluster5 | DF20_cluster6 |
|---|---|---|---|---|---|
| BIRC5 | FAM83A | CBX3 | SOX17 | LRPAP1 | ADM |
| NUSAP1 | CNN3 | CACUL1 | TSEN34 | PPIC | PFKFB3 |
| UBE2C | CHP2 | RAP2B | TOMM40 | TMED10 | VEGFA |
| TOP2A | NDUFB3 | PAFAH1B2 | MTCH1 | MUC20 | ANGPTL4 |
| CDKN3 | ARPC2 | HNRNPH1 | MAF1 | NPC2 | EGLN3 |
| AURKB | PRDX5 | EIF4G2 | GTF2I | CTSD | BNIP3 |
| TPX2 | TMSB10 | CAPZA1 | TMEM160 | PDIA3 | PFKFB4 |
| NUF2 | BZW1 | C6orf62 | HNRNPA0 | CDH6 | DDIT4 |
| PLK1 | IDH1 | PPP1CB | PPP4C | IGFBP2 | SLC2A1 |
| KIAA0101 | CYCS | SRSF6 | SDF4 | NUCB1 | NDRG1 |
| RRM2 | FAM3B | CNN3 | CCNI | CLU | PLOD2 |
| PRC1 | SUB1 | BCL10 | GADD45GIP1 | PDIA4 | STC1 |
| CDK1 | NDUFB2 | LRRC16A | HSF1 | HSP90B1 | P4HA1 |
| PBK | TBCA | SKIL | MAGEF1 | DHCR24 | ERO1L |
| CDCA3 | PTGR1 | EHE | EPS8L1 | APOA1 | NDUFA4L2 |
| KIF20A | FTH1 | PKM | GLTSCR2 | CD63 | FUT11 |
| DLGAP5 | TMOD1 | ANXA2 | RAB11B | PPIB | HK2 |
| CDCA8 | CYP4X1 | THAP5 | ZYX | TMEM59 | BHLHE40 |
| KIFC1 | ANXA2 | STRN3 | PKP3 | HM13 | PDK1 |
| NCAPG | GTSF1 | MOB1A | PRKCI | CALR | IGFBP3 |
| KIF2C | ATPIF1 | LIN7C | ANXA11 | HLA-A | RNF24 |
| TYMS | PTMA | FYTTDI | MRPL23 | SPARC | ENO2 |
| PTTGI | SUMO1 | BZWI | PKM | HYOU1 | LDHA |
| CCNA2 | XRCC5 | FAM83A | VPS51 | SERPINA5 | RNF183 |
| NDC80 | ACTB | CLIC | CBX3 | CANX | CXCR4 |
| SKA2 | OAT | MIER1 | CDK16 | TSPAN7 | SLC6A6 |
| CCNB2 | SCP2 | WTAP | RNF10 | CTSB | ELF3 |
| CDC20 | DCXR | PRKAR1A | METTL9 | BSG | PFKP |
| MKI67 | ATP5I | HNRNPL | UBE2D2 | CD151 | TUBB3 |
| CCNB1 | NREP | HIF1A | GOLPH3 | CD59 | SOX4 |
| DF20_cluster7 | DF20_cluster8 | DF20_cluster9 | DF20_cluster10 | DF68_cluster1 | DF68_cluster2 |
| LOC100131257 | PTPRS | PLTP | C1orf192 | FBLN1 | LGALS3BP |
| KCNQ1OT1 | XYLT2 | SNCG | FAM183A | SRD5A3 | MSLN |
| LOC646214 | LPIN3 | CMTM7 | TPPP3 | GPRC5B | IFI6 |
| SHISA9 | CCNL2 | KRTCAP3 | PIFO | CYP4X1 | B2M |
| ABCC9 | RBM6 | CMTM6 | ROPN1L | CHRNA1 | HLA-A |
| LOC643406 | SLC35F6 | HPN | CCDCI9 | CD52 | TMEM59 |
| REXO1L1 | SLC39A11 | HIST1H1C | ZMYND10 | AZGP1 | PSAP |
| TMEM212 | SNAR-B2 | RPL32 | ARMC3 | EGR1 | BSG |
| MAB21L3 | SNAR-B1 | SUSD3 | TEKT2 | ENTPD2 | SPINT2 |
| UGDH-AS1 | B4GALT1 | ADAMTS1 | C9orf135 | FOSB | TMED10 |
| ASTN2 | DPP7 | MUC16 | UBXN10 | PRRT3 | UPK3B |
| ARHGEF26-AS1 | HNRNPL | HIST2H2AA3 | CAPS | DUSP1 | MUC1 |
| LOC286437 | MARCH6 | HIST2H2AA4 | DNALI1 | CYP4B1 | ATP6AP2 |
| LOC90834 | NPIPL3 | TCF25 | SPAG8 | LOC654433 | P4HB |
| ODF2L | C1orf56 | TSEN2 | MS4A8B | ALDH3B2 | BST2 |
| ORC4 | POU5F1 | FAM107A | C9orf24 | GALNT2 | FOLR1 |
| C14orf23 | C16orf54 | RPL14 | FAM81B | GALNT6 | SLC44A2 |
| CCL5 | HNRNPH1 | EXOSC5 | SPEF1 | CYP4Z1 | CYP4B1 |
| GATM-AS1 | TMEM33 | RPSA | FANK1 | ATP2B4 | RPN1 |
| RAMP2-AS1 | ATP6V0C | RPL15 | C9orf117 | MUC20 | ITM2B |
| L2HGDH | CTNNB1 | RPUSD3 | TUBA1A | PAX8 | NUCB1 |
| UGT8 | MIR4461 | CRTAP | C9orf116 | SCFD2 | PPIC |
| OPHN1 | METTL21A | LOC650226 | CCDC40 | TSC2 | PRDX4 |
| POU5F1 | EXOSC6 | C1orf186 | SPAG6 | SUSD4 | PTGDS |
| INGX | MBOAT1 | MYC | C11orf70 | HPCAL1 | SEC61A1 |
| LINC00294 | TCTN2 | C1orf85 | LRRC23 | PDIA5 | GRN |
| GLIPR1L2 | DSG2 | EMP2 | RSPH1 | PTGDS | RABAC1 |
| HERC2P4 | CLSTN1 | AGPAT2 | IQCG | GPX3 | PTTG1IP |
| SCD5 | MSLN | MRPS25 | NEK11 | FAM107A | CD46 |
| NCRUPAR | CXADR | GAA | KIF9 | ALG8 | IGFBP5 |
| DF68_cluster3 | DF68_cluster4 | DF68_cluster5 | DF68_cluster6 | DF68_cluster7 | DF68_cluster1 |
| OA52 | SLC2A1 | TUBB3 | GADD45B | TPX2 | NDRGI |
| XAF1 | VEGFA | KRT6A | EDN1 | KIFC1 | ENO2 |
| MX1 | PFKFB3 | PLAU | HBEGF | KIF20A | BNIP3 |
| ISG15 | AHNAK2 | S100A10 | CYR61 | BIRC5 | NDUFA4L2 |
| PARP14 | CXCR4 | CD24 | IER3 | TACC3 | ANGPTL4 |
| IFIT1 | ESYT2 | IER3 | RND3 | DLGAP5 | CA9 |
| DDX60 | GPNMB | CPA4 | ID2 | CDC20 | SOX4 |
| C19orf66 | SPAG4 | S100A6 | PLK2 | CCNA2 | PFKFB4 |
| OAS3 | ANGPTL4 | KRT5 | PPP1R15A | TROAP | MUC16 |
| STAT1 | P4HA1 | ZYX | HES1 | CENPF | KRT19 |
| OAS1 | BHLHE40 | KRT17 | KLF10 | CDKN3 | ALDOA |
| IFI44L | ADM | TGM2 | SFN | TYMS | SLPI |
| PARP9 | CD24 | SFN | HEXIM1 | CCNB2 | AHNAK2 |
| PLSCR1 | IGFBP2 | S100A2 | ZFAND2A | TOP2A | TMSB10 |
| IFI44 | FBLIM1 | KRT7 | NR1D1 | MAD2L1 | RNF24 |
| IFIT3 | EGLN3 | TNC | RASD1 | PTTG1 | BEND5 |
| OASL | DDIT4 | FHL2 | HSPA1B | CDK1 | GPNMB |
| TRIM22 | PKD1P1 | TPM2 | CTGF | KIF23 | SLC16A3 |
| DTX3L | CPE | SOX4 | JUNB | ZWINT | PGK1 |
| IFITM1 | EGFR | TACSTD2 | PHLDA2 | NUSAP1 | ADM |
| PSMB8 | FTH1 | LOC541471 | CCNL1 | PRC1 | INSIG2 |
| RTP4 | WSB1 | TNFRSF12A | ID1 | ECT2 | PRELID2 |
| RSAD2 | NEAT1 | CAPN2 | HSPB8 | RACGAP1 | DDIT4 |
| DDX58 | ENO2 | COTL1 | H2AFX | ASF1B | VIM |
| IFITM3 | NOV | PLAUR | ID3 | NUF2 | ENO1 |
| NMI | S100A10 | IGFBP3 | ZFP36 | TK1 | SLC2A1 |
| IFI6 | ELF3 | AQP3 | AREG | CDCA8 | ALDOC |
| UBE2L6 | PDK1 | ITGA3 | TUBB2A | AURKB | RNF183 |
| UBA7 | SOX4 | FTH1 | IFRD1 | HIST1H3G | PDK1 |
| SP100 | S1PR2 | S100A11 | SERTAD1 | FAM64A | CA12 |
| DF101_cluster2 | DF101_cluster3 | DF101_cluster4 | DF101_cluster5 | DF101_cluster6 | DF101_cluster7 |
| TACSTD2 | IFIT3 | ANXA3 | ESRG | BIRC5 | SPON1 |
| LOC100505633 | ISG15 | IFRD1 | ROCK1P1 | UBE2C | PTPRF |
| CLU | IFIT1 | RND3 | SPC25 | PRC1 | CYP4B1 |
| HLA-H | ISG20 | DDIT3 | MRPL41 | MKI67 | PDIA5 |
| ISG15 | IFIT2 | IL32 | LOC643406 | TOP2A | GPRC5B |
| CLDN1 | MX1 | ANXA1 | L2HGDH | NDC80 | LGR5 |
| HLA-C | OAS1 | CALCB | GLIPR1L2 | TPX2 | SELENBP1 |
| CD24 | HLA-H | SBDSP1 | LOC646214 | CDKN3 | WT1 |
| TSPAN15 | RSAD2 | RAB32 | SHISA9 | PTTG1 | MTRNR2L5 |
| HLA-B | IF144L | LINC00152 | LOC100131257 | CCNB2 | CHRM3 |
| CCL28 | OA52 | ZNF410 | ARHGEF26-AS1 | CCNA2 | MTRNR2L4 |
| FAM107A | C19orf66 | TNFRSF12A | MRPL18 | PBK | MTRNR2L2 |
| IFI6 | PARP14 | CNN3 | ATP5I | CDC20 | TRO |
| RARRES3 | STAT1 | SDAD1 | ATP5G1 | KIF2C | KIAA1210 |
| INC | HLA-A | NUPR1 | MRPL27 | TACC3 | MTRNR2L1 |
| GPX1 | EPSTI1 | PRPF3 | ABCC9 | CCNB1 | METTL21A |
| KLK5 | CMPK2 | ARHGDIA | KCNQ1OT1 | NUSAP1 | MTRNR2L8 |
| IFI27 | KRT6A | CRYAB | HIGD1A | AURKB | SULF1 |
| SEPP1 | IFI35 | SBDS | IFI27 | CDK1 | LINC00649 |
| IFIT1 | HLA-F | PTP4A1 | MRPL48 | HMGB2 | PGM5P2 |
| CCDC3 | HLA-B | SLC3A2 | KLK10 | NCAPG | CCL5 |
| PLA2G16 | OAS3 | SNHG15 | ORC4 | ASF1B | CD81 |
| MAL | B2M | CCT6P3 | MAB21L3 | TRIP13 | MTRNR2L3 |
| CNN3 | DDX58 | TES | HERC2P4 | PLK1 | MTRNR2L6 |
| CMBL | CFB | PLK2 | DCUN1D2 | TK1 | PROM2 |
| PSMB10 | ANXA3 | RIPK2 | C16orf91 | NUF2 | MTRNR2L10 |
| CRIP1 | TIMP1 | AARD | BRIX1 | RRM2 | SLC2A9 |
| KRT5 | DTX3L | SLC35B1 | CCDC167 | MAD2L1 | AMY1A |
| C3orf55 | PARP9 | EIF1 | ODF2L | KIF18A | NEK5 |
| HLA-A | GPRC5A | CLIC4 | TMEM212 | CDC25C | AMY1B |

TABLE 5
15 compounds used in this study targeting different nodes of the JAK/STAT
pathway or its effectors and platinum-chemotherapies.

| Compound | Manufacturer | Nominal target |
|---|---|---|
| AZD1480 | AstraZeneca | JAK2 |
| CE P33779 | Selleck Chemicals | JAK2 |
| Cyt387 | Selleck Chemicals | JAK1/2 |
| NVP-BSK805 | Selleck Chemicals | JAK2 |
| Ruxolitinib | Novartis | JAK1/2 |
| SRuxolitinib | Selleck Chemicals | JAK1/2 |
| TG101348 | StemCell Technologies | JAK2 |
| Tofacitinib | Pfizer | JAK3 |
| AZD1208 | AstraZeneca | Pim Kinases |
| CX-6258 | Selleck Chemicals | Pim Kinases |
| SGI-1776 | Sigma Aldreich, Inc. | Pim 1 Kinase |
| HO-3867 | Cayman Chemicals | STAT3 |
| SH-4-54 | Selleck Chemicals | STAT3/5 |
| JSI-124 | Sigma Aldreich, Inc. | JAK2/STAT3 |
| Cisplatin | APP Pharmaceuticals, Inc. | DNA-alkylating |
| Carboplatin | Hospira, Inc. | DNA-alkylating |

Various modifications and variations of the described methods, pharmaceutical compositions, and kits of the invention will be apparent to those skilled in the art without departing from the scope and spirit of the invention. Although the invention has been described in connection with specific embodiments, it will be understood that it is capable of further modifications and that the invention as claimed should not be unduly limited to such specific embodiments. Indeed, various modifications of the described modes for carrying out the invention that are obvious to those skilled in the art are intended to be within the scope of the invention. This application is intended to cover any variations, uses, or adaptations of the invention following, in general, the principles of the invention and including such departures from the present disclosure come within known customary practice within the art to which the invention pertains and may be applied to the essential features herein before set forth.

1. A population of ex vivo T cells comprising an exogenous nucleic acid sequence encoding a binding protein specific for binding a surface protein selected from Table 2, wherein the surface protein has at least two-fold higher average expression in any one or more of clusters 1 to 5 as compared to the average expression in any one or more of clusters 6 to 18, preferably,
wherein the binding protein is a chimeric antigen receptor (CAR) or a T cell receptor (TCR); and/or
wherein the T cells are specific for a surface protein selected from the group consisting of CLDN3, CLDN7, CLDN4, EPCAM, TACSTD2, MAL2, LSR, CD9, SPINT2, TM4SF1, TMEM205, TNFRSF12A and CD47, more preferably,
wherein the T cells are CAR T cells specific for CLDN3; or
wherein the T cells are CAR T cells specific for CLDN7; or
wherein the T cells are CAR T cells specific for CLDN4.

2-6. (canceled)

7. A method of treating a cancer in a subject in need thereof comprising administering the population of T cells according to claim 1 to the subject.

8. A method of treating a cancer in a subject in need thereof comprising:
administering to the subject a therapeutically effective amount of one or more agents capable of binding to or modulating the expression, activity, and/or function of one or more genes or polypeptides selected from Table 2, wherein the one or more genes or polypeptides have at least two-fold higher average expression in any one or more of clusters 1 to 5 as compared to the average expression in any one or more of clusters 6 to 18; or
administering to the subject a therapeutically effective amount of one or more agents capable of modulating the expression, activity, and/or function of one or more biological programs comprising one or more genes or polypeptides selected from the group consisting of:
a) CLU, TACSTD2, MFAP2, LGALS3BP, FBLN2, LY6E, VTCN1, CLIC5, PTPRS, RAB25, SH3BGRL, BST2, SERPING1, MFGE8, ANKRD65, UCA1, THEM6, HSPG2, MSLN, NDRG2, TAPBP, CDH6, PLD3, RBMS3, COMP, SEPP1, RNF213, CD74, IFIT3 and IFI6; or
b) CCBP2, TACSTD2, OAS1, MX1, GPNMB, OAS2, KRT23, MSLN, TXNIP, C15orf48, UPK3BL, PTGES, LCN2, CD82, SAT1, VTCN1, ITGB2, NCCRP1, CEACAM6, AGR2, PSCA, PARP14, HERC6, GABRP, IFI44L, IFIT3, IFI6, IFI44, ISG15 and IFIT1; or
c) XAF1, MX1, PARP14, DDX60, C19orf66, OAS3, STAT1, OAS1, PARP9, PLSCR1, IFI44, IFIT3, OASL, TRIM22, DTX3L, IFITM1, PSMB8, RTP4, RSAD2, DDX58, IFITM3, NMI, UBE2L6, UBA7, SP100, OAS2, ISG15, IFIT1, IFI44L and IFI6; or
d) TACSTD2, LOC100505633, CLU, HLA-H, CLDN1, HLA-C, CD24, TSPAN15, HLA-B, CCL28, FAM107A, RARRES3, TNC, GPX1, KLK5, IFI27, SEPP1, CCDC3, PLA2G16, MAL, CNN3, CMBL, PSMB10, CRIP1, KRT5, C3orf55, HLA-A, ISG15, IFIT1 and IFI6; or
e) IFIT3, ISG20, IFIT2, MX1, OAS1, HLA-H, RSAD2, C19orf66, PARP14, STAT1, HLA-A, EPSTI1, CMPK2, KRT6A, IFI35, HLA-F, HLA-B, OAS3, B2M, DDX58, CFB, ANXA3, TIMP1, DTX3L, PARP9, GPRC5A, ISG15, IFIT1, IFI44L and OAS2; or
f) LYNX1, LYPD2, CLU, TRIM29, MMP7, MUC4, TSPAN1, ATP6V1B1, SERPINA1, SLPI, KCNN4, SYT8, RARRES3, PTGES, RASAL1, CP, FOLR1, UNC5B, ALOX5, MUC20, FOS, SLC4A11, FXYD3, C3, UNC5B-AS1, DEFB1, COL12A1, CD74, HLA-DRB1 and HLA-DRA; or
g) RARRES1, CRYAB, NNMT, PLA2G16, MGST1, HLA-DRB6, MT1F, CLU, CD14, NFIB, C3, BNIP3L, OAT, SLC34A2, NUPR1, ANXA1, MT1X, CDC42EP2, TUBB6, SAA1, CSTB, VIM, GPX3, MT1E, HLA-DRB1, HLA-DRA, HLA-DRB5, HLA-DMA, CD74 and HLA-DPA1; or
h) GPX3, LCN2, CLDN4, GPR56, DHRS3, SLC44A4, LYPD2, MUC20, TMPRSS4, MUC1, ELF3, SRGAP1, FOLR1, B2M, MDK, WFDC2, CMTM7, FTH1, CP, ESR1, CLDN3, RBP1, TNFAIP2, RNF213, MUC4, TACSTD2, CD74, HLA-DRA, HLA-DRB1 and HLA-DPA1; or
i) SOD2, CXCL3, TNFAIP3, CXCL1, TUBA1A, TPM1, EDN1, TAGLN, CLDN1, CXCL2, TNFAIP2, NEDD9, ADAMTS9, FLNA, ARHGAP29, CTHRC1, RGS10, UBD, THBS1, CALD1, PTX3, RELB, CYR61, NFKBIA, CCL2, TNF, ICAM1, CCL20, IL8 and IL32; or
j) UBD, TAP1, TNFAIP2, CRYAB, MARCO, LGALS14, PSMB9, CD74, TUBB2B, KRT23, IL4I1, TAPBP, C10orf10, CLDN1, SOD2, SELM, JAK3, TNFSF10, B2M, HLA-H, COL4A2, HLA-B, IL8, IL23A, ICAM1, TNF, CCL20, CCL2, IL32 and CD40.

9. The method of claim 8, wherein the one or more agents bind to or modulate the expression, activity, and/or function of CLDN3, preferably,
wherein the one or more agents comprise a CAR T cell that binds CLDN3; or
wherein the one or more agents comprise an antibody that binds CLDN3, more preferably,
wherein the antibody is a bi-specific antibody, more preferably, wherein the bi-specific antibody binds CLDN3 and an immune cell marker, more preferably, wherein the immune cell marker is selected from the group consisting of CD3, CD8, CD28 and CD16, or wherein the bi-specific antibody binds CLDN3 and a surface protein selected from the group consisting of CLDN7, CLDN4, EPCAM, TACSTD2, MAL2, LSR, CD9, SPINT2, TM4SF1, TMEM205, TNFRSF12A and CD47; or
wherein the antibody is an antibody-drug conjugate that binds CLDN3; or
wherein the one or more agents bind to or modulate the expression, activity, and/or function of CLDN7, preferably,
wherein the one or more agents comprise a CAR T cell that binds CLDN7; or
wherein the one or more agents comprise an antibody that binds CLDN7, more preferably,
wherein the antibody is a bi-specific antibody, more preferably, wherein the bi-specific antibody binds CLDN7 and an immune cell marker, more preferably, wherein the immune cell marker is selected from the group consisting of CD3, CD8, CD28 and CD16, or wherein the bi-specific antibody binds CLDN7 and a surface protein selected from the group consisting of CLDN3, CLDN4, EPCAM, TACSTD2, MAL2, LSR, CD9, SPINT2, TM4SF1, TMEM205, TNFRSF12A and CD47; or
wherein the antibody is an antibody-drug conjugate that binds CLDN7; or
wherein the one or more agents bind to or modulate the expression, activity, and/or function of CLDN4, preferably,
wherein the one or more agents comprise a CAR T cell that binds CLDN4; or
wherein the one or more agents comprise an antibody that binds CLDN4, more preferably,
wherein the antibody is a bi-specific antibody, more preferably, wherein the bi-specific antibody binds CLDN4 and an immune cell marker, more preferably, wherein the immune cell marker is selected from the group consisting of CD3, CD8, CD28 and CD16, or wherein the bi-specific antibody binds CLDN4 and a surface protein selected from the group consisting of CLDN3, CLDN7, EPCAM, TACSTD2, MAL2, LSR, CD9, SPINT2, TM4SF1, TMEM205, TNFRSF12A and CD47; or
wherein the antibody is an antibody-drug conjugate that binds CLDN4.

10-32. (canceled)

33. The method of claim 8, wherein the one or more agents bind to or modulate the expression, activity, and/or function of CLDN3, CLDN7, and/or CLDN4 in combination with any of the other genes or polypeptides according to claim 8.

34. The method of claim 8, wherein the one or more genes or polypeptides are selected from the group consisting of CLDN3, CLDN7, CLDN4, EPCAM, TACSTD2, MAL2, LCN2, CKB, RBP1, CDKN2A, C19orf33, WFDC2, LSR, CRABP2, S100A13, KRT7, CRIP2, MDK, CD9, SPINT2, SLPI, KRT19, KRT18, KRT8, TM4SF1, NGFRAP1, S100A16, PCBD1, OCIAD2, ZNF428, TMEM205, TSTD1, TNFRSF12A, MARCKSL1, IFI27, CD47, POLR2I, CCDC124, PDCD5 and DPY30, preferably, wherein the one or more genes or polypeptides are selected from the group of surface proteins consisting of CLDN3, CLDN7, CLDN4, EPCAM, TACSTD2, MAL2, LSR, CD9, SPINT2, TM4SF1, TMEM205, TNFRSF12A and CD47.

35-36. (canceled)

37. The method of claim 8, comprising decreasing expression, activity, and/or function of an interferon response gene program comprising one or more genes or polypeptides selected from the group consisting of:
a) CLU, TACSTD2, MFAP2, LGALS3BP, FBLN2, LY6E, VTCN1, CLIC5, PTPRS, RAB25, SH3BGRL, BST2, SERPING1, MFGE8, ANKRD65, UCA1, THEM6, HSPG2, MSLN, NDRG2, TAPBP, CDH6, PLD3, RBMS3, COMP, SEPP1, RNF213, CD74, IFIT3 and IFI6; or
b) CCBP2, TACSTD2, OAS1, MX1, GPNMB, OAS2, KRT23, MSLN, TXNIP, C15orf48, UPK3BL, PTGES, LCN2, CD82, SAT1, VTCN1, ITGB2, NCCRP1, CEACAM6, AGR2, PSCA, PARP14, HERC6, GABRP, IFI44L, IFIT3, IFI6, IFI44, ISG15 and IFIT1; or
c) XAF1, MX1, PARP14, DDX60, C19orf66, OAS3, STAT1, OAS1, PARP9, PLSCR1, IFI44, IFIT3, OASL, TRIM22, DTX3L, IFITM1, PSMB8, RTP4, RSAD2, DDX58, IFITM3, NMI, UBE2L6, UBA7, SP100, OAS2, ISG15, IFIT1, IFI44L and IFI6; or
d) TACSTD2, LOC100505633, CLU, HLA-H, CLDN1, HLA-C, CD24, TSPAN15, HLA-B, CCL28, FAM107A, RARRES3, TNC, GPX1, KLK5, IFI27, SEPP1, CCDC3, PLA2G16, MAL, CNN3, CMBL, PSMB10, CRIP1, KRT5, C3orf55, HLA-A, ISG15, IFIT1 and IFI6; or
e) IFIT3, ISG20, IFIT2, MX1, OAS1, HLA-H, RSAD2, C19orf66, PARP14, STAT1, HLA-A, EPSTI1, CMPK2, KRT6A, IFI35, HLA-F, HLA-B, OAS3, B2M, DDX58, CFB, ANXA3, TIMP1, DTX3L, PARP9, GPRC5A, ISG15, IFIT1, IFI44L and OAS2; or
increasing expression, activity, and/or function of an MHC class II gene program comprising one or more genes or polypeptides selected from the group consisting of:
a) LYNX1, LYPD2, CLU, TRIM29, MMP7, MUC4, TSPAN1, ATP6V1B1, SERPINA1, SLPI, KCNN4, SYT8, RARRES3, PTGES, RASAL1, CP, FOLR1, UNC5B, ALOX5, MUC20, FOS, SLC4A11, FXYD3, C3, UNC5B-AS1, DEFB1, COL12A1, CD74, HLA-DRB1 and HLA-DRA; or
b) RARRES1, CRYAB, NNMT, PLA2G16, MGST1, HLA-DRB6, MT1F, CLU, CD14, NFIB, C3, BNIP3L, OAT, SLC34A2, NUPR1, ANXA1, MT1X, CDC42EP2, TUBB6, SAA1, CSTB, VIM, GPX3, MT1E, HLA-DRB1, HLA-DRA, HLA-DRB5, HLA-DMA, CD74 and HLA-DPA1; or
c) GPX3, LCN2, CLDN4, GPR56, DHRS3, SLC44A4, LYPD2, MUC20, TMPRSS4, MUC1, ELF3, SRGAP1, FOLR1, B2M, MDK, WFDC2, CMTM7, FTH1, CP, ESR1, CLDN3, RBP1, TNFAIP2, RNF213, MUC4, TACSTD2, CD74, HLA-DRA, HLA-DRB1 and HLA-DPA1, optionally,
further comprising administering checkpoint blockade (CPB) therapy; or
decreasing expression, activity, and/or function of an inflammatory cytokine gene program comprising one or more genes or polypeptides selected from the group consisting of:
a) SOD2, CXCL3, TNFAIP3, CXCL1, TUBA1A, TPM1, EDN1, TAGLN, CLDN1, CXCL2, TNFAIP2, NEDD9, ADAMTS9, FLNA, ARHGAP29, CTHRC1, RGS10, UBD, THBS1, CALD1, PTX3, RELB, CYR61, NFKBIA, CCL2, TNF, ICAM1, CCL20, IL8 and IL32; or
b) UBD, TAP1, TNFAIP2, CRYAB, MARCO, LGALS14, PSMB9, CD74, TUBB2B, KRT23, IL4I1, TAPBP, C10orf10, CLDN1, SOD2, SELM, JAK3, TNFSF10, B2M, HLA-H, COL4A2, HLA-B, IL8, IL23A, ICAM1, TNF, CCL20, CCL2, IL32 and CD40.

38-40. (canceled)

41. The method of claim 8, wherein the one or more agents target one or more cell surface exposed genes or polypeptides; or
wherein the one or more agents target one or more receptors or ligands specific for one or more cell surface exposed genes or polypeptides; or
wherein the one or more agents target one or more secreted genes or polypeptides; or
wherein the one or more agents target one or more receptors specific for one or more secreted genes or polypeptides.

42-44. (canceled)

45. The method of claim 8, wherein the one or more agents comprise an antibody, antibody-like protein scaffold, aptamer, small molecule, genetic modifying agent, protein, nucleic acid or any combination thereof, preferably,
wherein the antibody is an antibody-drug conjugate or a bispecific antibody, more preferably, wherein the bi-specific antibody is capable of targeting an immune cell to the tumor cell or capable of targeting two surface proteins expressed on the tumor cell; and/or
wherein the genetic modifying agent comprises a CRISPR system, RNAi system, a zinc finger nuclease system, a TALE system, or a meganuclease, more preferably, wherein the CRISPR system is a Class 1 or Class 2 CRISPR system, more preferably, wherein the Class 2 system comprises a Type II Cas polypeptide, more preferably, wherein the Type II Cas is a Cas9, or wherein the Class 2 system comprises a Type V Cas polypeptide, more preferably, wherein the Type V Cas is Cas12a, Cas12b, Cas12c, Cas12d (CasY), Cas12e(CasX), or Cas14, or wherein the Class 2 system comprises a Type VI Cas polypeptide, more preferably, wherein the Type VI Cas is Cas13a, Cas13b, Cas13c or Cas13d.

46-57. (canceled)

58. The method of any of claim 45, wherein the CRISPR system comprises a dCas fused or otherwise linked to a nucleotide deaminase, preferably, wherein the nucleotide deaminase is a cytidine deaminase or an adenosine deaminase.

59. (canceled)

60. A method for detecting, monitoring or prognosing a cancer in a subject in need thereof comprising:
detecting in a tumor sample obtained from the subject the expression or activity of one or more genes or polypeptides selected from the group consisting of CLDN3, CLDN7, CLDN4, EPCAM, TACSTD2, MAL2, LCN2, CKB, RBP1, CDKN2A, C19orf33, WFDC2, LSR, CRABP2, S100A13, KRT7, CRIP2, MDK, CD9, SPINT2, SLPI, KRT19, KRT18, KRT8, TM4SF1, NGFRAP1, S100A16, PCBD1, OCIAD2, ZNF428, TMEM205, TSTD1, TNFRSF12A, MARCKSL1, IFI27, CD47, POLR2I, CCDC124, PDCD5 and DPY30; or
detecting in a tumor sample obtained from the subject the expression or activity of one or more biological programs comprising one or more genes or polypeptides selected from the group consisting of:
a) CLU, TACSTD2, MFAP2, LGALS3BP, FBLN2, LY6E, VTCN1, CLIC5, PTPRS, RAB25, SH3BGRL, BST2, SERPING1, MFGE8, ANKRD65, UCA1, THEM6, HSPG2, MSLN, NDRG2, TAPBP, CDH6, PLD3, RBMS3, COMP, SEPP1, RNF213, CD74, IFIT3 and IFI6; or
b) CCBP2, TACSTD2, OAS1, MX1, GPNMB, OAS2, KRT23, MSLN, TXNIP, C15orf48, UPK3BL, PTGES, LCN2, CD82, SAT1, VTCN1, ITGB2, NCCRP1, CEACAM6, AGR2, PSCA, PARP14, HERC6, GABRP, IFI44L, IFIT3, IFI6, IFI44, ISG15 and IFIT1; or
c) XAF1, MX1, PARP14, DDX60, C19orf66, OAS3, STAT1, OAS1, PARP9, PLSCR1, IFI44, IFIT3, OASL, TRIM22, DTX3L, IFITM1, PSMB8, RTP4, RSAD2, DDX58, IFITM3, NMI, UBE2L6, UBA7, SP100, OAS2, ISG15, IFIT1, IFI44L and IFI6; or
d) TACSTD2, LOC100505633, CLU, HLA-H, CLDN1, HLA-C, CD24, TSPAN15, HLA-B, CCL28, FAM107A, RARRES3, TNC, GPX1, KLK5, IFI27, SEPP1, CCDC3, PLA2G16, MAL, CNN3, CMBL, PSMB10, CRIP1, KRT5, C3orf55, HLA-A, ISG15, IFIT1 and IFI6; or
e) IFIT3, ISG20, IFIT2, MX1, OAS1, HLA-H, RSAD2, C19orf66, PARP14, STAT1, HLA-A, EPSTI1, CMPK2, KRT6A, IFI35, HLA-F, HLA-B, OAS3, B2M, DDX58, CFB, ANXA3, TIMP1, DTX3L, PARP9, GPRC5A, ISG15, IFIT1, IFI44L and OAS2; or
f) LYNX1, LYPD2, CLU, TRIM29, MMP7, MUC4, TSPAN1, ATP6V1B1, SERPINA1, SLPI, KCNN4, SYT8, RARRES3, PTGES, RASAL1, CP, FOLR1, UNC5B, ALOX5, MUC20, FOS, SLC4A11, FXYD3, C3, UNC5B-AS1, DEFB1, COL12A1, CD74, HLA-DRB1 and HLA-DRA; or
g) RARRES1, CRYAB, NNMT, PLA2G16, MGST1, HLA-DRB6, MT1F, CLU, CD14, NFIB, C3, BNIP3L, OAT, SLC34A2, NUPR1, ANXA1, MT1X, CDC42EP2, TUBB6, SAA1, CSTB, VIM, GPX3, MT1E, HLA-DRB1, HLA-DRA, HLA-DRB5, HLA-DMA, CD74 and HLA-DPA1; or
h) GPX3, LCN2, CLDN4, GPR56, DHRS3, SLC44A4, LYPD2, MUC20, TMPRSS4, MUC1, ELF3, SRGAP1, FOLR1, B2M, MDK, WFDC2, CMTM7, FTH1, CP, ESR1, CLDN3, RBP1, TNFAIP2, RNF213, MUC4, TACSTD2, CD74, HLA-DRA, HLA-DRB1 and HLA-DPA1; or
i) SOD2, CXCL3, TNFAIP3, CXCL1, TUBA1A, TPM1, EDN1, TAGLN, CLDN1, CXCL2, TNFAIP2, NEDD9, ADAMTS9, FLNA, ARHGAP29, CTHRC1, RGS10, UBD, THBS1, CALD1, PTX3, RELB, CYR61, NFKBIA, CCL2, TNF, ICAM1, CCL20, IL8 and IL32; or
j) UBD, TAP1, TNFAIP2, CRYAB, MARCO, LGALS14, PSMB9, CD74, TUBB2B, KRT23, IL4I1, TAPBP, C10orf10, CLDN1, SOD2, SELM, JAK3, TNFSF10, B2M, HLA-H, COL4A2, HLA-B, IL8, IL23A, ICAM1, TNF, CCL20, CCL2, IL32 and CD40; or
detecting in a tumor sample obtained from the subject a mesenchymal phenotype by detecting CAFs in the tumor, preferably, wherein detecting the mesenchymal phenotype comprises detecting in CAFs one or more genes selected from the group consisting of: PDPN, C1QA/B/C, CFB, CXCL12, CXCL1, CXCL2, CXCL10, IL6, IL10, ALDH1A2, ACTA2, COL1A2, LUM, COL3A1, DCN and COL1A1; or clusters 6-9 of Table 2; or
detecting in a tumor sample obtained from the subject an immunoreactive phenotype by detecting macrophages in the tumor, preferably, wherein detecting an immunoreactive phenotype comprises detecting in macrophages one or more genes selected from the group consisting of: CD52, CD14, AIF1, CSF1R, C1QB, C1QA, CD163, CD36, FCGR3A, CCL3, CCL4, IFNGR1, CD1D, C2, APOE, APOC1, CTSD, CTSZ, LYZ, FCN1, DDX5, MNDA, C3AR1, VISG4, SERPINA1, HLA-DPA1, HLA-DRA, HLA-DPB1 and HLA-DRB5; or clusters 10-13 of Table 2.

61. The method of claim 60, wherein the one or more genes or polypeptides are detected in single cells from the biological sample; or wherein the one or more genes or polypeptides are detected in a tissue sample by immunohistochemistry or RNA FISH.

62-63. (canceled)

64. The method of claim 60, wherein detection of an interferon response gene program comprising one or more genes or polypeptides selected from the group consisting of:
a) CLU, TACSTD2, MFAP2, LGALS3BP, FBLN2, LY6E, VTCN1, CLIC5, PTPRS, RAB25, SH3BGRL, BST2, SERPING1, MFGE8, ANKRD65, UCA1, THEM6, HSPG2, MSLN, NDRG2, TAPBP, CDH6, PLD3, RBMS3, COMP, SEPP1, RNF213, CD74, IFIT3 and IFI6; or
b) CCBP2, TACSTD2, OAS1, MX1, GPNMB, OAS2, KRT23, MSLN, TXNIP, C15orf48, UPK3BL, PTGES, LCN2, CD82, SAT1, VTCN1, ITGB2, NCCRP1, CEACAM6, AGR2, PSCA, PARP14, HERC6, GABRP, IFI44L, IFIT3, IFI6, IFI44, ISG15 and IFIT1; or
c) XAF1, MX1, PARP14, DDX60, C19orf66, OAS3, STAT1, OAS1, PARP9, PLSCR1, IFI44, IFIT3, OASL, TRIM22, DTX3L, IFITM1, PSMB8, RTP4, RSAD2, DDX58, IFITM3, NMI, UBE2L6, UBA7, SP100, OAS2, ISG15, IFIT1, IFI44L and IFI6; or
d) TACSTD2, LOC100505633, CLU, HLA-H, CLDN1, HLA-C, CD24, TSPAN15, HLA-B, CCL28, FAM107A, RARRES3, TNC, GPX1, KLK5, IFI27, SEPP1, CCDC3, PLA2G16, MAL, CNN3, CMBL, PSMB10, CRIP1, KRT5, C3orf55, HLA-A, ISG15, IFIT1 and IFI6; or
e) IFIT3, ISG20, IFIT2, MX1, OAS1, HLA-H, RSAD2, C19orf66, PARP14, STAT1, HLA-A, EPSTI1, CMPK2, KRT6A, IFI35, HLA-F, HLA-B, OAS3, B2M, DDX58, CFB, ANXA3, TIMP1, DTX3L, PARP9, GPRC5A, ISG15, IFIT1, IFI44L and OAS2,
indicates that a subject should be treated with a signal transducer and activator of transcription 3 (STAT3) activity inhibitor.

65. The method of claim 64, further comprising treating with a STAT3 inhibitor, preferably,
wherein the STAT3 activity inhibitor is administered intraperitoneally; or
wherein the STAT3 activity is selected from the group consisting of STAT3 phosphorylation, STAT3 dimerization, STAT3 binding to a polynucleotide comprising a STAT3 binding site, STAT3 binding to genomic DNA, activation of a STAT3 responsive gene and STAT3 nuclear translocation; or
wherein the STAT3 inhibitor comprises pyrimethamine, atovaquone, pimozide, guanabenz acetate, alprenolol hydrochloride, nifuroxazide, solanine alpha, fluoxetine hydrochloride, ifosfamide, pyrvinium pamoate, moricizine hydrochloride, 3,3′-oxybis[tetrahydrothiophene, 1,1,1′,1′-tetraoxide], 3-(1,3-benzodioxol-5-yl)-1,6-dimethyl-pyrimido[5,4-e]-1,2,4-triazine-5,7(-1H,6H)-dione, 2-(1,8-Naphthyridin-2-yl)phenol, or 3-(2-hydroxyphenyl)-3-phenyl-N,N-dipropylpropanamide, as well as any derivatives of these compounds or analogues thereof; or
wherein the STAT3 activity inhibitor comprises JSI-124 (cucurbitacin I), more preferably, wherein the JSI-124 is administered at a dose of about 0.1 μM.

66-70. (canceled)

71. The method of claim 60, wherein detection of an MHC class II gene program comprising one or more genes or polypeptides selected from the group consisting of:
a) LYNX1, LYPD2, CLU, TRIM29, MMP7, MUC4, TSPAN1, ATP6V1B1, SERPINA1, SLPI, KCNN4, SYT8, RARRES3, PTGES, RASAL1, CP, FOLR1, UNC5B, ALOX5, MUC20, FOS, SLC4A11, FXYD3, C3, UNC5B-AS1, DEFB1, COL12A1, CD74, HLA-DRB1 and HLA-DRA; or
b) RARRES1, CRYAB, NNMT, PLA2G16, MGST1, HLA-DRB6, MT1F, CLU, CD14, NFIB, C3, BNIP3L, OAT, SLC34A2, NUPR1, ANXA1, MT1X, CDC42EP2, TUBB6, SAA1, CSTB, VIM, GPX3, MT1E, HLA-DRB1, HLA-DRA, HLA-DRB5, HLA-DMA, CD74 and HLA-DPA1; or
c) GPX3, LCN2, CLDN4, GPR56, DHRS3, SLC44A4, LYPD2, MUC20, TMPRSS4, MUC1, ELF3, SRGAP1, FOLR1, B2M, MDK, WFDC2, CMTM7, FTH1, CP, ESR1, CLDN3, RBP1, TNFAIP2, RNF213, MUC4, TACSTD2, CD74, HLA-DRA, HLA-DRB1 and HLA-DPA1,
indicates the patient is responsive to checkpoint blockade (CPB) therapy, preferably, wherein the method further comprises treating with CPB therapy.

72. (canceled)

73. The method of claim 64, wherein the one or more genes or polypeptides are detected in single cells from the tumor sample; or wherein the one or more genes or polypeptides are detected in a tissue sample by immunohistochemistry or RNA FISH.

74. (canceled)

75. A method of screening for agents capable of modulating a biological program in ovarian cancer comprising:
a. applying a candidate agent to an ovarian cancer cell or cell population; and
b. detecting modulation of one or more biological programs according to claim 60, thereby identifying the agent, preferably, wherein the agent is applied to an animal model, more preferably, wherein the animal model is a patient-derived xenograft (PDX).

76-77. (canceled)

78. A method of treating a subject with ovarian cancer comprising detecting in a tumor sample obtained from the subject the expression or activity of one or more biological programs according to claim 60, wherein
if the ovarian tumor expresses one or more of the biological programs, administering a therapeutic regimen that comprises a signal transducer and activator of transcription 3 (STAT3) activity inhibitor, optionally, in combination with chemotherapy; or
if the ovarian tumor does not express one or more of the biological programs, administering a therapeutic regimen that comprises chemotherapy and does not comprise a STAT3 inhibitor.

79. The method of claim 78, wherein one or more biological programs according to claim 64 are detected; and/or
wherein the STAT3 activity inhibitor is administered intraperitoneally; and/or
wherein the STAT3 activity is selected from the group consisting of STAT3 phosphorylation, STAT3 dimerization, STAT3 binding to a polynucleotide comprising a STAT3 binding site, STAT3 binding to genomic DNA, activation of a STAT3 responsive gene and STAT3 nuclear translocation; and/or
wherein the STAT3 inhibitor comprises pyrimethamine, atovaquone, pimozide, guanabenz acetate, alprenolol hydrochloride, nifuroxazide, solanine alpha, fluoxetine hydrochloride, ifosfamide, pyrvinium pamoate, moricizine hydrochloride, 3,3′-oxybis[tetrahydrothiophene, 1,1,1′,1′-tetraoxide], 3-(1,3-benzodioxol-5-yl)-1,6-dimethyl-pyrimido[5,4-e]-1,2,4-triazine-5,7(-1H,6H)-dione, 2-(1,8-Naphthyridin-2-yl)phenol, or 3-(2-hydroxyphenyl)-3-phenyl-N,N-dipropylpropanamide, as well as any derivatives of these compounds or analogues thereof; and/or
wherein the STAT3 activity inhibitor comprises JSI-124 (cucurbitacin I), preferably, wherein the JSI-124 is administered at a dose of about 0.1 μM.

80-84. (canceled)

85. A method of treating a subject with ovarian cancer comprising detecting in a tumor sample obtained from the subject the expression or activity of one or more MHC class II biological programs according to claim 71, wherein
if the ovarian tumor expresses one or more of the biological programs, administering a therapeutic regimen that comprises an immunotherapy; or
if the ovarian tumor does not express one or more of the biological programs, administering a therapeutic regimen that does not comprise an immunotherapy, preferably, wherein the immunotherapy comprises CPB therapy.

86. (canceled)

87. A method of treating a subject with ovarian cancer comprising determining whether the ovarian tumor exhibits a mesenchymal phenotype by detecting CAFs in the tumor according to claim 60, wherein
if the ovarian tumor exhibits increased CAFs characteristic of a mesenchymal phenotype, administering a therapeutic regimen that does not comprise an immunotherapy, preferably, administering a therapeutic regimen that comprises an agent capable of modulating CAF activation; and
if the ovarian tumor does not exhibit increased CAFs characteristic of a mesenchymal phenotype, administering a therapeutic regimen that comprises an immunotherapy, preferably, wherein the immunotherapy comprises CPB therapy.

88-89. (canceled)

90. The method of claim 87, wherein the agent modulates the expression, activity and/or function of one or more genes expressed in CAFs associated with the mesenchymal subtype selected from the group consisting of:
a) PDPN, C1QA/B/C, CFB, CXCL12, CXCL1, CXCL2, CXCL10, IL6, I10, ALDH1A2, ACTA2, COL1A2, LUM, COL3A1, DCN and COL1A1; or
b) clusters 6-9 of Table 2.

91. (canceled)

92. A method of treating a subject with ovarian cancer comprising determining whether the ovarian tumor exhibits an immunoreactive phenotype by detecting macrophages in the tumor according to claim 60, wherein
if the ovarian tumor exhibits increased macrophages characteristic of an immunoreactive phenotype, administering a therapeutic regimen that comprises an immunotherapy; and
if the ovarian tumor does not exhibit increased macrophages characteristic of an immunoreactive phenotype, administering a therapeutic regimen that does not comprise an immunotherapy, preferably,
wherein the immunotherapy comprises CPB therapy.

93-94. (canceled)

95. The method of any of claim 7, wherein the cancer is ovarian cancer, preferably, wherein the ovarian cancer is high-grade serous ovarian cancer (HGSOC).

96. (canceled)

97. A kit comprising reagents to detect at least one gene or polypeptide according to claim 60.
